(19) United States
(12) Patent Application Publication
(10) Pub. No.: US 2026/0362972 A1
(43) Pub. Date: Nov. 12, 2026

(54) ORGANIC COMPOUND, ORGANIC ELECTROLUMINESCENT DEVICE CONTAINING SAID COMPOUND, AND ELECTRONIC APPARATUS

(71) Applicant: Shaanxi Lighte Optoelectronics Material Co., Ltd., Xi'an (CN)

(72) Inventors: Zhiyan JIA, Xi'an (CN); Heming ZHANG, Xi'an (CN)

(21) Appl. No.: 19/485,044

(22) PCT Filed: Sep. 5, 2024
(86) PCT No.: PCT/CN2024/117281

§ 371 Date: Nov. 17, 2025

(30) Foreign Application Priority Data
Dec. 21, 2023 (CN) 202311780849.0

(51) Int. Cl. H10K 85/60; C07B 59/00; C07D 471/04; H10K 50/16

(52) U.S. Cl. CPC H10K 85/6572; C07B 59/002; C07D 471/04; H10K 85/615; H10K 85/626; H10K 85/654; H10K 50/16

(57) ABSTRACT

The present application relates to an organic compound, an organic electroluminescent device containing said compound, and an electronic apparatus. The organic compound has a structure as represented by Formula 1. Applying the organic compound in an organic electroluminescent device can significantly improve the performance of the device.

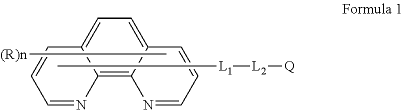

### CROSS REFERENCE TO RELATED APPLICATIONS

The present application claims the priority of Chinese patent application No. CN202311780849.0, filed on Dec. 21, 2023, the contents of which are incorporated here by reference in its entirety as a part of the present application.

### TECHNICAL FIELD

The present application relates to the technical field of organic electroluminescence, in particular to an organic compound, an organic electroluminescent device including the compound, and an electronic apparatus.

### BACKGROUND

Currently, an organic electroluminescent device is regarded as a next generation display and lighting technology due to the advantages of active luminescence, higher current efficiency, low power consumption, lightness, thinness, fast response speed, large viewing angle, and the like. The organic electroluminescent device generally includes a cathode and an anode which are disposed oppositely, and a functional layer disposed between the cathode and the anode. When voltage is applied to the cathode and the anode, an electric field is generated between the two electrodes, electrons on the cathode side move towards the organic light-emitting layer and holes on the anode side also move towards the organic light-emitting layer under the action of the electric field, the electrons and the holes are combined in the organic light-emitting layer to form excitons, the excitons are in an excited state and release energy outwards, and then the organic light-emitting layer emits light outwards.

The organic electroluminescent device may be of various structures, such as a single-layer structure and a stacked structure. A single-layer organic electroluminescent device includes only one light-emitting unit between the cathode and the anode, and a stacked organic electroluminescent device is formed by stacking a plurality of light-emitting units. A light-emitting unit usually includes at least one organic light-emitting layer, a hole transport layer and an electron transport layer. On this basis, the light-emitting unit may further include a hole injection layer, an electron injection layer, a hole blocking layer and an electron blocking layer. There is a charge generation layer (CGL) between adjacent light-emitting units for generation and movement of charges. The CGL is constructed in a p-n form and includes an n-type charge generation layer (n-CGL) and a p-type charge generation layer (p-CGL). p-type materials mainly generate holes, while n-type materials are used to generate electrons by doping electron transport layer materials with low-work-function metals.

In the existing organic electroluminescent devices, the most important problems are the service life and efficiency, and as the area of a display becomes larger, the voltage also increases. Thus, it is necessary to continue to develop new materials to further improve the performance of the organic electroluminescent device.

### SUMMARY

An object of the present application is to provide an organic compound, an organic electroluminescent device including the compound, and an electronic apparatus. When the organic compound is applied to an organic electroluminescent device, the performance of the device can be improved.

A first aspect of the present application provids an organic compound, having a structure represented by a Formula 1:

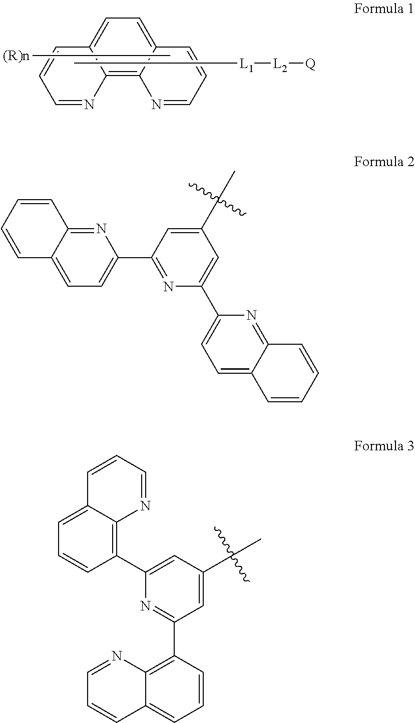

where Q is a Formula 2 or a Formula 3;
L₁ and L₂ are the same or different, and are each independently selected from a single bond, a substituted or unsubstituted arylene with 6 to 30 carbon atoms, or a substituted or unsubstituted heteroarylene with 3 to 30 carbon atoms;
substituent(s) of L₁ and L₂ are the same or different, and are each independently selected from a deuterium, a halogen group, a cyano, a haloalkyl with 1 to 10 carbon atoms, a trialkylsilyl with 3 to 12 carbon atoms, an alkyl with 1 to 10 carbon atoms, an aryl with 6 to 20 carbon atoms, or a heteroaryl with 3 to 20 carbon atoms;
each R is the same or different, and is independently selected from a deuterium, a halogen group, a cyano, an alkyl with 1 to 10 carbon atoms, a substituted or unsubstituted aryl with 6 to 20 carbon atoms, or a substituted or unsubstituted heteroaryl with 3 to 20 carbon atoms;
substituent(s) of R are the same or different, and are each independently selected from a deuterium, a halogen group, a cyano, an alkyl with 1 to 10 carbon atoms, an aryl with 6 to 12 carbon atoms, or a heteroaryl with 3 to 12 carbon atoms; and
n is the number of R, n is selected from 0, 1, 2, 3, 4, 5, 6 or 7, and when n is greater than 1, any two Rs are the same or different; and alternatively, any two adjacent Rs form an aromatic ring with 6 to 14 carbon atoms.

A second aspect of the present application provides an organic electroluminescent device, including an anode and a cathode which are oppositely disposed, and a functional layer disposed between the anode and the cathode; and the functional layer includes the organic compound in the first aspect of the present application.

A third aspect of the present application provides an electronic apparatus, including the organic electroluminescent device in the second aspect of the present application.

The compound of the present application has a structure formed by 1,10-phenanthrolinyl and pyridyl substituted by two quinolyl, and this structure has a large conjugation plane and effectively adjusts intermolecular stacking, thus improving electron mobility and facilitating charge transport. At the same time, the compound of the present application can effectively undergo complexation reaction with a metal, and when applied as a charge generation layer material, the compound can increase the efficiency of charge generation, thus increasing the current efficiency, and prolonging the service life of the organic electroluminescent device.

Other features and advantages of the present application will be described in detail in the subsequent Detailed Description.

### BRIEF DESCRIPTION OF THE DRAWINGS

The accompanying drawings are used to provide a further understanding of the present application and constitute a part of the description, and are used to explain the present application together with the following specific embodiments, but do not constitute limitations on the present application.

FIG. 1 is a schematic structural diagram of an organic electroluminescent device according to one embodiment of the present application.

FIG. 2 is a schematic structural diagram of an organic electroluminescent device according to another embodiment of the present application.

FIG. 3 is a schematic structural diagram of an electronic apparatus according to an embodiment of the present application.

### DESCRIPTION OF REFERENCE NUMERALS

100, anode; 200, cathode; 300, functional layer; 310, hole injection layer; 321, hole transport layer; 322, electron blocking layer; 330, organic light-emitting layer; 340, electron transport layer; 350, electron injection layer; 411, first hole transport layer; 412, first hole adjustment layer; 413, first organic light-emitting layer; 414, first electron transport layer; 421, n-type charge generation layer; 422, p-type charge generation layer; 431, second hole transport layer; 432, second hole adjustment layer; 433, second organic light-emitting layer; 434, second electron transport layer; 410, first light-emitting unit; 420, charge generation layer; 430, second light-emitting unit; and 500, electronic apparatus.

### DETAILED DESCRIPTION

Examples will now be described more fully with reference to the accompanying drawings. However, the examples can be implemented in a variety of forms, and should not be construed as limited to the instances set forth here; and on the contrary, these examples are provided such that the present application will be more comprehensive and complete, and the concepts of the examples are comprehensively conveyed to those skilled in the art. The described features, structures, or characteristics may be combined in any suitable manner in one or more embodiments. In the following description, many specific details are provided to obtain a thorough understanding of the examples of the present application.

In a first aspect, the present application provides an organic compound, having a structure represented by a Formula 1:

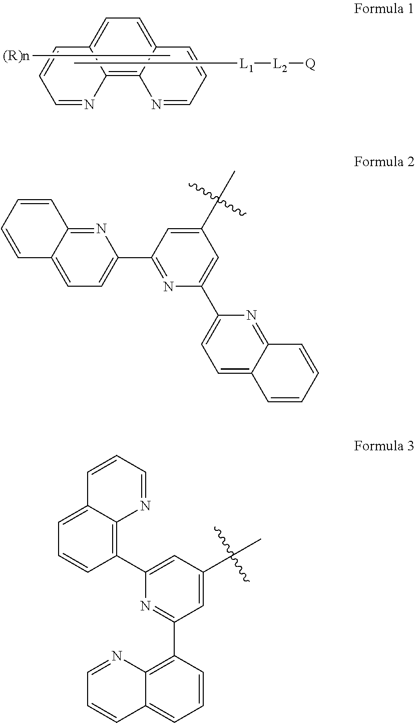

where Q is a Formula 2 or a Formula 3;
L₁ and L₂ are the same or different, and are each independently selected from a single bond, a substituted or unsubstituted arylene with 6 to 30 carbon atoms, or a substituted or unsubstituted heteroarylene with 3 to 30 carbon atoms;
substituent(s) of L₁ and L₂ are the same or different, and are each independently selected from a deuterium, a halogen group, a cyano, a haloalkyl with 1 to 10 carbon atoms, a trialkylsilyl with 3 to 12 carbon atoms, an alkyl with 1 to 10 carbon atoms, an aryl with 6 to 20 carbon atoms, or a heteroaryl with 3 to 20 carbon atoms;
each R is the same or different, and is independently selected from a deuterium, a halogen group, a cyano, an alkyl with 1 to 10 carbon atoms, a substituted or unsubstituted aryl with 6 to 20 carbon atoms, or a substituted or unsubstituted heteroaryl with 3 to 20 carbon atoms;
substituent(s) of R are the same or different, and are each independently selected from a deuterium, a halogen group, a cyano, an alkyl with 1 to 10 carbon atoms, an aryl with 6 to 12 carbon atoms, or a heteroaryl with 3 to 12 carbon atoms; and
n is the number of R, n is selected from 0, 1, 2, 3, 4, 5, 6 or 7, and when n is greater than 1, any two Rs are the same or different; and alternatively, any two adjacent Rs form an aromatic ring with 6 to 14 carbon atoms.

In the present application, the adopted description modes “each independently selected from”, and “respectively and independently selected from” can be interchanged, and should be understood in a broad sense, which means that in different groups, specific options expressed between the same symbols do not influence each other, or in a same group, specific options expressed between the same symbols do not influence each other. For example, the meaning of “

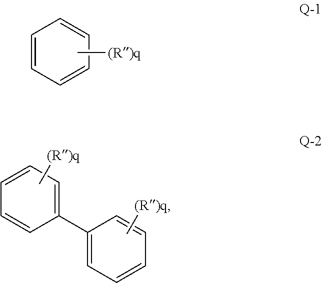

where each q is independently 0, 1, 2 or 3, and each R″ is independently selected from hydrogen, deuterium, fluorine and chlorine” is as follows: a formula Q-1 represents that q substituent R″(s) exist on a benzene ring, each R″ can be the same or different, and options of each R″ do not influence each other; and a formula Q-2 represents that each benzene ring of biphenyl has q substituent R″(s), the number q of the substituent R″(s) on the two benzene rings can be the same or different, each R″ can be the same or different, and options of each R″ do not influence each other.

In the present application, the terms “alternative’ and “alternatively” mean that the subsequently described event or circumstance may but need not occur, and that the description includes instances where the event or circumstance occurs or does not occur. For example, “alternatively, any two adjacent substituents form an xx ring”, which means that the two substituents can, but need not, form a ring, including scenarios in which two adjacent substituents form a ring and scenarios in which two adjacent substituents do not form a ring. For another example, “alternatively, any two adjacent R form an aromatic ring with 6 to 14 carbon atoms”, which means that two adjacent R together with carbon atoms to which they are connected form an aromatic ring with 6 to 14 carbon atoms, or two R may each independently be present.

In the present application, the term such as “substituted or unsubstituted” means that a functional group described behind the term may have or may not have a substituent (in the following, the substituent is collectively referred to as Rc in order to facilitate description). For example, the “substituted or unsubstituted aryl” refers to aryl having the substituent Rc or unsubstituted aryl. The above substituent, i.e., Rc, may be, for example, a deuterium, a halogen group, a cyano, an alkyl, a trialkylsilyl, a haloalkyl, a cycloalkyl, an aryl, a heteroaryl, or the like.

In the present application, the number of carbon atoms of a substituted or unsubstituted functional group refers to the number of all carbon atoms. For example, if L₁ is substituted arylene with 12 carbon atoms, then the number of all carbon atoms of the arylene and substituents on the arylene is 12.

In the present application, aryl refers to an optional functional group or substituent derived from an aromatic carbocyclic ring. The aryl may be monocyclic aryl (e.g., phenyl) or polycyclic aryl, in other words, the aryl may be monocyclic aryl, fused aryl, two or more monocyclic aryl connected by carbon-carbon bonds, monocyclic aryl and fused aryl which are connected by a carbon-carbon bond, or two or more fused aryl connected by carbon-carbon bonds. That is, unless otherwise specified, two or more aromatic groups connected by carbon-carbon bonds may also be regarded as the aryl of the present application. The fused aryl may include, for example, bicyclic fused aryl (e.g., naphthyl), tricyclic fused aryl (e.g., phenanthryl, fluorenyl, and anthryl), and the like. The aryl does not contain heteroatoms such as B, N, O, S, P, Se and Si. For example, in the present application, biphenyl, terphenyl, and the like are aryl. Examples of the aryl can include, but are not limited to, phenyl, naphthyl, fluorenyl, anthryl, phenanthryl, biphenyl, terphenyl, benzo[9,10]phenanthryl, pyrenyl, benzofluoranthenyl, chrysenyl, tetralinyl, and the like. In the present application, the arylene involved refers to a divalent group formed by further loss of one hydrogen atom from the aryl.

In the present application, the substituted aryl can be that one or two or more hydrogen atoms in the aryl are substituted by groups such as a deuterium atom, a halogen group, a cyano, an aryl, a heteroaryl, a trialkylsilyl, an alkyl, a cycloalkyl, a haloalkyl, and a deuteroalkyl. Specific examples of heteroaryl-substituted aryl include, but are not limited to, dibenzofuranyl-substituted phenyl, dibenzothienyl-substituted phenyl, pyridyl-substituted phenyl, and the like. It should be understood that the number of carbon atoms of the substituted aryl refers to the total number of carbon atoms of the aryl and substituents on the aryl, for example, substituted aryl with 18 carbon atoms means that the total number of carbon atoms of the aryl and substituents is 18.

In the present application, the heteroaryl refers to a monovalent aromatic ring containing at least one heteroatom in the ring or its derivative, and the heteroatom may be one or more of B, O, N, P, Si, Se and S. The heteroaryl may be monocyclic heteroaryl or polycyclic heteroaryl, in other words, the heteroaryl may be a single aromatic ring system or a plurality of aromatic ring systems connected by carbon-carbon bonds, and any aromatic ring system is one aromatic monocyclic ring or one aromatic fused ring. For example, the heteroaryl may include thienyl, furyl, pyrrolyl, imidazolyl, thiazolyl, oxazolyl, oxadiazolyl, triazolyl, pyridyl, bipyridyl, pyrimidinyl, triazinyl, acridinyl, pyridazinyl, pyrazinyl, quinolyl, quinazolinyl, quinoxalinyl, phenoxazinyl, phthalazinyl, pyridopyrimidinyl, pyridopyrazinyl, pyrazinopyrazinyl, isoquinolyl, indolyl, carbazolyl, benzoxazolyl, benzimidazolyl, benzothiazolyl, benzocarbazolyl, benzothienyl, dibenzothienyl, thienothienyl, benzofuranyl, phenanthrolinyl, isoxazolyl, thiadiazolyl, phenothiazinyl, silafluorenyl, dibenzofuranyl, as well as N-phenylcarbazolyl, N-pyridylcarbazolyl, N-methylcarbazolyl and the like, but is not limited to this. In the present application, the heteroarylene involved refers to a divalent group formed by further loss of one hydrogen atom from the heteroaryl.

In the present application, the substituted heteroaryl may be that one or two or more hydrogen atoms in the heteroaryl are substituted by groups such as a deuterium atom, a halogen group, cyano, aryl, heteroaryl, trialkylsilyl, alkyl, and haloalkyl. Specific examples of aryl-substituted heteroaryl include, but are not limited to, phenyl-substituted dibenzofuranyl, phenyl-substituted dibenzothienyl, phenyl-substituted pyridyl, and the like. It should be understood that the number of carbon atoms of the substituted heteroaryl refers to the total number of carbon atoms of heteroaryl and substituents on the heteroaryl.

In the present application, the number of carbon atoms of the aryl as a substituent can be 6 to 20, for example, the number of carbon atoms can be 6, 7, 8, 9, 10, 11, 12, 13, 14, 15, 16, 17, 18, 19, or 20, and specific examples of the aryl as the substituent include, but are not limited to, phenyl, biphenyl, naphthyl, anthryl, and chrysenyl.

In the present application, the number of carbon atoms of the heteroaryl as a substituent can be 3 to 20, for example, the number of carbon atoms can be 3, 4, 5, 6, 7, 8, 9, 10, 11, 12, 13, 14, 15, 16, 17, 18, 19, or 20, and specific examples of the heteroaryl as the substituent include, but are not limited to, pyridyl, pyrimidinyl, carbazolyl, dibenzofuranyl, dibenzothienyl, quinolyl, quinazolinyl, quinoxalinyl, and isoquinolyl.

In the present application, the alkyl with 1 to 10 carbon atoms may include linear alkyl with 1 to 10 carbon atoms and branched alkyl with 3 to 10 carbon atoms. The number of carbon atoms of the alkyl can be, for example, 1, 2, 3, 4, 5, 6, 7, 8, 9, or 10, and specific examples of the alkyl include, but are not limited to, methyl, ethyl, n-propyl, isopropyl, n-butyl, isobutyl, tert-butyl, n-pentyl, isopentyl, neopentyl, n-hexyl, n-heptyl, n-octyl, 2-ethylhexyl, nonyl, decyl, 3,7-dimethyloctyl, and the like.

In the present application, the halogen group may be, for example, fluorine, chlorine, bromine or iodine.

In the present application, specific examples of the trialkylsilyl include, but are not limited to, trimethylsilyl, triethylsilyl, and the like.

In the present application, specific examples of the haloalkyl include, but are not limited to, trifluoromethyl.

In the present application, an unpositioned connecting bond refers to a single bond

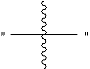

extending from a ring system, which means that one end of the connecting bond may be connected to any position in the ring system through which the bond penetrates, and the other end of the connecting bond is connected to the remaining part of a compound molecule. For example, as shown in the following formula (f), naphthyl represented by the formula (f) is connected to other positions of a molecule through two unpositioned connecting bonds penetrating a dicyclic ring, and its meaning includes any one possible connecting mode represented by formulae (f-1) to (f-10):

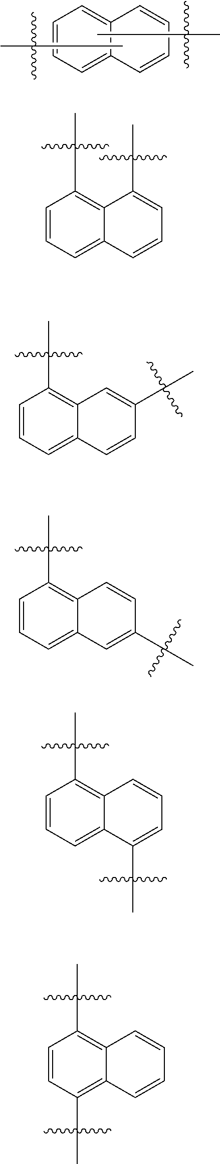

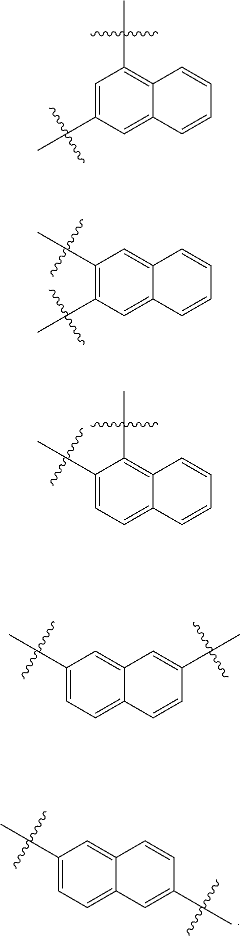

For another example, as shown in the following formula (X′), dibenzofuranyl represented by the formula (X′) is connected with other positions of a molecule through one unpositioned connecting bond extending from the middle of a benzene ring on one side, and its meaning includes any one possible connecting mode represented by formulae (X′-1) to (X′-4):

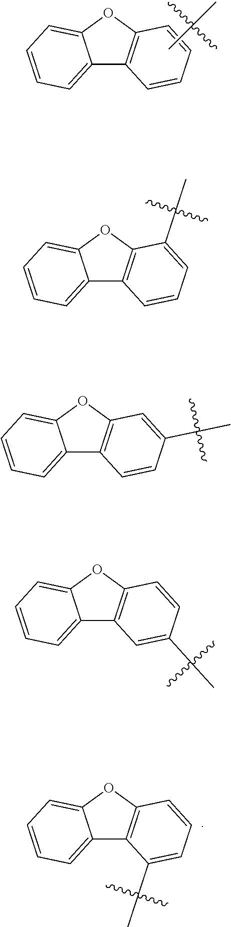

In some embodiments of the present application, the organic compound has a structure represented by a Formula I-I or a Formula I-II below:

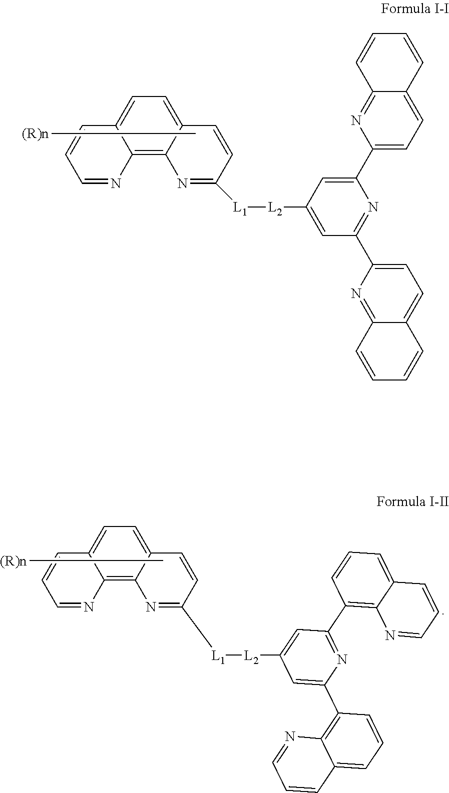

R, L₁, L₂ and n in the Formula I-I and the Formula I-II are as defined in the Formula 1.

In some embodiments of the present application, L₁ and L₂ are the same or different, and are each independently selected from a single bond, a substituted or unsubstituted arylene with 6 to 22 carbon atoms, or a substituted or unsubstituted heteroarylene with 3 to 18 carbon atoms. For example, L₁ and L₂ are the same or different, and are each independently selected from a single bond; a substituted or unsubstituted arylene with 6, 7, 8, 9, 10, 11, 12, 13, 14, 15, 16, 17, 18, 19, 20, 21 or 22 carbon atoms; or a substituted or unsubstituted heteroarylene with 3, 4, 5, 6, 7, 8, 9, 10, 11, 12, 13, 14, 15, 16, 17 or 18 carbon atoms.

In some embodiments of the present application, substituent(s) of L₁ and L₂ are the same or different, and are each independently selected from a deuterium, a fluorine, a cyano, a haloalkyl with 1 to 5 carbon atoms, a trialkylsilyl with 3 to 6 carbon atoms, an alkyl with 1 to 5 carbon atoms, an aryl with 6 to 16 carbon atoms or a heteroaryl with 3 to 12 carbon atoms.

In some embodiments of the present application, L₁ and L₂ are the same or different, and are each independently selected from a single bond, a substituted or unsubstituted phenylene, a substituted or unsubstituted naphthylene, a substituted or unsubstituted biphenylene, a substituted or unsubstituted anthrylene, a substituted or unsubstituted phenanthrylene, a substituted or unsubstituted pyrenylene, a substituted or unsubstituted dibenzofurylidene, a substituted or unsubstituted dibenzothienylidene, a substituted or unsubstituted carbazolylidene, a substituted or unsubstituted triazinylene, a substituted or unsubstituted pyrimidinylidene, a substituted or unsubstituted pyridylidene, and a substituted or unsubstituted pyrazinylene.

Alternatively, the substituent(s) of L₁ and L₂ are the same or different, and are each independently selected from a deuterium, a fluorine, a cyano, a trifluoromethyl, a trimethylsilyl, a methyl, an ethyl, an isopropyl, a tert-butyl, a phenyl, a naphthyl, a biphenyl, a phenanthryl, an anthryl, a pyrenyl, a dibenzofuranyl, a dibenzothienyl, a carbazolyl, a pyridyl, a pyrimidinyl, a pyrazinyl or a quinolyl.

In some embodiments of the present application, L₁ and L₂ are the same or different, and are each independently selected from a single bond or the group consisting of:

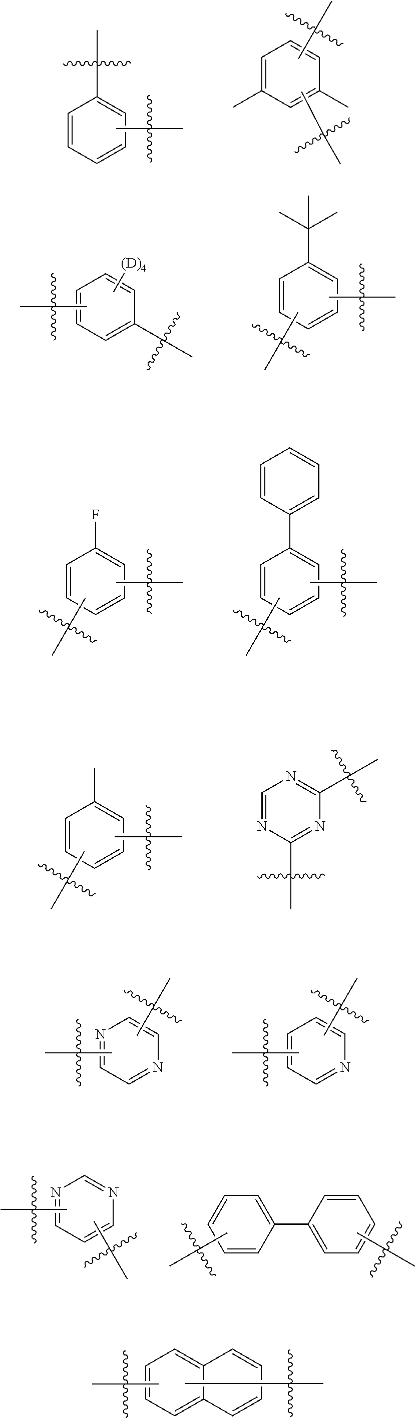

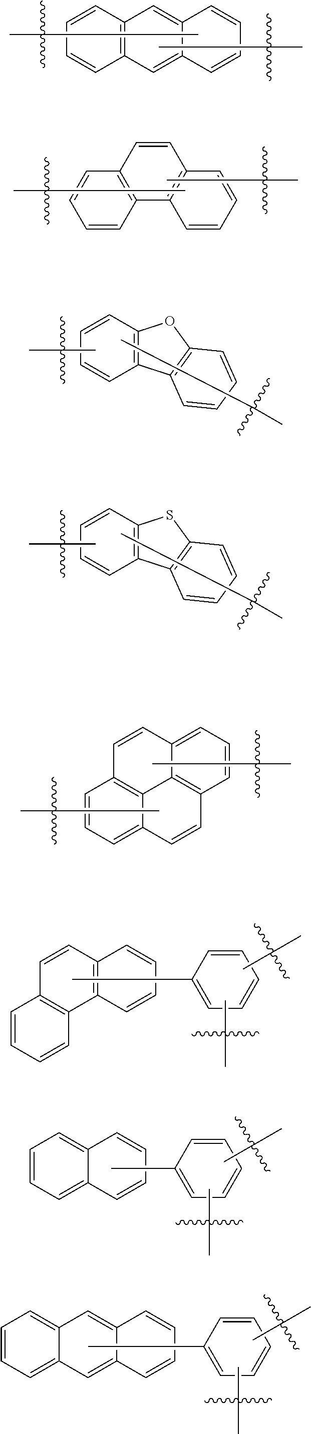

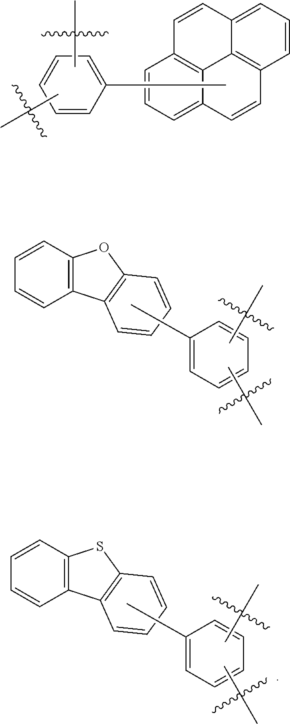

In some embodiments of the present application, L₁ and L₂ are the same or different, and are each independently selected from a single bond or the group consisting of:

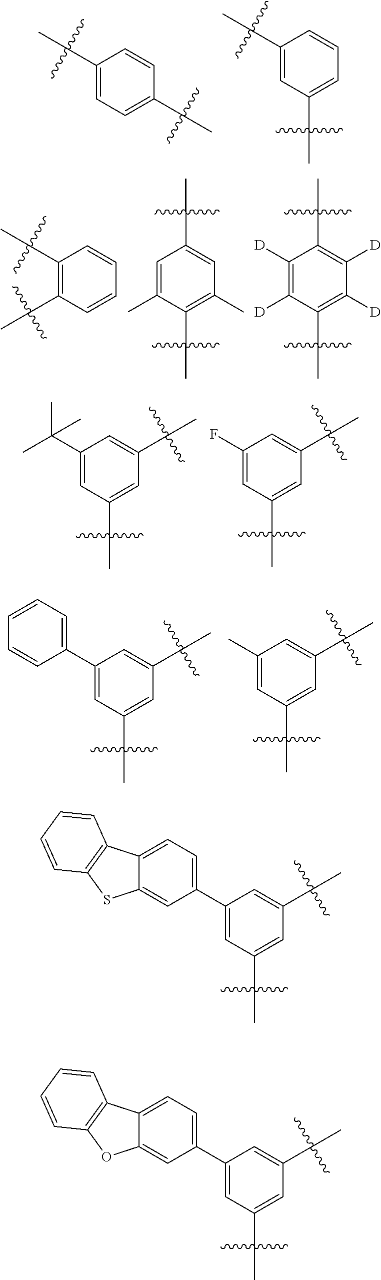

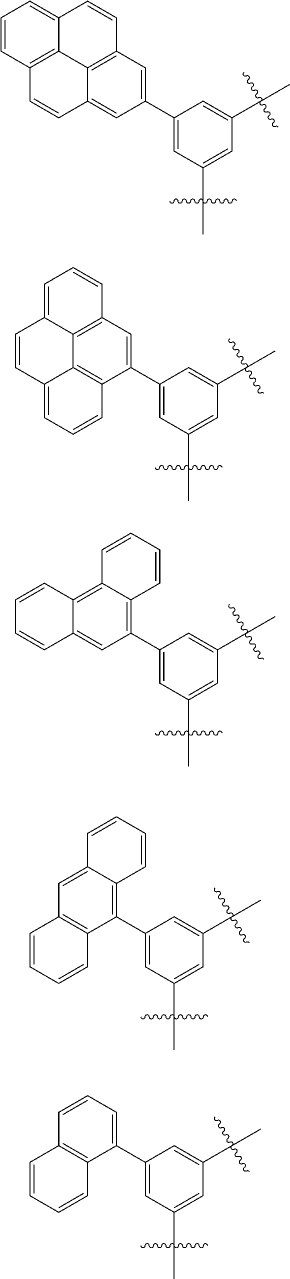 [SMILES: *c1cc(*)cc(-c2cc3ccc4cccc5ccc(c2)c3c45)c1 ; *c1cc(*)cc(-c2cc3cccc4ccc5cccc2c5c43)c1 ; *c1cc(*)cc(-c2cc3ccccc3c3ccccc23)c1 ; *c1cc(*)cc(-c2c3ccccc3cc3ccccc23)c1 ; *c1cc(*)cc(-c2cccc3ccccc23)c1]

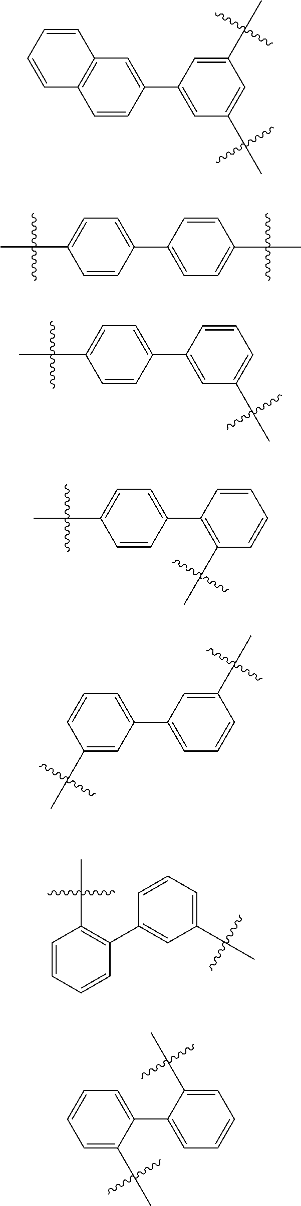

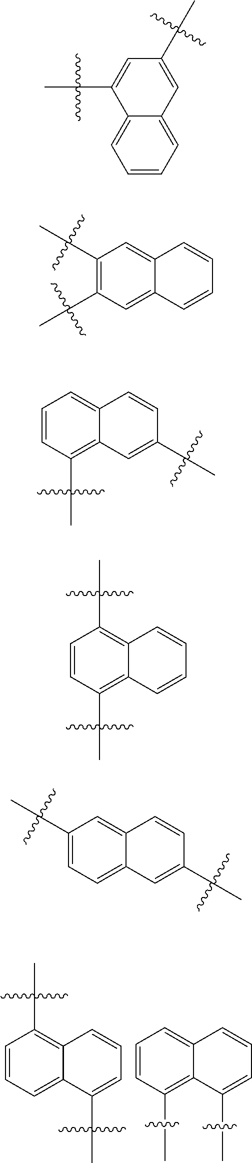

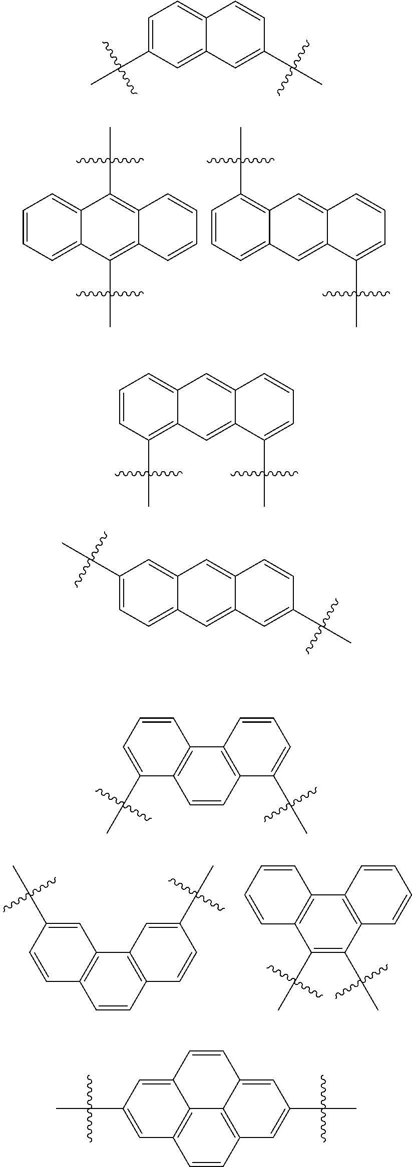

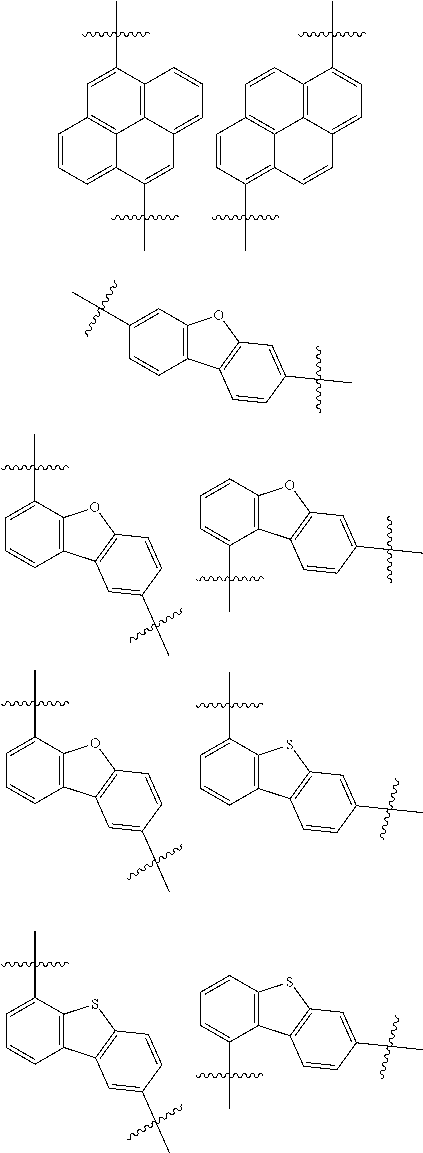

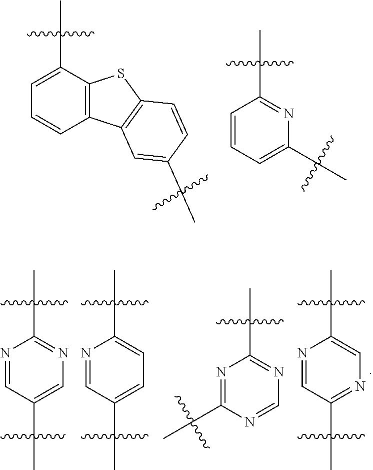

In some embodiments of the present application,

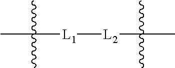

is selected from a single bond or the group consisting of:

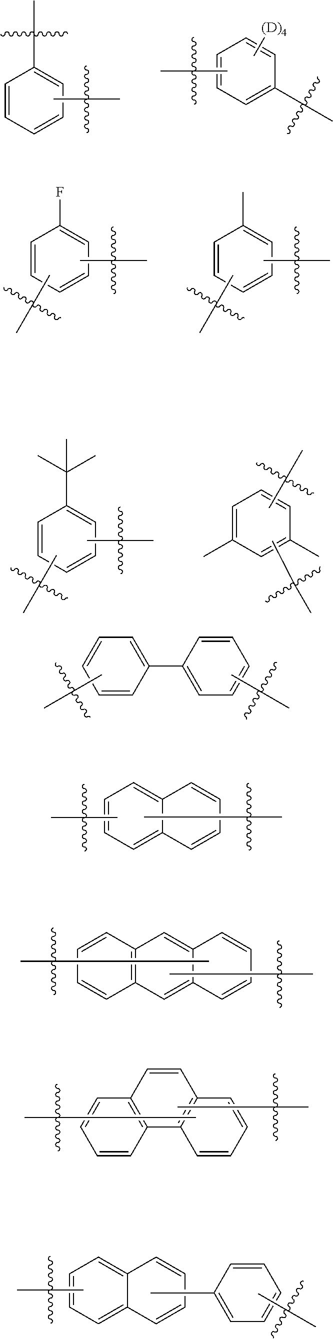

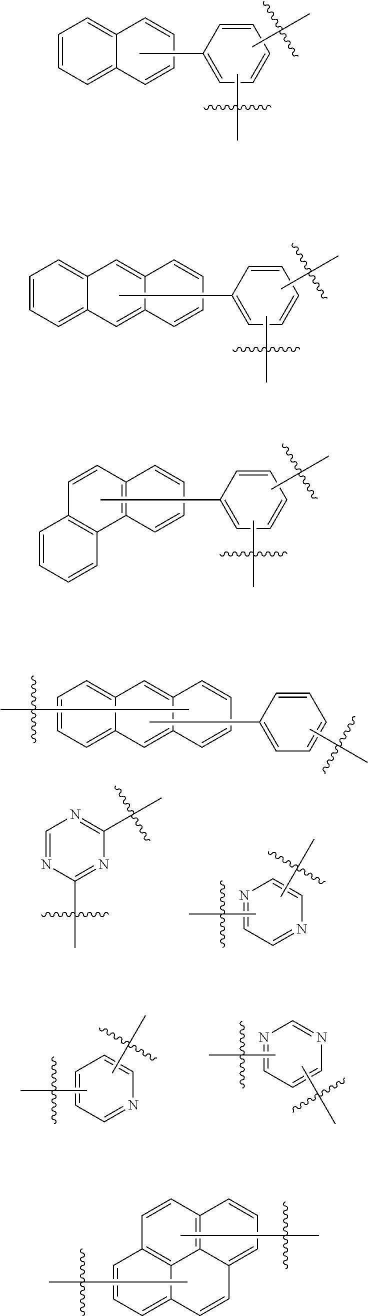

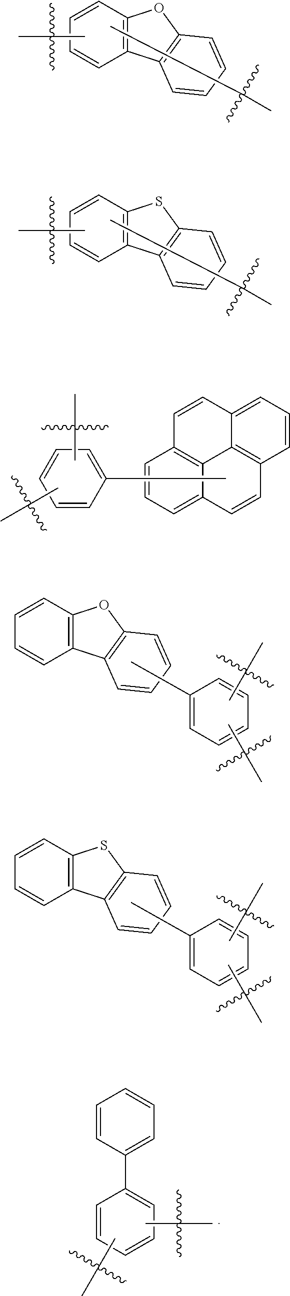

In some embodiments of the present application,

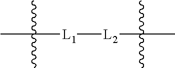

is selected from a single bond or the group consisting of:

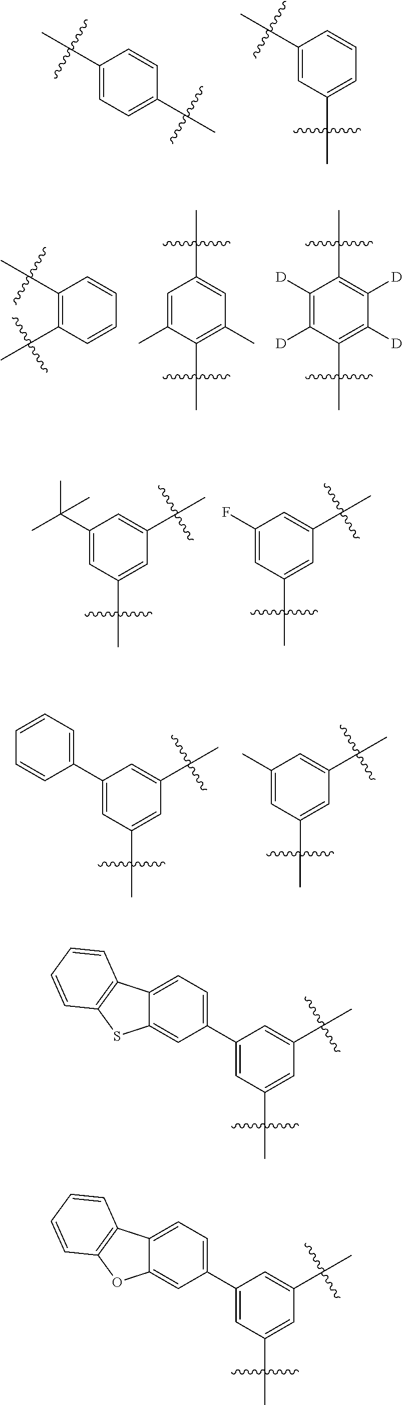

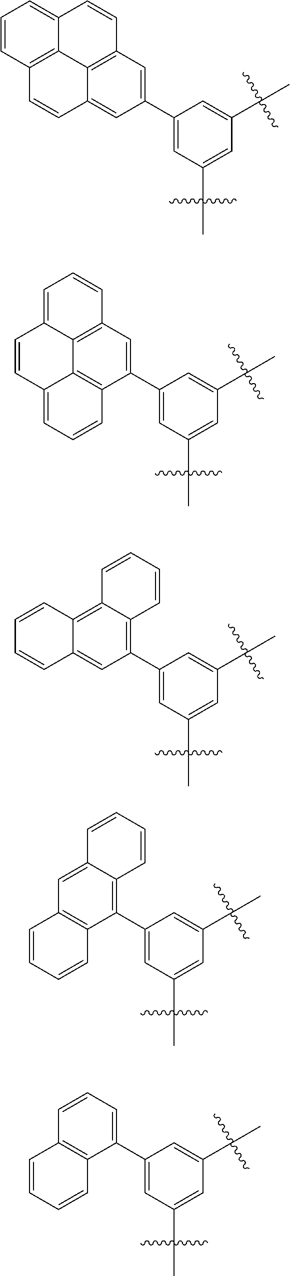 [SMILES: *c1cc(*)cc(-c2cc3ccc4cccc5ccc(c2)c3c45)c1 ; *c1cc(*)cc(-c2cc3cccc4ccc5cccc2c5c43)c1 ; *c1cc(*)cc(-c2cc3ccccc3c3ccccc23)c1 ; *c1cc(*)cc(-c2c3ccccc3cc3ccccc23)c1 ; *c1cc(*)cc(-c2cccc3ccccc23)c1]

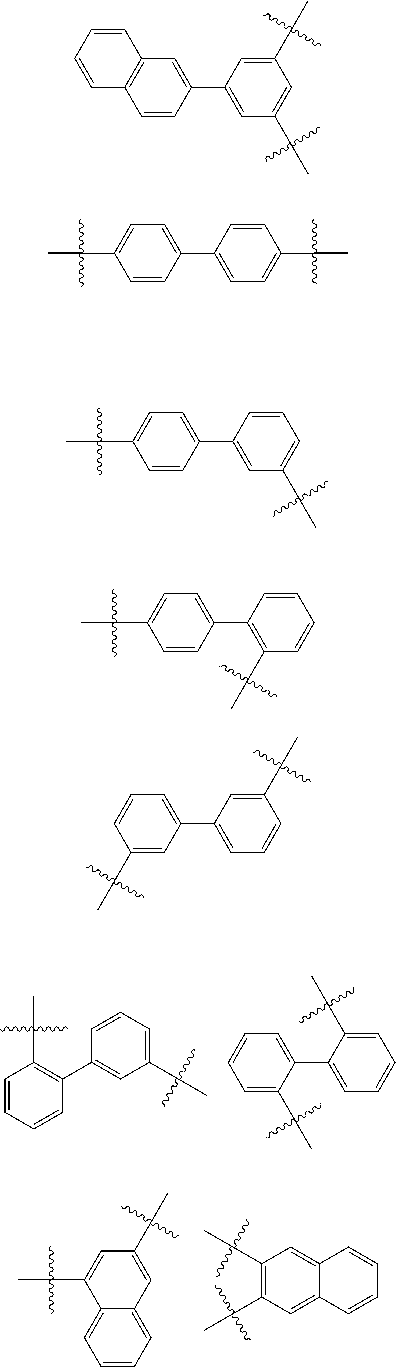

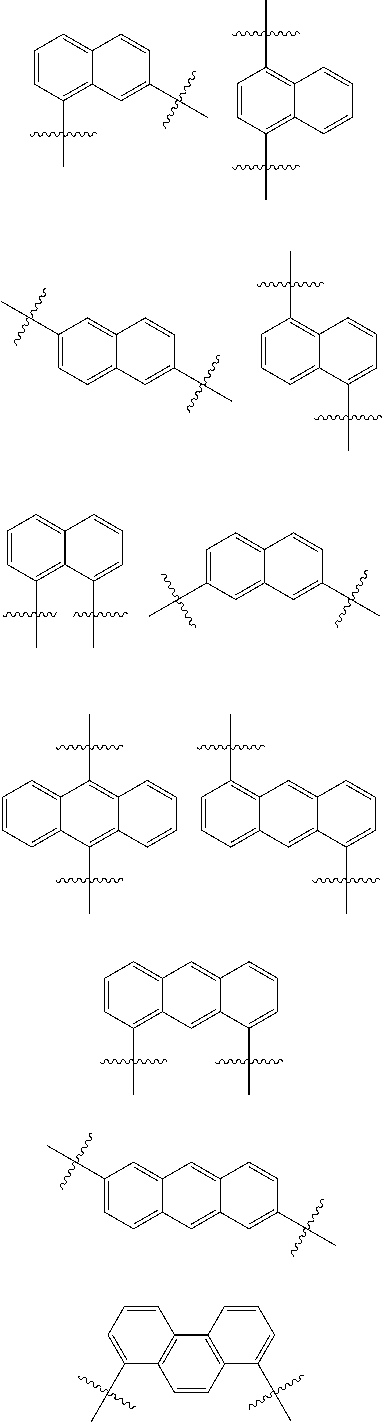

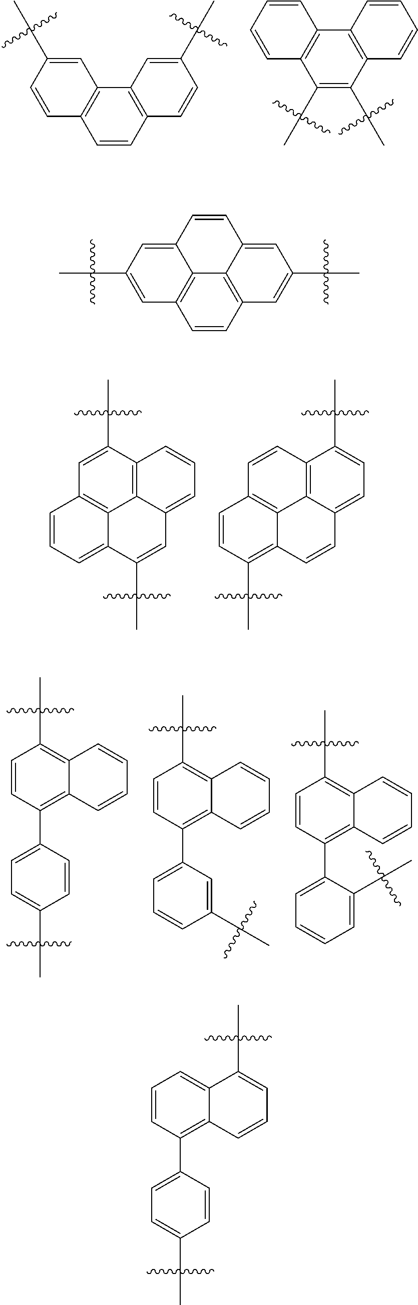

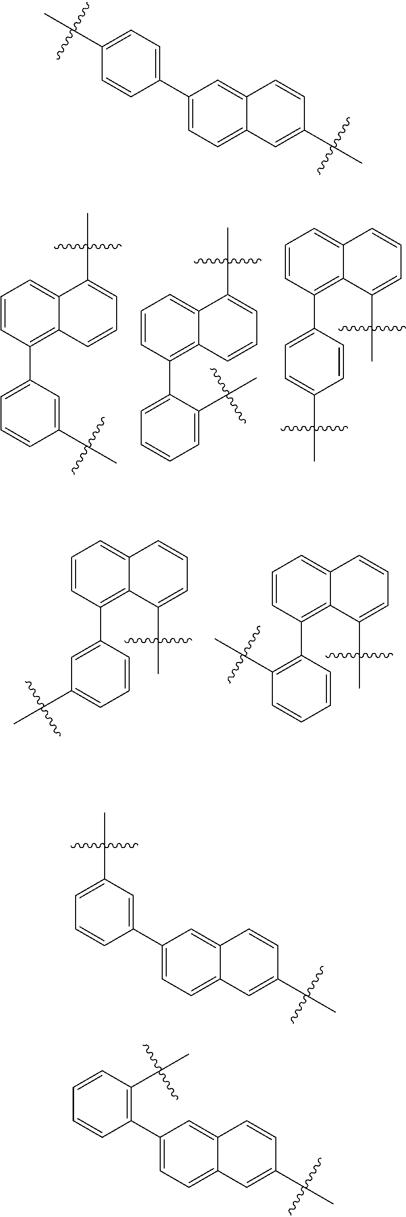

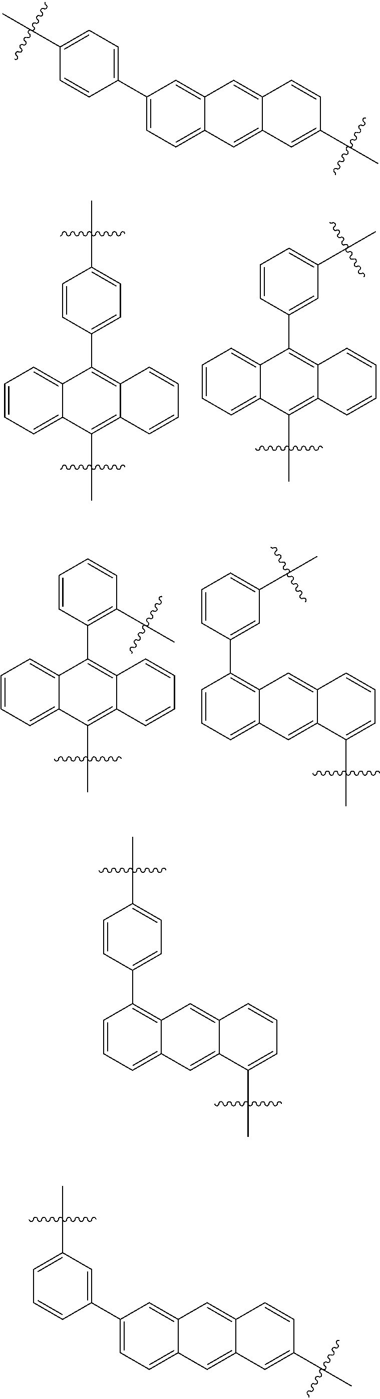

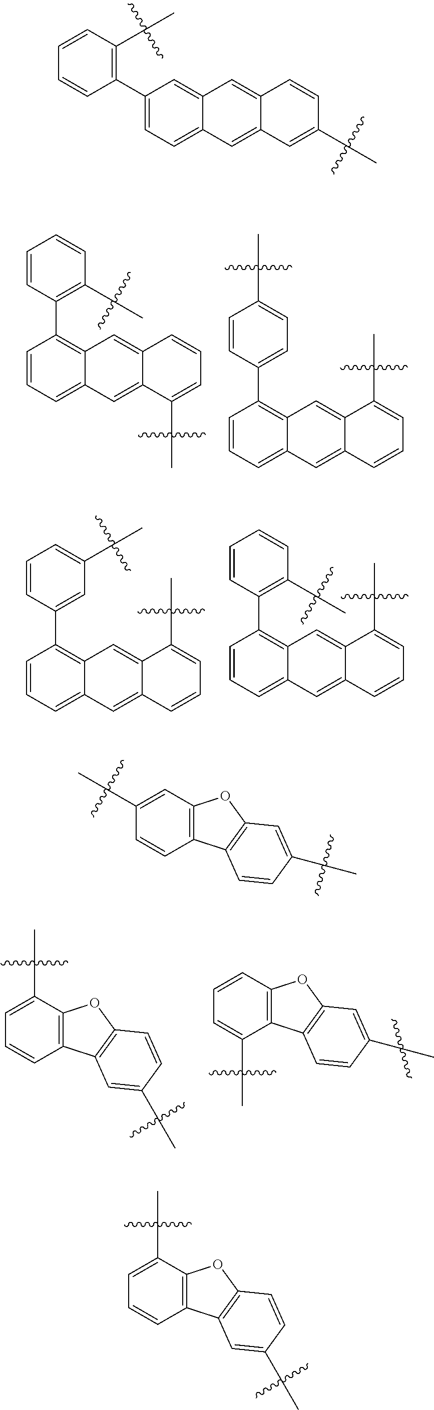

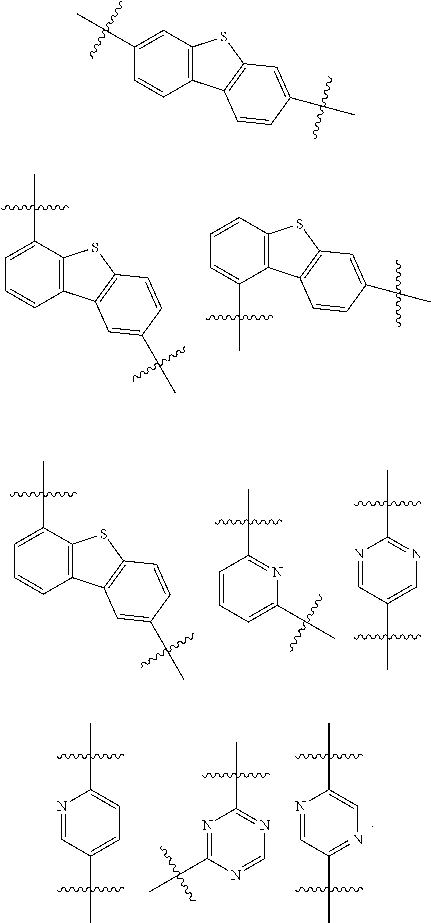

In one embodiment of the present application, each R is independently selected from a deuterium, a fluorine, a cyano, a methyl, an ethyl, an isopropyl, a tert-butyl, a substituted or unsubstituted phenyl, a substituted or unsubstituted naphthyl, a substituted or unsubstituted phenanthryl, a substituted or unsubstituted anthryl, a substituted or unsubstituted tetralinyl, a substituted or unsubstituted pyridyl, a substituted or unsubstituted pyrimidinyl, a substituted or unsubstituted triazinyl, a substituted or unsubstituted pyrazinyl, a substituted or unsubstituted quinolyl, or a substituted or unsubstituted isoquinolyl.

Alternatively, substituent(s) of R are the same or different, and are each independently selected from a deuterium, a fluorine, a cyano, a methyl, an ethyl, an isopropyl, a tert-butyl or a phenyl, and alternatively, any two adjacent Rs form a benzene ring or a naphthalene ring.

In one embodiment of the present application, each R is independently selected from a deuterium, a fluorine, a cyano, a methyl, an ethyl, an isopropyl, a tert-butyl, a phenyl, a pyridyl, a pyrimidinyl, a triazinyl, a naphthyl, a phenanthryl, an anthryl, a deuterophenyl, a pentadeuterophenyl, a pentamethyl-substituted phenyl, a methyl-substituted phenyl, a pentafluoro-substituted phenyl, a fluoro-substituted phenyl, a cyano-substituted phenyl, a tert-butyl-substituted phenyl, or a tetramethyl-substituted tetralinyl.

In particular, the organic compound is selected from the group consisting of the following compounds:

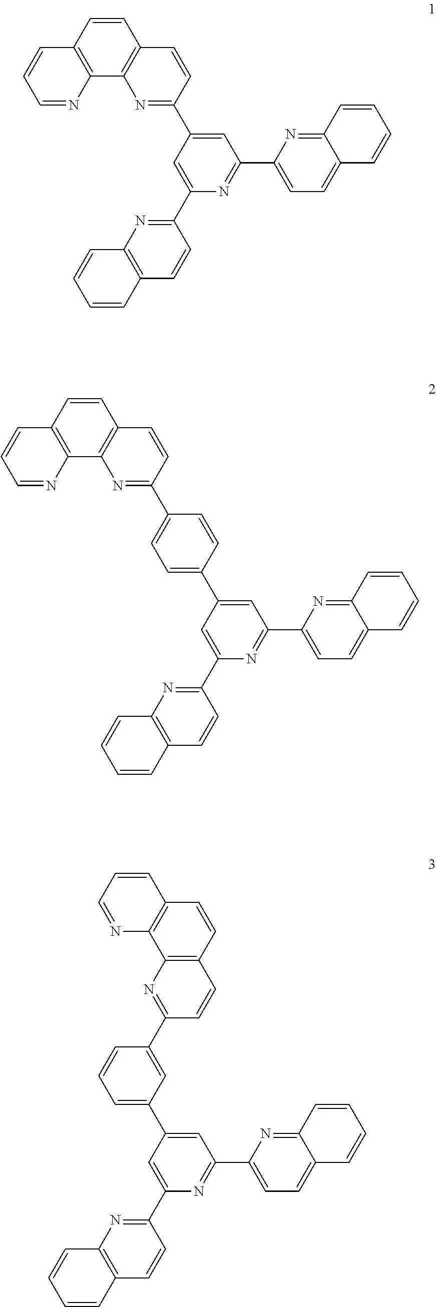 [SMILES: c1ccc2nc(-c3cc(-c4ccc5ccc6cccnc6c5n4)cc(-c4ccc5ccccc5n4)n3)ccc2c1 ; c1ccc2nc(-c3cc(-c4ccc(-c5ccc6ccc7cccnc7c6n5)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc3ccc4cccnc4c3n2)c1]

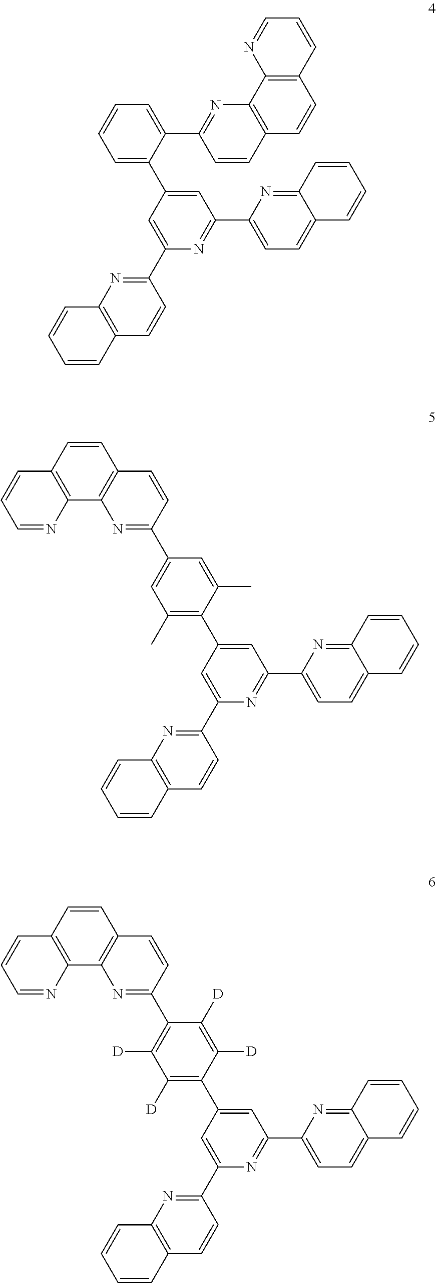 [SMILES: c1ccc(-c2ccc3ccc4cccnc4c3n2)c(-c2cc(-c3ccc4ccccc4n3)nc(-c3ccc4ccccc4n3)c2)c1 ; Cc1cc(-c2ccc3ccc4cccnc4c3n2)cc(C)c1-c1cc(-c2ccc3ccccc3n2)nc(-c2ccc3ccccc3n2)c1 ; [2H]c1c([2H])c(-c2ccc3ccc4cccnc4c3n2)c([2H])c([2H])c1-c1cc(-c2ccc3ccccc3n2)nc(-c2ccc3ccccc3n2)c1]

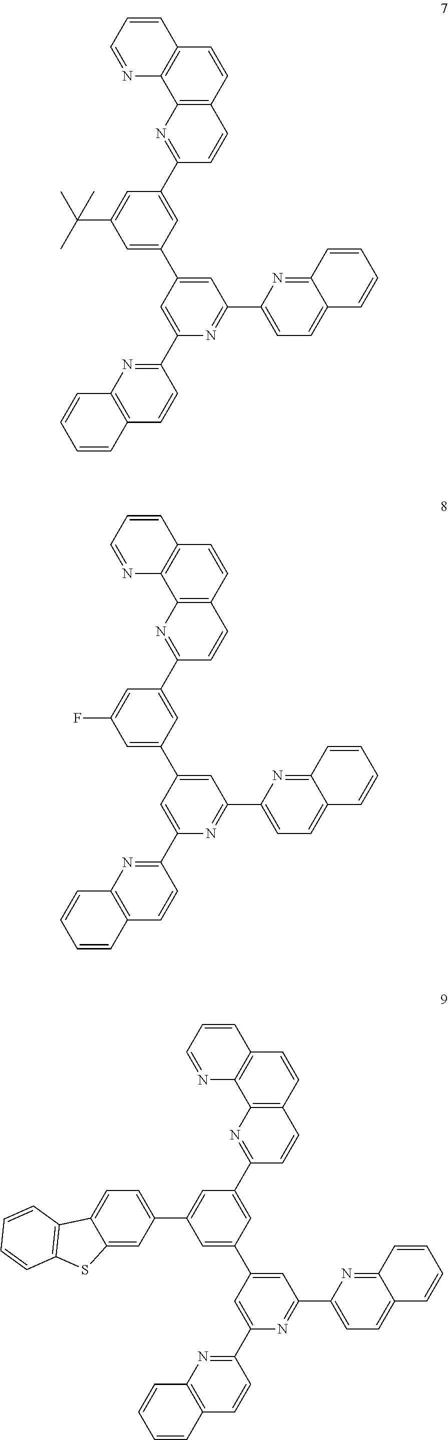 [SMILES: CC(C)(C)c1cc(-c2cc(-c3ccc4ccccc4n3)nc(-c3ccc4ccccc4n3)c2)cc(-c2ccc3ccc4cccnc4c3n2)c1 ; Fc1cc(-c2cc(-c3ccc4ccccc4n3)nc(-c3ccc4ccccc4n3)c2)cc(-c2ccc3ccc4cccnc4c3n2)c1 ; c1ccc2nc(-c3cc(-c4cc(-c5ccc6c(c5)sc5ccccc56)cc(-c5ccc6ccc7cccnc7c6n5)c4)cc(-c4ccc5ccccc5n4)n3)ccc2c1]

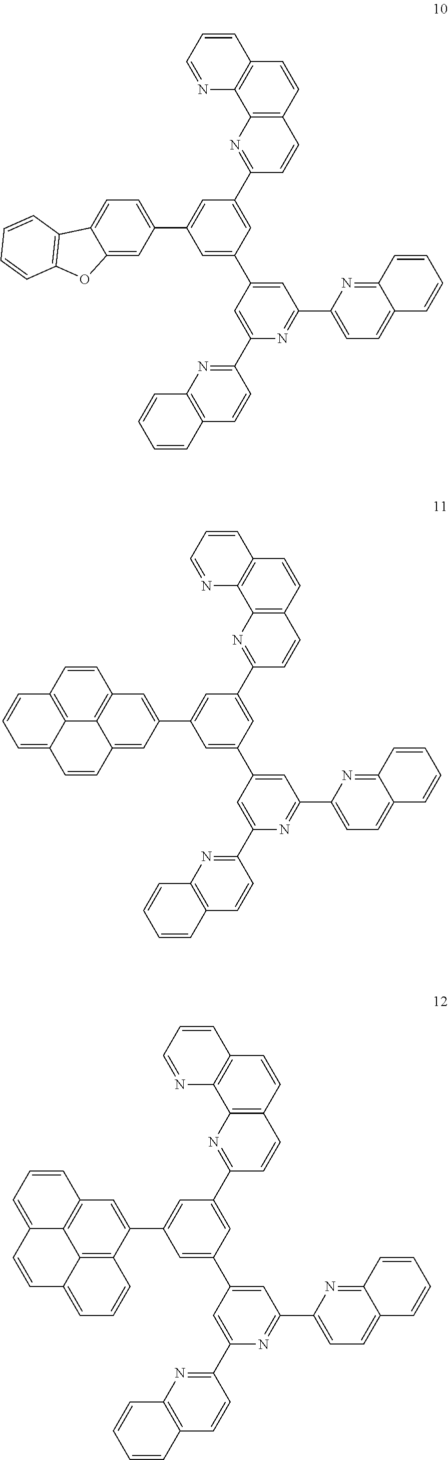 [SMILES: c1ccc2nc(-c3cc(-c4cc(-c5ccc6c(c5)oc5ccccc56)cc(-c5ccc6ccc7cccnc7c6n5)c4)cc(-c4ccc5ccccc5n4)n3)ccc2c1 ; c1ccc2nc(-c3cc(-c4cc(-c5cc6ccc7cccc8ccc(c5)c6c78)cc(-c5ccc6ccc7cccnc7c6n5)c4)cc(-c4ccc5ccccc5n4)n3)ccc2c1 ; c1ccc2nc(-c3cc(-c4cc(-c5ccc6ccc7cccnc7c6n5)cc(-c5cc6cccc7ccc8cccc5c8c76)c4)cc(-c4ccc5ccccc5n4)n3)ccc2c1]

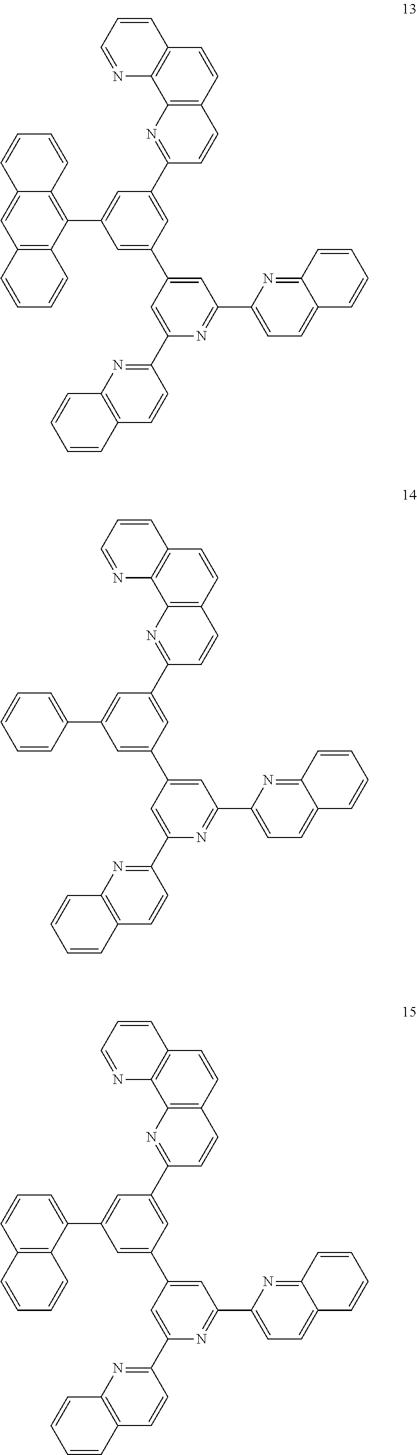 [SMILES: c1ccc2nc(-c3cc(-c4cc(-c5ccc6ccc7cccnc7c6n5)cc(-c5c6ccccc6cc6ccccc56)c4)cc(-c4ccc5ccccc5n4)n3)ccc2c1 ; c1ccc(-c2cc(-c3cc(-c4ccc5ccccc5n4)nc(-c4ccc5ccccc5n4)c3)cc(-c3ccc4ccc5cccnc5c4n3)c2)cc1 ; c1ccc2nc(-c3cc(-c4cc(-c5ccc6ccc7cccnc7c6n5)cc(-c5cccc6ccccc56)c4)cc(-c4ccc5ccccc5n4)n3)ccc2c1]

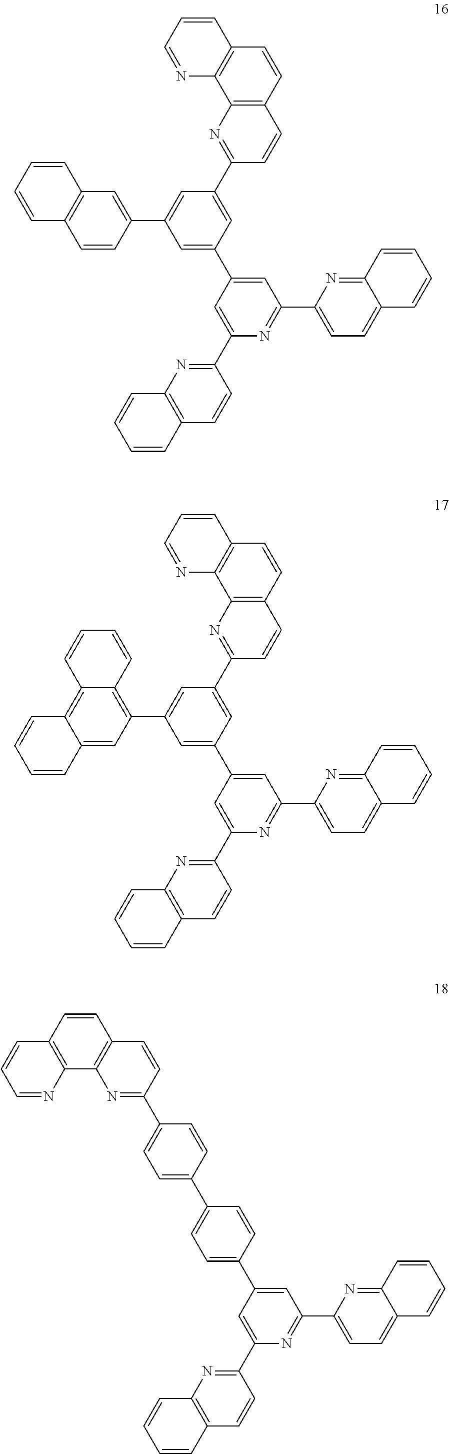 [SMILES: c1ccc2cc(-c3cc(-c4cc(-c5ccc6ccccc6n5)nc(-c5ccc6ccccc6n5)c4)cc(-c4ccc5ccc6cccnc6c5n4)c3)ccc2c1 ; c1ccc2nc(-c3cc(-c4cc(-c5ccc6ccc7cccnc7c6n5)cc(-c5cc6ccccc6c6ccccc56)c4)cc(-c4ccc5ccccc5n4)n3)ccc2c1 ; c1ccc2nc(-c3cc(-c4ccc(-c5ccc(-c6ccc7ccc8cccnc8c7n6)cc5)cc4)cc(-c4ccc5ccccc5n4)n3)ccc2c1]

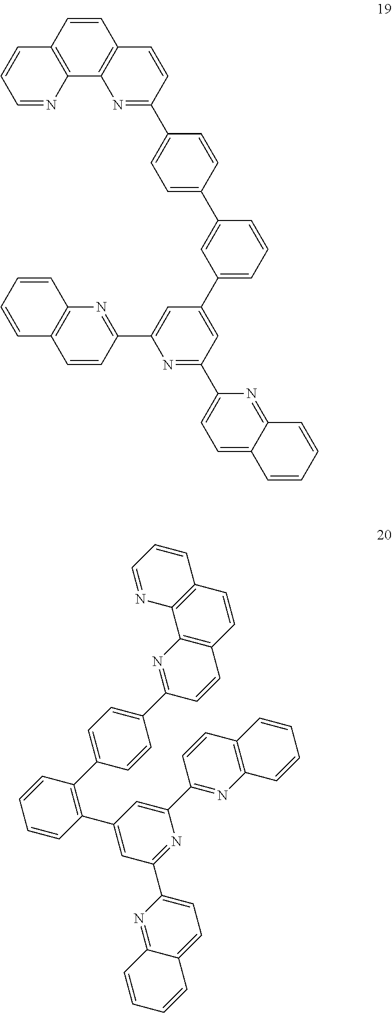 [SMILES: c1cc(-c2ccc(-c3ccc4ccc5cccnc5c4n3)cc2)cc(-c2cc(-c3ccc4ccccc4n3)nc(-c3ccc4ccccc4n3)c2)c1 ; c1ccc(-c2cc(-c3ccc4ccccc4n3)nc(-c3ccc4ccccc4n3)c2)c(-c2ccc(-c3ccc4ccc5cccnc5c4n3)cc2)c1]

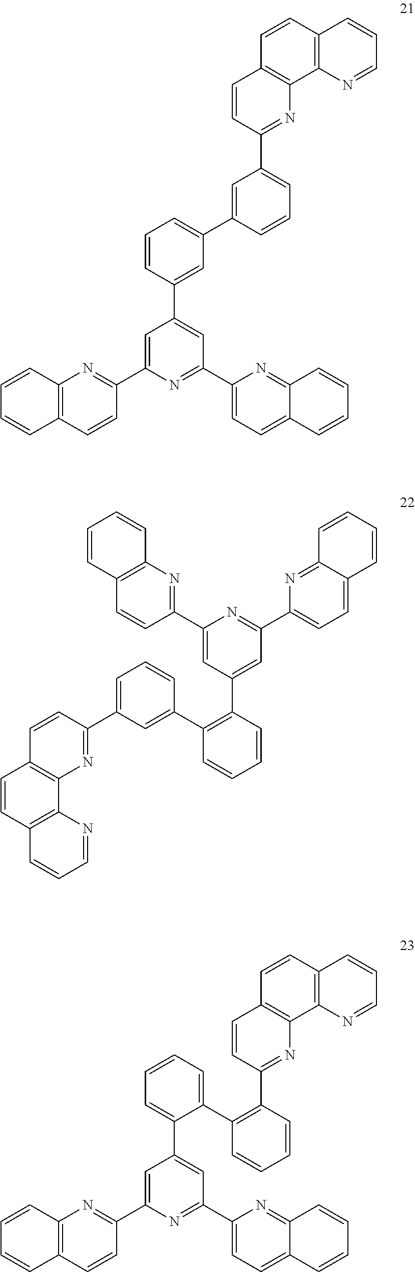 [SMILES: c1cc(-c2cccc(-c3ccc4ccc5cccnc5c4n3)c2)cc(-c2cc(-c3ccc4ccccc4n3)nc(-c3ccc4ccccc4n3)c2)c1 ; c1cc(-c2ccc3ccc4cccnc4c3n2)cc(-c2ccccc2-c2cc(-c3ccc4ccccc4n3)nc(-c3ccc4ccccc4n3)c2)c1 ; c1ccc(-c2ccccc2-c2ccc3ccc4cccnc4c3n2)c(-c2cc(-c3ccc4ccccc4n3)nc(-c3ccc4ccccc4n3)c2)c1]

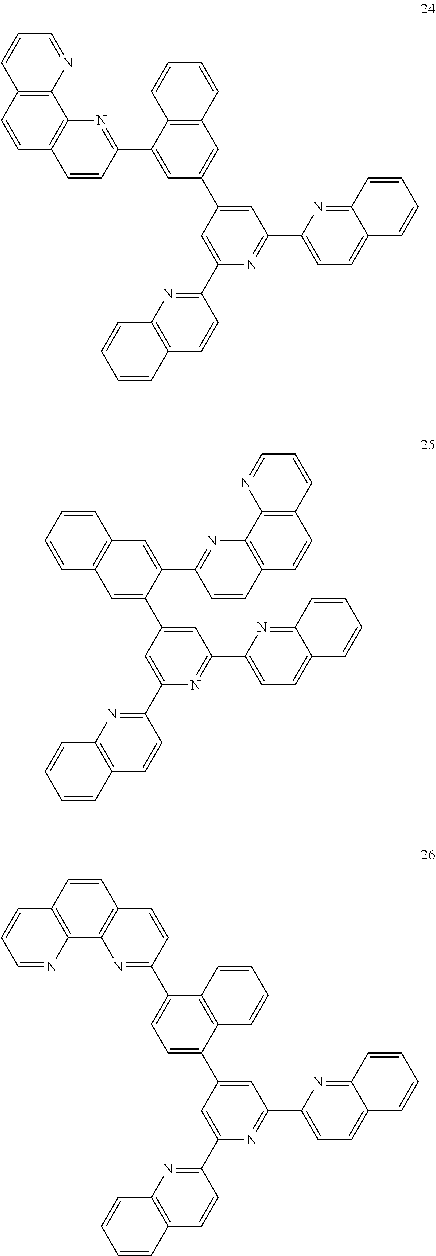 [SMILES: c1ccc2nc(-c3cc(-c4cc(-c5ccc6ccc7cccnc7c6n5)c5ccccc5c4)cc(-c4ccc5ccccc5n4)n3)ccc2c1 ; c1ccc2cc(-c3ccc4ccc5cccnc5c4n3)c(-c3cc(-c4ccc5ccccc5n4)nc(-c4ccc5ccccc5n4)c3)cc2c1 ; c1ccc2nc(-c3cc(-c4ccc(-c5ccc6ccc7cccnc7c6n5)c5ccccc45)cc(-c4ccc5ccccc5n4)n3)ccc2c1]

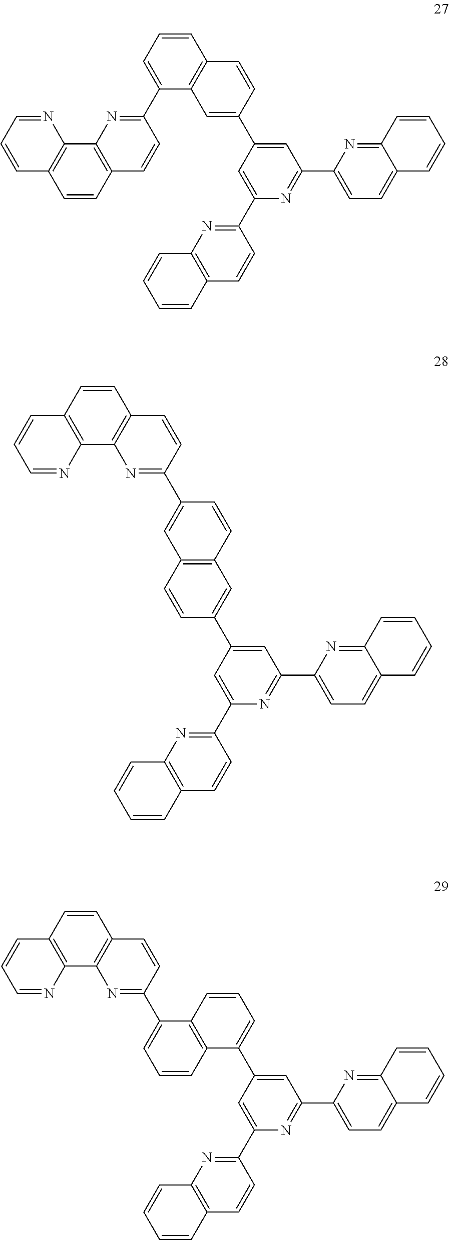 [SMILES: c1ccc2nc(-c3cc(-c4ccc5cccc(-c6ccc7ccc8cccnc8c7n6)c5c4)cc(-c4ccc5ccccc5n4)n3)ccc2c1 ; c1ccc2nc(-c3cc(-c4ccc5cc(-c6ccc7ccc8cccnc8c7n6)ccc5c4)cc(-c4ccc5ccccc5n4)n3)ccc2c1 ; c1ccc2nc(-c3cc(-c4cccc5c(-c6ccc7ccc8cccnc8c7n6)cccc45)cc(-c4ccc5ccccc5n4)n3)ccc2c1]

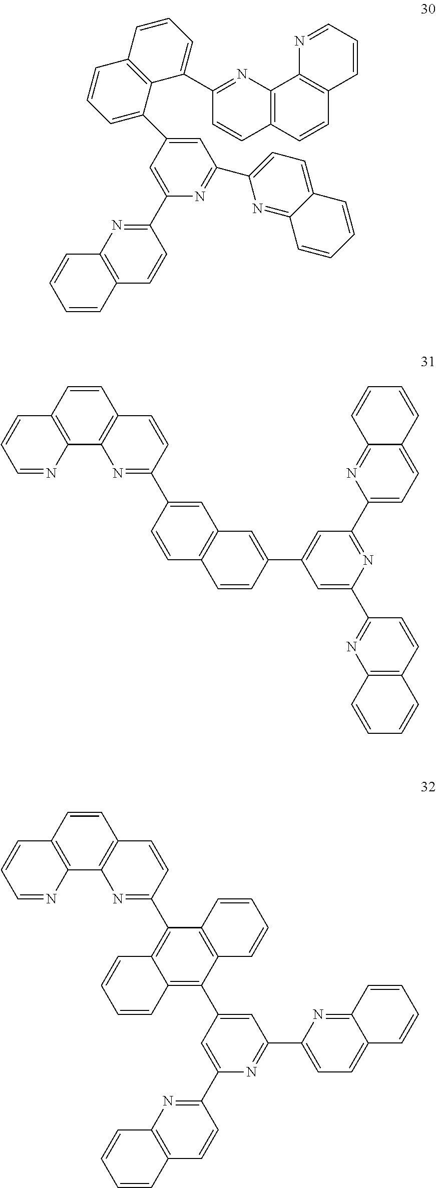 [SMILES: c1ccc2nc(-c3cc(-c4cccc5cccc(-c6ccc7ccc8cccnc8c7n6)c45)cc(-c4ccc5ccccc5n4)n3)ccc2c1 ; c1ccc2nc(-c3cc(-c4ccc5ccc(-c6ccc7ccc8cccnc8c7n6)cc5c4)cc(-c4ccc5ccccc5n4)n3)ccc2c1 ; c1ccc2nc(-c3cc(-c4c5ccccc5c(-c5ccc6ccc7cccnc7c6n5)c5ccccc45)cc(-c4ccc5ccccc5n4)n3)ccc2c1]

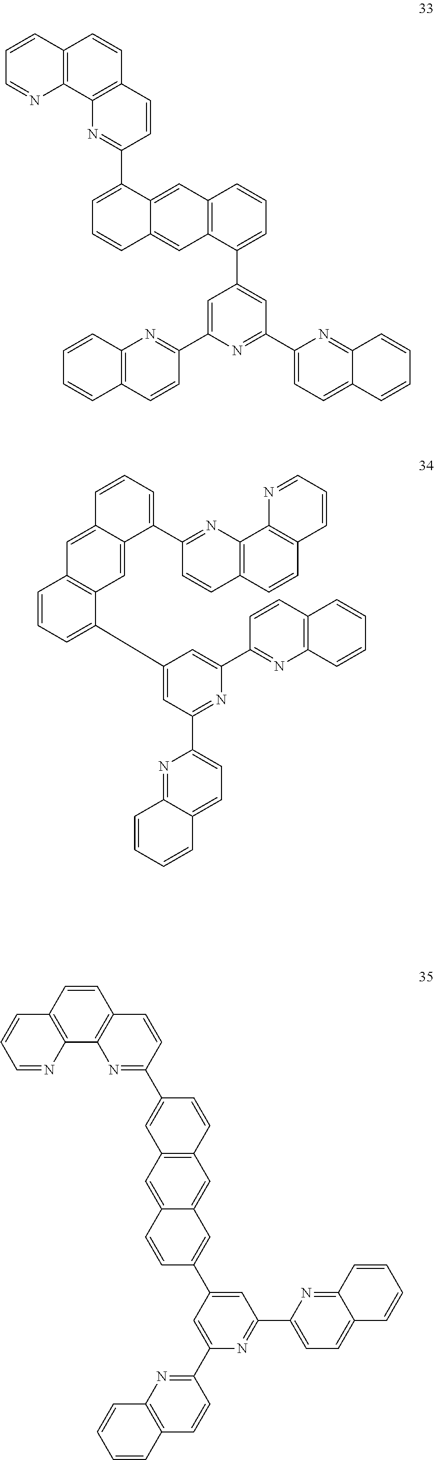 [SMILES: c1ccc2nc(-c3cc(-c4cccc5cc6c(-c7ccc8ccc9cccnc9c8n7)cccc6cc45)cc(-c4ccc5ccccc5n4)n3)ccc2c1 ; c1ccc2nc(-c3cc(-c4cccc5cc6cccc(-c7ccc8ccc9cccnc9c8n7)c6cc45)cc(-c4ccc5ccccc5n4)n3)ccc2c1 ; c1ccc2nc(-c3cc(-c4ccc5cc6cc(-c7ccc8ccc9cccnc9c8n7)ccc6cc5c4)cc(-c4ccc5ccccc5n4)n3)ccc2c1]

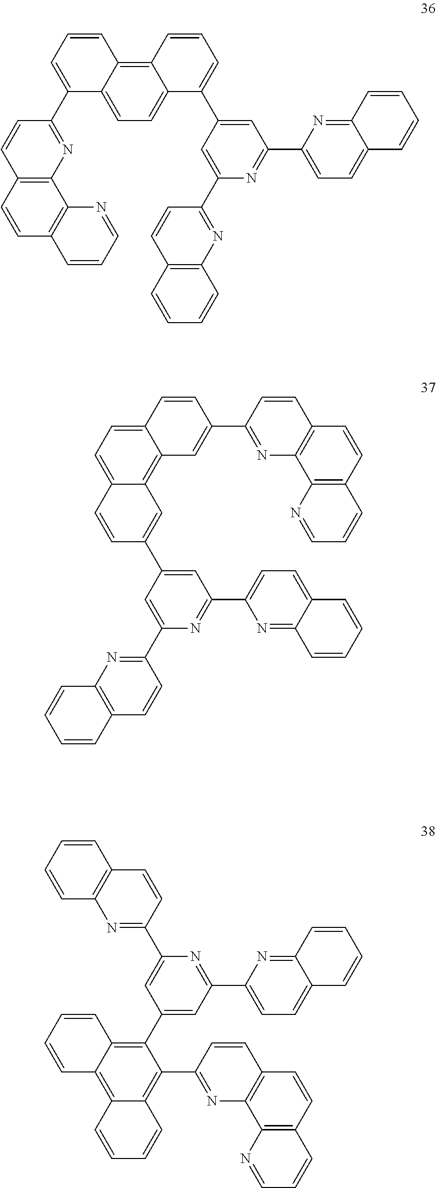 [SMILES: c1ccc2nc(-c3cc(-c4cccc5c4ccc4c(-c6ccc7ccc8cccnc8c7n6)cccc45)cc(-c4ccc5ccccc5n4)n3)ccc2c1 ; c1ccc2nc(-c3cc(-c4ccc5ccc6ccc(-c7ccc8ccc9cccnc9c8n7)cc6c5c4)cc(-c4ccc5ccccc5n4)n3)ccc2c1 ; c1ccc2nc(-c3cc(-c4c(-c5ccc6ccc7cccnc7c6n5)c5ccccc5c5ccccc45)cc(-c4ccc5ccccc5n4)n3)ccc2c1]

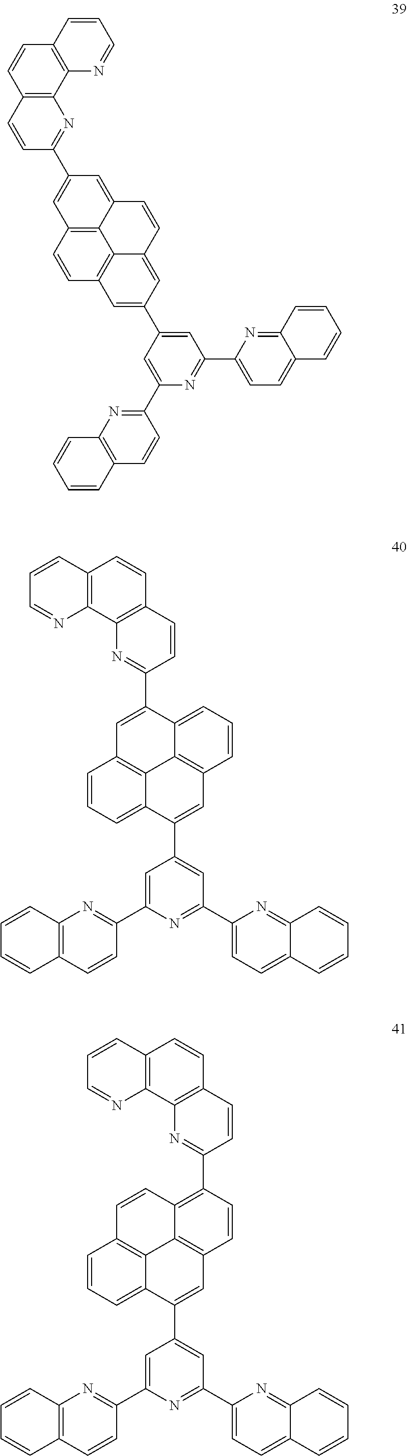 [SMILES: c1ccc2nc(-c3cc(-c4cc5ccc6cc(-c7ccc8ccc9cccnc9c8n7)cc7ccc(c4)c5c67)cc(-c4ccc5ccccc5n4)n3)ccc2c1 ; c1ccc2nc(-c3cc(-c4cc5cccc6c(-c7ccc8ccc9cccnc9c8n7)cc7cccc4c7c56)cc(-c4ccc5ccccc5n4)n3)ccc2c1 ; c1ccc2nc(-c3cc(-c4cc5ccc(-c6ccc7ccc8cccnc8c7n6)c6ccc7cccc4c7c56)cc(-c4ccc5ccccc5n4)n3)ccc2c1]

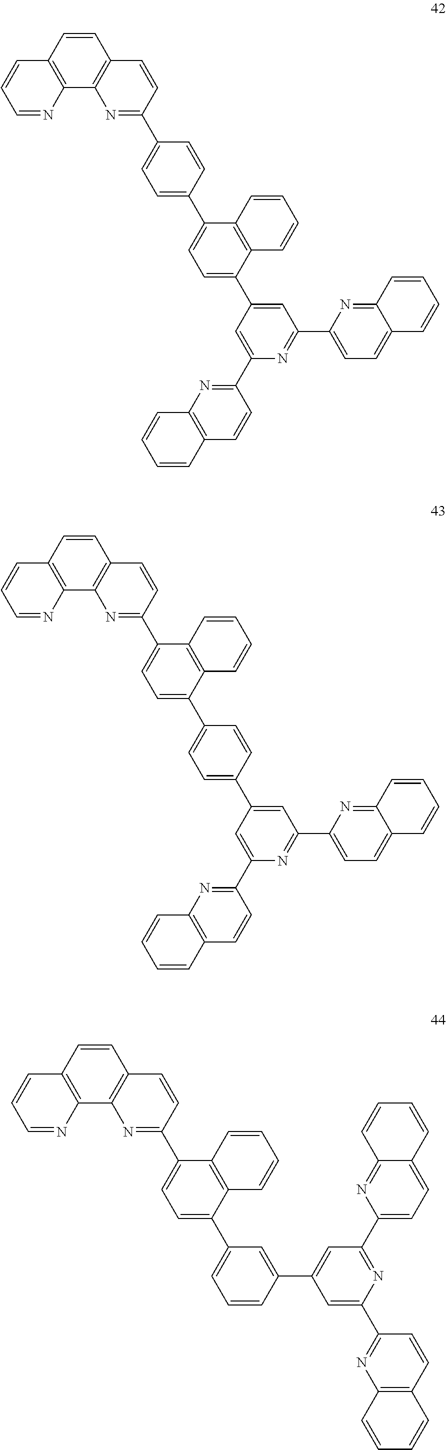 [SMILES: c1ccc2nc(-c3cc(-c4ccc(-c5ccc(-c6ccc7ccc8cccnc8c7n6)cc5)c5ccccc45)cc(-c4ccc5ccccc5n4)n3)ccc2c1 ; c1ccc2nc(-c3cc(-c4ccc(-c5ccc(-c6ccc7ccc8cccnc8c7n6)c6ccccc56)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc(-c3ccc4ccc5cccnc5c4n3)c3ccccc23)c1]

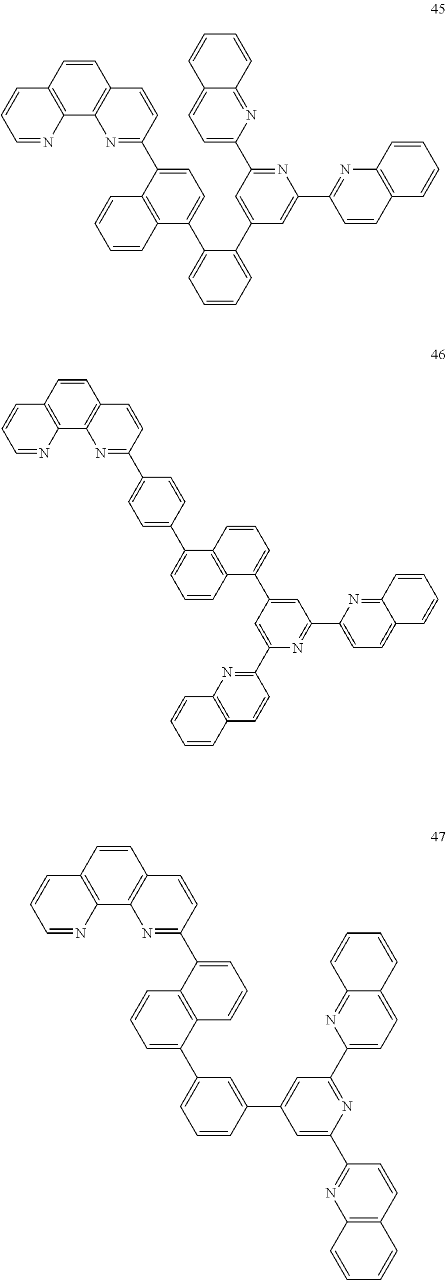 [SMILES: c1ccc(-c2ccc(-c3ccc4ccc5cccnc5c4n3)c3ccccc23)c(-c2cc(-c3ccc4ccccc4n3)nc(-c3ccc4ccccc4n3)c2)c1 ; c1ccc2nc(-c3cc(-c4cccc5c(-c6ccc(-c7ccc8ccc9cccnc9c8n7)cc6)cccc45)cc(-c4ccc5ccccc5n4)n3)ccc2c1 ; c1cc(-c2cc(-c3ccc4ccccc4n3)nc(-c3ccc4ccccc4n3)c2)cc(-c2cccc3c(-c4ccc5ccc6cccnc6c5n4)cccc23)c1]

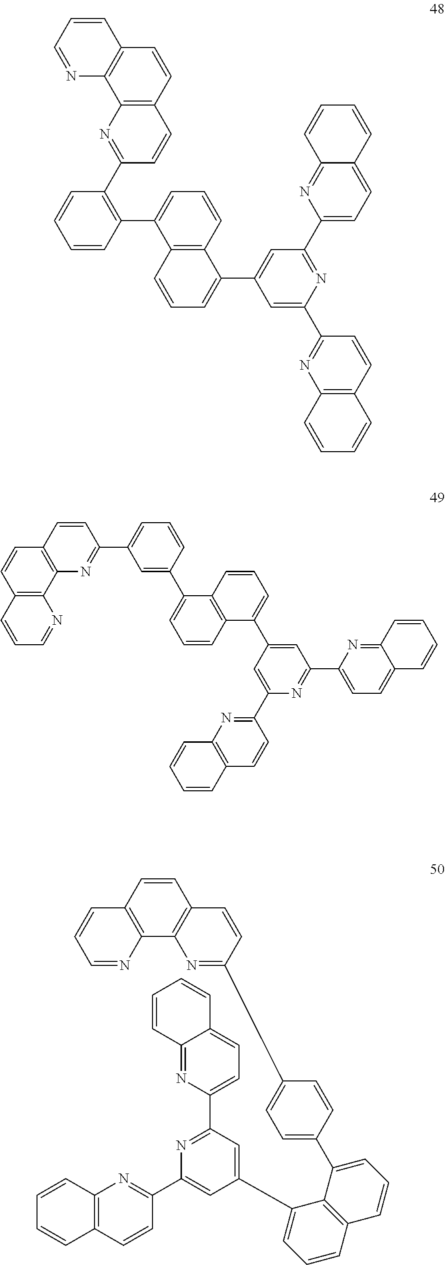 [SMILES: c1ccc(-c2cccc3c(-c4cc(-c5ccc6ccccc6n5)nc(-c5ccc6ccccc6n5)c4)cccc23)c(-c2ccc3ccc4cccnc4c3n2)c1 ; c1cc(-c2ccc3ccc4cccnc4c3n2)cc(-c2cccc3c(-c4cc(-c5ccc6ccccc6n5)nc(-c5ccc6ccccc6n5)c4)cccc23)c1 ; c1ccc2nc(-c3cc(-c4cccc5cccc(-c6ccc(-c7ccc8ccc9cccnc9c8n7)cc6)c45)cc(-c4ccc5ccccc5n4)n3)ccc2c1]

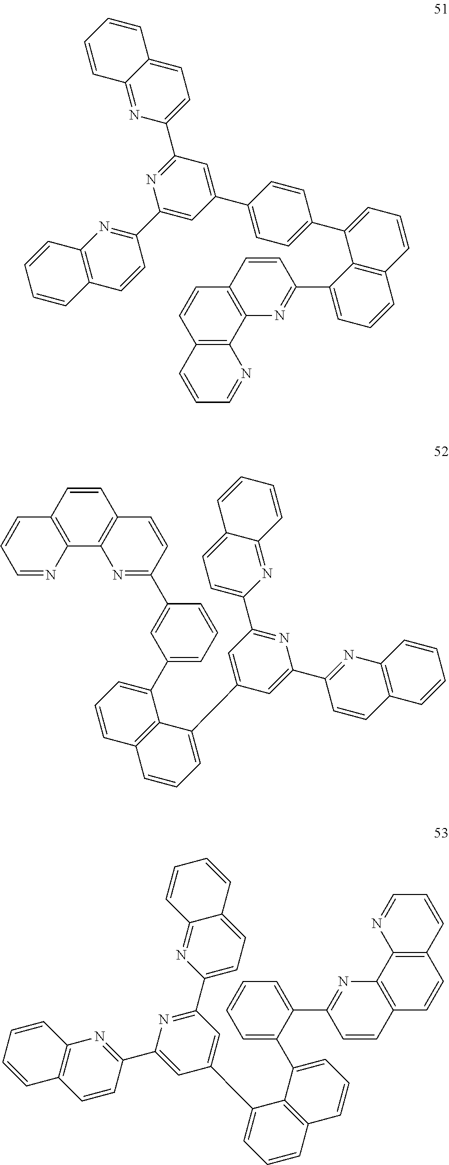 [SMILES: c1ccc2nc(-c3cc(-c4ccc(-c5cccc6cccc(-c7ccc8ccc9cccnc9c8n7)c56)cc4)cc(-c4ccc5ccccc5n4)n3)ccc2c1 ; c1cc(-c2ccc3ccc4cccnc4c3n2)cc(-c2cccc3cccc(-c4cc(-c5ccc6ccccc6n5)nc(-c5ccc6ccccc6n5)c4)c23)c1 ; c1ccc(-c2cccc3cccc(-c4cc(-c5ccc6ccccc6n5)nc(-c5ccc6ccccc6n5)c4)c23)c(-c2ccc3ccc4cccnc4c3n2)c1]

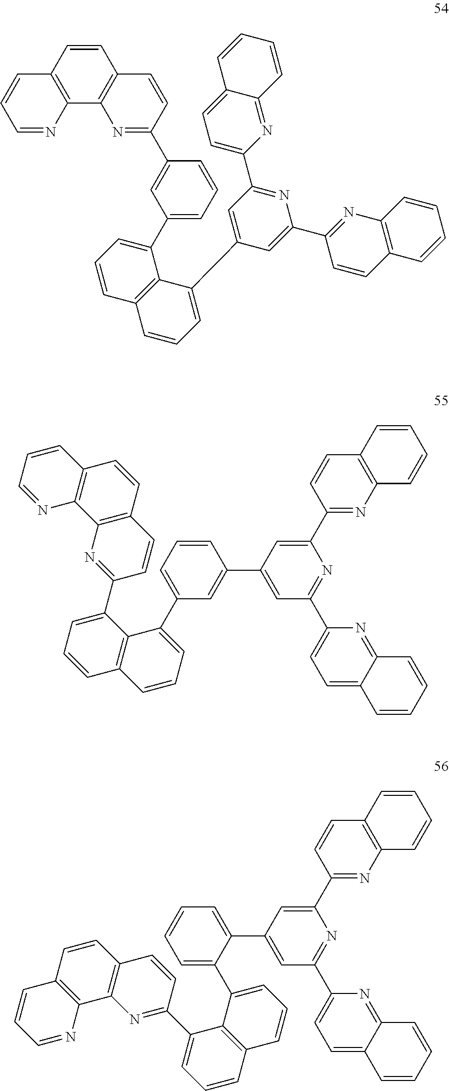 [SMILES: c1cc(-c2ccc3ccc4cccnc4c3n2)cc(-c2cccc3cccc(-c4cc(-c5ccc6ccccc6n5)nc(-c5ccc6ccccc6n5)c4)c23)c1 ; c1cc(-c2cc(-c3ccc4ccccc4n3)nc(-c3ccc4ccccc4n3)c2)cc(-c2cccc3cccc(-c4ccc5ccc6cccnc6c5n4)c23)c1 ; c1ccc(-c2cccc3cccc(-c4ccc5ccc6cccnc6c5n4)c23)c(-c2cc(-c3ccc4ccccc4n3)nc(-c3ccc4ccccc4n3)c2)c1]

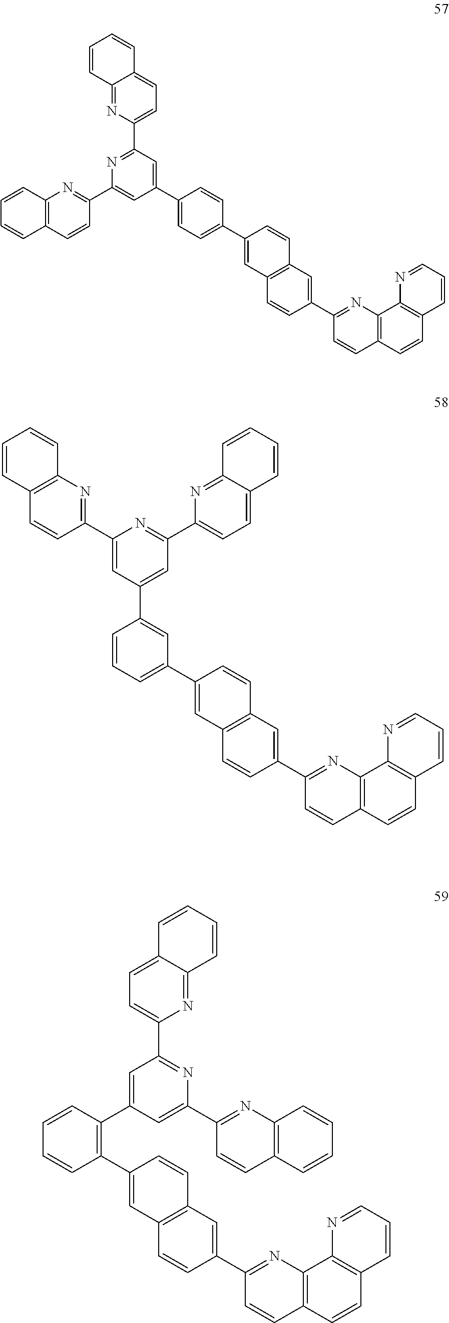 [SMILES: c1ccc2nc(-c3cc(-c4ccc(-c5ccc6cc(-c7ccc8ccc9cccnc9c8n7)ccc6c5)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc3cc(-c4ccc5ccc6cccnc6c5n4)ccc3c2)c1 ; c1ccc(-c2ccc3cc(-c4ccc5ccc6cccnc6c5n4)ccc3c2)c(-c2cc(-c3ccc4ccccc4n3)nc(-c3ccc4ccccc4n3)c2)c1]

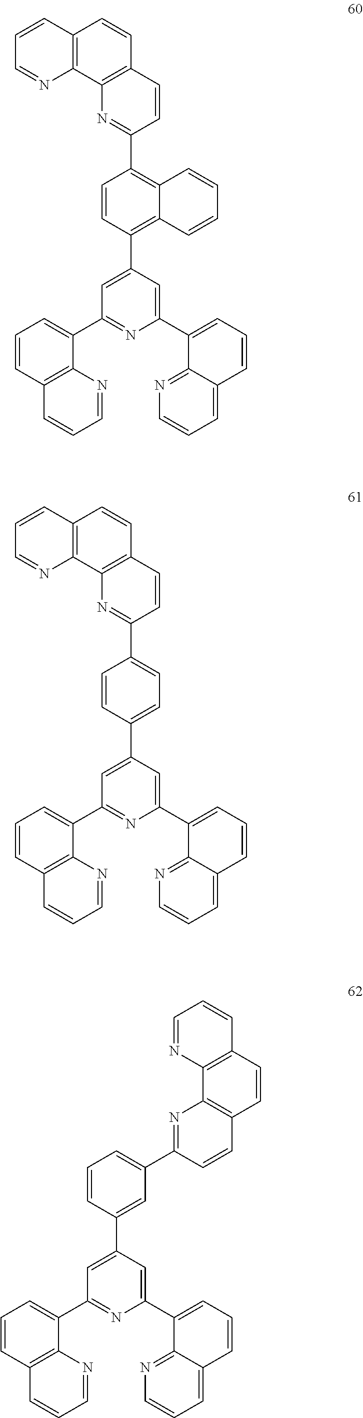 [SMILES: c1cnc2c(-c3cc(-c4ccc(-c5ccc6ccc7cccnc7c6n5)c5ccccc45)cc(-c4cccc5cccnc45)n3)cccc2c1 ; c1cnc2c(-c3cc(-c4ccc(-c5ccc6ccc7cccnc7c6n5)cc4)cc(-c4cccc5cccnc45)n3)cccc2c1 ; c1cc(-c2cc(-c3cccc4cccnc34)nc(-c3cccc4cccnc34)c2)cc(-c2ccc3ccc4cccnc4c3n2)c1]

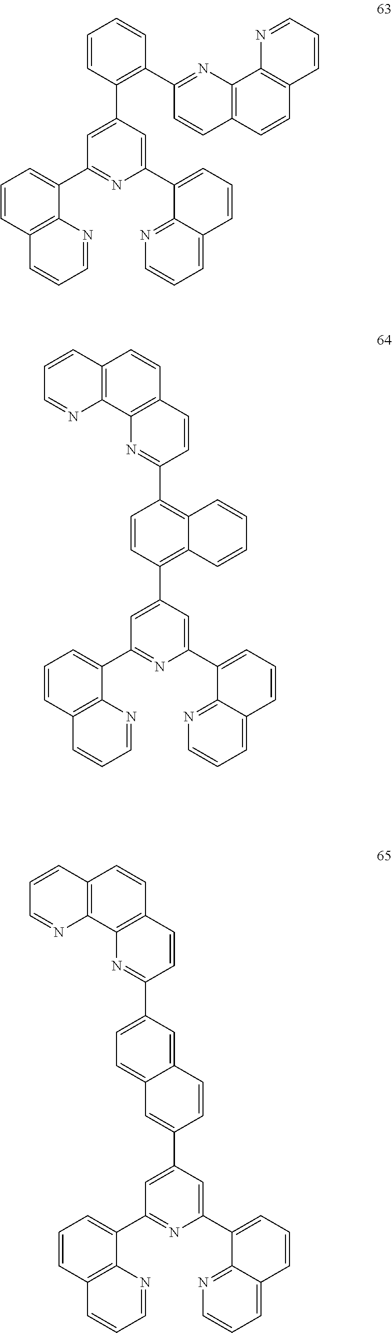 [SMILES: c1ccc(-c2ccc3ccc4cccnc4c3n2)c(-c2cc(-c3cccc4cccnc34)nc(-c3cccc4cccnc34)c2)c1 ; c1cnc2c(-c3cc(-c4ccc(-c5ccc6ccc7cccnc7c6n5)c5ccccc45)cc(-c4cccc5cccnc45)n3)cccc2c1 ; c1cnc2c(-c3cc(-c4ccc5cc(-c6ccc7ccc8cccnc8c7n6)ccc5c4)cc(-c4cccc5cccnc45)n3)cccc2c1]

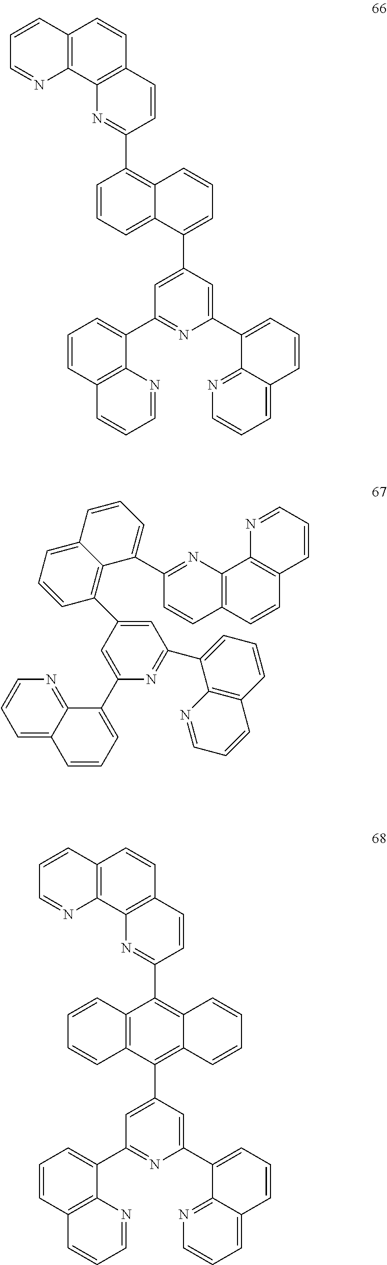 [SMILES: c1cnc2c(-c3cc(-c4cccc5c(-c6ccc7ccc8cccnc8c7n6)cccc45)cc(-c4cccc5cccnc45)n3)cccc2c1 ; c1cnc2c(-c3cc(-c4cccc5cccc(-c6ccc7ccc8cccnc8c7n6)c45)cc(-c4cccc5cccnc45)n3)cccc2c1 ; c1cnc2c(-c3cc(-c4c5ccccc5c(-c5ccc6ccc7cccnc7c6n5)c5ccccc45)cc(-c4cccc5cccnc45)n3)cccc2c1]

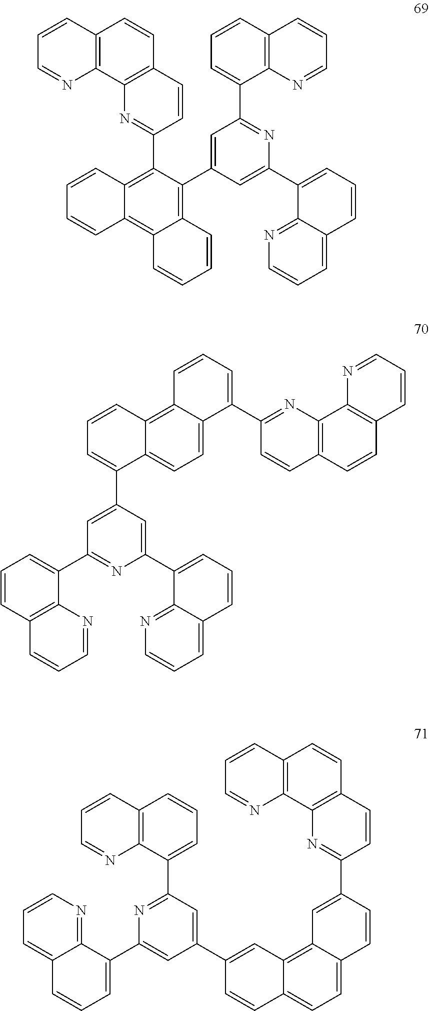 [SMILES: c1cnc2c(-c3cc(-c4c(-c5ccc6ccc7cccnc7c6n5)c5ccccc5c5ccccc45)cc(-c4cccc5cccnc45)n3)cccc2c1 ; c1cnc2c(-c3cc(-c4cccc5c4ccc4c(-c6ccc7ccc8cccnc8c7n6)cccc45)cc(-c4cccc5cccnc45)n3)cccc2c1 ; c1cnc2c(-c3cc(-c4ccc5ccc6ccc(-c7ccc8ccc9cccnc9c8n7)cc6c5c4)cc(-c4cccc5cccnc45)n3)cccc2c1]

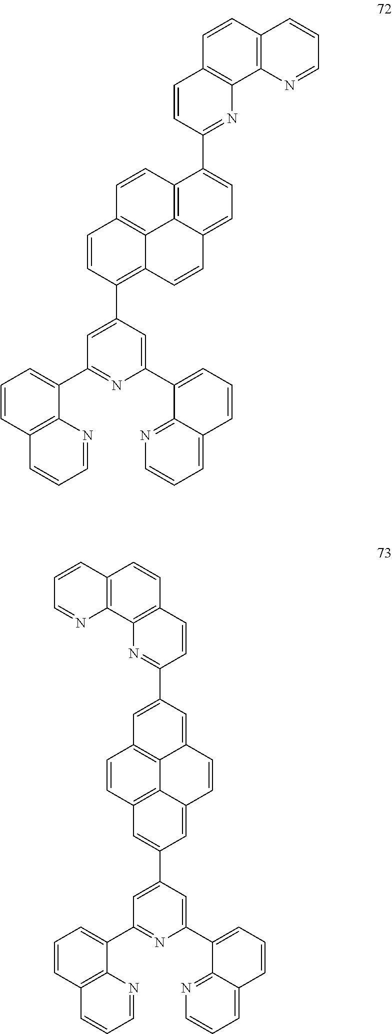 [SMILES: c1cnc2c(-c3cc(-c4ccc5ccc6c(-c7ccc8ccc9cccnc9c8n7)ccc7ccc4c5c76)cc(-c4cccc5cccnc45)n3)cccc2c1 ; c1cnc2c(-c3cc(-c4cc5ccc6cc(-c7ccc8ccc9cccnc9c8n7)cc7ccc(c4)c5c67)cc(-c4cccc5cccnc45)n3)cccc2c1]

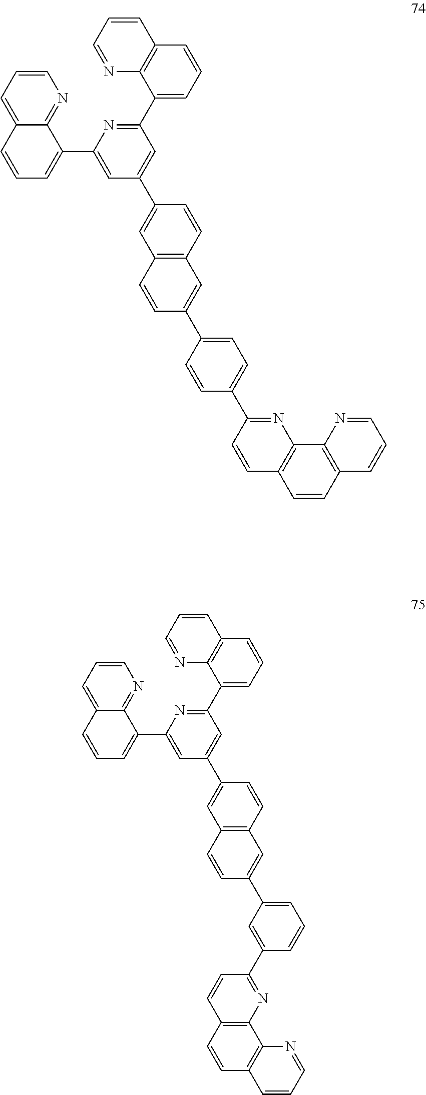 [SMILES: c1cnc2c(-c3cc(-c4ccc5cc(-c6ccc(-c7ccc8ccc9cccnc9c8n7)cc6)ccc5c4)cc(-c4cccc5cccnc45)n3)cccc2c1 ; c1cc(-c2ccc3cc(-c4cc(-c5cccc6cccnc56)nc(-c5cccc6cccnc56)c4)ccc3c2)cc(-c2ccc3ccc4cccnc4c3n2)c1]

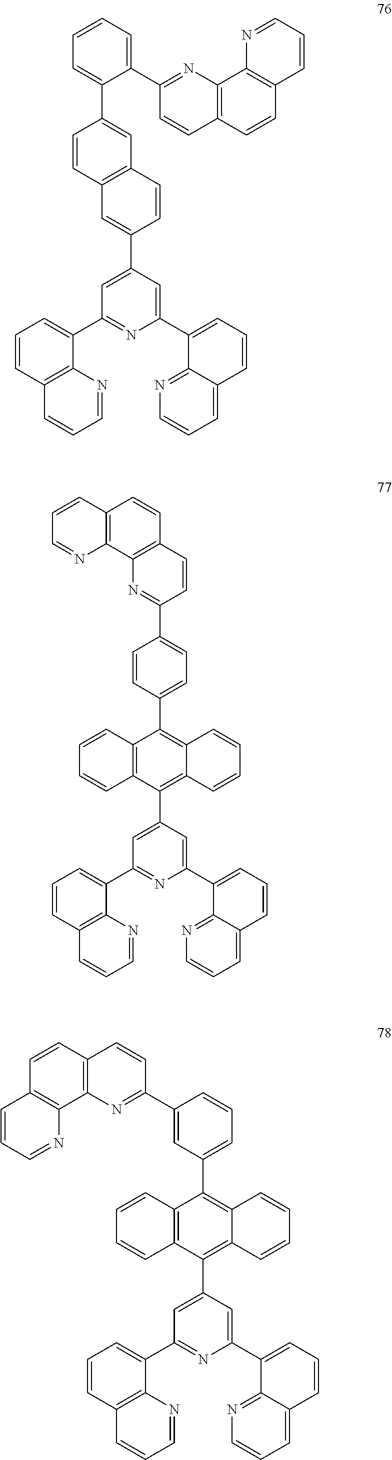 [SMILES: c1ccc(-c2ccc3ccc4cccnc4c3n2)c(-c2ccc3cc(-c4cc(-c5cccc6cccnc56)nc(-c5cccc6cccnc56)c4)ccc3c2)c1 ; c1cnc2c(-c3cc(-c4c5ccccc5c(-c5ccc(-c6ccc7ccc8cccnc8c7n6)cc5)c5ccccc45)cc(-c4cccc5cccnc45)n3)cccc2c1 ; c1cc(-c2ccc3ccc4cccnc4c3n2)cc(-c2c3ccccc3c(-c3cc(-c4cccc5cccnc45)nc(-c4cccc5cccnc45)c3)c3ccccc23)c1]

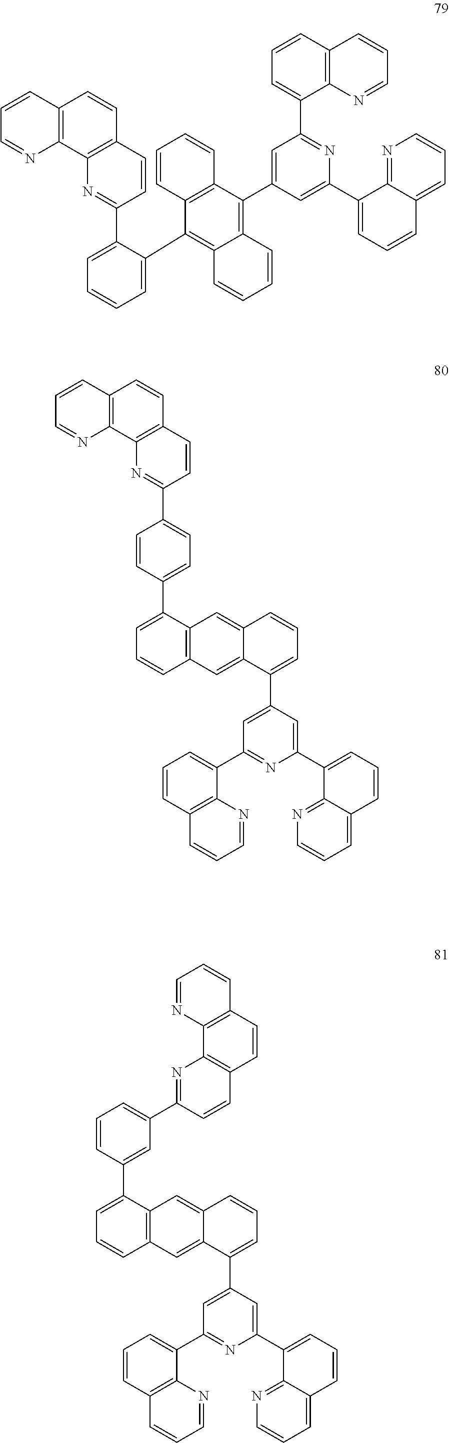 [SMILES: c1ccc(-c2c3ccccc3c(-c3cc(-c4cccc5cccnc45)nc(-c4cccc5cccnc45)c3)c3ccccc23)c(-c2ccc3ccc4cccnc4c3n2)c1 ; c1cc(-c2ccc(-c3ccc4ccc5cccnc5c4n3)cc2)c2cc3cccc(-c4cc(-c5cccc6cccnc56)nc(-c5cccc6cccnc56)c4)c3cc2c1 ; c1cc(-c2ccc3ccc4cccnc4c3n2)cc(-c2cccc3cc4c(-c5cc(-c6cccc7cccnc67)nc(-c6cccc7cccnc67)c5)cccc4cc23)c1]

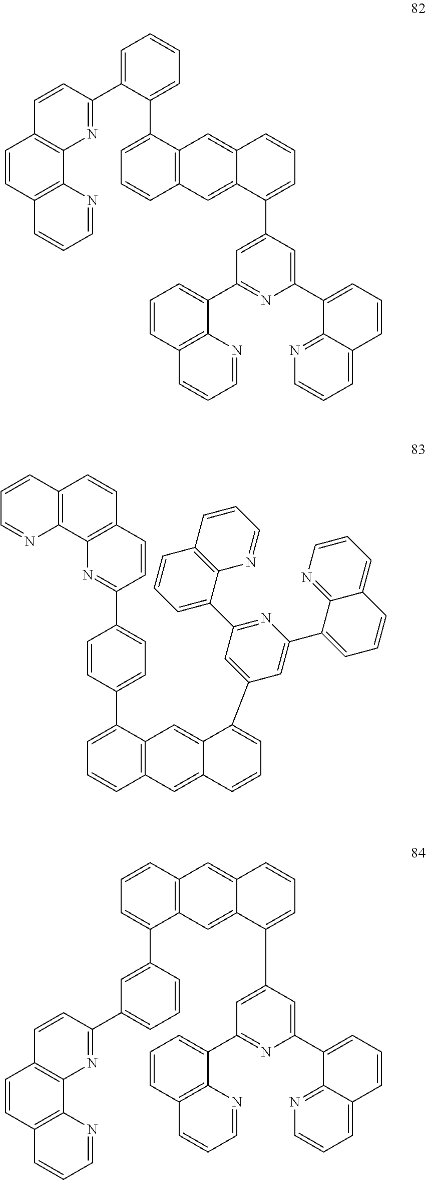 [SMILES: c1ccc(-c2cccc3cc4c(-c5cc(-c6cccc7cccnc67)nc(-c6cccc7cccnc67)c5)cccc4cc23)c(-c2ccc3ccc4cccnc4c3n2)c1 ; c1cc(-c2ccc(-c3ccc4ccc5cccnc5c4n3)cc2)c2cc3c(-c4cc(-c5cccc6cccnc56)nc(-c5cccc6cccnc56)c4)cccc3cc2c1 ; c1cc(-c2ccc3ccc4cccnc4c3n2)cc(-c2cccc3cc4cccc(-c5cc(-c6cccc7cccnc67)nc(-c6cccc7cccnc67)c5)c4cc23)c1]

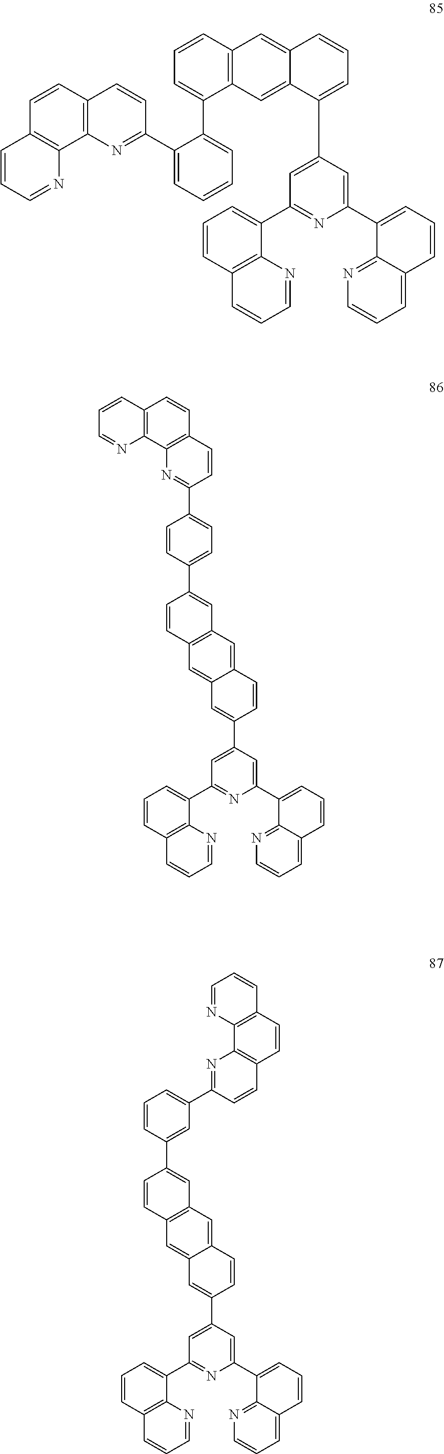 [SMILES: c1ccc(-c2cccc3cc4cccc(-c5cc(-c6cccc7cccnc67)nc(-c6cccc7cccnc67)c5)c4cc23)c(-c2ccc3ccc4cccnc4c3n2)c1 ; c1cnc2c(-c3cc(-c4ccc5cc6cc(-c7ccc(-c8ccc9ccc%10cccnc%10c9n8)cc7)ccc6cc5c4)cc(-c4cccc5cccnc45)n3)cccc2c1 ; c1cc(-c2ccc3cc4cc(-c5cc(-c6cccc7cccnc67)nc(-c6cccc7cccnc67)c5)ccc4cc3c2)cc(-c2ccc3ccc4cccnc4c3n2)c1]

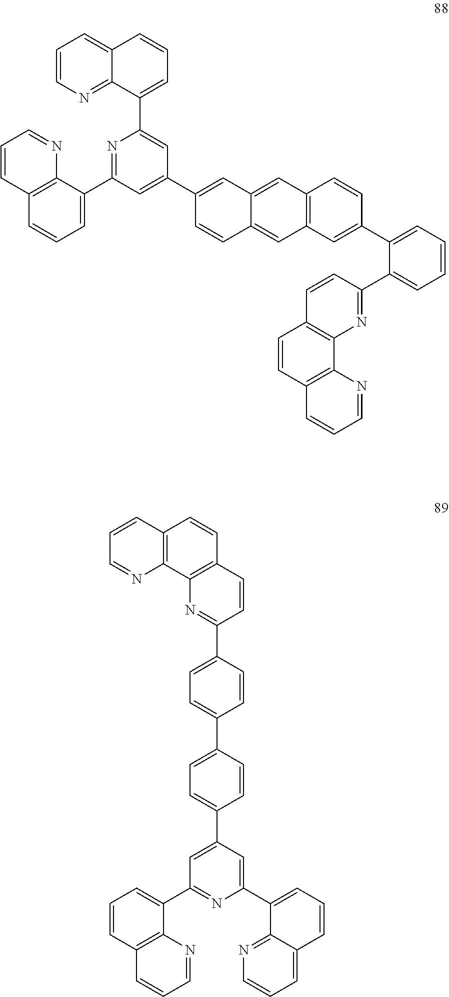 [SMILES: c1ccc(-c2ccc3ccc4cccnc4c3n2)c(-c2ccc3cc4cc(-c5cc(-c6cccc7cccnc67)nc(-c6cccc7cccnc67)c5)ccc4cc3c2)c1 ; c1cnc2c(-c3cc(-c4ccc(-c5ccc(-c6ccc7ccc8cccnc8c7n6)cc5)cc4)cc(-c4cccc5cccnc45)n3)cccc2c1]

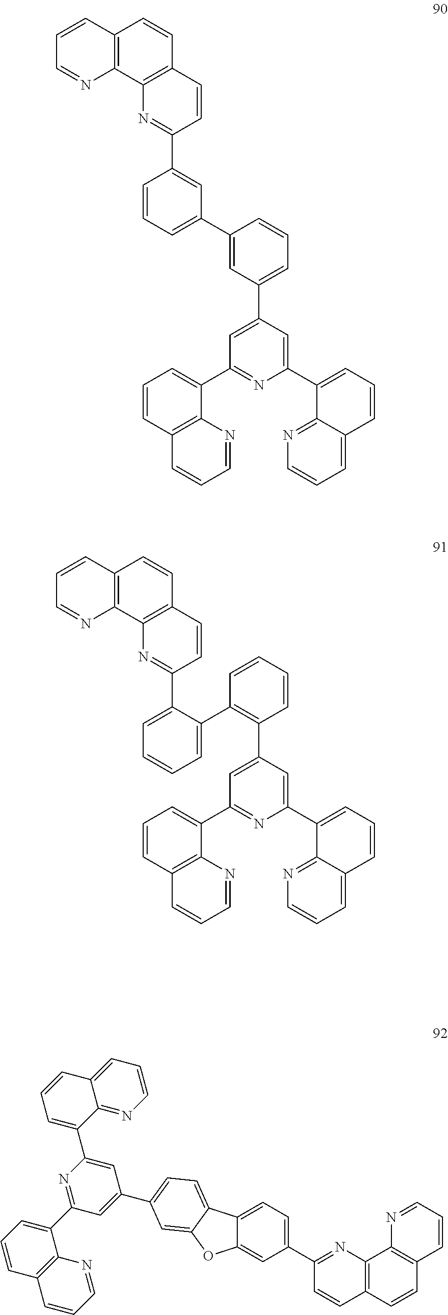 [SMILES: c1cc(-c2cccc(-c3ccc4ccc5cccnc5c4n3)c2)cc(-c2cc(-c3cccc4cccnc34)nc(-c3cccc4cccnc34)c2)c1 ; c1ccc(-c2ccccc2-c2ccc3ccc4cccnc4c3n2)c(-c2cc(-c3cccc4cccnc34)nc(-c3cccc4cccnc34)c2)c1 ; c1cnc2c(-c3cc(-c4ccc5c(c4)oc4cc(-c6ccc7ccc8cccnc8c7n6)ccc45)cc(-c4cccc5cccnc45)n3)cccc2c1]

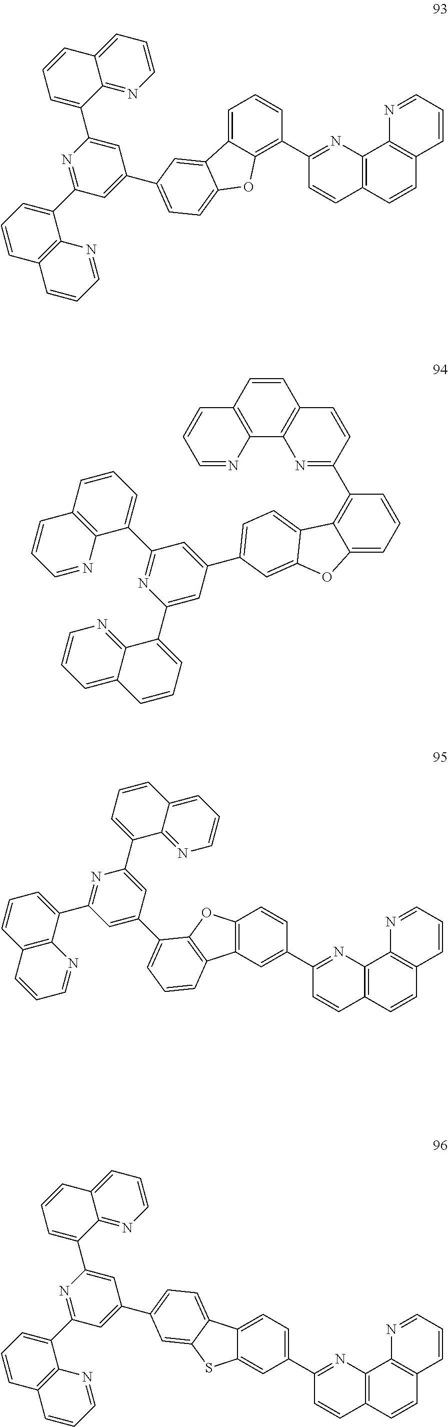 [SMILES: c1cnc2c(-c3cc(-c4ccc5oc6c(-c7ccc8ccc9cccnc9c8n7)cccc6c5c4)cc(-c4cccc5cccnc45)n3)cccc2c1 ; c1cnc2c(-c3cc(-c4ccc5c(c4)oc4cccc(-c6ccc7ccc8cccnc8c7n6)c45)cc(-c4cccc5cccnc45)n3)cccc2c1 ; c1cnc2c(-c3cc(-c4cccc5c4oc4ccc(-c6ccc7ccc8cccnc8c7n6)cc45)cc(-c4cccc5cccnc45)n3)cccc2c1 ; c1cnc2c(-c3cc(-c4ccc5c(c4)sc4cc(-c6ccc7ccc8cccnc8c7n6)ccc45)cc(-c4cccc5cccnc45)n3)cccc2c1]

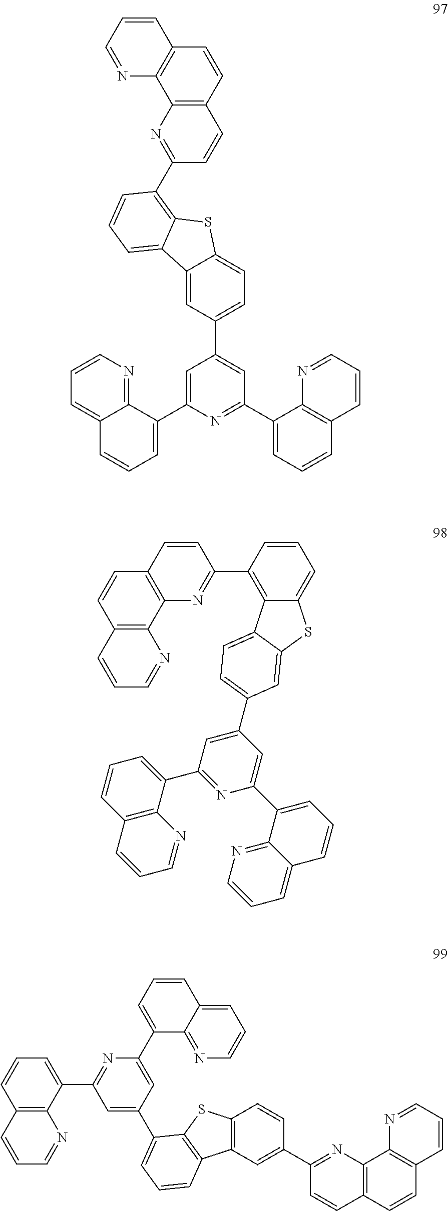 [SMILES: c1cnc2c(-c3cc(-c4ccc5sc6c(-c7ccc8ccc9cccnc9c8n7)cccc6c5c4)cc(-c4cccc5cccnc45)n3)cccc2c1 ; c1cnc2c(-c3cc(-c4ccc5c(c4)sc4cccc(-c6ccc7ccc8cccnc8c7n6)c45)cc(-c4cccc5cccnc45)n3)cccc2c1 ; c1cnc2c(-c3cc(-c4cccc5c4sc4ccc(-c6ccc7ccc8cccnc8c7n6)cc45)cc(-c4cccc5cccnc45)n3)cccc2c1]

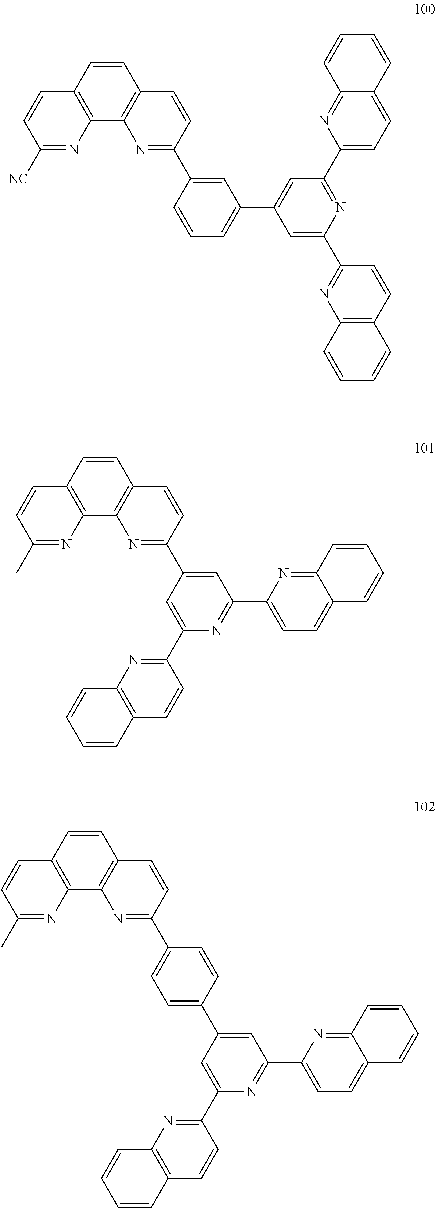 [SMILES: N#Cc1ccc2ccc3ccc(-c4cccc(-c5cc(-c6ccc7ccccc7n6)nc(-c6ccc7ccccc7n6)c5)c4)nc3c2n1 ; Cc1ccc2ccc3ccc(-c4cc(-c5ccc6ccccc6n5)nc(-c5ccc6ccccc6n5)c4)nc3c2n1 ; Cc1ccc2ccc3ccc(-c4ccc(-c5cc(-c6ccc7ccccc7n6)nc(-c6ccc7ccccc7n6)c5)cc4)nc3c2n1]

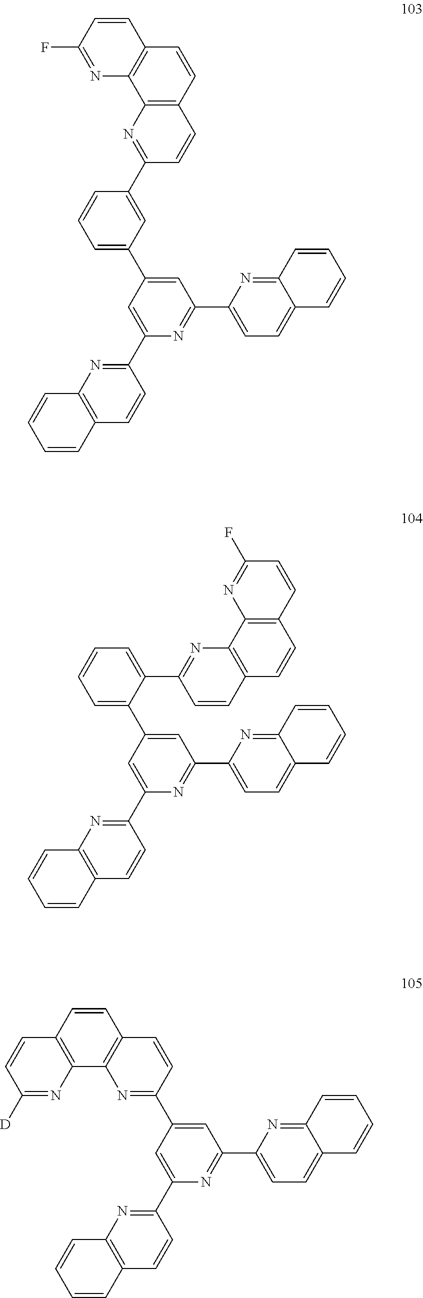 [SMILES: Fc1ccc2ccc3ccc(-c4cccc(-c5cc(-c6ccc7ccccc7n6)nc(-c6ccc7ccccc7n6)c5)c4)nc3c2n1 ; Fc1ccc2ccc3ccc(-c4ccccc4-c4cc(-c5ccc6ccccc6n5)nc(-c5ccc6ccccc6n5)c4)nc3c2n1 ; [2H]c1ccc2ccc3ccc(-c4cc(-c5ccc6ccccc6n5)nc(-c5ccc6ccccc6n5)c4)nc3c2n1]

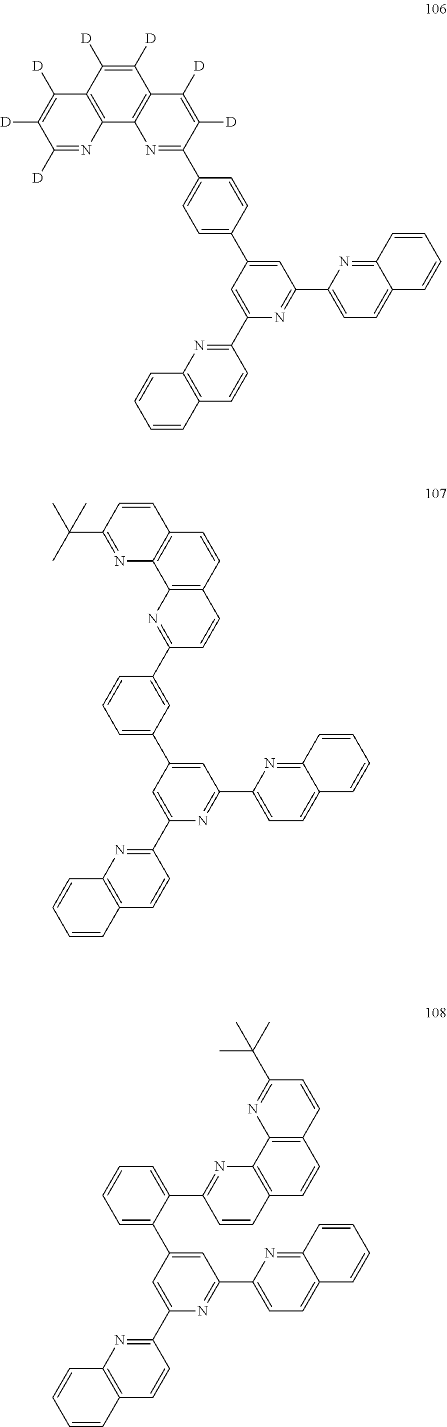 [SMILES: [2H]c1nc2c(c([2H])c1[2H])c([2H])c([2H])c1c([2H])c([2H])c(-c3ccc(-c4cc(-c5ccc6ccccc6n5)nc(-c5ccc6ccccc6n5)c4)cc3)nc12 ; CC(C)(C)c1ccc2ccc3ccc(-c4cccc(-c5cc(-c6ccc7ccccc7n6)nc(-c6ccc7ccccc7n6)c5)c4)nc3c2n1 ; CC(C)(C)c1ccc2ccc3ccc(-c4ccccc4-c4cc(-c5ccc6ccccc6n5)nc(-c5ccc6ccccc6n5)c4)nc3c2n1]

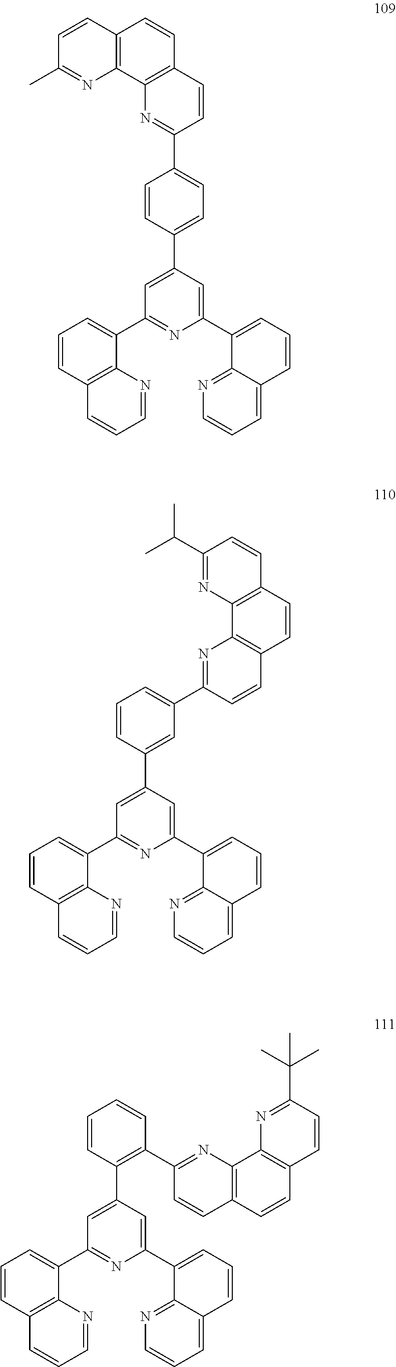 [SMILES: Cc1ccc2ccc3ccc(-c4ccc(-c5cc(-c6cccc7cccnc67)nc(-c6cccc7cccnc67)c5)cc4)nc3c2n1 ; CC(C)c1ccc2ccc3ccc(-c4cccc(-c5cc(-c6cccc7cccnc67)nc(-c6cccc7cccnc67)c5)c4)nc3c2n1 ; CC(C)(C)c1ccc2ccc3ccc(-c4ccccc4-c4cc(-c5cccc6cccnc56)nc(-c5cccc6cccnc56)c4)nc3c2n1]

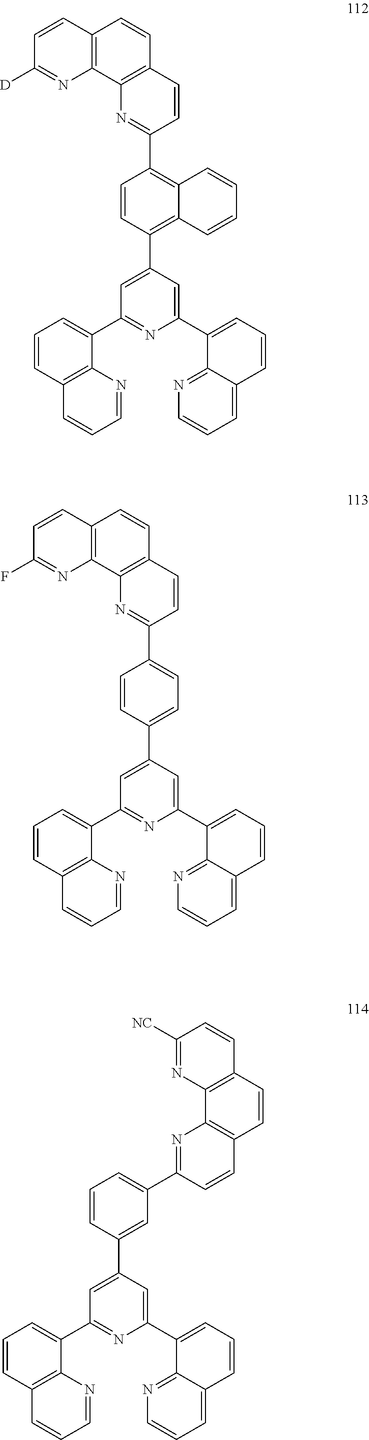 [SMILES: [2H]c1ccc2ccc3ccc(-c4ccc(-c5cc(-c6cccc7cccnc67)nc(-c6cccc7cccnc67)c5)c5ccccc45)nc3c2n1 ; Fc1ccc2ccc3ccc(-c4ccc(-c5cc(-c6cccc7cccnc67)nc(-c6cccc7cccnc67)c5)cc4)nc3c2n1 ; N#Cc1ccc2ccc3ccc(-c4cccc(-c5cc(-c6cccc7cccnc67)nc(-c6cccc7cccnc67)c5)c4)nc3c2n1]

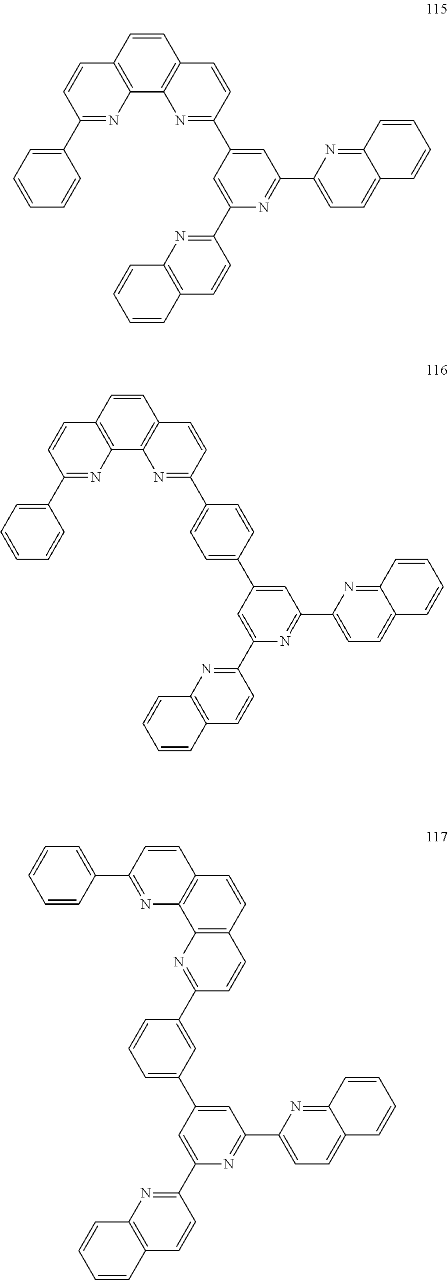 [SMILES: c1ccc(-c2ccc3ccc4ccc(-c5cc(-c6ccc7ccccc7n6)nc(-c6ccc7ccccc7n6)c5)nc4c3n2)cc1 ; c1ccc(-c2ccc3ccc4ccc(-c5ccc(-c6cc(-c7ccc8ccccc8n7)nc(-c7ccc8ccccc8n7)c6)cc5)nc4c3n2)cc1 ; c1ccc(-c2ccc3ccc4ccc(-c5cccc(-c6cc(-c7ccc8ccccc8n7)nc(-c7ccc8ccccc8n7)c6)c5)nc4c3n2)cc1]

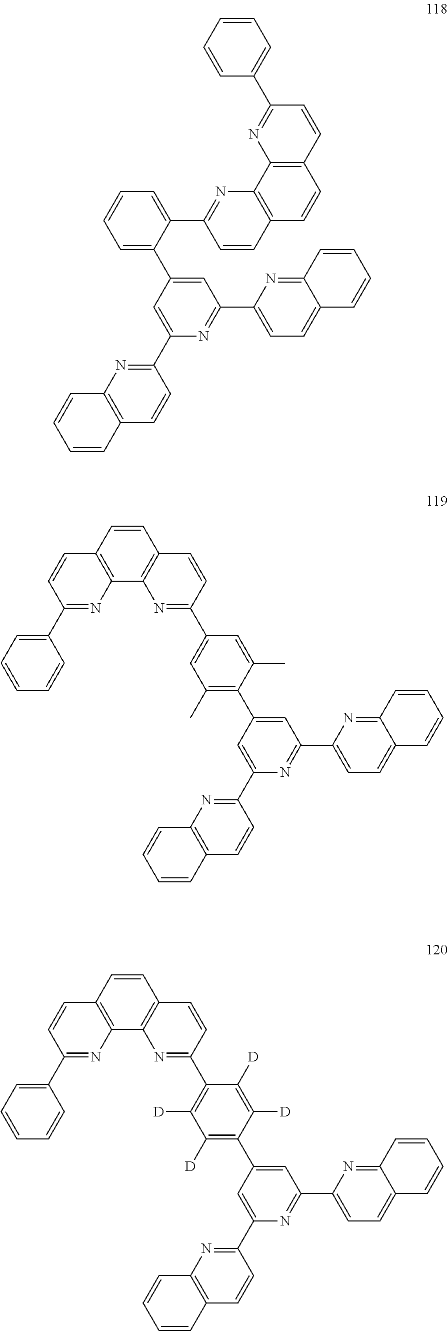 [SMILES: c1ccc(-c2ccc3ccc4ccc(-c5ccccc5-c5cc(-c6ccc7ccccc7n6)nc(-c6ccc7ccccc7n6)c5)nc4c3n2)cc1 ; Cc1cc(-c2ccc3ccc4ccc(-c5ccccc5)nc4c3n2)cc(C)c1-c1cc(-c2ccc3ccccc3n2)nc(-c2ccc3ccccc3n2)c1 ; [2H]c1c([2H])c(-c2ccc3ccc4ccc(-c5ccccc5)nc4c3n2)c([2H])c([2H])c1-c1cc(-c2ccc3ccccc3n2)nc(-c2ccc3ccccc3n2)c1]

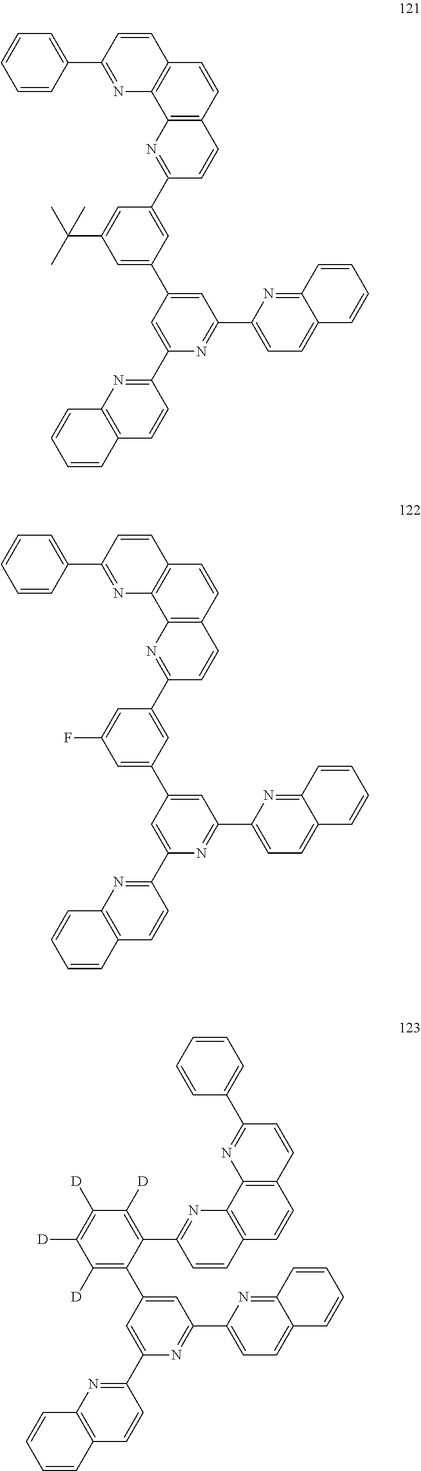 [SMILES: CC(C)(C)c1cc(-c2cc(-c3ccc4ccccc4n3)nc(-c3ccc4ccccc4n3)c2)cc(-c2ccc3ccc4ccc(-c5ccccc5)nc4c3n2)c1 ; Fc1cc(-c2cc(-c3ccc4ccccc4n3)nc(-c3ccc4ccccc4n3)c2)cc(-c2ccc3ccc4ccc(-c5ccccc5)nc4c3n2)c1 ; [2H]c1c([2H])c([2H])c(-c2ccc3ccc4ccc(-c5ccccc5)nc4c3n2)c(-c2cc(-c3ccc4ccccc4n3)nc(-c3ccc4ccccc4n3)c2)c1[2H]]

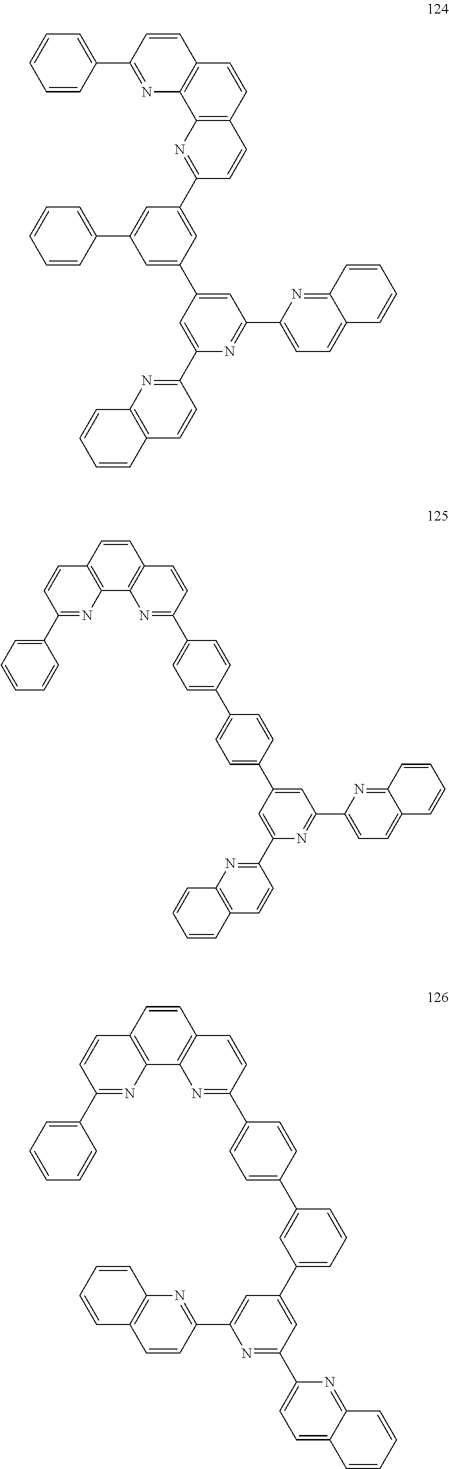 [SMILES: c1ccc(-c2cc(-c3cc(-c4ccc5ccccc5n4)nc(-c4ccc5ccccc5n4)c3)cc(-c3ccc4ccc5ccc(-c6ccccc6)nc5c4n3)c2)cc1 ; c1ccc(-c2ccc3ccc4ccc(-c5ccc(-c6ccc(-c7cc(-c8ccc9ccccc9n8)nc(-c8ccc9ccccc9n8)c7)cc6)cc5)nc4c3n2)cc1 ; c1ccc(-c2ccc3ccc4ccc(-c5ccc(-c6cccc(-c7cc(-c8ccc9ccccc9n8)nc(-c8ccc9ccccc9n8)c7)c6)cc5)nc4c3n2)cc1]

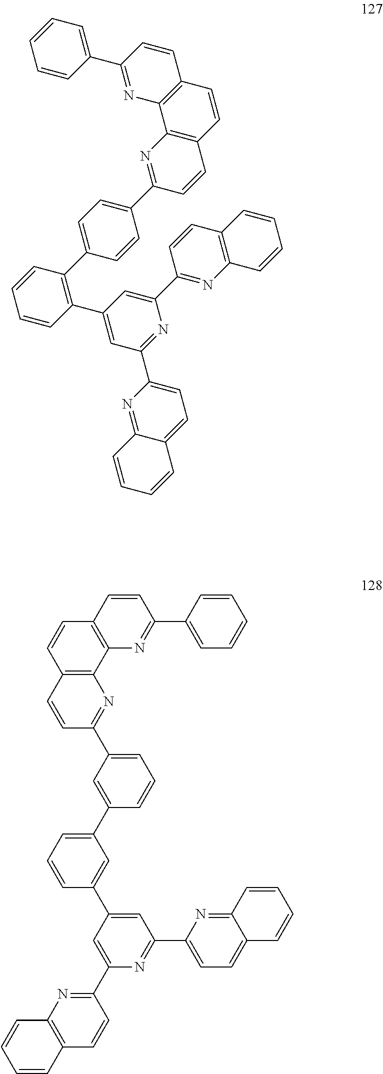 [SMILES: c1ccc(-c2ccc3ccc4ccc(-c5ccc(-c6ccccc6-c6cc(-c7ccc8ccccc8n7)nc(-c7ccc8ccccc8n7)c6)cc5)nc4c3n2)cc1 ; c1ccc(-c2ccc3ccc4ccc(-c5cccc(-c6cccc(-c7cc(-c8ccc9ccccc9n8)nc(-c8ccc9ccccc9n8)c7)c6)c5)nc4c3n2)cc1]

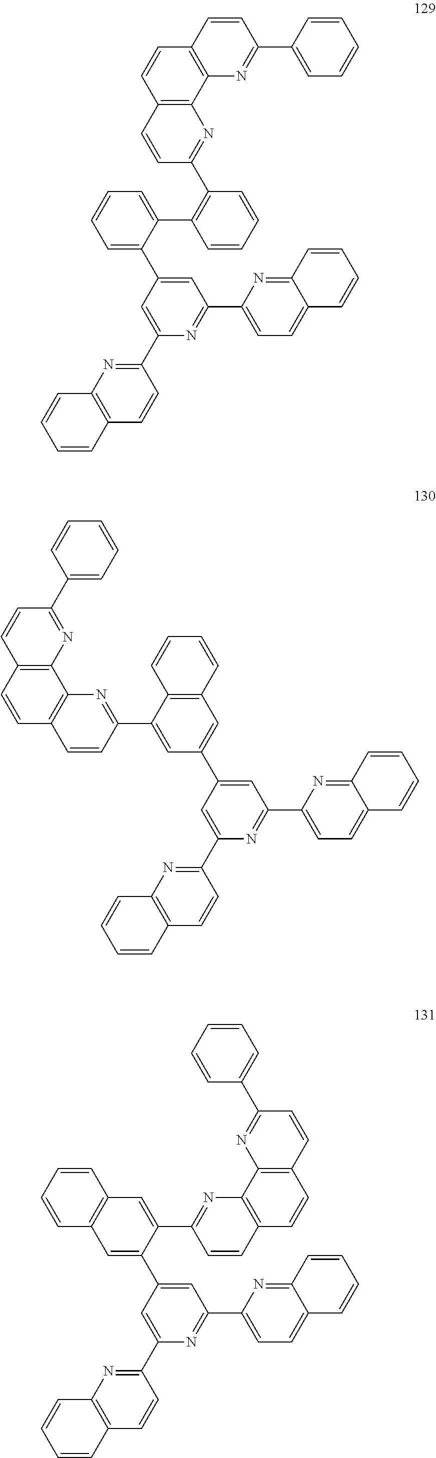 [SMILES: c1ccc(-c2ccc3ccc4ccc(-c5ccccc5-c5ccccc5-c5cc(-c6ccc7ccccc7n6)nc(-c6ccc7ccccc7n6)c5)nc4c3n2)cc1 ; c1ccc(-c2ccc3ccc4ccc(-c5cc(-c6cc(-c7ccc8ccccc8n7)nc(-c7ccc8ccccc8n7)c6)cc6ccccc56)nc4c3n2)cc1 ; c1ccc(-c2ccc3ccc4ccc(-c5cc6ccccc6cc5-c5cc(-c6ccc7ccccc7n6)nc(-c6ccc7ccccc7n6)c5)nc4c3n2)cc1]

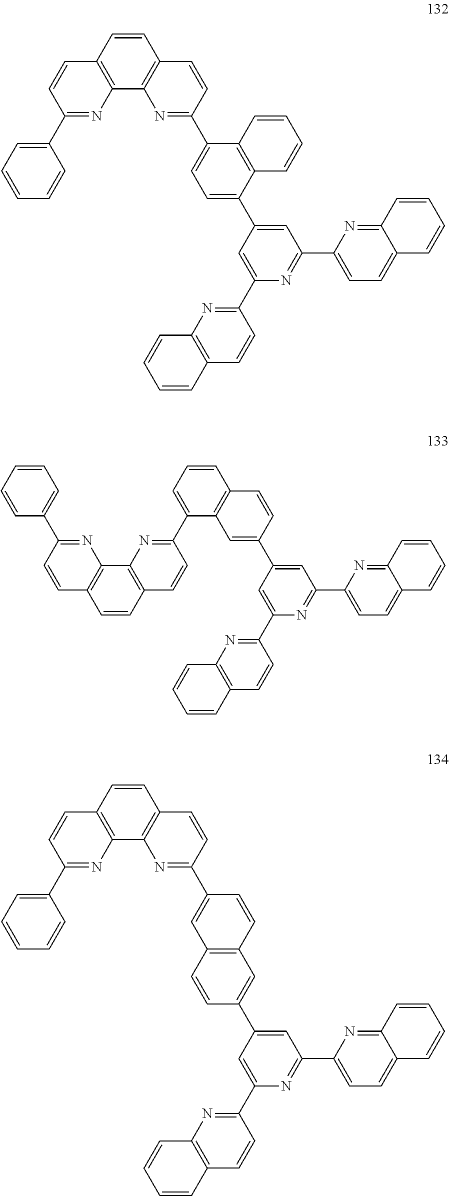 [SMILES: c1ccc(-c2ccc3ccc4ccc(-c5ccc(-c6cc(-c7ccc8ccccc8n7)nc(-c7ccc8ccccc8n7)c6)c6ccccc56)nc4c3n2)cc1 ; c1ccc(-c2ccc3ccc4ccc(-c5cccc6ccc(-c7cc(-c8ccc9ccccc9n8)nc(-c8ccc9ccccc9n8)c7)cc56)nc4c3n2)cc1 ; c1ccc(-c2ccc3ccc4ccc(-c5ccc6cc(-c7cc(-c8ccc9ccccc9n8)nc(-c8ccc9ccccc9n8)c7)ccc6c5)nc4c3n2)cc1]

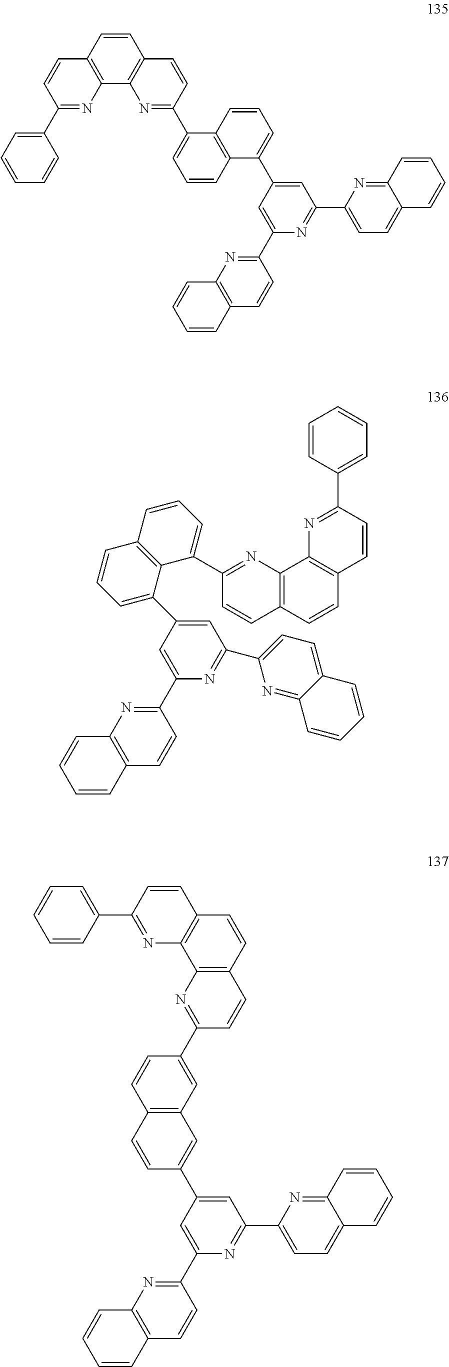 [SMILES: c1ccc(-c2ccc3ccc4ccc(-c5cccc6c(-c7cc(-c8ccc9ccccc9n8)nc(-c8ccc9ccccc9n8)c7)cccc56)nc4c3n2)cc1 ; c1ccc(-c2ccc3ccc4ccc(-c5cccc6cccc(-c7cc(-c8ccc9ccccc9n8)nc(-c8ccc9ccccc9n8)c7)c56)nc4c3n2)cc1 ; c1ccc(-c2ccc3ccc4ccc(-c5ccc6ccc(-c7cc(-c8ccc9ccccc9n8)nc(-c8ccc9ccccc9n8)c7)cc6c5)nc4c3n2)cc1]

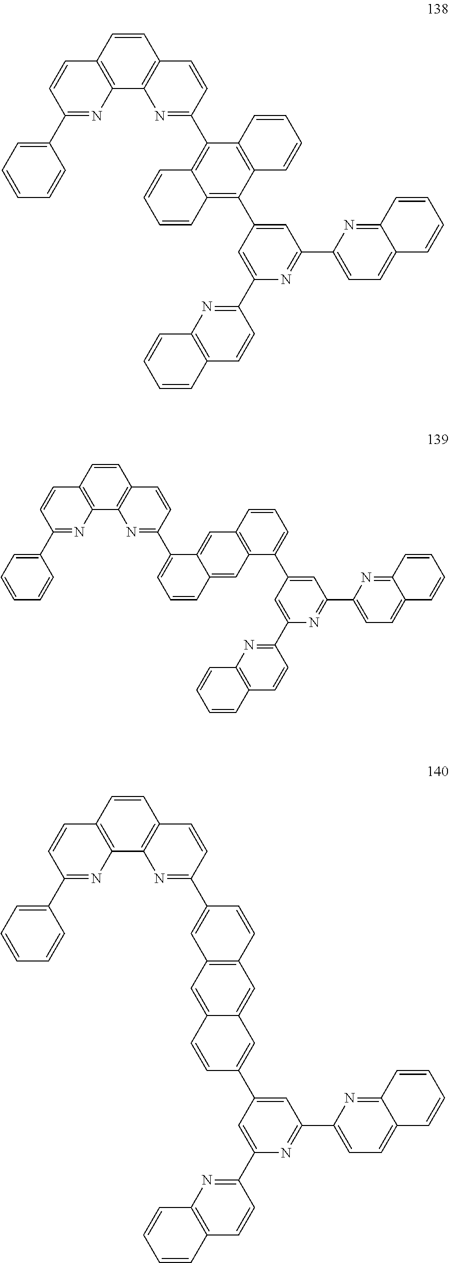 [SMILES: c1ccc(-c2ccc3ccc4ccc(-c5c6ccccc6c(-c6cc(-c7ccc8ccccc8n7)nc(-c7ccc8ccccc8n7)c6)c6ccccc56)nc4c3n2)cc1 ; c1ccc(-c2ccc3ccc4ccc(-c5cccc6cc7c(-c8cc(-c9ccc%10ccccc%10n9)nc(-c9ccc%10ccccc%10n9)c8)cccc7cc56)nc4c3n2)cc1 ; c1ccc(-c2ccc3ccc4ccc(-c5ccc6cc7cc(-c8cc(-c9ccc%10ccccc%10n9)nc(-c9ccc%10ccccc%10n9)c8)ccc7cc6c5)nc4c3n2)cc1]

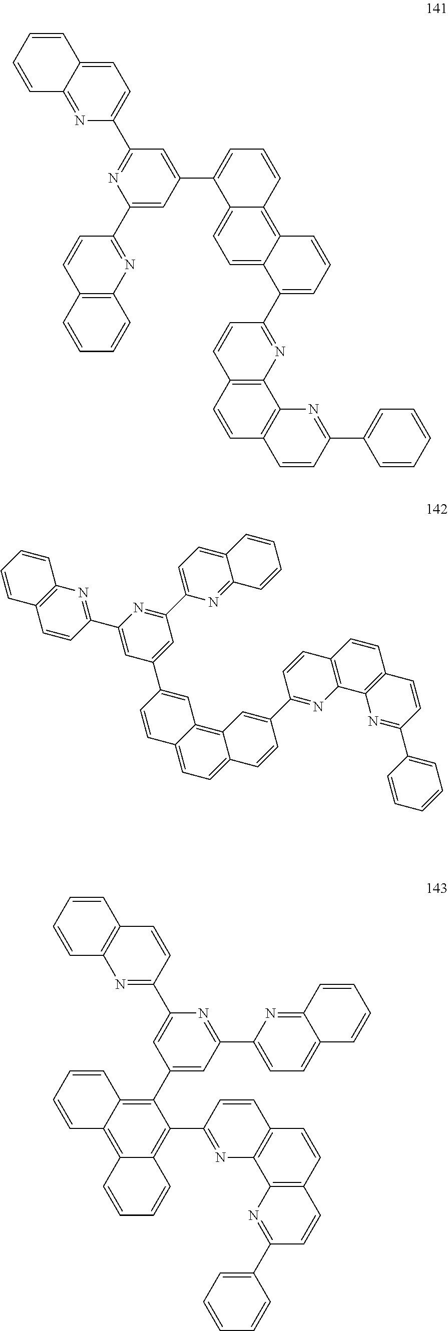 [SMILES: c1ccc(-c2ccc3ccc4ccc(-c5cccc6c5ccc5c(-c7cc(-c8ccc9ccccc9n8)nc(-c8ccc9ccccc9n8)c7)cccc56)nc4c3n2)cc1 ; c1ccc(-c2ccc3ccc4ccc(-c5ccc6ccc7ccc(-c8cc(-c9ccc%10ccccc%10n9)nc(-c9ccc%10ccccc%10n9)c8)cc7c6c5)nc4c3n2)cc1 ; c1ccc(-c2ccc3ccc4ccc(-c5c(-c6cc(-c7ccc8ccccc8n7)nc(-c7ccc8ccccc8n7)c6)c6ccccc6c6ccccc56)nc4c3n2)cc1]

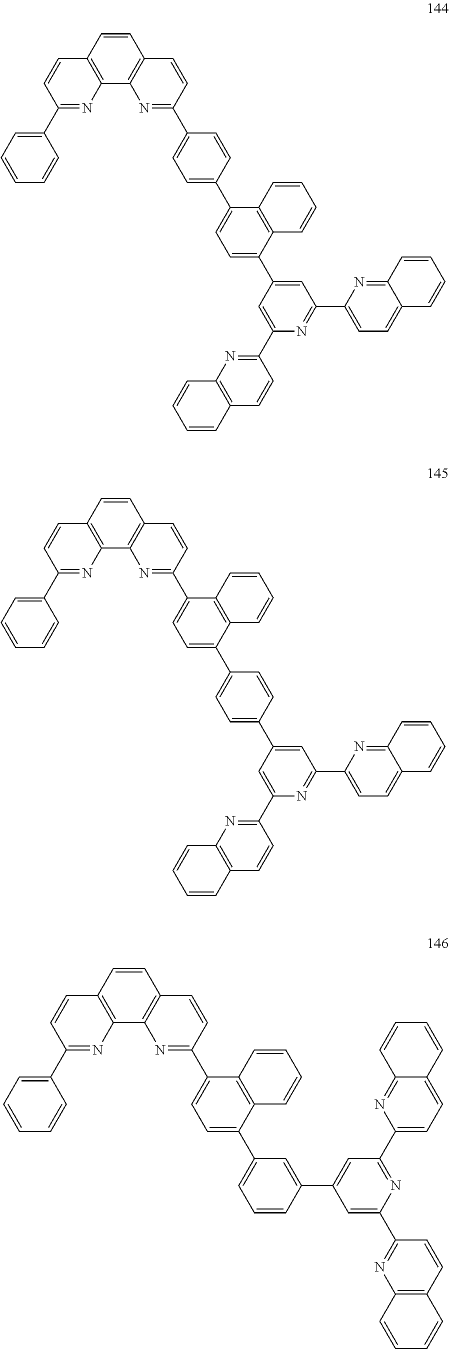 [SMILES: c1ccc(-c2ccc3ccc4ccc(-c5ccc(-c6ccc(-c7cc(-c8ccc9ccccc9n8)nc(-c8ccc9ccccc9n8)c7)c7ccccc67)cc5)nc4c3n2)cc1 ; c1ccc(-c2ccc3ccc4ccc(-c5ccc(-c6ccc(-c7cc(-c8ccc9ccccc9n8)nc(-c8ccc9ccccc9n8)c7)cc6)c6ccccc56)nc4c3n2)cc1 ; c1ccc(-c2ccc3ccc4ccc(-c5ccc(-c6cccc(-c7cc(-c8ccc9ccccc9n8)nc(-c8ccc9ccccc9n8)c7)c6)c6ccccc56)nc4c3n2)cc1]

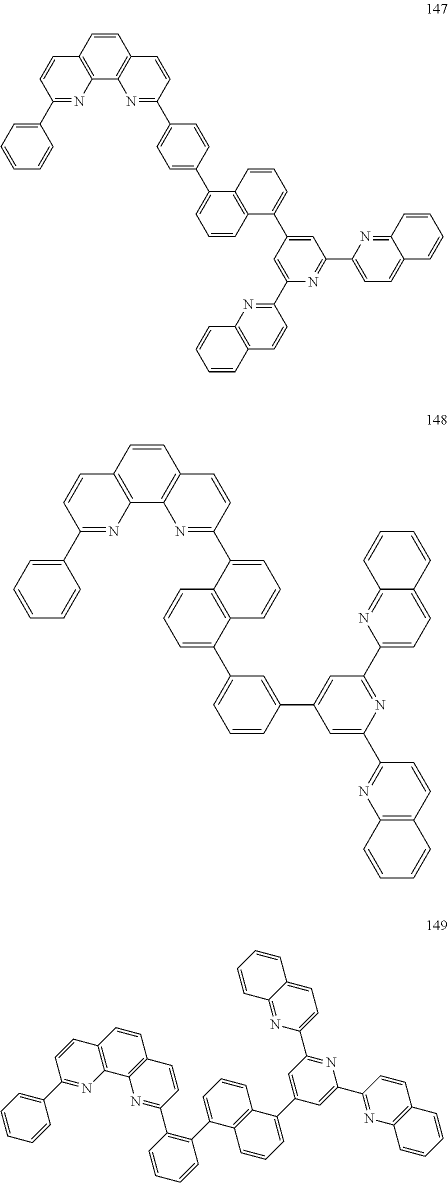 [SMILES: c1ccc(-c2ccc3ccc4ccc(-c5ccc(-c6cccc7c(-c8cc(-c9ccc%10ccccc%10n9)nc(-c9ccc%10ccccc%10n9)c8)cccc67)cc5)nc4c3n2)cc1 ; c1ccc(-c2ccc3ccc4ccc(-c5cccc6c(-c7cccc(-c8cc(-c9ccc%10ccccc%10n9)nc(-c9ccc%10ccccc%10n9)c8)c7)cccc56)nc4c3n2)cc1 ; c1ccc(-c2ccc3ccc4ccc(-c5ccccc5-c5cccc6c(-c7cc(-c8ccc9ccccc9n8)nc(-c8ccc9ccccc9n8)c7)cccc56)nc4c3n2)cc1]

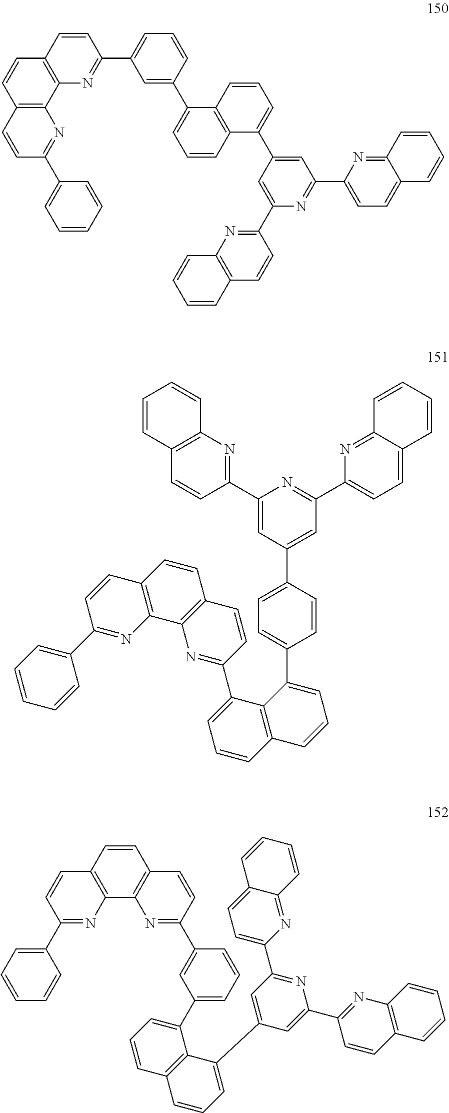 [SMILES: c1ccc(-c2ccc3ccc4ccc(-c5cccc(-c6cccc7c(-c8cc(-c9ccc%10ccccc%10n9)nc(-c9ccc%10ccccc%10n9)c8)cccc67)c5)nc4c3n2)cc1 ; c1ccc(-c2ccc3ccc4ccc(-c5cccc6cccc(-c7ccc(-c8cc(-c9ccc%10ccccc%10n9)nc(-c9ccc%10ccccc%10n9)c8)cc7)c56)nc4c3n2)cc1 ; c1ccc(-c2ccc3ccc4ccc(-c5cccc(-c6cccc7cccc(-c8cc(-c9ccc%10ccccc%10n9)nc(-c9ccc%10ccccc%10n9)c8)c67)c5)nc4c3n2)cc1]

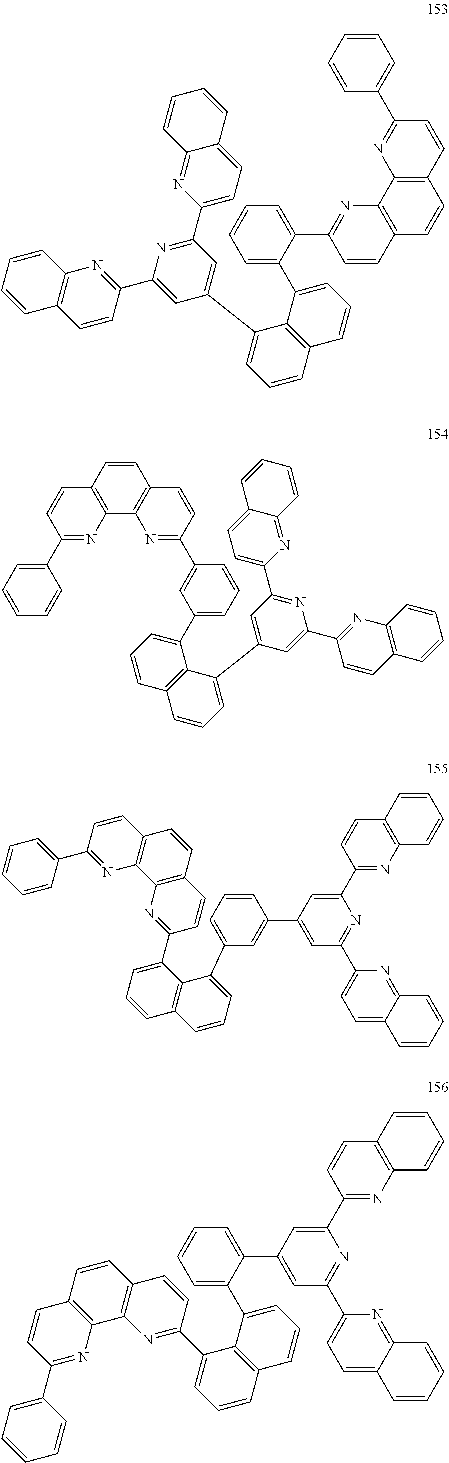 [SMILES: c1ccc(-c2ccc3ccc4ccc(-c5ccccc5-c5cccc6cccc(-c7cc(-c8ccc9ccccc9n8)nc(-c8ccc9ccccc9n8)c7)c56)nc4c3n2)cc1 ; c1ccc(-c2ccc3ccc4ccc(-c5cccc(-c6cccc7cccc(-c8cc(-c9ccc%10ccccc%10n9)nc(-c9ccc%10ccccc%10n9)c8)c67)c5)nc4c3n2)cc1 ; c1ccc(-c2ccc3ccc4ccc(-c5cccc6cccc(-c7cccc(-c8cc(-c9ccc%10ccccc%10n9)nc(-c9ccc%10ccccc%10n9)c8)c7)c56)nc4c3n2)cc1 ; c1ccc(-c2ccc3ccc4ccc(-c5cccc6cccc(-c7ccccc7-c7cc(-c8ccc9ccccc9n8)nc(-c8ccc9ccccc9n8)c7)c56)nc4c3n2)cc1]

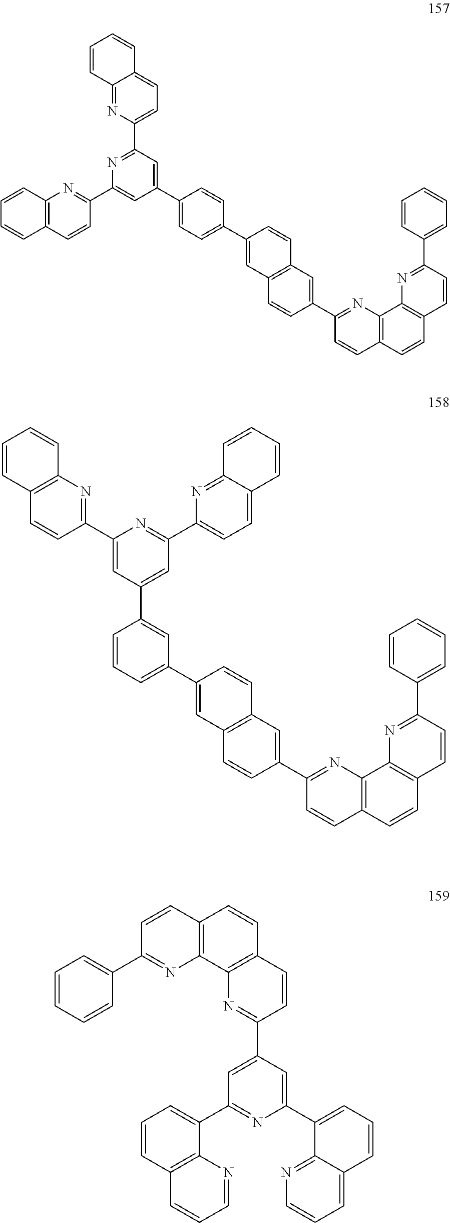 [SMILES: c1ccc(-c2ccc3ccc4ccc(-c5ccc6cc(-c7ccc(-c8cc(-c9ccc%10ccccc%10n9)nc(-c9ccc%10ccccc%10n9)c8)cc7)ccc6c5)nc4c3n2)cc1 ; c1ccc(-c2ccc3ccc4ccc(-c5ccc6cc(-c7cccc(-c8cc(-c9ccc%10ccccc%10n9)nc(-c9ccc%10ccccc%10n9)c8)c7)ccc6c5)nc4c3n2)cc1 ; c1ccc(-c2ccc3ccc4ccc(-c5cc(-c6cccc7cccnc67)nc(-c6cccc7cccnc67)c5)nc4c3n2)cc1]

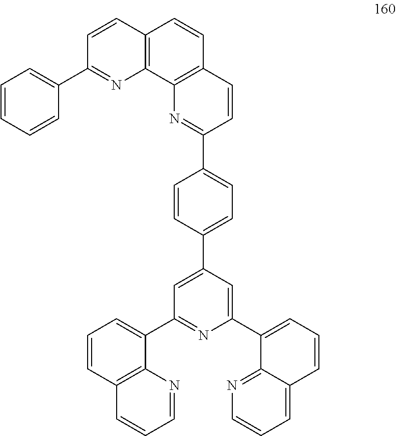 [SMILES: c1ccc(-c2ccc3ccc4ccc(-c5ccc(-c6cc(-c7cccc8cccnc78)nc(-c7cccc8cccnc78)c6)cc5)nc4c3n2)cc1]

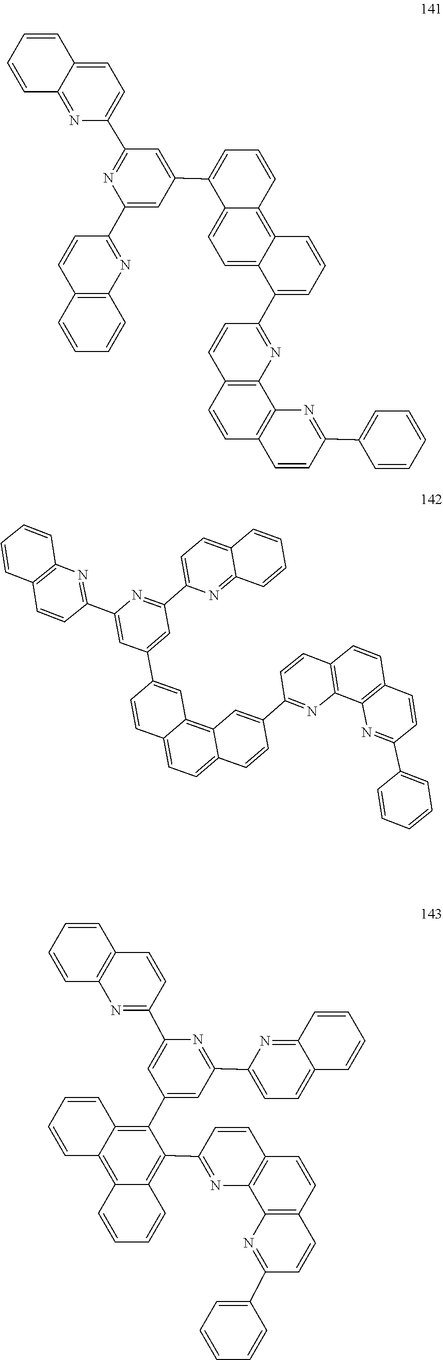 [SMILES: c1ccc(-c2ccc3ccc4ccc(-c5cccc6c5ccc5c(-c7cc(-c8ccc9ccccc9n8)nc(-c8ccc9ccccc9n8)c7)cccc56)nc4c3n2)cc1 ; c1ccc(-c2ccc3ccc4ccc(-c5ccc6ccc7ccc(-c8cc(-c9ccc%10ccccc%10n9)nc(-c9ccc%10ccccc%10n9)c8)cc7c6c5)nc4c3n2)cc1 ; c1ccc(-c2ccc3ccc4ccc(-c5c(-c6cc(-c7ccc8ccccc8n7)nc(-c7ccc8ccccc8n7)c6)c6ccccc6c6ccccc56)nc4c3n2)cc1]

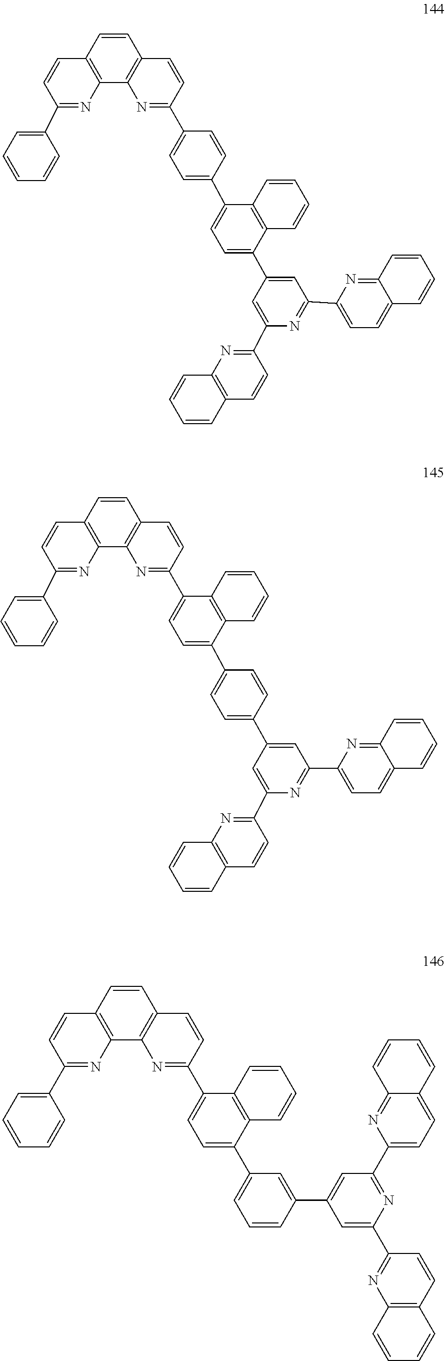 [SMILES: c1ccc(-c2ccc3ccc4ccc(-c5ccc(-c6ccc(-c7cc(-c8ccc9ccccc9n8)nc(-c8ccc9ccccc9n8)c7)c7ccccc67)cc5)nc4c3n2)cc1 ; c1ccc(-c2ccc3ccc4ccc(-c5ccc(-c6ccc(-c7cc(-c8ccc9ccccc9n8)nc(-c8ccc9ccccc9n8)c7)cc6)c6ccccc56)nc4c3n2)cc1 ; c1ccc(-c2ccc3ccc4ccc(-c5ccc(-c6cccc(-c7cc(-c8ccc9ccccc9n8)nc(-c8ccc9ccccc9n8)c7)c6)c6ccccc56)nc4c3n2)cc1]

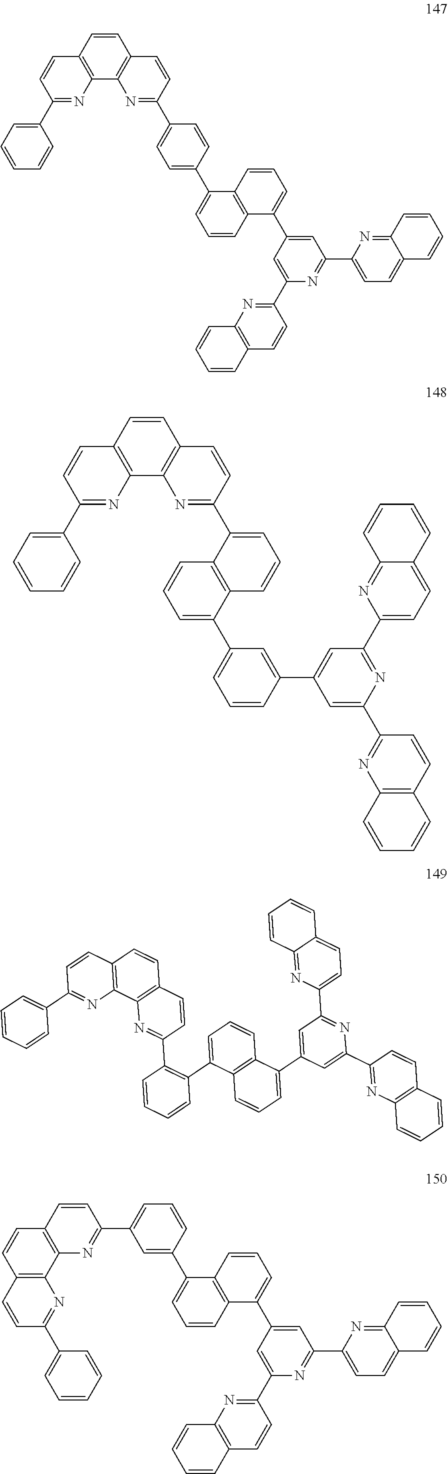 [SMILES: c1ccc(-c2ccc3ccc4ccc(-c5ccc(-c6cccc7c(-c8cc(-c9ccc%10ccccc%10n9)nc(-c9ccc%10ccccc%10n9)c8)cccc67)cc5)nc4c3n2)cc1 ; c1ccc(-c2ccc3ccc4ccc(-c5cccc6c(-c7cccc(-c8cc(-c9ccc%10ccccc%10n9)nc(-c9ccc%10ccccc%10n9)c8)c7)cccc56)nc4c3n2)cc1 ; c1ccc(-c2ccc3ccc4ccc(-c5ccccc5-c5cccc6c(-c7cc(-c8ccc9ccccc9n8)nc(-c8ccc9ccccc9n8)c7)cccc56)nc4c3n2)cc1 ; c1ccc(-c2ccc3ccc4ccc(-c5cccc(-c6cccc7c(-c8cc(-c9ccc%10ccccc%10n9)nc(-c9ccc%10ccccc%10n9)c8)cccc67)c5)nc4c3n2)cc1]

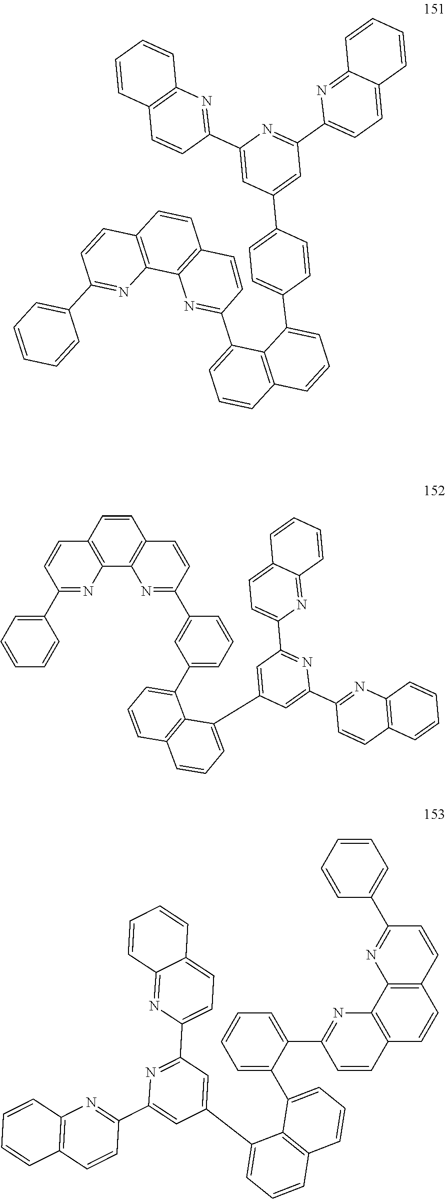 [SMILES: c1ccc(-c2ccc3ccc4ccc(-c5cccc6cccc(-c7ccc(-c8cc(-c9ccc%10ccccc%10n9)nc(-c9ccc%10ccccc%10n9)c8)cc7)c56)nc4c3n2)cc1 ; c1ccc(-c2ccc3ccc4ccc(-c5cccc(-c6cccc7cccc(-c8cc(-c9ccc%10ccccc%10n9)nc(-c9ccc%10ccccc%10n9)c8)c67)c5)nc4c3n2)cc1 ; c1ccc(-c2ccc3ccc4ccc(-c5ccccc5-c5cccc6cccc(-c7cc(-c8ccc9ccccc9n8)nc(-c8ccc9ccccc9n8)c7)c56)nc4c3n2)cc1]

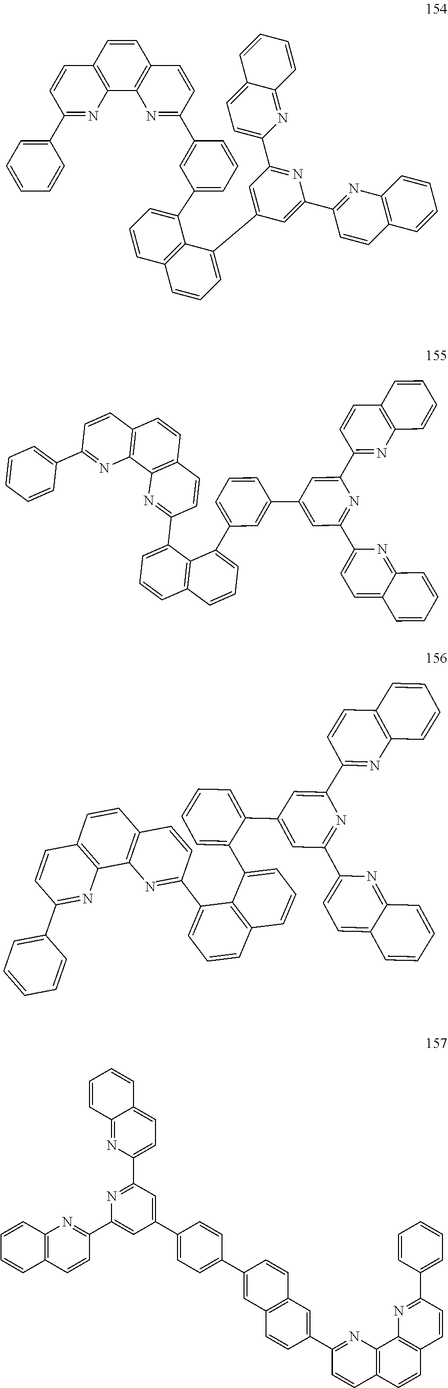 [SMILES: c1ccc(-c2ccc3ccc4ccc(-c5cccc(-c6cccc7cccc(-c8cc(-c9ccc%10ccccc%10n9)nc(-c9ccc%10ccccc%10n9)c8)c67)c5)nc4c3n2)cc1 ; c1ccc(-c2ccc3ccc4ccc(-c5cccc6cccc(-c7cccc(-c8cc(-c9ccc%10ccccc%10n9)nc(-c9ccc%10ccccc%10n9)c8)c7)c56)nc4c3n2)cc1 ; c1ccc(-c2ccc3ccc4ccc(-c5cccc6cccc(-c7ccccc7-c7cc(-c8ccc9ccccc9n8)nc(-c8ccc9ccccc9n8)c7)c56)nc4c3n2)cc1 ; c1ccc(-c2ccc3ccc4ccc(-c5ccc6cc(-c7ccc(-c8cc(-c9ccc%10ccccc%10n9)nc(-c9ccc%10ccccc%10n9)c8)cc7)ccc6c5)nc4c3n2)cc1]

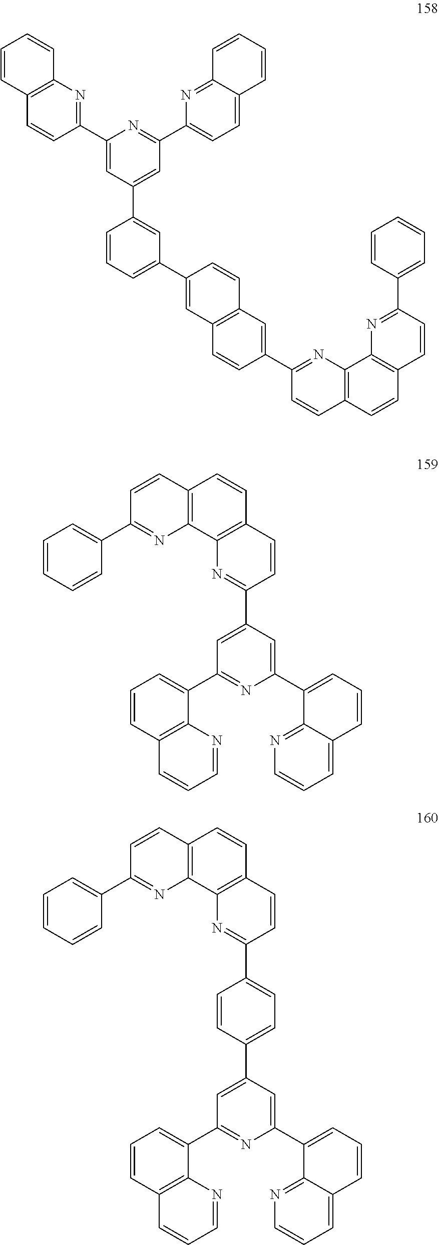 [SMILES: c1ccc(-c2ccc3ccc4ccc(-c5ccc6cc(-c7cccc(-c8cc(-c9ccc%10ccccc%10n9)nc(-c9ccc%10ccccc%10n9)c8)c7)ccc6c5)nc4c3n2)cc1 ; c1ccc(-c2ccc3ccc4ccc(-c5cc(-c6cccc7cccnc67)nc(-c6cccc7cccnc67)c5)nc4c3n2)cc1 ; c1ccc(-c2ccc3ccc4ccc(-c5ccc(-c6cc(-c7cccc8cccnc78)nc(-c7cccc8cccnc78)c6)cc5)nc4c3n2)cc1]

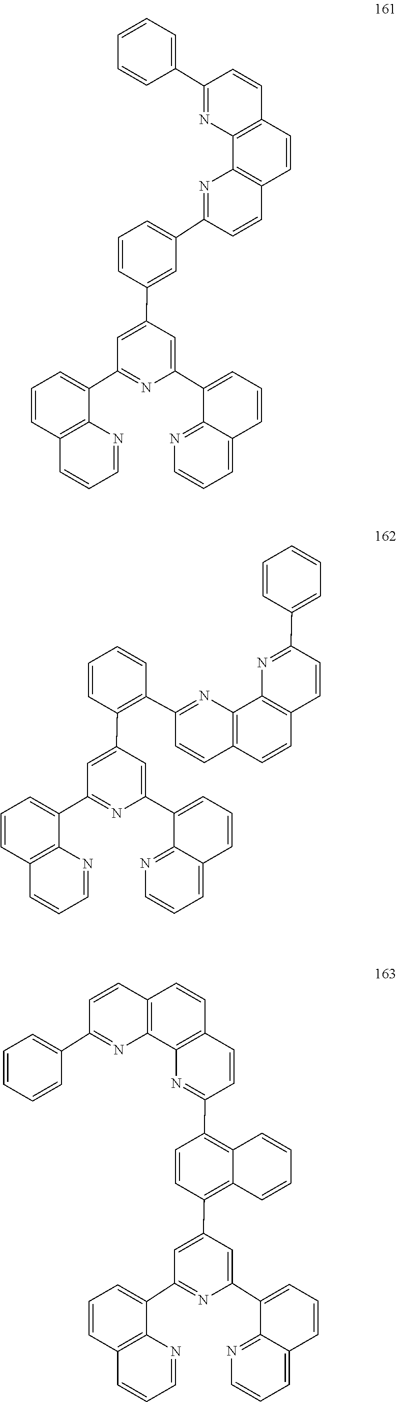 [SMILES: c1ccc(-c2ccc3ccc4ccc(-c5cccc(-c6cc(-c7cccc8cccnc78)nc(-c7cccc8cccnc78)c6)c5)nc4c3n2)cc1 ; c1ccc(-c2ccc3ccc4ccc(-c5ccccc5-c5cc(-c6cccc7cccnc67)nc(-c6cccc7cccnc67)c5)nc4c3n2)cc1 ; c1ccc(-c2ccc3ccc4ccc(-c5ccc(-c6cc(-c7cccc8cccnc78)nc(-c7cccc8cccnc78)c6)c6ccccc56)nc4c3n2)cc1]

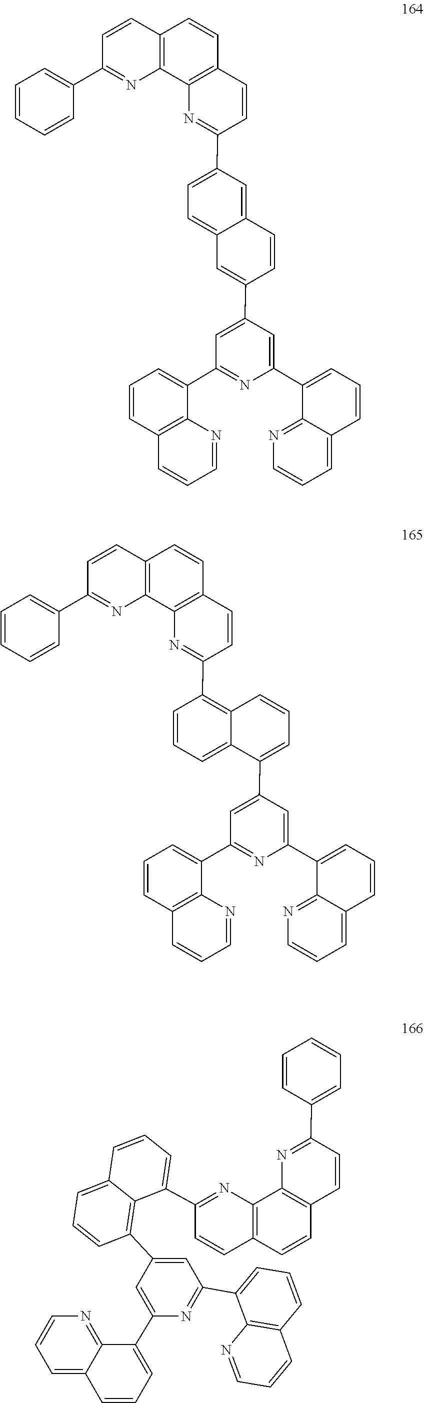 [SMILES: c1ccc(-c2ccc3ccc4ccc(-c5ccc6cc(-c7cc(-c8cccc9cccnc89)nc(-c8cccc9cccnc89)c7)ccc6c5)nc4c3n2)cc1 ; c1ccc(-c2ccc3ccc4ccc(-c5cccc6c(-c7cc(-c8cccc9cccnc89)nc(-c8cccc9cccnc89)c7)cccc56)nc4c3n2)cc1 ; c1ccc(-c2ccc3ccc4ccc(-c5cccc6cccc(-c7cc(-c8cccc9cccnc89)nc(-c8cccc9cccnc89)c7)c56)nc4c3n2)cc1]

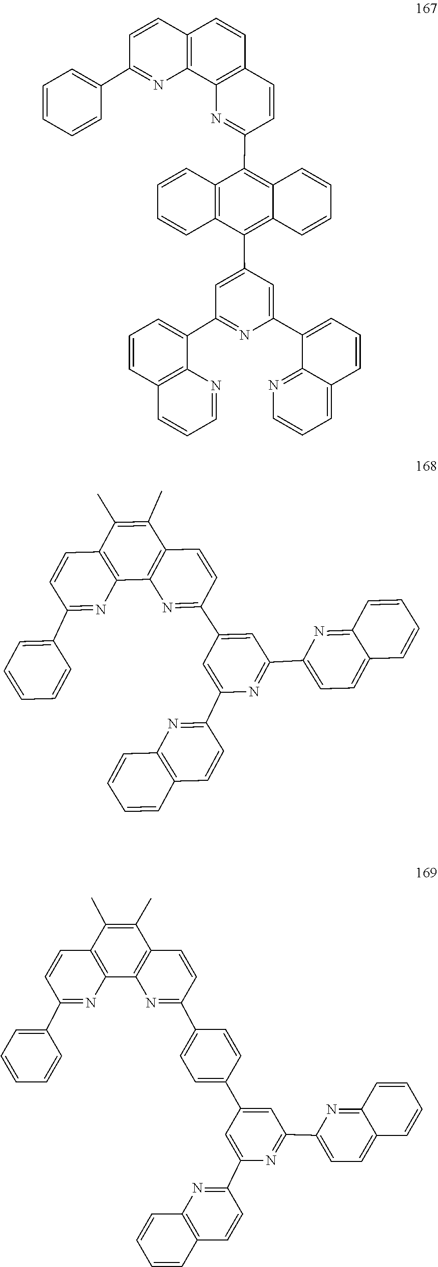 [SMILES: c1ccc(-c2ccc3ccc4ccc(-c5c6ccccc6c(-c6cc(-c7cccc8cccnc78)nc(-c7cccc8cccnc78)c6)c6ccccc56)nc4c3n2)cc1 ; Cc1c(C)c2ccc(-c3cc(-c4ccc5ccccc5n4)nc(-c4ccc5ccccc5n4)c3)nc2c2nc(-c3ccccc3)ccc12 ; Cc1c(C)c2ccc(-c3ccc(-c4cc(-c5ccc6ccccc6n5)nc(-c5ccc6ccccc6n5)c4)cc3)nc2c2nc(-c3ccccc3)ccc12]

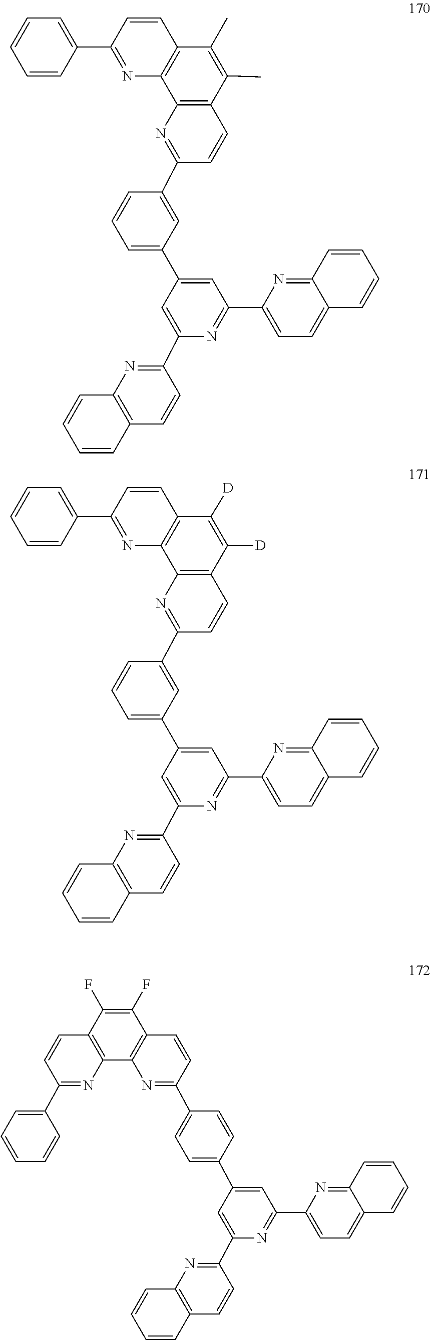

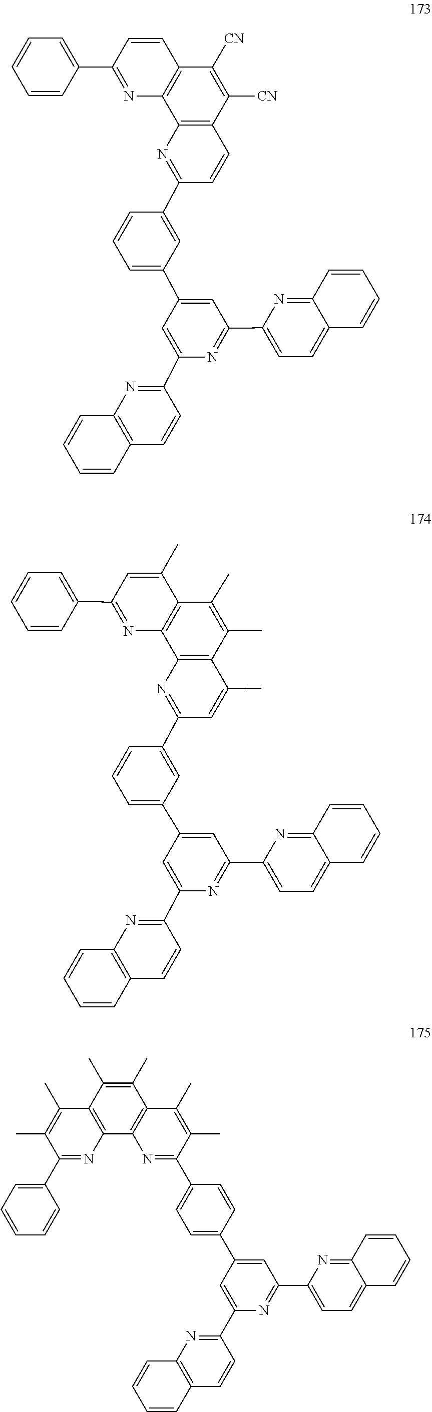 [SMILES: N#Cc1c(C#N)c2ccc(-c3cccc(-c4cc(-c5ccc6ccccc6n5)nc(-c5ccc6ccccc6n5)c4)c3)nc2c2nc(-c3ccccc3)ccc12 ; Cc1cc(-c2ccccc2)nc2c1c(C)c(C)c1c(C)cc(-c3cccc(-c4cc(-c5ccc6ccccc6n5)nc(-c5ccc6ccccc6n5)c4)c3)nc12 ; Cc1c(-c2ccccc2)nc2c(c1C)c(C)c(C)c1c(C)c(C)c(-c3ccc(-c4cc(-c5ccc6ccccc6n5)nc(-c5ccc6ccccc6n5)c4)cc3)nc12]

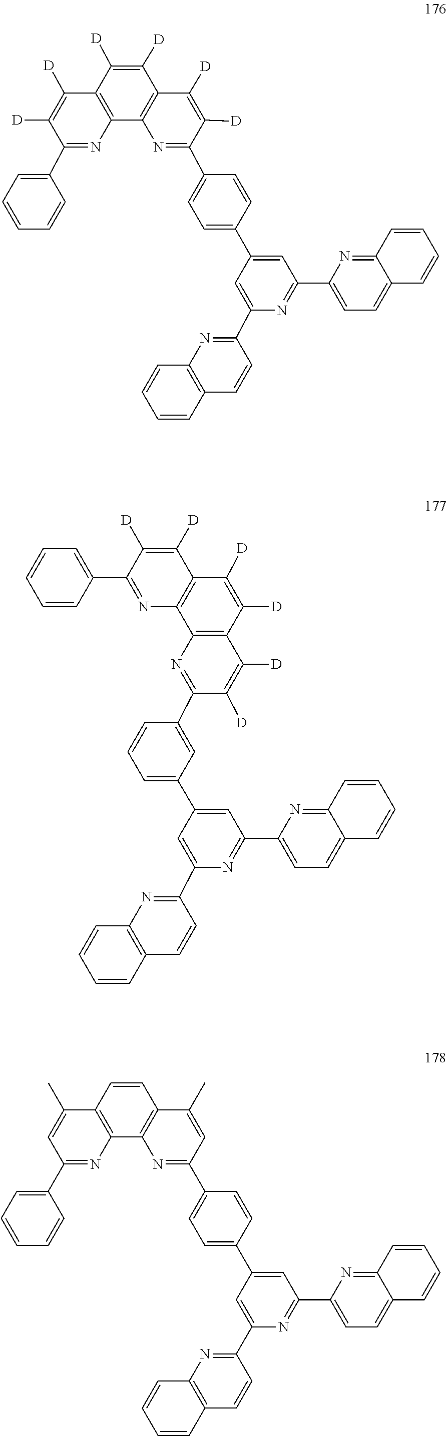 [SMILES: [2H]c1c(-c2ccccc2)nc2c(c1[2H])c([2H])c([2H])c1c([2H])c([2H])c(-c3ccc(-c4cc(-c5ccc6ccccc6n5)nc(-c5ccc6ccccc6n5)c4)cc3)nc12 ; [2H]c1c(-c2ccccc2)nc2c(c1[2H])c([2H])c([2H])c1c([2H])c([2H])c(-c3cccc(-c4cc(-c5ccc6ccccc6n5)nc(-c5ccc6ccccc6n5)c4)c3)nc12 ; Cc1cc(-c2ccccc2)nc2c1ccc1c(C)cc(-c3ccc(-c4cc(-c5ccc6ccccc6n5)nc(-c5ccc6ccccc6n5)c4)cc3)nc12]

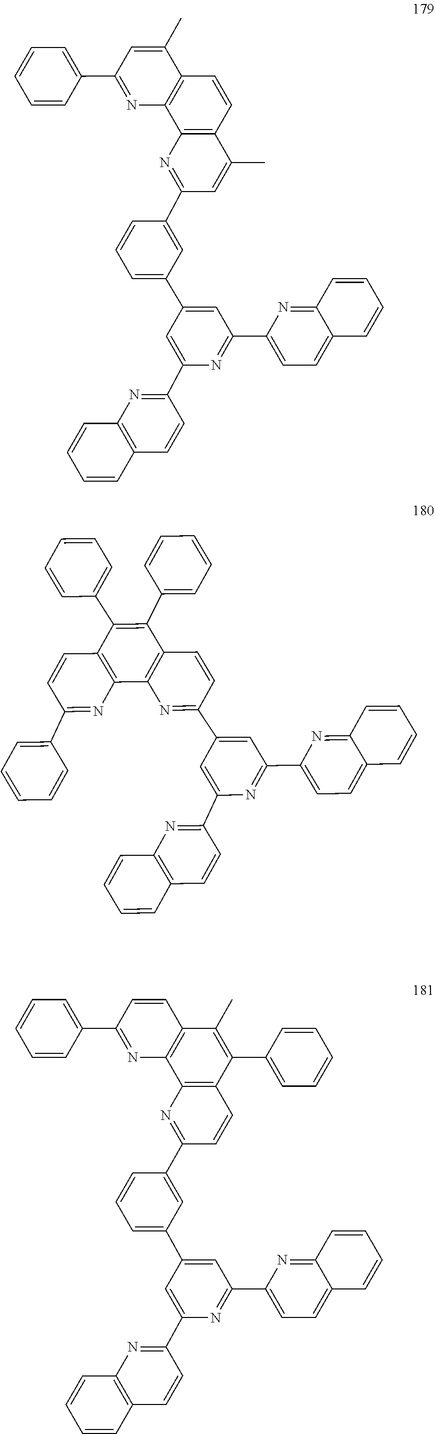 [SMILES: Cc1cc(-c2ccccc2)nc2c1ccc1c(C)cc(-c3cccc(-c4cc(-c5ccc6ccccc6n5)nc(-c5ccc6ccccc6n5)c4)c3)nc12 ; c1ccc(-c2ccc3c(-c4ccccc4)c(-c4ccccc4)c4ccc(-c5cc(-c6ccc7ccccc7n6)nc(-c6ccc7ccccc7n6)c5)nc4c3n2)cc1 ; Cc1c(-c2ccccc2)c2ccc(-c3cccc(-c4cc(-c5ccc6ccccc6n5)nc(-c5ccc6ccccc6n5)c4)c3)nc2c2nc(-c3ccccc3)ccc12]

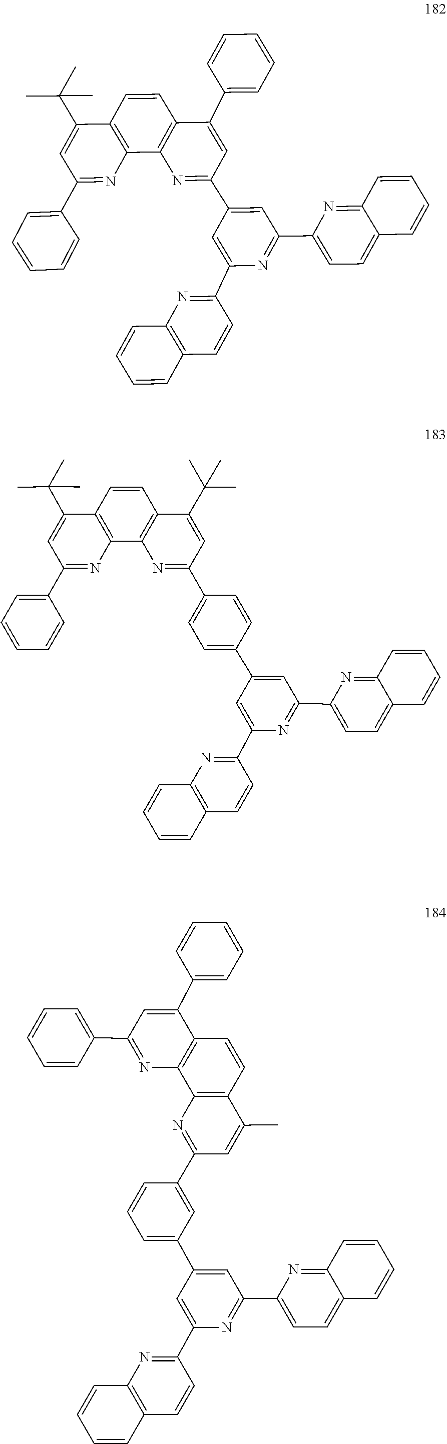 [SMILES: CC(C)(C)c1cc(-c2ccccc2)nc2c1ccc1c(-c3ccccc3)cc(-c3cc(-c4ccc5ccccc5n4)nc(-c4ccc5ccccc5n4)c3)nc12 ; CC(C)(C)c1cc(-c2ccccc2)nc2c1ccc1c(C(C)(C)C)cc(-c3ccc(-c4cc(-c5ccc6ccccc6n5)nc(-c5ccc6ccccc6n5)c4)cc3)nc12 ; Cc1cc(-c2cccc(-c3cc(-c4ccc5ccccc5n4)nc(-c4ccc5ccccc5n4)c3)c2)nc2c1ccc1c(-c3ccccc3)cc(-c3ccccc3)nc12]

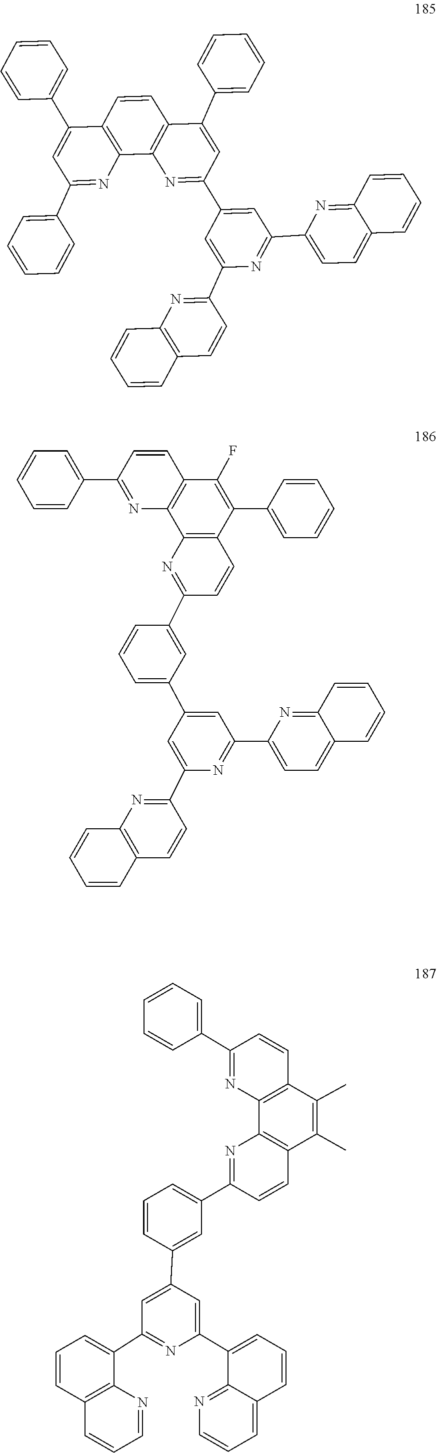 [SMILES: c1ccc(-c2cc(-c3ccccc3)c3ccc4c(-c5ccccc5)cc(-c5cc(-c6ccc7ccccc7n6)nc(-c6ccc7ccccc7n6)c5)nc4c3n2)cc1 ; Fc1c(-c2ccccc2)c2ccc(-c3cccc(-c4cc(-c5ccc6ccccc6n5)nc(-c5ccc6ccccc6n5)c4)c3)nc2c2nc(-c3ccccc3)ccc12 ; Cc1c(C)c2ccc(-c3cccc(-c4cc(-c5cccc6cccnc56)nc(-c5cccc6cccnc56)c4)c3)nc2c2nc(-c3ccccc3)ccc12]

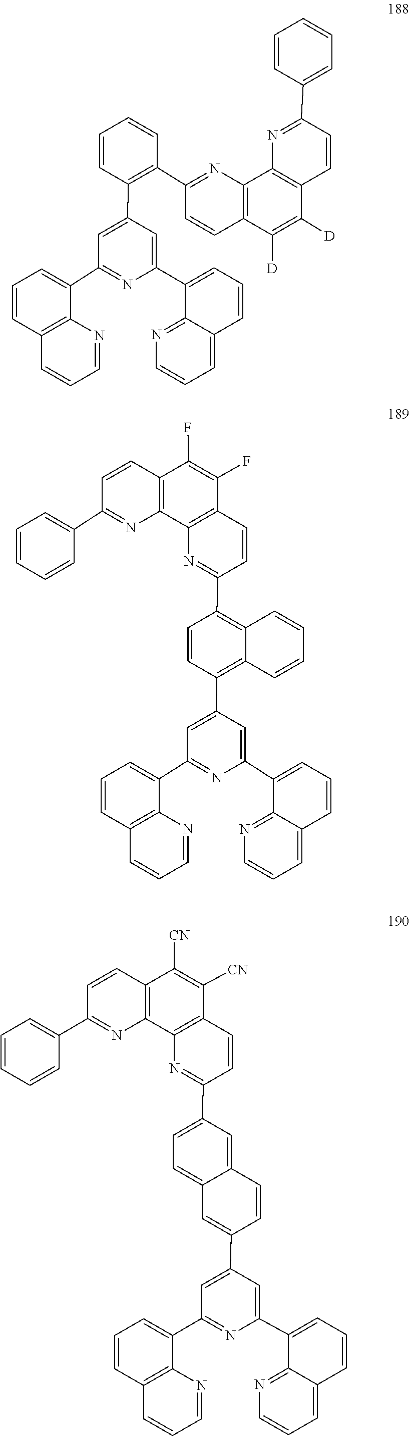

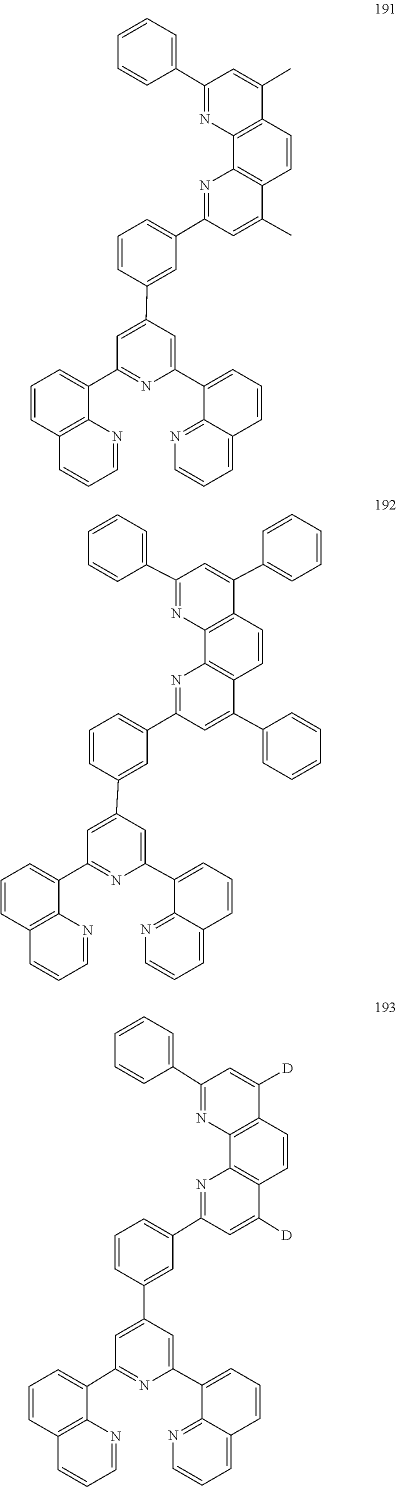

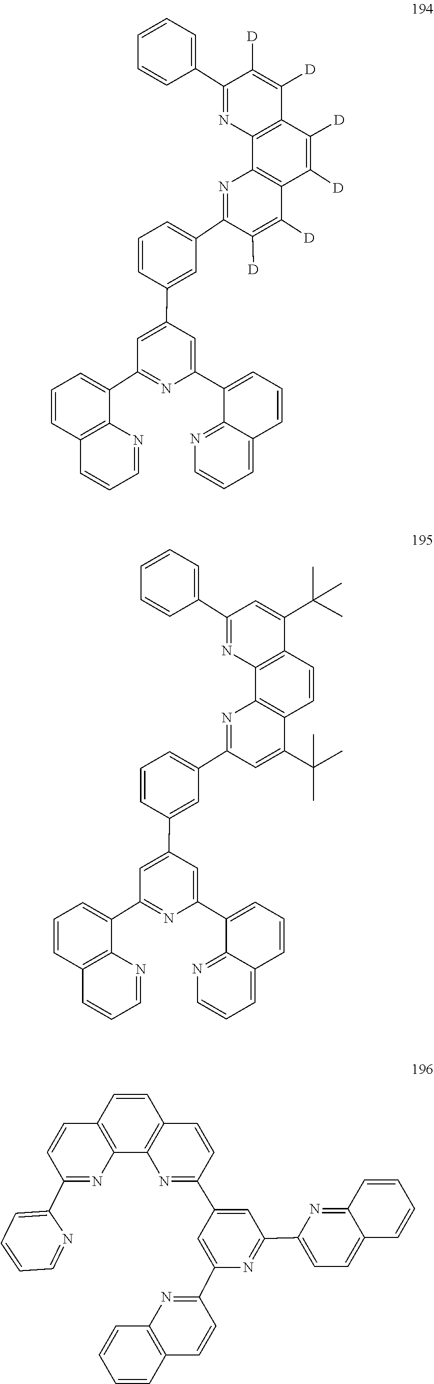 [SMILES: [2H]c1c(-c2ccccc2)nc2c(c1[2H])c([2H])c([2H])c1c([2H])c([2H])c(-c3cccc(-c4cc(-c5cccc6cccnc56)nc(-c5cccc6cccnc56)c4)c3)nc12 ; CC(C)(C)c1cc(-c2ccccc2)nc2c1ccc1c(C(C)(C)C)cc(-c3cccc(-c4cc(-c5cccc6cccnc56)nc(-c5cccc6cccnc56)c4)c3)nc12 ; c1ccc(-c2ccc3ccc4ccc(-c5cc(-c6ccc7ccccc7n6)nc(-c6ccc7ccccc7n6)c5)nc4c3n2)nc1]

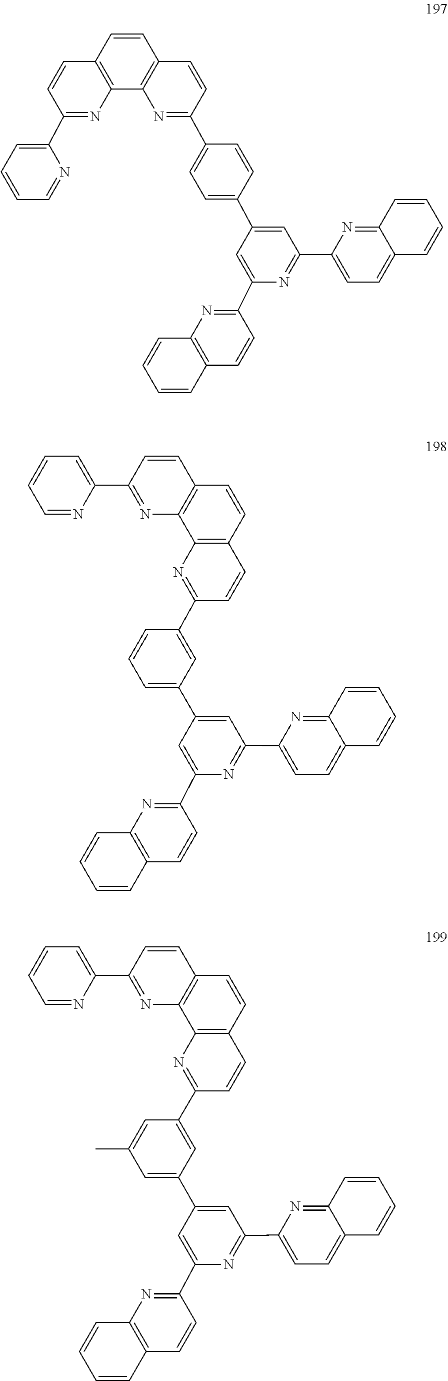 [SMILES: c1ccc(-c2ccc3ccc4ccc(-c5ccc(-c6cc(-c7ccc8ccccc8n7)nc(-c7ccc8ccccc8n7)c6)cc5)nc4c3n2)nc1 ; c1ccc(-c2ccc3ccc4ccc(-c5cccc(-c6cc(-c7ccc8ccccc8n7)nc(-c7ccc8ccccc8n7)c6)c5)nc4c3n2)nc1 ; Cc1cc(-c2cc(-c3ccc4ccccc4n3)nc(-c3ccc4ccccc4n3)c2)cc(-c2ccc3ccc4ccc(-c5ccccn5)nc4c3n2)c1]

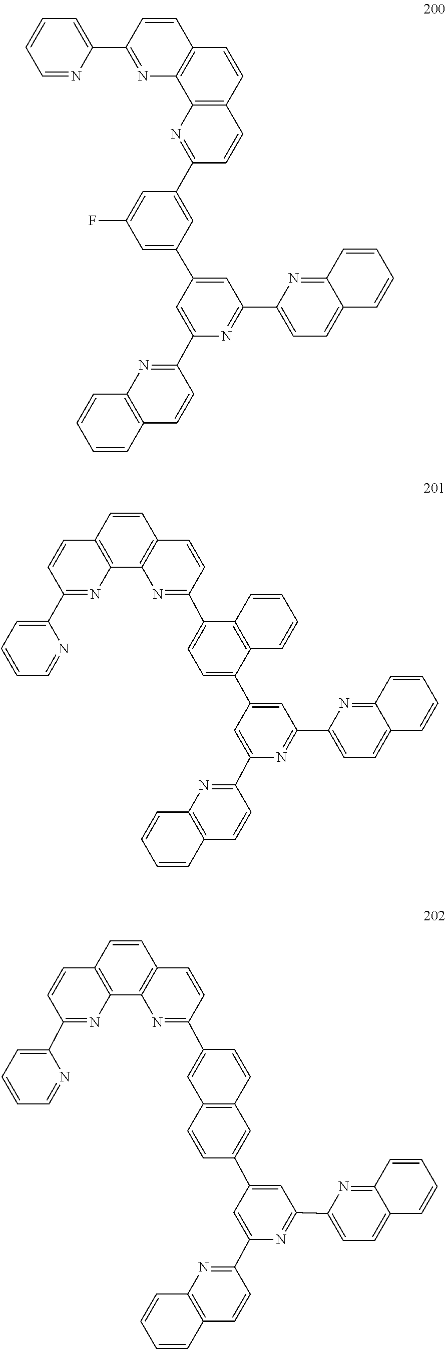 [SMILES: Fc1cc(-c2cc(-c3ccc4ccccc4n3)nc(-c3ccc4ccccc4n3)c2)cc(-c2ccc3ccc4ccc(-c5ccccn5)nc4c3n2)c1 ; c1ccc(-c2ccc3ccc4ccc(-c5ccc(-c6cc(-c7ccc8ccccc8n7)nc(-c7ccc8ccccc8n7)c6)c6ccccc56)nc4c3n2)nc1 ; c1ccc(-c2ccc3ccc4ccc(-c5ccc6cc(-c7cc(-c8ccc9ccccc9n8)nc(-c8ccc9ccccc9n8)c7)ccc6c5)nc4c3n2)nc1]

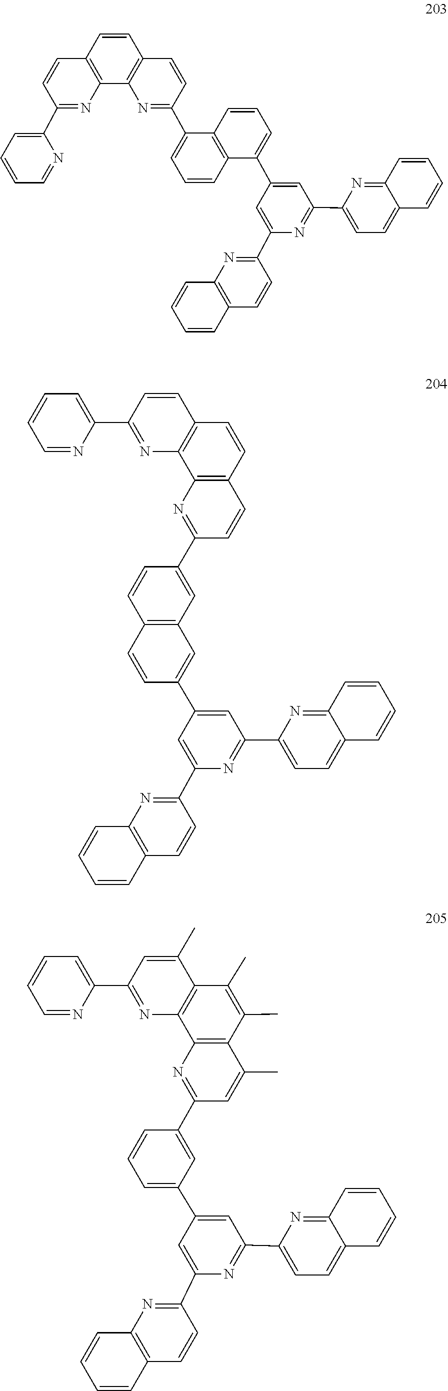 [SMILES: c1ccc(-c2ccc3ccc4ccc(-c5cccc6c(-c7cc(-c8ccc9ccccc9n8)nc(-c8ccc9ccccc9n8)c7)cccc56)nc4c3n2)nc1 ; c1ccc(-c2ccc3ccc4ccc(-c5ccc6ccc(-c7cc(-c8ccc9ccccc9n8)nc(-c8ccc9ccccc9n8)c7)cc6c5)nc4c3n2)nc1 ; Cc1cc(-c2cccc(-c3cc(-c4ccc5ccccc5n4)nc(-c4ccc5ccccc5n4)c3)c2)nc2c1c(C)c(C)c1c(C)cc(-c3ccccn3)nc12]

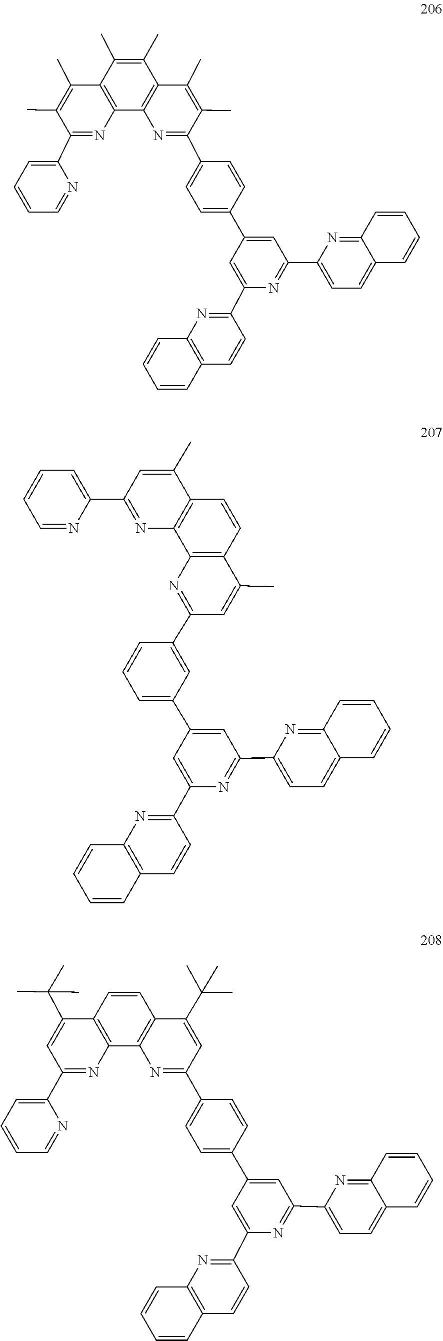 [SMILES: Cc1c(-c2ccc(-c3cc(-c4ccc5ccccc5n4)nc(-c4ccc5ccccc5n4)c3)cc2)nc2c(c1C)c(C)c(C)c1c(C)c(C)c(-c3ccccn3)nc12 ; Cc1cc(-c2cccc(-c3cc(-c4ccc5ccccc5n4)nc(-c4ccc5ccccc5n4)c3)c2)nc2c1ccc1c(C)cc(-c3ccccn3)nc12 ; CC(C)(C)c1cc(-c2ccc(-c3cc(-c4ccc5ccccc5n4)nc(-c4ccc5ccccc5n4)c3)cc2)nc2c1ccc1c(C(C)(C)C)cc(-c3ccccn3)nc12]

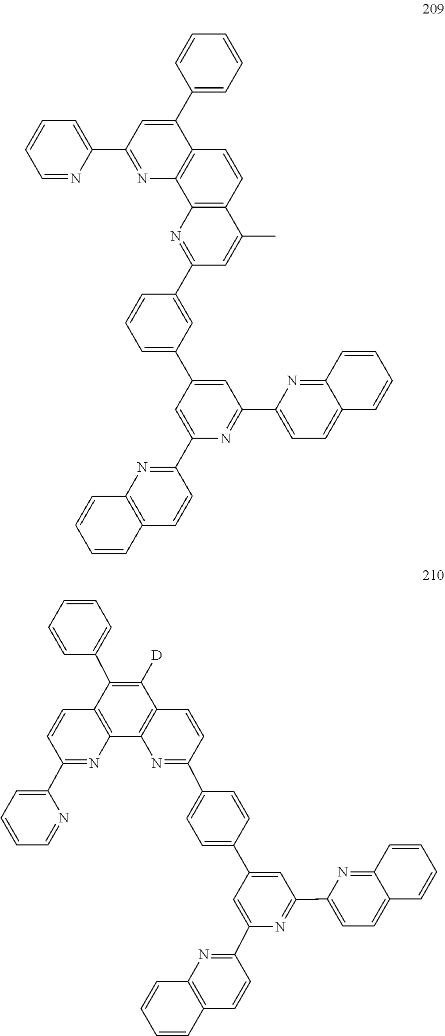 [SMILES: Cc1cc(-c2cccc(-c3cc(-c4ccc5ccccc5n4)nc(-c4ccc5ccccc5n4)c3)c2)nc2c1ccc1c(-c3ccccc3)cc(-c3ccccn3)nc12 ; [2H]c1c(-c2ccccc2)c2ccc(-c3ccccn3)nc2c2nc(-c3ccc(-c4cc(-c5ccc6ccccc6n5)nc(-c5ccc6ccccc6n5)c4)cc3)ccc12]

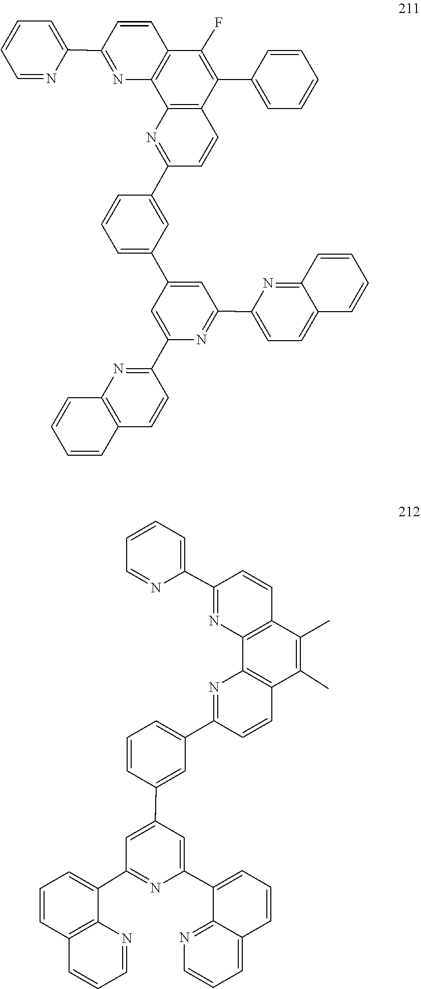 [SMILES: Fc1c(-c2ccccc2)c2ccc(-c3cccc(-c4cc(-c5ccc6ccccc6n5)nc(-c5ccc6ccccc6n5)c4)c3)nc2c2nc(-c3ccccn3)ccc12 ; Cc1c(C)c2ccc(-c3ccccn3)nc2c2nc(-c3cccc(-c4cc(-c5cccc6cccnc56)nc(-c5cccc6cccnc56)c4)c3)ccc12]

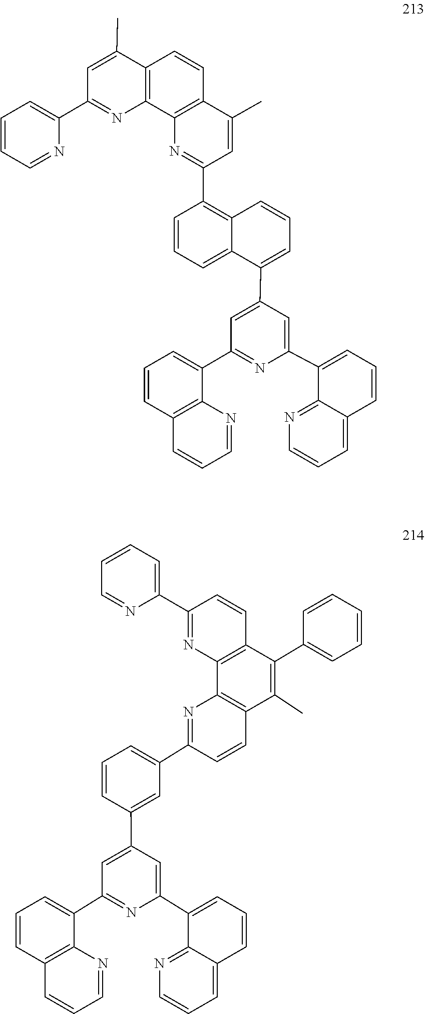 [SMILES: Cc1cc(-c2ccccn2)nc2c1ccc1c(C)cc(-c3cccc4c(-c5cc(-c6cccc7cccnc67)nc(-c6cccc7cccnc67)c5)cccc34)nc12 ; Cc1c(-c2ccccc2)c2ccc(-c3ccccn3)nc2c2nc(-c3cccc(-c4cc(-c5cccc6cccnc56)nc(-c5cccc6cccnc56)c4)c3)ccc12]

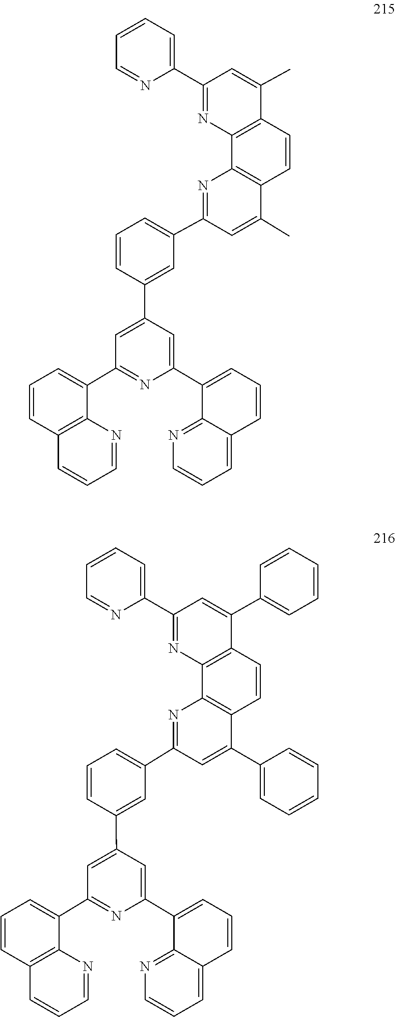 [SMILES: Cc1cc(-c2cccc(-c3cc(-c4cccc5cccnc45)nc(-c4cccc5cccnc45)c3)c2)nc2c1ccc1c(C)cc(-c3ccccn3)nc12 ; c1ccc(-c2cc(-c3cccc(-c4cc(-c5cccc6cccnc56)nc(-c5cccc6cccnc56)c4)c3)nc3c2ccc2c(-c4ccccc4)cc(-c4ccccn4)nc23)cc1]

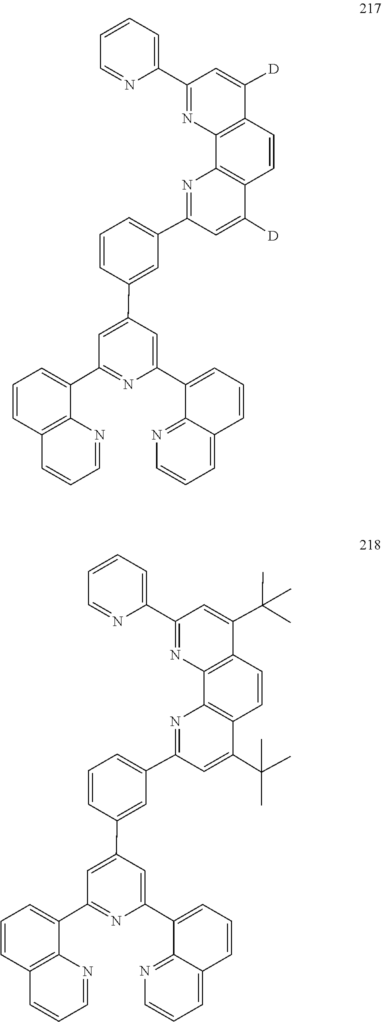 [SMILES: [2H]c1cc(-c2cccc(-c3cc(-c4cccc5cccnc45)nc(-c4cccc5cccnc45)c3)c2)nc2c1ccc1c([2H])cc(-c3ccccn3)nc12 ; CC(C)(C)c1cc(-c2cccc(-c3cc(-c4cccc5cccnc45)nc(-c4cccc5cccnc45)c3)c2)nc2c1ccc1c(C(C)(C)C)cc(-c3ccccn3)nc12]

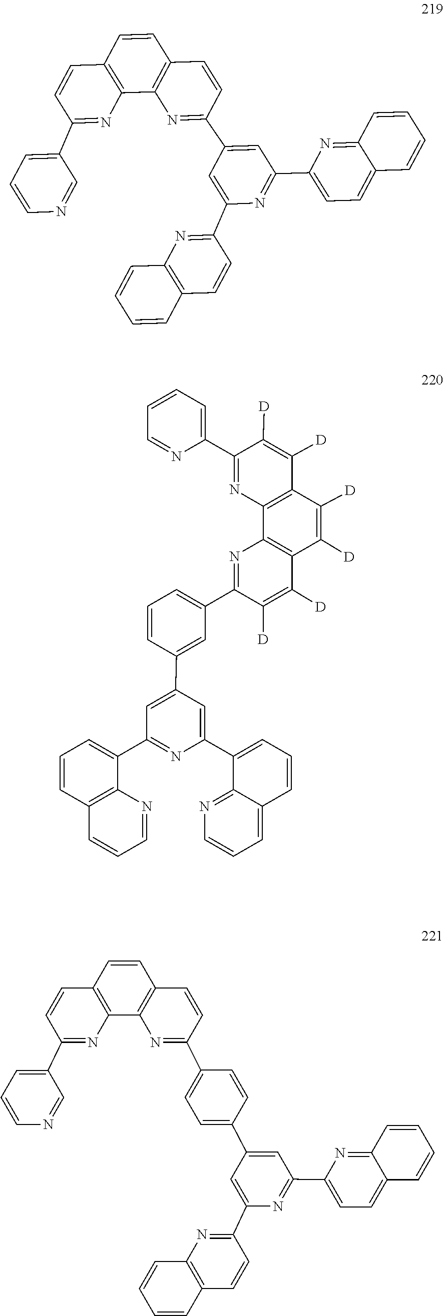 [SMILES: c1cncc(-c2ccc3ccc4ccc(-c5cc(-c6ccc7ccccc7n6)nc(-c6ccc7ccccc7n6)c5)nc4c3n2)c1 ; [2H]c1c(-c2cccc(-c3cc(-c4cccc5cccnc45)nc(-c4cccc5cccnc45)c3)c2)nc2c(c1[2H])c([2H])c([2H])c1c([2H])c([2H])c(-c3ccccn3)nc12 ; c1cncc(-c2ccc3ccc4ccc(-c5ccc(-c6cc(-c7ccc8ccccc8n7)nc(-c7ccc8ccccc8n7)c6)cc5)nc4c3n2)c1]

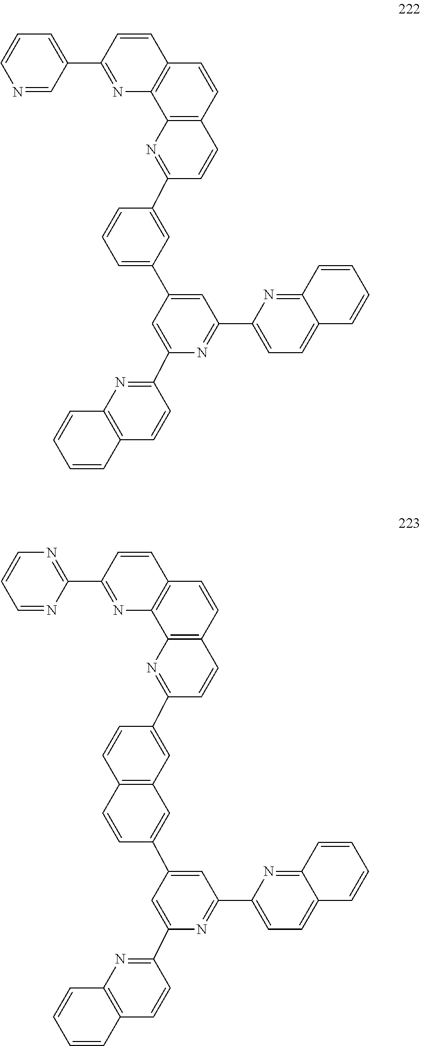 [SMILES: c1cncc(-c2ccc3ccc4ccc(-c5cccc(-c6cc(-c7ccc8ccccc8n7)nc(-c7ccc8ccccc8n7)c6)c5)nc4c3n2)c1 ; c1cnc(-c2ccc3ccc4ccc(-c5ccc6ccc(-c7cc(-c8ccc9ccccc9n8)nc(-c8ccc9ccccc9n8)c7)cc6c5)nc4c3n2)nc1]

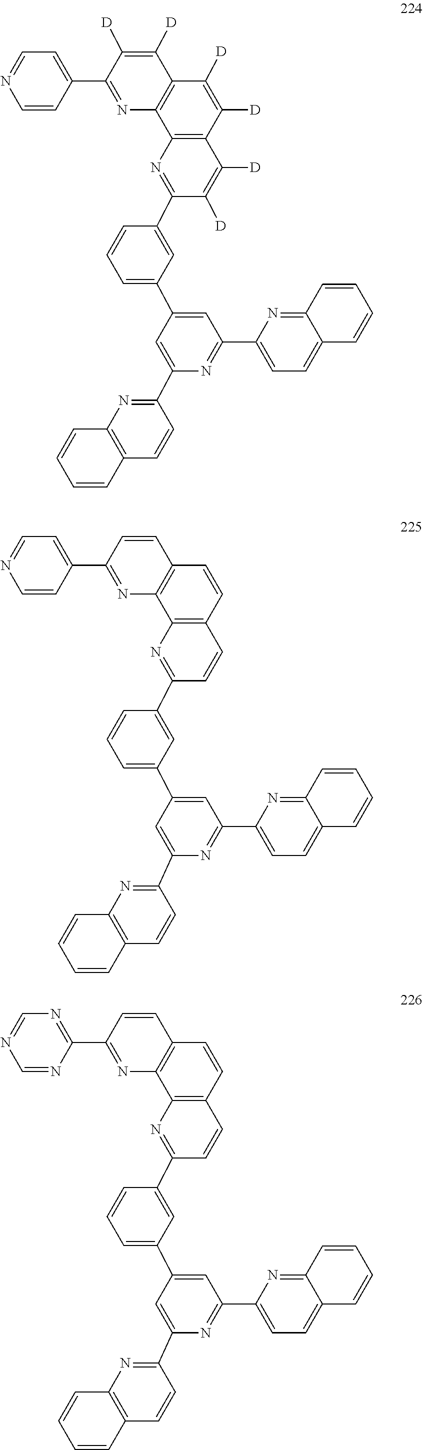

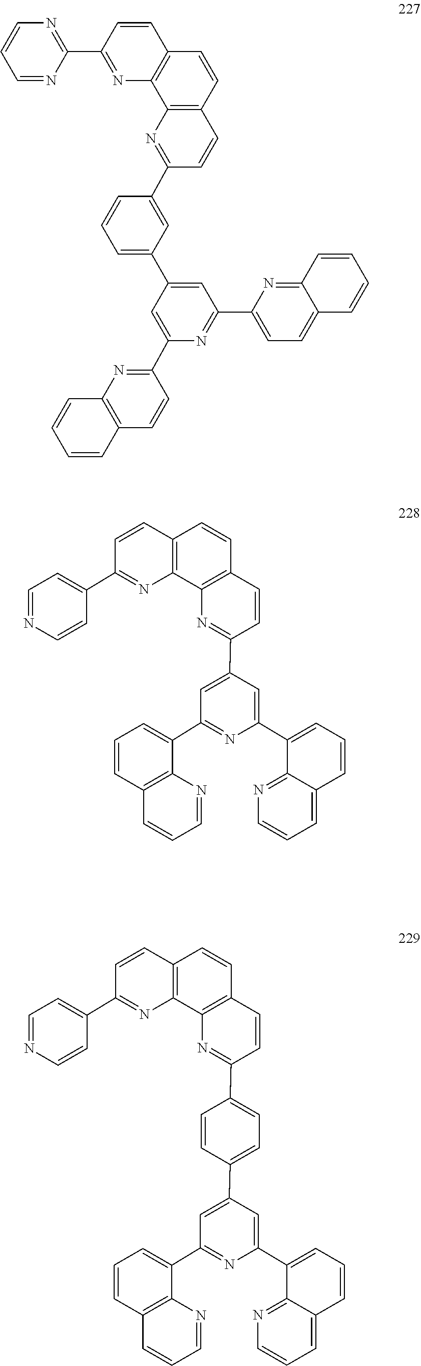 [SMILES: c1cnc(-c2ccc3ccc4ccc(-c5cccc(-c6cc(-c7ccc8ccccc8n7)nc(-c7ccc8ccccc8n7)c6)c5)nc4c3n2)nc1 ; c1cnc2c(-c3cc(-c4ccc5ccc6ccc(-c7ccncc7)nc6c5n4)cc(-c4cccc5cccnc45)n3)cccc2c1 ; c1cnc2c(-c3cc(-c4ccc(-c5ccc6ccc7ccc(-c8ccncc8)nc7c6n5)cc4)cc(-c4cccc5cccnc45)n3)cccc2c1]

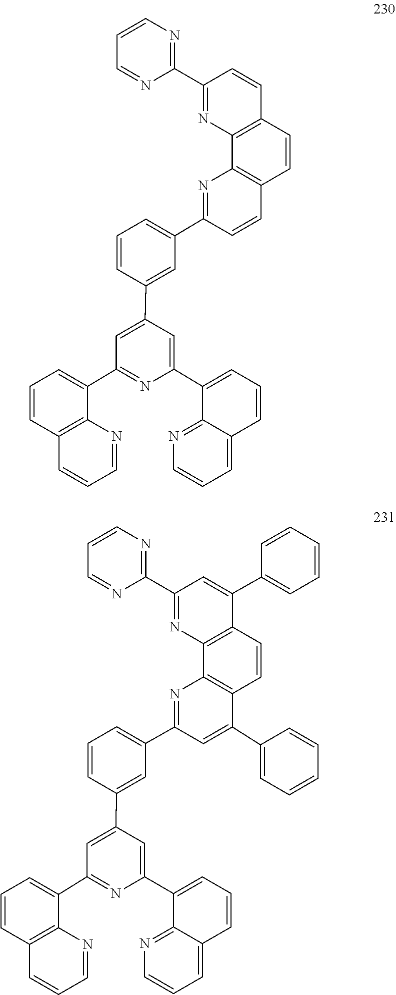 [SMILES: c1cnc(-c2ccc3ccc4ccc(-c5cccc(-c6cc(-c7cccc8cccnc78)nc(-c7cccc8cccnc78)c6)c5)nc4c3n2)nc1 ; c1ccc(-c2cc(-c3cccc(-c4cc(-c5cccc6cccnc56)nc(-c5cccc6cccnc56)c4)c3)nc3c2ccc2c(-c4ccccc4)cc(-c4ncccn4)nc23)cc1]

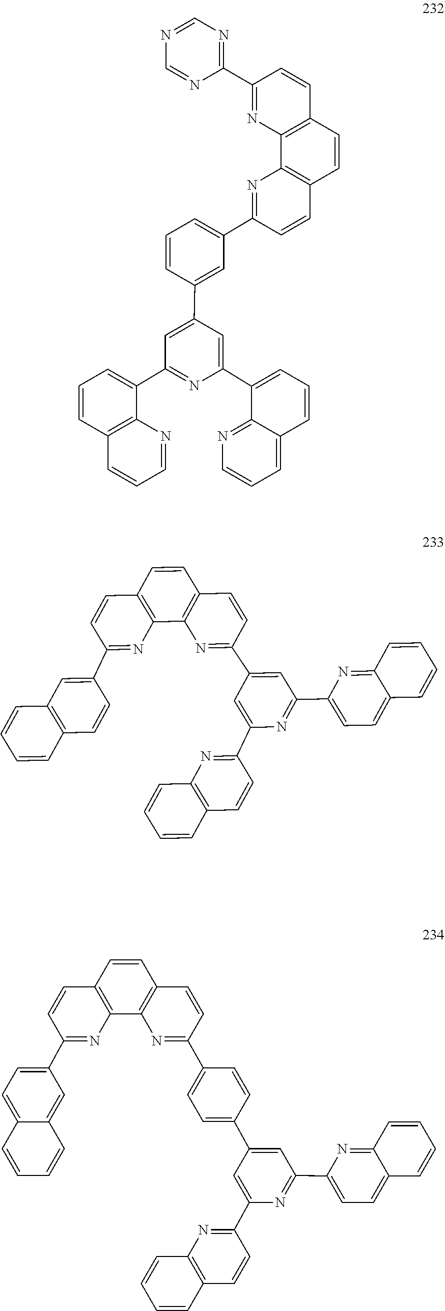 [SMILES: c1cc(-c2cc(-c3cccc4cccnc34)nc(-c3cccc4cccnc34)c2)cc(-c2ccc3ccc4ccc(-c5ncncn5)nc4c3n2)c1 ; c1ccc2cc(-c3ccc4ccc5ccc(-c6cc(-c7ccc8ccccc8n7)nc(-c7ccc8ccccc8n7)c6)nc5c4n3)ccc2c1 ; c1ccc2cc(-c3ccc4ccc5ccc(-c6ccc(-c7cc(-c8ccc9ccccc9n8)nc(-c8ccc9ccccc9n8)c7)cc6)nc5c4n3)ccc2c1]

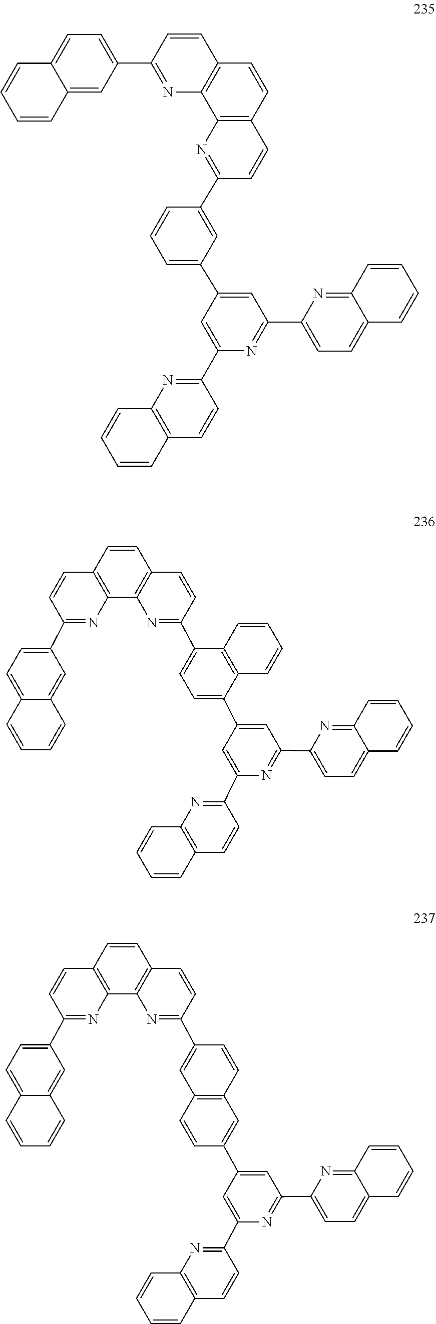 [SMILES: c1cc(-c2cc(-c3ccc4ccccc4n3)nc(-c3ccc4ccccc4n3)c2)cc(-c2ccc3ccc4ccc(-c5ccc6ccccc6c5)nc4c3n2)c1 ; c1ccc2cc(-c3ccc4ccc5ccc(-c6ccc(-c7cc(-c8ccc9ccccc9n8)nc(-c8ccc9ccccc9n8)c7)c7ccccc67)nc5c4n3)ccc2c1 ; c1ccc2cc(-c3ccc4ccc5ccc(-c6ccc7cc(-c8cc(-c9ccc%10ccccc%10n9)nc(-c9ccc%10ccccc%10n9)c8)ccc7c6)nc5c4n3)ccc2c1]

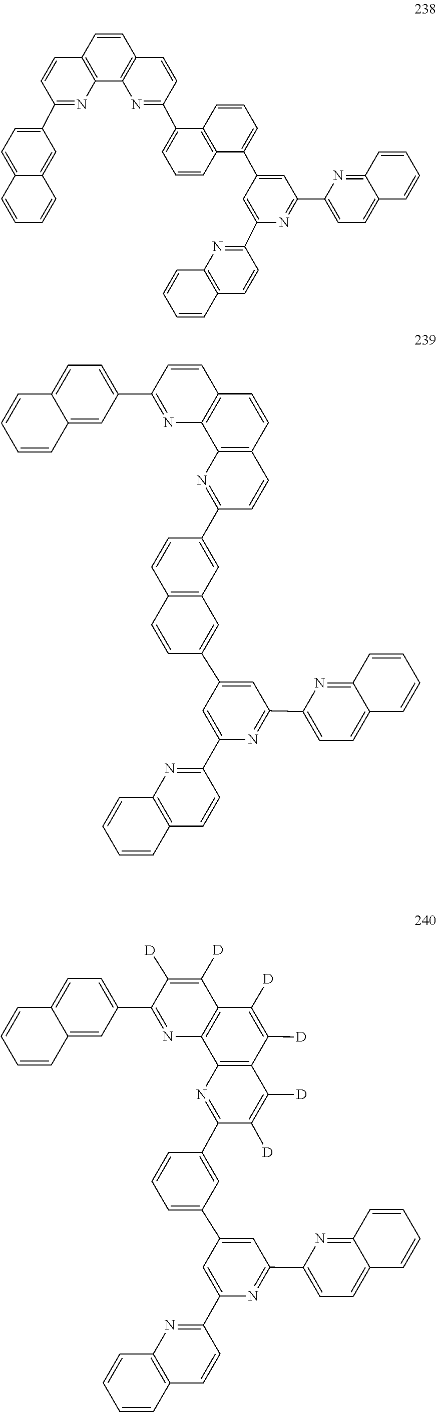 [SMILES: c1ccc2cc(-c3ccc4ccc5ccc(-c6cccc7c(-c8cc(-c9ccc%10ccccc%10n9)nc(-c9ccc%10ccccc%10n9)c8)cccc67)nc5c4n3)ccc2c1 ; c1ccc2cc(-c3ccc4ccc5ccc(-c6ccc7ccc(-c8cc(-c9ccc%10ccccc%10n9)nc(-c9ccc%10ccccc%10n9)c8)cc7c6)nc5c4n3)ccc2c1 ; [2H]c1c(-c2cccc(-c3cc(-c4ccc5ccccc5n4)nc(-c4ccc5ccccc5n4)c3)c2)nc2c(c1[2H])c([2H])c([2H])c1c([2H])c([2H])c(-c3ccc4ccccc4c3)nc12]

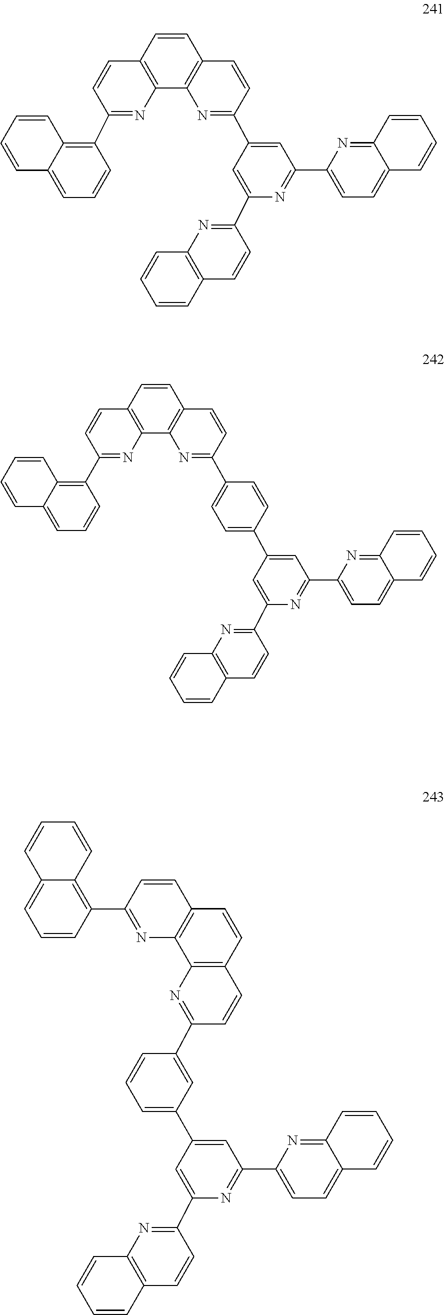 [SMILES: c1ccc2nc(-c3cc(-c4ccc5ccc6ccc(-c7cccc8ccccc78)nc6c5n4)cc(-c4ccc5ccccc5n4)n3)ccc2c1 ; c1ccc2nc(-c3cc(-c4ccc(-c5ccc6ccc7ccc(-c8cccc9ccccc89)nc7c6n5)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc3ccc4ccc(-c5cccc6ccccc56)nc4c3n2)c1]

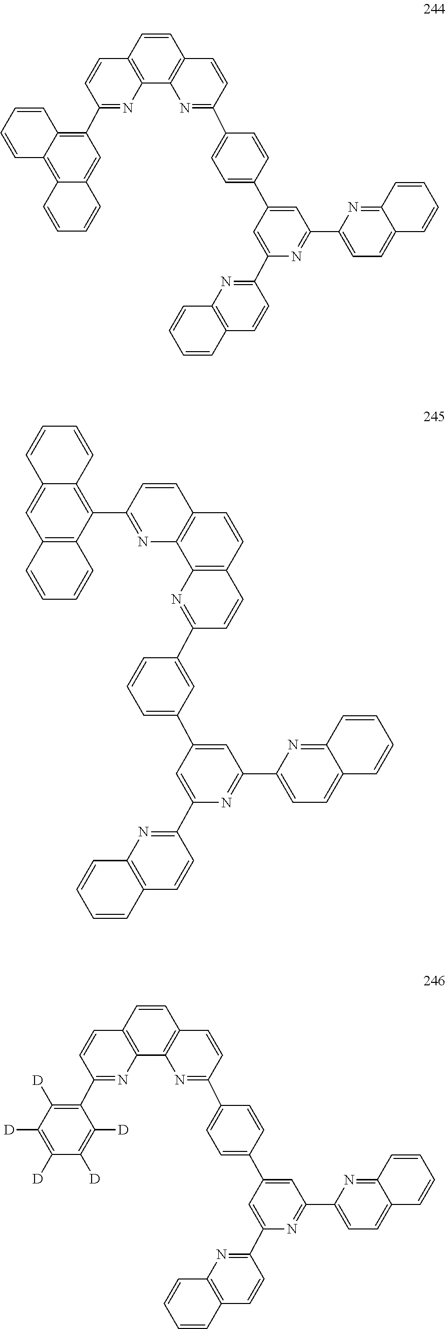 [SMILES: c1ccc2nc(-c3cc(-c4ccc(-c5ccc6ccc7ccc(-c8cc9ccccc9c9ccccc89)nc7c6n5)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc3ccc4ccc(-c5c6ccccc6cc6ccccc56)nc4c3n2)c1 ; [2H]c1c([2H])c([2H])c(-c2ccc3ccc4ccc(-c5ccc(-c6cc(-c7ccc8ccccc8n7)nc(-c7ccc8ccccc8n7)c6)cc5)nc4c3n2)c([2H])c1[2H]]

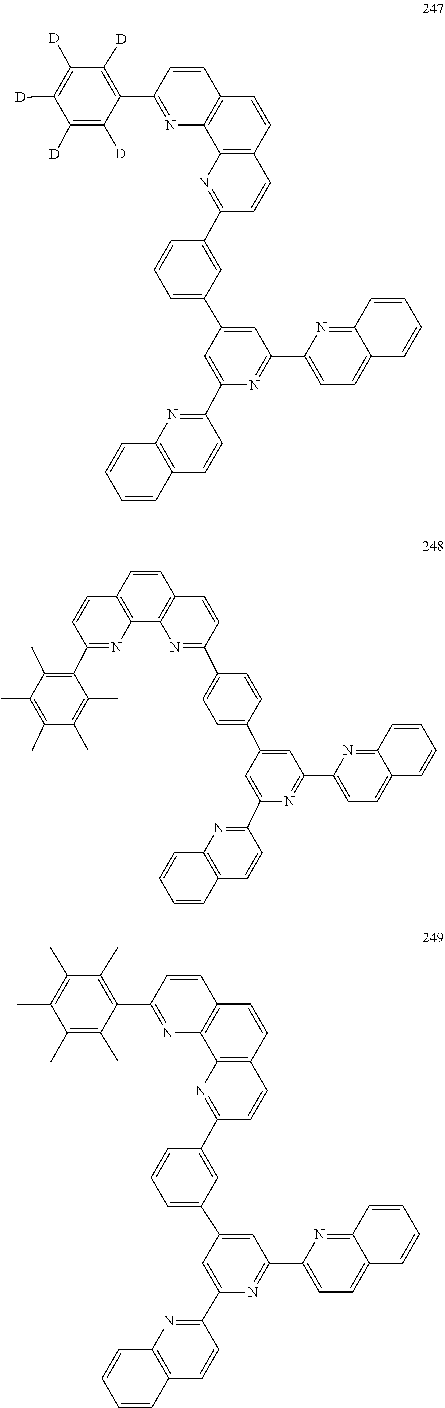 [SMILES: [2H]c1c([2H])c([2H])c(-c2ccc3ccc4ccc(-c5cccc(-c6cc(-c7ccc8ccccc8n7)nc(-c7ccc8ccccc8n7)c6)c5)nc4c3n2)c([2H])c1[2H] ; Cc1c(C)c(C)c(-c2ccc3ccc4ccc(-c5ccc(-c6cc(-c7ccc8ccccc8n7)nc(-c7ccc8ccccc8n7)c6)cc5)nc4c3n2)c(C)c1C ; Cc1c(C)c(C)c(-c2ccc3ccc4ccc(-c5cccc(-c6cc(-c7ccc8ccccc8n7)nc(-c7ccc8ccccc8n7)c6)c5)nc4c3n2)c(C)c1C]

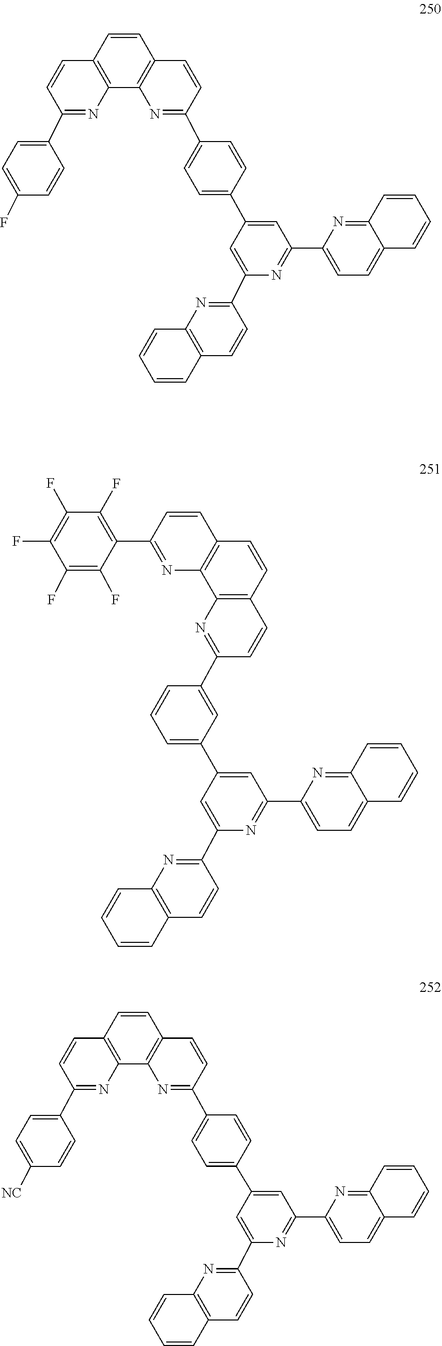 [SMILES: Fc1ccc(-c2ccc3ccc4ccc(-c5ccc(-c6cc(-c7ccc8ccccc8n7)nc(-c7ccc8ccccc8n7)c6)cc5)nc4c3n2)cc1 ; Fc1c(F)c(F)c(-c2ccc3ccc4ccc(-c5cccc(-c6cc(-c7ccc8ccccc8n7)nc(-c7ccc8ccccc8n7)c6)c5)nc4c3n2)c(F)c1F ; N#Cc1ccc(-c2ccc3ccc4ccc(-c5ccc(-c6cc(-c7ccc8ccccc8n7)nc(-c7ccc8ccccc8n7)c6)cc5)nc4c3n2)cc1]

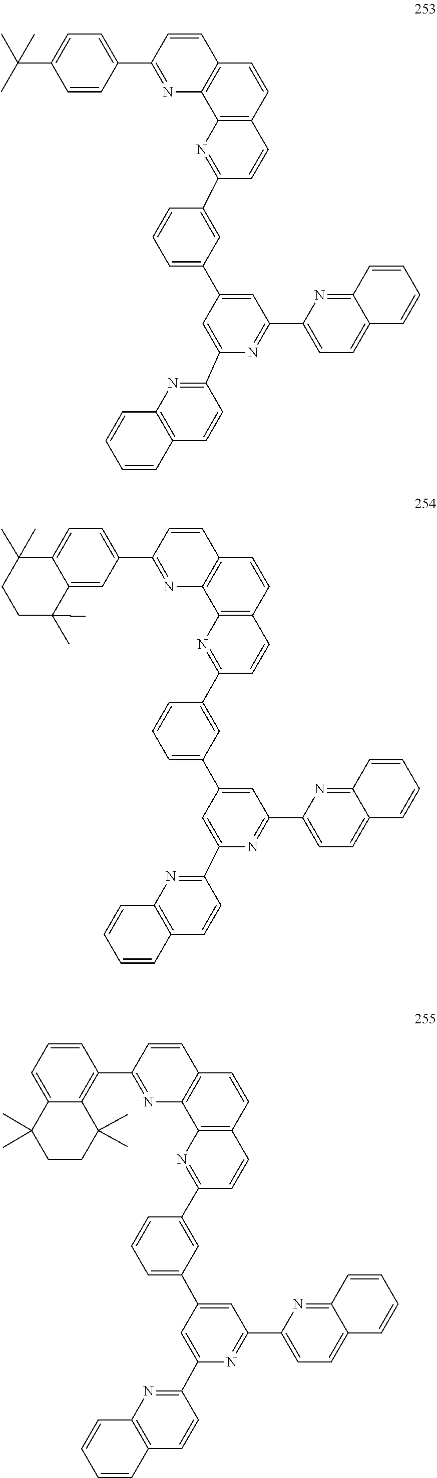 [SMILES: CC(C)(C)c1ccc(-c2ccc3ccc4ccc(-c5cccc(-c6cc(-c7ccc8ccccc8n7)nc(-c7ccc8ccccc8n7)c6)c5)nc4c3n2)cc1 ; CC1(C)CCC(C)(C)c2cc(-c3ccc4ccc5ccc(-c6cccc(-c7cc(-c8ccc9ccccc9n8)nc(-c8ccc9ccccc9n8)c7)c6)nc5c4n3)ccc21 ; CC1(C)CCC(C)(C)c2c(-c3ccc4ccc5ccc(-c6cccc(-c7cc(-c8ccc9ccccc9n8)nc(-c8ccc9ccccc9n8)c7)c6)nc5c4n3)cccc21]

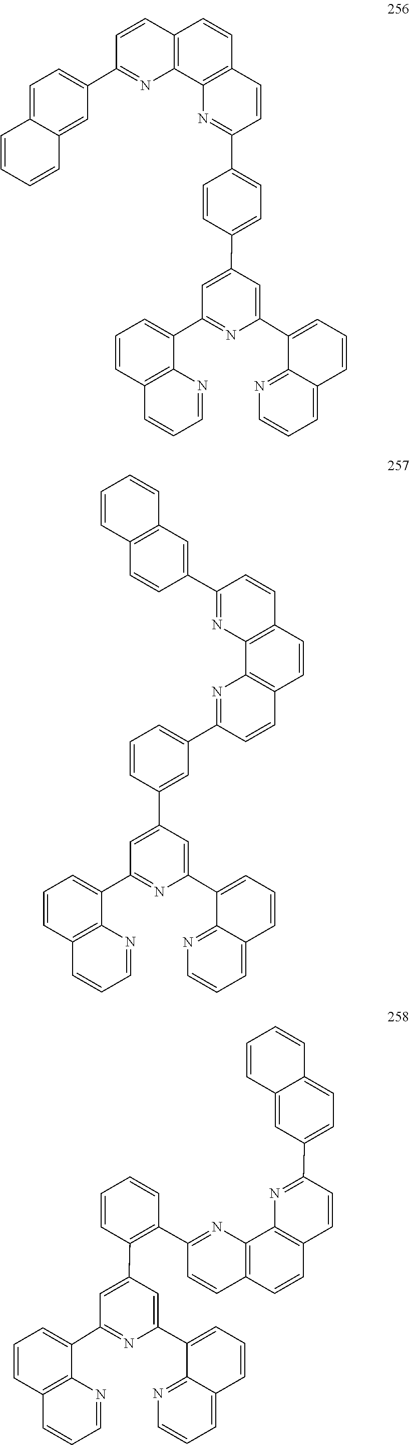 [SMILES: c1ccc2cc(-c3ccc4ccc5ccc(-c6ccc(-c7cc(-c8cccc9cccnc89)nc(-c8cccc9cccnc89)c7)cc6)nc5c4n3)ccc2c1 ; c1cc(-c2cc(-c3cccc4cccnc34)nc(-c3cccc4cccnc34)c2)cc(-c2ccc3ccc4ccc(-c5ccc6ccccc6c5)nc4c3n2)c1 ; c1ccc(-c2ccc3ccc4ccc(-c5ccc6ccccc6c5)nc4c3n2)c(-c2cc(-c3cccc4cccnc34)nc(-c3cccc4cccnc34)c2)c1]

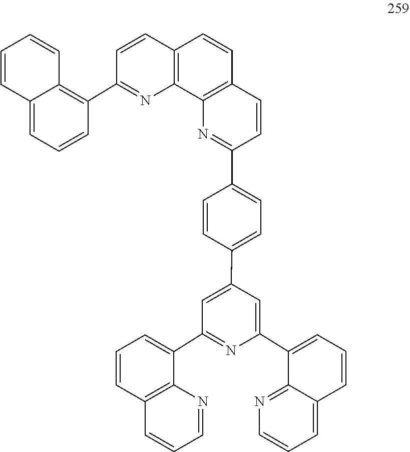 [SMILES: c1ccc2c(-c3ccc4ccc5ccc(-c6ccc(-c7cc(-c8cccc9cccnc89)nc(-c8cccc9cccnc89)c7)cc6)nc5c4n3)cccc2c1]

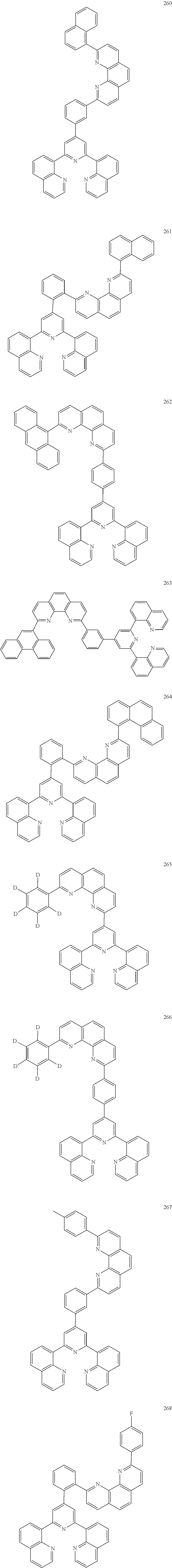

[chemical structure]

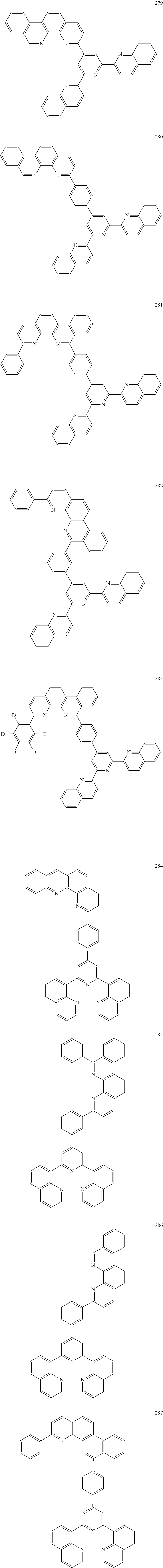

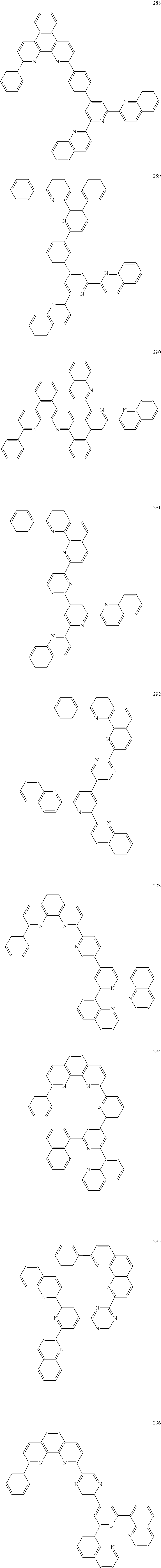

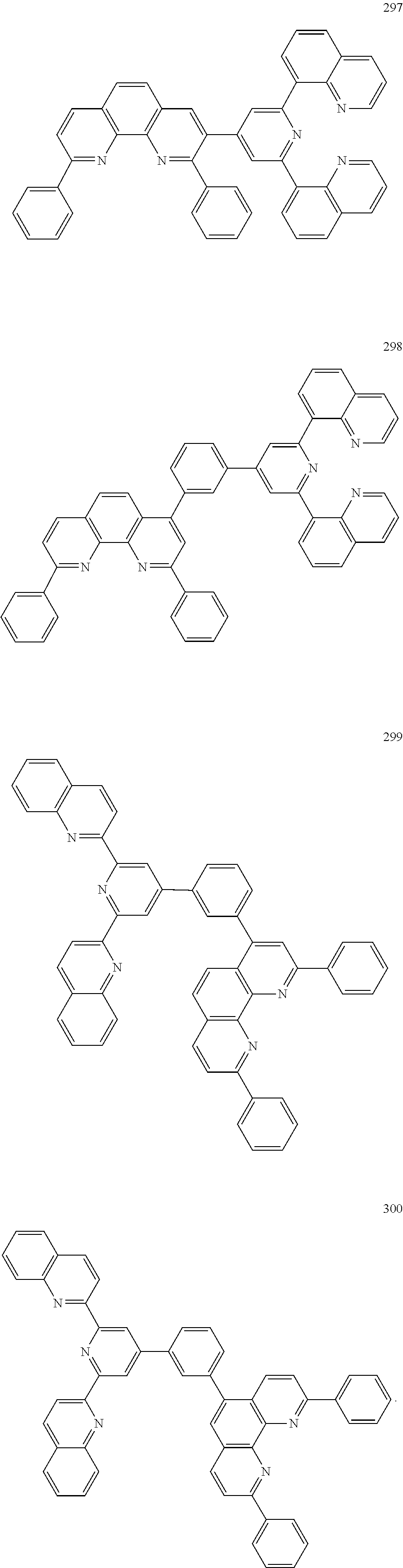 [SMILES: c1ccc(-c2ccc3ccc4cc(-c5cc(-c6cccc7cccnc67)nc(-c6cccc7cccnc67)c5)c(-c5ccccc5)nc4c3n2)cc1 ; c1ccc(-c2ccc3ccc4c(-c5cccc(-c6cc(-c7cccc8cccnc78)nc(-c7cccc8cccnc78)c6)c5)cc(-c5ccccc5)nc4c3n2)cc1 ; c1ccc(-c2ccc3ccc4c(-c5cccc(-c6cc(-c7ccc8ccccc8n7)nc(-c7ccc8ccccc8n7)c6)c5)cc(-c5ccccc5)nc4c3n2)cc1 ; c1ccc(-c2ccc3cc(-c4cccc(-c5cc(-c6ccc7ccccc7n6)nc(-c6ccc7ccccc7n6)c5)c4)c4ccc(-c5ccccc5)nc4c3n2)cc1]

In a second aspect, the present application provides an organic electroluminescent device, including an anode and a cathode which are oppositely disposed, and a functional layer disposed between the anode and the cathode, where the functional layer includes the organic compound provided in the first aspect of the present application.

Alternatively, the functional layer includes an electron transport layer including the organic compound described in the present application.

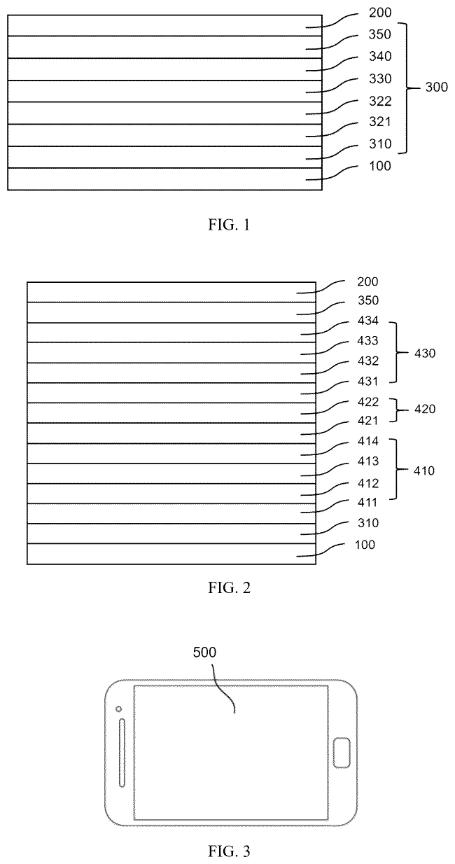

In an embodiment of the present application, the organic electroluminescent device has a structure as shown in FIG. 1 and includes an anode 100 and a cathode 200 which are disposed oppositely, and a functional layer 300 disposed between the anode 100 and the cathode 200; and the functional layer 300 includes a hole injection layer 310, a hole transport layer 321, an electron blocking layer 322, an organic light-emitting layer 330, an electron transport layer 340 and an electron injection layer 350, the transport layer 340 includes the organic compound described in the present application.

In the present application, the anode 100 includes an anode material, which is preferably a material having a large work function that facilitates hole injection into the functional layer. Specific examples of the anode material include metals such as nickel, platinum, vanadium, chromium, copper, zinc, and gold, or its alloy; metal oxides such as zinc oxide, indium oxide, indium tin oxide (ITO) and indium zinc oxide (IZO); combined metals and oxides, such as ZnO:Al or SnO₂:Sb; or a conductive polymer such as poly(3-methylthiophene), poly[3,4-(ethylene-1,2-dioxy)thiophene](PEDT), polypyrrole, and polyaniline, but are not limited to this. Alternatively, a transparent electrode including indium tin oxide (ITO) as the anode is included.

In the present application, the hole injection layer 310 may be made of a benzidine derivative, a starburst arylamine compound, a phthalocyanine derivative or other materials, which is not particularly limited in the present application. A material of the hole injection layer 310 is, for example, selected from the following compounds or any combination of them;

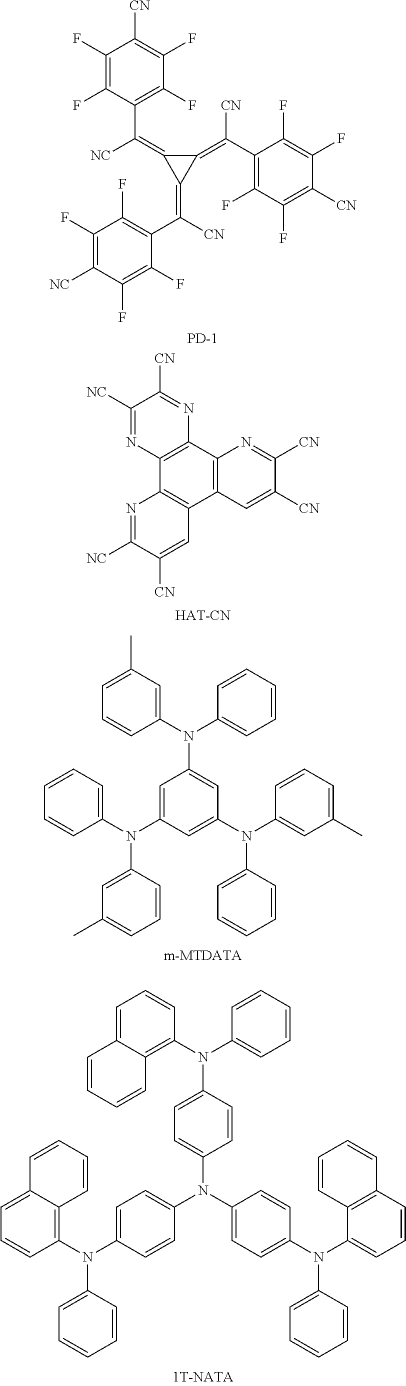

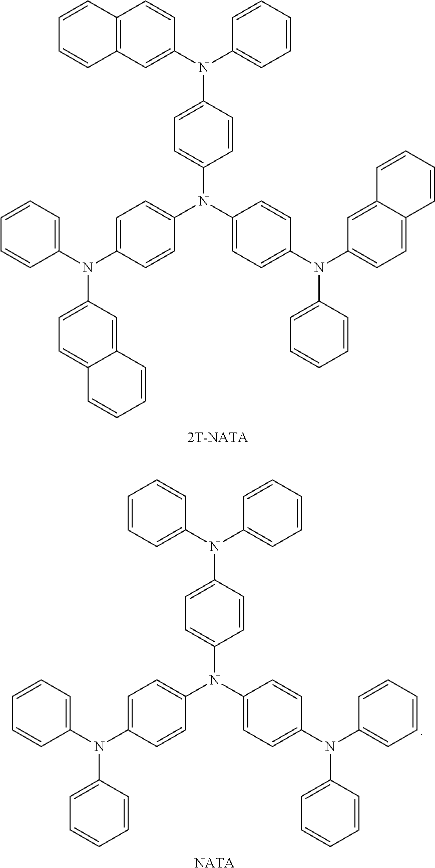

In one embodiment of the present application, the hole injection layer 310 consists of HT-1 and PD-1.

In some embodiments of the present application, a hole transport material may be selected from triarylamine compounds or other types of compounds, which can be selected by those skilled in the art with reference to the prior art. For example, a material of the hole transport layer is selected from the group consisting of the following compounds.

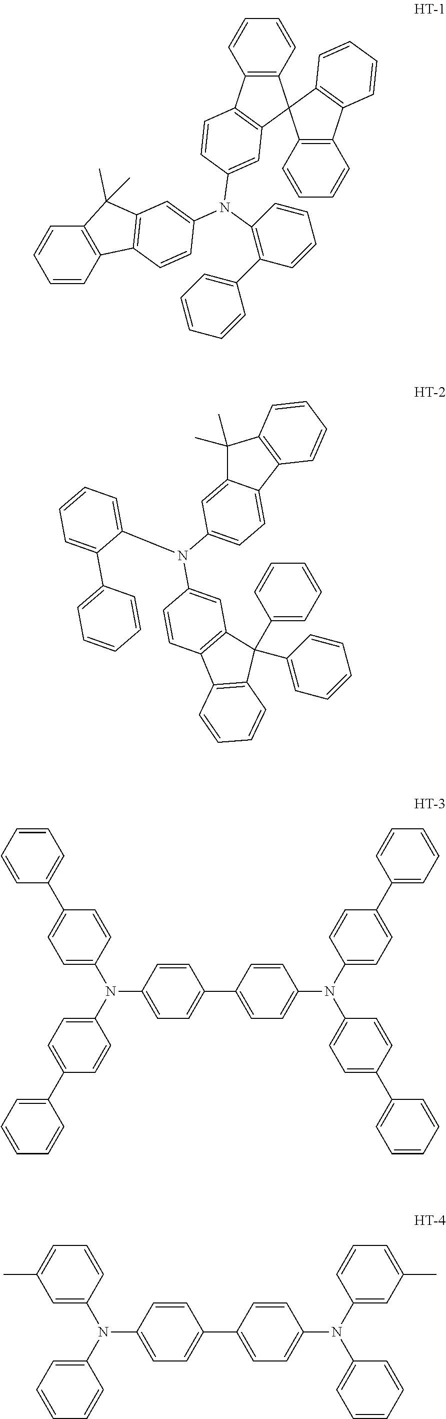

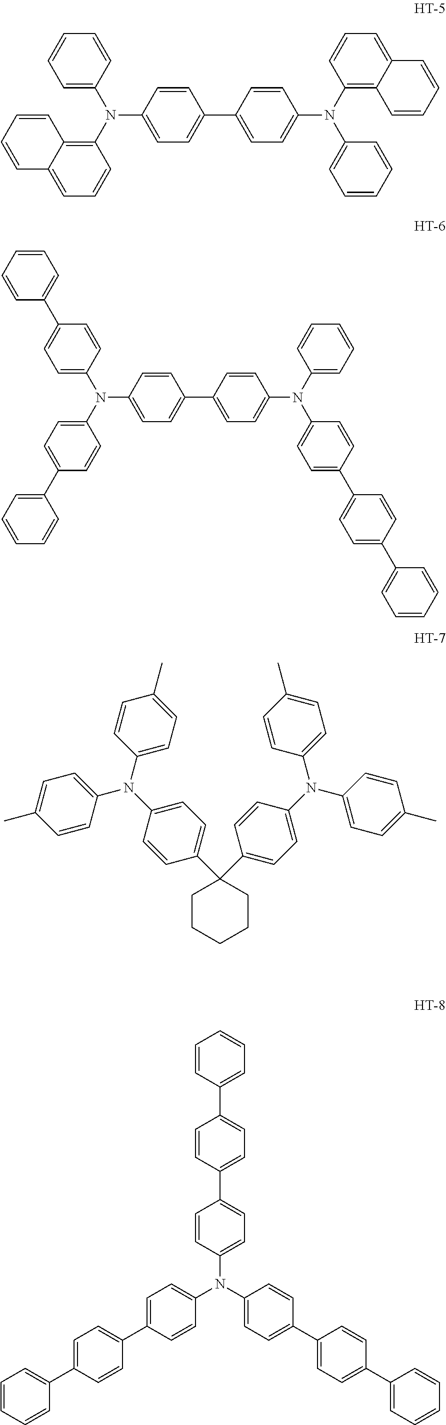

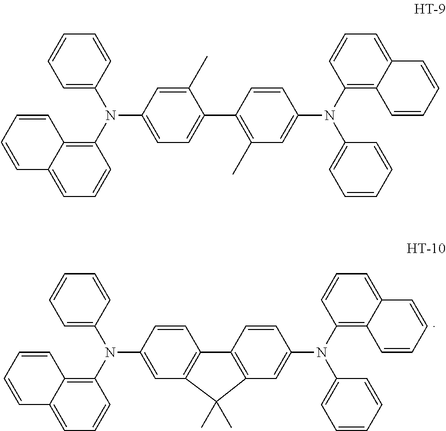 [SMILES: Cc1cc(N(c2ccccc2)c2cccc3ccccc23)ccc1-c1ccc(N(c2ccccc2)c2cccc3ccccc23)cc1C ; CC1(C)c2cc(N(c3ccccc3)c3cccc4ccccc34)ccc2-c2ccc(N(c3ccccc3)c3cccc4ccccc34)cc21]

In one embodiment of the present application, a material of the hole transport layer 321 includes HT-1.

In one embodiment of the present application, the electron blocking layer 322 includes one or more electron blocking materials, and the electron blocking materials may be selected from a carbazole polymer or other types of compounds, which are not particularly limited in the present application. For example, in some embodiments of the present application, the electron blocking layer 322 is made of a compound EB-1

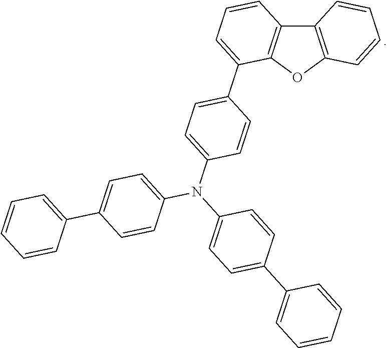 [SMILES: c1ccc(-c2ccc(N(c3ccc(-c4ccccc4)cc3)c3ccc(-c4cccc5c4oc4ccccc45)cc3)cc2)cc1]

Alternatively, the organic light-emitting layer material may consist of a single light-emitting material or may include a host material and a guest material. Alternatively, the organic light-emitting layer 330 is composed of the host material and the guest material, holes injected into the organic light-emitting layer 330 and electrons injected into the organic light-emitting layer 330 can be recombined in the organic light-emitting layer 330 to form excitons, the excitons transfer energy to the host material, the host material transfers energy to the guest material, and then the guest material can emit light.

Alternatively, the host material of the organic light-emitting layer 330 may include a metal chelated compound, a distyryl derivative, an aromatic amine derivative, a dibenzofuran derivative, or other types of materials. The host material of the organic light-emitting layer 330 may be a single host material or a mixed host material. In an embodiment of the present application, the host material of the organic light-emitting layer 330 is BH-1

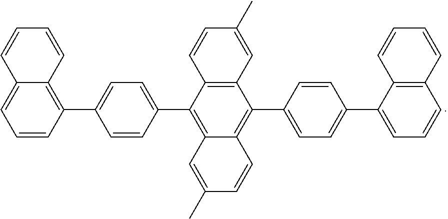 [SMILES: Cc1ccc2c(-c3ccc(-c4cccc5ccccc45)cc3)c3cc(C)ccc3c(-c3ccc(-c4cccc5ccccc45)cc3)c2c1]

In a specific embodiment of the present application, the guest material of the organic light-emitting layer 330 is BD-1

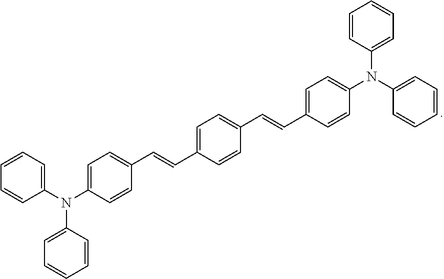 [SMILES: C(=C\c1ccc(N(c2ccccc2)c2ccccc2)cc1)/c1ccc(/C=C/c2ccc(N(c3ccccc3)c3ccccc3)cc2)cc1]

In the present application, the electron injection layer 350 may include an inorganic material such as an alkali metal sulfide and an alkali metal halide, or may include a complex of an alkali metal and an organic substance. In an embodiment of the present application, the electron injection layer 350 includes Yb.

Alternatively, the electron transport layer 340 may be of a single-layer structure or a multi-layer structure, and may include one or more electron transport materials, and the electron transport materials may generally include a metal complex or a nitrogen-containing heterocyclic derivative, where a material of the metal complex may be selected from, for example, LiQ, Alq₃, and the like. In one embodiment of the present application, the electron transport layer 340 consists of the compound of the present application and LiQ.

In the present application, the cathode 200 includes a cathode material, which is a material having a small work function that facilitates electron injection into the functional layer. Specific examples of the cathode material include, but are not limited to, metals such as magnesium, calcium, sodium, potassium, titanium, indium, yttrium, lithium, gadolinium, aluminum, silver, tin, and lead, or its alloy; or multilayer materials such as LiF/Al, Liq/Al, LiO₂/Al, LiF/Ca, LiF/Al, and BaF₂/Ca. Alternatively, a metal electrode including magnesium and silver as the cathode is included.

In addition, the present application also provides another organic electroluminescent device as shown in FIG. 2, the organic electroluminescent device includes an anode and a cathode, and a functional layer disposed between the anode and the cathode, the functional layer including a first light-emitting unit, a second light-emitting unit, and a charge generation layer; and the charge generation layer includes the organic compound described in the present application. The organic electroluminescent device shown in FIG. 2 is, in the following, also referred to as a stacked organic electroluminescent device.

The stacked organic electroluminescent device shown in FIG. 2 includes an anode 100, a cathode 200, a hole injection layer 310, an electron injection layer 350, a first light-emitting unit 410, a second light-emitting unit 430, and a charge generation layer 420. The first light-emitting unit 410, the second light-emitting unit 430, and the charge generation layer 420 are positioned between the cathode 100 and the anode 200, and the charge generation layer 420 is positioned between the first light-emitting unit 410 and the second light-emitting unit 430, and the charge generation layer 420 includes the organic compound described in the present application.

In an embodiment of the present application, the first light-emitting unit 410 includes a first hole transport layer 411, a first hole adjustment layer 412, a first organic light-emitting layer 413 and a first electron transport layer 414; the second light-emitting unit 430 includes a second hole transport layer 431, a second hole adjustment layer 432, a second organic light-emitting layer 433, and a second electron transport layer 434.

In an embodiment of the present application, the charge generation layer 420 includes an n-type charge generation layer (n-CGL) 421 that provides electrons to the first electron transport layer 414 of the first light-emitting unit 410 and a p-type charge generation layer (p-CGL) 422 that provides holes to the second hole transport layer 431 of the second light-emitting unit 430. In one embodiment of the present application, the n-type charge generation layer includes the organic compound described in the present application.

In one embodiment of the present application, the n-type charge generation layer consists of the organic compound described in the present application and a metal doping material. Alternatively, the metal doping material is Li, Ca, Ag, Cs or Yb.

In one embodiment of the present application, the p-CGL layer includes HT-1 and PD-1.

In the present application, the anode 100 includes an anode material, and alternatively, the anode material includes indium tin oxide (ITO).

In one embodiment of the present application, the hole injection layer 310 consists of HT-1 and PD-1.

In an embodiment of the present application, a material of the first hole transport layer 411 and the second hole transport layer 431 includes RP-1.

In an embodiment of the present application, a material of the first hole adjustment layer 412 and the second hole adjustment layer 432 includes RP-1.

In the present application, the first organic light-emitting layer of the first light-emitting unit and the second organic light-emitting layer of the second light-emitting unit may each include the same or different host materials and the same or different guest materials.

In a specific embodiment of the present application, a host material of the first organic light-emitting layer 413 and the second organic light-emitting layer 433 is RH-1

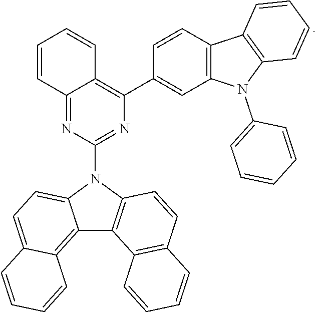 [SMILES: c1ccc(-n2c3ccccc3c3ccc(-c4nc(-n5c6ccc7ccccc7c6c6c7ccccc7ccc65)nc5ccccc45)cc32)cc1]

In a specific embodiment of the present application, a guest material of the first organic light-emitting layer 413 and the second organic light-emitting layer 433 is RD-1

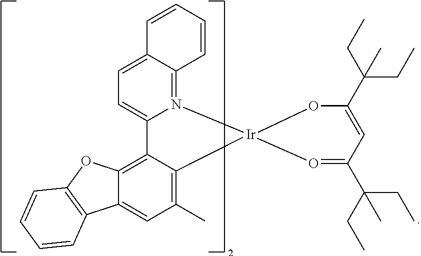

In a specific embodiment of the present application, a material of the first electron transport layer 414 and the second electron transport layer 434 includes ET-1

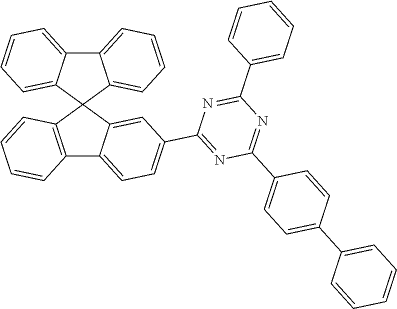 [SMILES: c1ccc(-c2ccc(-c3nc(-c4ccccc4)nc(-c4ccc5c(c4)C4(c6ccccc6-c6ccccc64)c4ccccc4-5)n3)cc2)cc1]

### and LiQ.

In an embodiment of the present application, the electron injection layer 350 includes Yb.

In a specific embodiment of the present application, the cathode 200 includes a cathode material including magnesium (Mg) and silver (Ag).

In a third aspect, the present application provides an electronic apparatus, including the organic electroluminescent device in the second aspect of the present application.

According to one embodiment, as shown in FIG. 3, the electronic apparatus provided is an electronic apparatus 500 including the organic electroluminescent device described above. The electronic apparatus 500 may be, for example, a display device, a lighting device, an optical communication device, or other type of electronic devices, and may include, for example, but is not limited to, a computer screen, a mobile phone screen, a television, electronic paper, an emergency light, an optical module, and the like.

A synthesis method of the organic compound of the present application is specifically described below with reference to the synthesis examples, but the present application is not limited in any way accordingly.

Compounds of which synthesis methods are not mentioned in the present application are all commercially available raw material products.

### SYNTHESIS EXAMPLES

### 1. Synthesis of Intermediate IM A-1

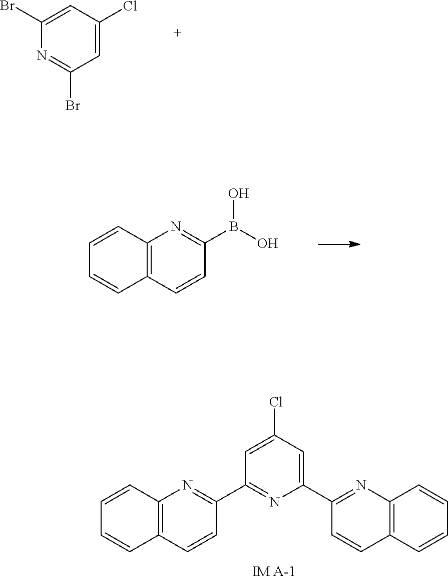

2,6-Dibromo-4-chloropyridine (10 g, 36.85 mmol) and quinoline-2-boronic acid (14.03 g, 81.08 mmol) were dissolved in 80 mL toluene, and tetrakis(triphenylphosphine) palladium (0.43 g, 0.369 mmol), K₂CO₃ (20.34 g, 147.42 mmol), 10 mL of ethanol and 10 mL of water were then added. Heating reflux was performed under a nitrogen atmosphere for 12 hours. After cooling to room temperature, the reaction solution was extracted with water and dichloromethane, dried over anhydrous magnesium sulfate, filtered, and concentrated under reduced pressure. The obtained solid was allowed to pass through a chromatography column using ethyl acetate:n-heptane=1:9 (v/v) to obtain IM A-1 (7.18 g, yield: 53%).

IM A-2 was synthesized by the same method as that of IM A-1 except that a Raw material 1 was used instead of quinoline-2-boronic acid, where the main raw material used, synthesized IM A-2 and their yields are shown in Table 1.

TABLE 1

| Raw material 1 | IM A-2 | Yield/% |
|---|---|---|
| 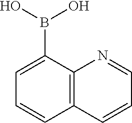 [SMILES: OB(O)c1cccc2cccnc12] | 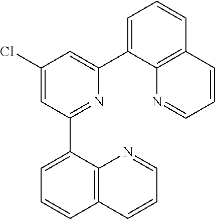 [SMILES: Clc1cc(-c2cccc3cccnc23)nc(-c2cccc3cccnc23)c1] | 49 |

### 2. Synthesis of IM B-1

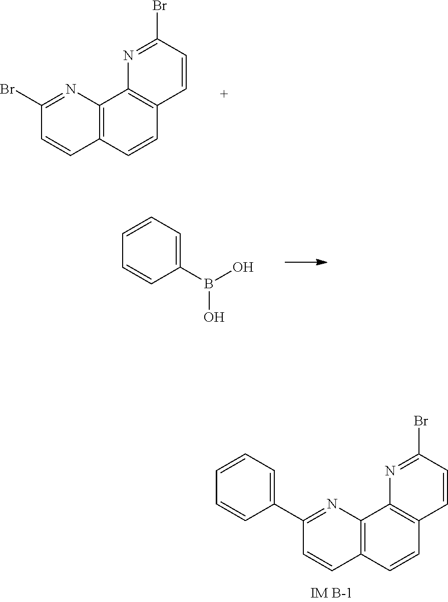 [SMILES: Brc1ccc2ccc3ccc(Br)nc3c2n1 ; OB(O)c1ccccc1 ; Brc1ccc2ccc3ccc(-c4ccccc4)nc3c2n1]

2,9-Dibromo-1,10-phenanthroline (20 g, 59.17 mmol) and phenylboronic acid (7.21 g, 59.17 mmol) were dissolved in 160 mL of toluene, and tetrakis(triphenylphosphine) palladium (0.34 g, 0.296 mmol), K₂CO₃ (16.33 g, 118.34 mmol), 20 mL of ethanol and 20 ml of water were then added. Heating reflux was performed under a nitrogen atmosphere for 12 hours. After cooling to room temperature, the reaction solution was extracted with water and dichloromethane, dried over anhydrous magnesium sulfate, filtered, and concentrated under reduced pressure. The obtained solid was allowed to pass through a chromatography column using ethyl acetate: n-heptane=1:9 (v/v) to obtain IM B-1 (8.13 g, yield: 41%).

IM B-X was synthesized by the same method as that of IM B-1, except that a Raw material 2 was used instead of 2,9-dibromo-1,10-phenanthroline and a Raw material 3 was used instead of phenylboronic acid, where the main raw materials used, IM B-X synthesized and their yields are shown in Table 2.

TABLE 2

| Raw material 2 | Raw material 3 | IM B-X | Yield/% |
|---|---|---|---|
| 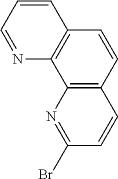 [SMILES: Brc1ccc2ccc3cccnc3c2n1] | 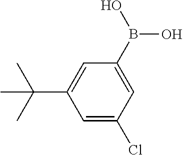 | 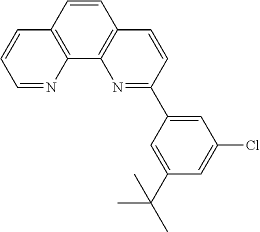 | 63 |
| | | IM B-2 | |
| 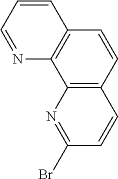 [SMILES: Brc1ccc2ccc3cccnc3c2n1] | 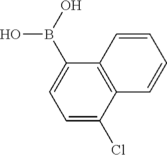 [SMILES: OB(O)c1ccc(Cl)c2ccccc12] | 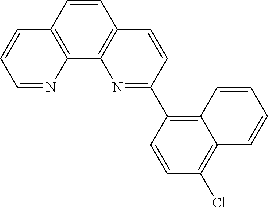 | 57 |
| | | IM B-3 | |
| 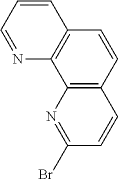 [SMILES: Brc1ccc2ccc3cccnc3c2n1] | 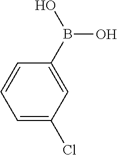 | 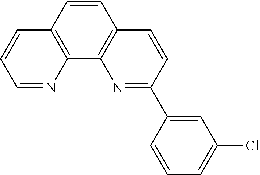 | 61 |
| | | IM B-4 | |
| 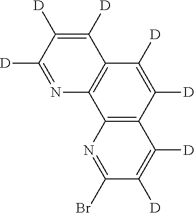 [SMILES: [2H]c1nc2c(c([2H])c1[2H])c([2H])c([2H])c1c([2H])c([2H])c(Br)nc12] | 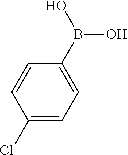 | 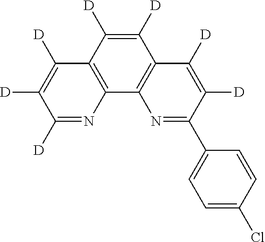 | 64 |
| | | IM B-6 | |
| 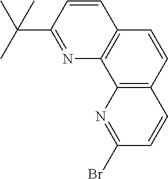 [SMILES: CC(C)(C)c1ccc2ccc3ccc(Br)nc3c2n1] | 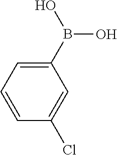 | 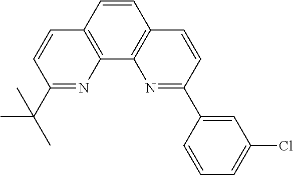 | 71 |
| | | IM B-7 | |
| 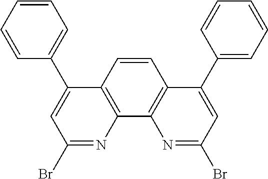 [SMILES: Brc1cc(-c2ccccc2)c2ccc3c(-c4ccccc4)cc(Br)nc3c2n1] | 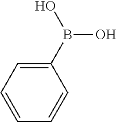 | 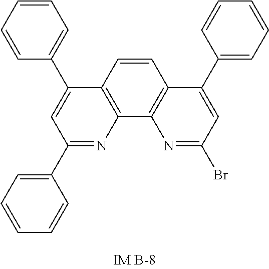 | 40 |
| 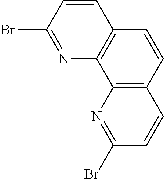 [SMILES: Brc1ccc2ccc3ccc(Br)nc3c2n1] | 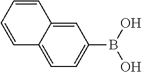 [SMILES: OB(O)c1ccc2ccccc2c1] | 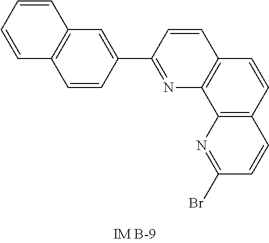 | 43 |
| 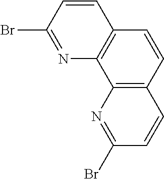 [SMILES: Brc1ccc2ccc3ccc(Br)nc3c2n1] | 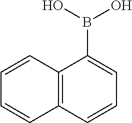 [SMILES: OB(O)c1cccc2ccccc12] | 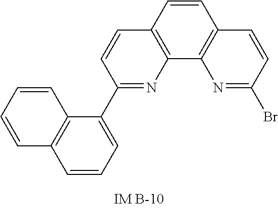 | 41 |
| 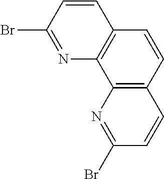 [SMILES: Brc1ccc2ccc3ccc(Br)nc3c2n1] | 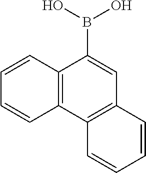 [SMILES: OB(O)c1cc2ccccc2c2ccccc12] | 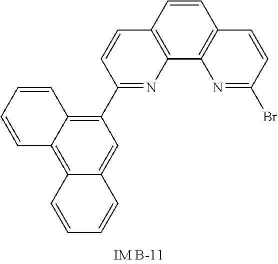 | 39 |
| 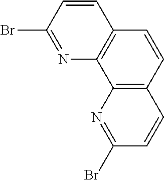 [SMILES: Brc1ccc2ccc3ccc(Br)nc3c2n1] | 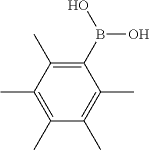 | 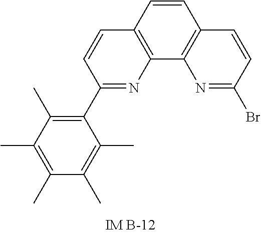 | 42 |
| 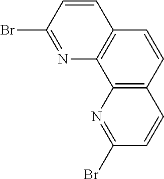 [SMILES: Brc1ccc2ccc3ccc(Br)nc3c2n1] | 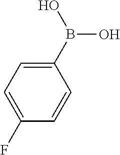 | 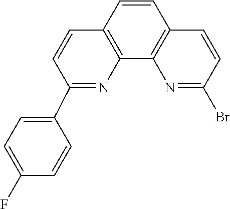 | 38 |
| | | IM B-13 | |
| 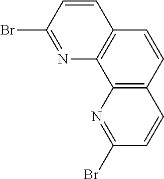 [SMILES: Brc1ccc2ccc3ccc(Br)nc3c2n1] | 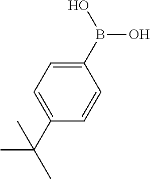 | 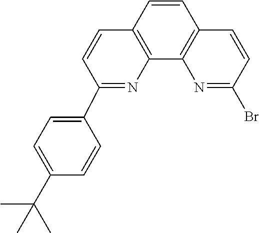 | 46 |
| | | IM B-14 | |
| 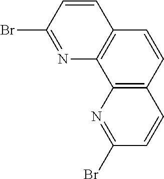 [SMILES: Brc1ccc2ccc3ccc(Br)nc3c2n1] | 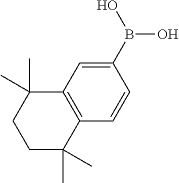 [SMILES: CC1(C)CCC(C)(C)c2cc(B(O)O)ccc21] | 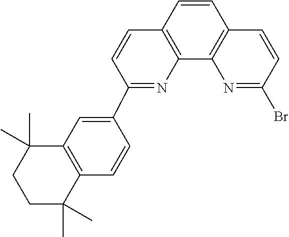 | 44 |
| | | IM B-15 | |
| 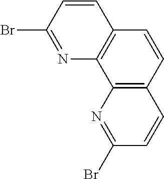 [SMILES: Brc1ccc2ccc3ccc(Br)nc3c2n1] | 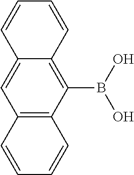 [SMILES: OB(O)c1c2ccccc2cc2ccccc12] | 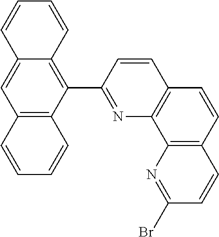 | 41 |
| | | IM B-16 | |
| 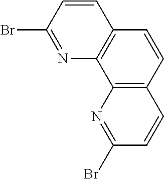 [SMILES: Brc1ccc2ccc3ccc(Br)nc3c2n1] | 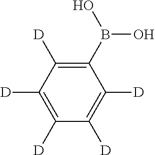 [SMILES: [2H]c1c([2H])c([2H])c(B(O)O)c([2H])c1[2H]] | 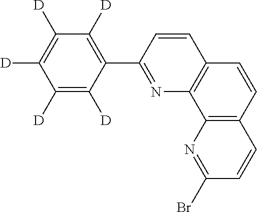 | 43 |
| | | IM B-17 | |
| 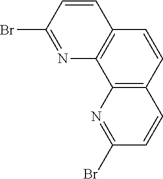 [SMILES: Brc1ccc2ccc3ccc(Br)nc3c2n1] | 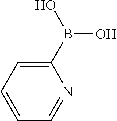 | 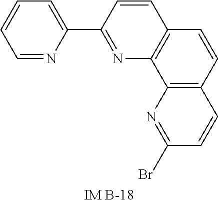 | 39 |
| 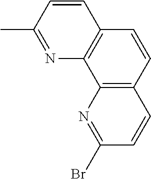 [SMILES: Cc1ccc2ccc3ccc(Br)nc3c2n1] | 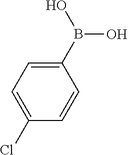 | 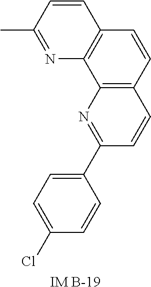 | 42 |
| 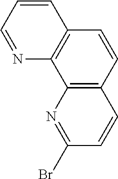 [SMILES: Brc1ccc2ccc3cccnc3c2n1] | 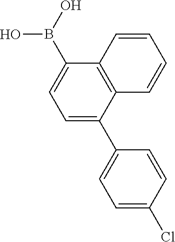 [SMILES: OB(O)c1ccc(-c2ccc(Cl)cc2)c2ccccc12] | 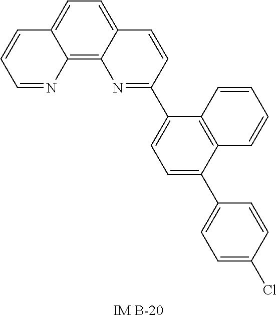 | 57 |

### 3. Synthesis of IM C-1

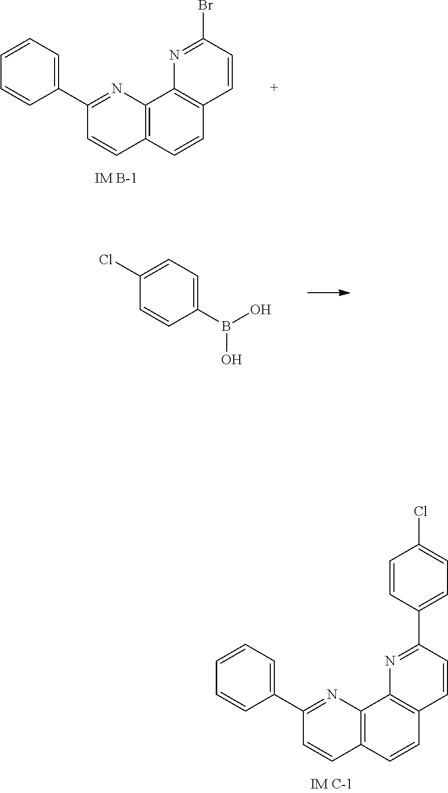 [SMILES: Brc1ccc2ccc3ccc(-c4ccccc4)nc3c2n1 ; OB(O)c1ccc(Cl)cc1 ; Clc1ccc(-c2ccc3ccc4ccc(-c5ccccc5)nc4c3n2)cc1]

IM B-1 (8 g, 23.87 mmol) and 4-chlorophenylboronic acid (3.73 g, 23.87 mmol) were dissolved in 64 mL of toluene, and tetrakis(triphenylphosphine) palladium (0.14 g, 0.119 mmol), K₂CO₃ (6.59 g, 47.73 mmol), 8 mL of ethanol and 8 mL of water were then added. Heating reflux was performed under a nitrogen atmosphere for 6 hours. After cooling to room temperature, the reaction solution was extracted with water and dichloromethane, dried over anhydrous magnesium sulfate, filtered, and concentrated under reduced pressure. The obtained solid was recrystallized by using dichloromethane:n-heptane=1:4 (v/v) to obtain IM C-1 (5.52 g, yield: 63%).

IM C-X was synthesized by the same method as that of IM C-1, except that a Raw material 4 was used instead of IM B-1 and a Raw material 5 was used instead of 4-chlorophenylboronic acid, where the main raw materials used, IM C-X synthesized and their yields are shown in Table 3.

TABLE 3

| Raw material 4 | Raw material 5 | IM C-X | Yield/% |
|---|---|---|---|
| 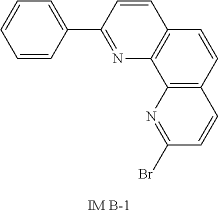 [SMILES: Brc1ccc2ccc3ccc(-c4ccccc4)nc3c2n1] | 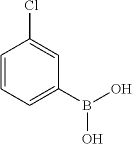 | 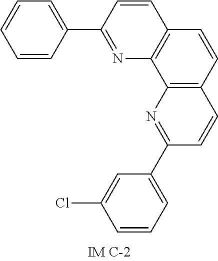 | 71 |
| 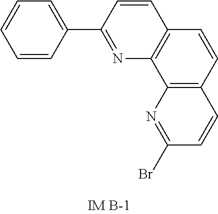 [SMILES: Brc1ccc2ccc3ccc(-c4ccccc4)nc3c2n1] | 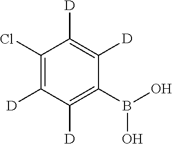 [SMILES: [2H]c1c([2H])c(B(O)O)c([2H])c([2H])c1Cl] | 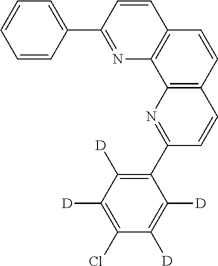 | 73 |
| | | IM C-3 | |
| 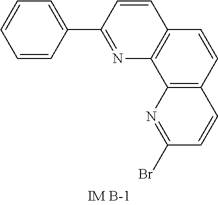 [SMILES: Brc1ccc2ccc3ccc(-c4ccccc4)nc3c2n1] | 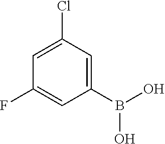 [SMILES: OB(O)c1cc(F)cc(Cl)c1] | 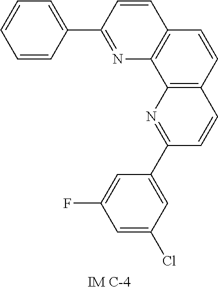 | 61 |
| 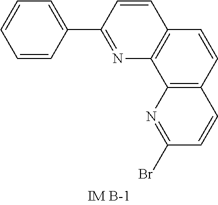 [SMILES: Brc1ccc2ccc3ccc(-c4ccccc4)nc3c2n1] | 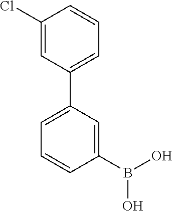 [SMILES: OB(O)c1cccc(-c2cccc(Cl)c2)c1] | 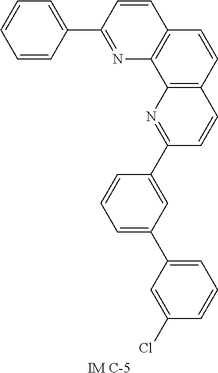 | 69 |
| 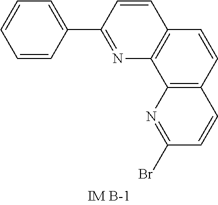 [SMILES: Brc1ccc2ccc3ccc(-c4ccccc4)nc3c2n1] | 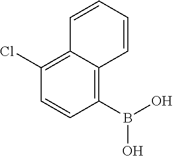 [SMILES: OB(O)c1ccc(Cl)c2ccccc12] | 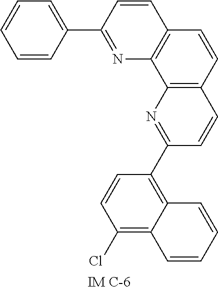 | 72 |
| 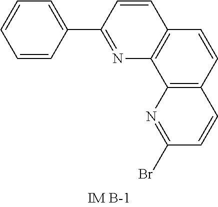 [SMILES: Brc1ccc2ccc3ccc(-c4ccccc4)nc3c2n1] | 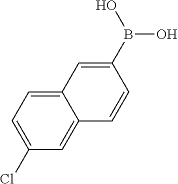 [SMILES: OB(O)c1ccc2cc(Cl)ccc2c1] | 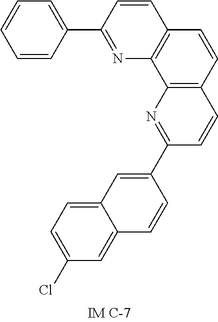 | 68 |
| 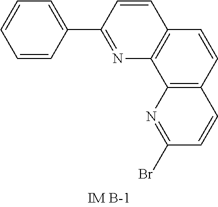 [SMILES: Brc1ccc2ccc3ccc(-c4ccccc4)nc3c2n1] | 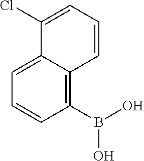 [SMILES: OB(O)c1cccc2c(Cl)cccc12] | 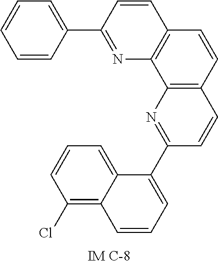 | 67 |
| 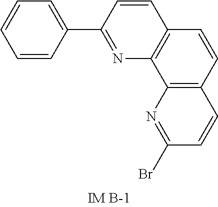 [SMILES: Brc1ccc2ccc3ccc(-c4ccccc4)nc3c2n1] | 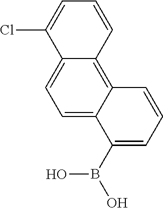 [SMILES: OB(O)c1cccc2c1ccc1c(Cl)cccc12] | 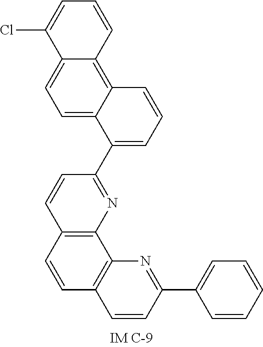 | 65 |
| 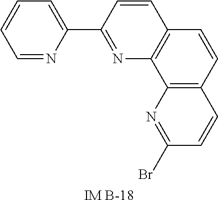 | 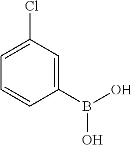 | 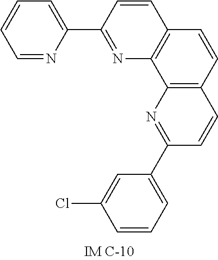 | 72 |
| 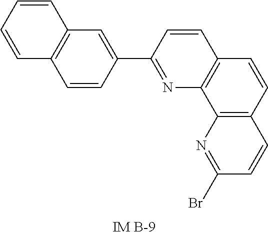 [SMILES: Brc1ccc2ccc3ccc(-c4ccc5ccccc5c4)nc3c2n1] | 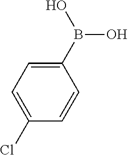 | 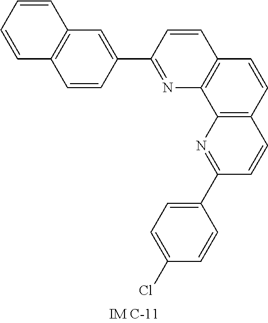 | 74 |
| 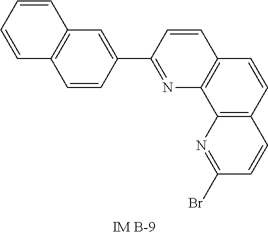 [SMILES: Brc1ccc2ccc3ccc(-c4ccc5ccccc5c4)nc3c2n1] | 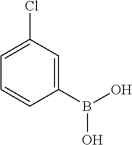 | 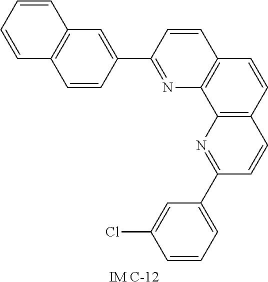 | 69 |
| 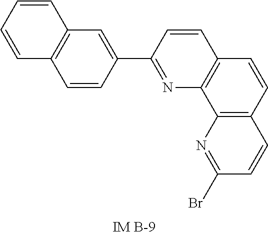 [SMILES: Brc1ccc2ccc3ccc(-c4ccc5ccccc5c4)nc3c2n1] | 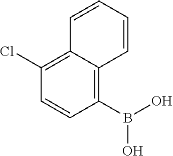 [SMILES: OB(O)c1ccc(Cl)c2ccccc12] | 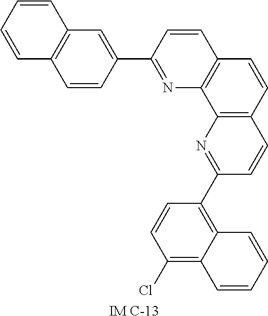 | 68 |
| 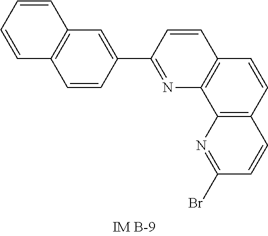 [SMILES: Brc1ccc2ccc3ccc(-c4ccc5ccccc5c4)nc3c2n1] | 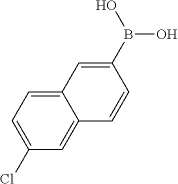 [SMILES: OB(O)c1ccc2cc(Cl)ccc2c1] | 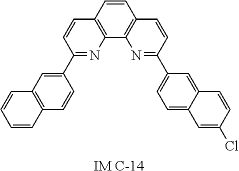 | 65 |
| 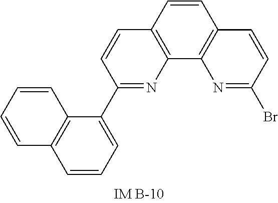 [SMILES: Brc1ccc2ccc3ccc(-c4cccc5ccccc45)nc3c2n1] | 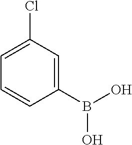 | 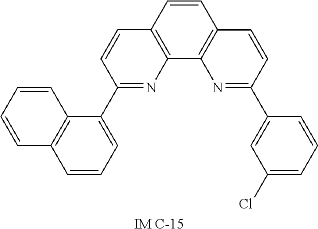 | 67 |
| 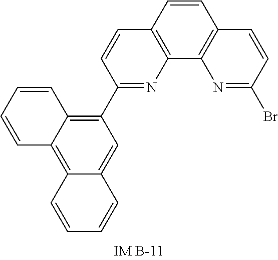 [SMILES: Brc1ccc2ccc3ccc(-c4cc5ccccc5c5ccccc45)nc3c2n1] | 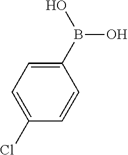 | 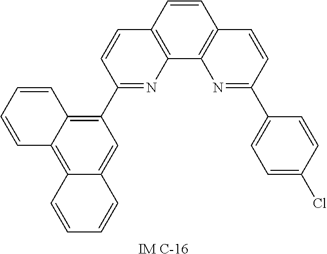 | 70 |
| 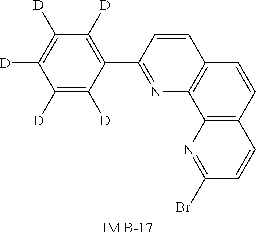 | 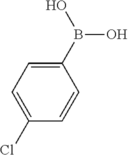 | 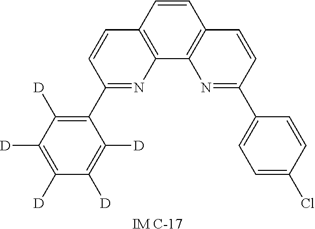 | 64 |
| 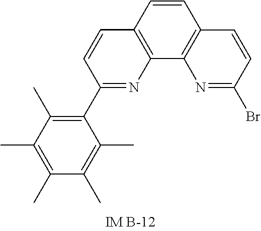 [SMILES: Cc1c(C)c(C)c(-c2ccc3ccc4ccc(Br)nc4c3n2)c(C)c1C] | 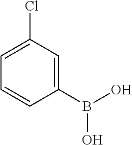 | 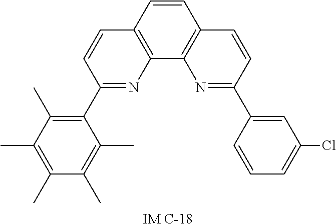 | 68 |
| 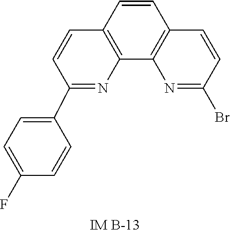 | 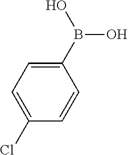 | 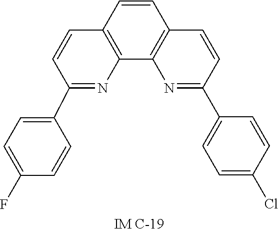 | 62 |
| 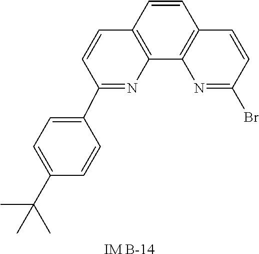 | 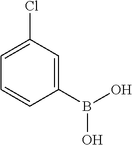 | 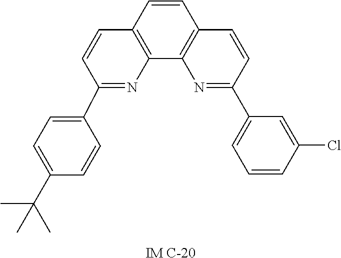 | 71 |
| 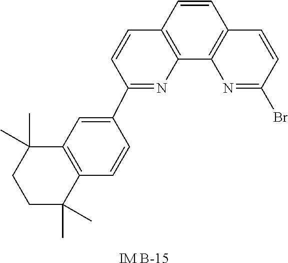 | 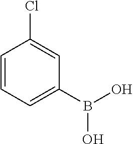 | 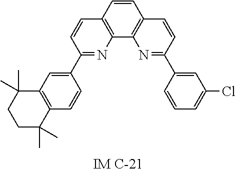 | 69 |
| 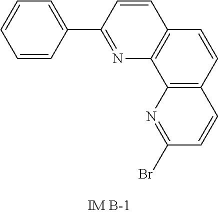 [SMILES: Brc1ccc2ccc3ccc(-c4ccccc4)nc3c2n1] | 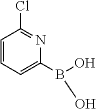 | 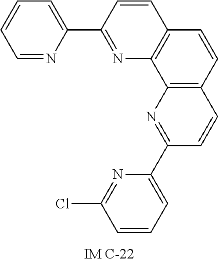 | 62 |
| 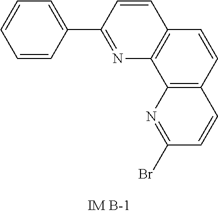 [SMILES: Brc1ccc2ccc3ccc(-c4ccccc4)nc3c2n1] | 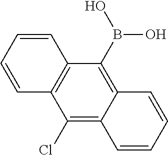 [SMILES: OB(O)c1c2ccccc2c(Cl)c2ccccc12] | 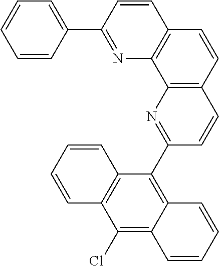 | 60 |
| | | IM C-23 | |
| 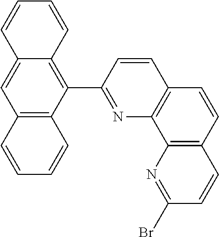 [SMILES: Brc1ccc2ccc3ccc(-c4c5ccccc5cc5ccccc45)nc3c2n1] | 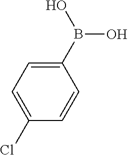 | 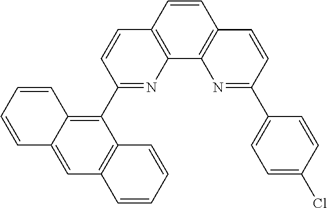 IM C-24 | 66 |
| IM B-16 | | | |
| 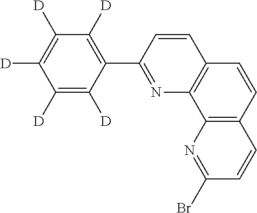 [SMILES: [2H]c1c([2H])c([2H])c(-c2ccc3ccc4ccc(Br)nc4c3n2)c([2H])c1[2H]] IM B-17 | 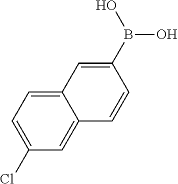 [SMILES: OB(O)c1ccc2cc(Cl)ccc2c1] | 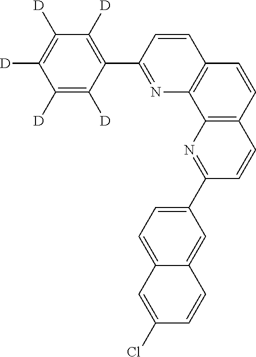 | 63 |
| | | IM C-25 | |

### 4. Synthesis of IM D-1

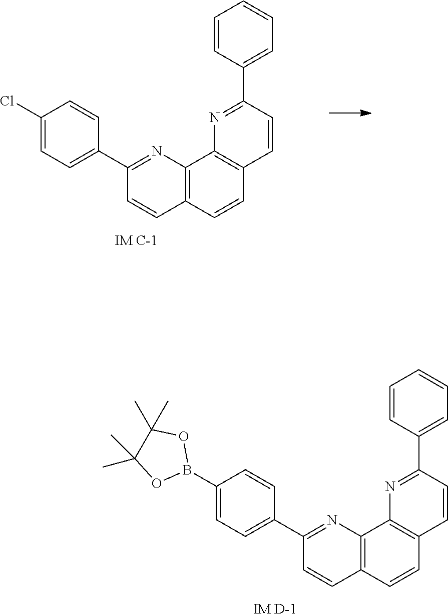

IM C-1 (5 g, 13.63 mmol) and bis(pinacolato)diboron (4.15 g, 16.36 mmol) were dissolved in 50 mL of 1,4-dioxane, and Pd(dba₂)₃ (0.12 g, 0.136 mmol), x-phos (0.13 g, 0.273 mmol) and potassium acetate (2.01 g, 20.44 mmol) were then added. Heating reflux was performed under a nitrogen atmosphere for 5 hours. After cooling to room temperature, the reaction solution was extracted with water and dichloromethane, dried over anhydrous magnesium sulfate, filtered, and concentrated under reduced pressure. The obtained solid was recrystallized by using dichloromethane:n-heptane=1:3 (v/v) to obtain IM D-1 (4.87 g, yield: 78%).

IM D-X was synthesized by the same method as that of IM D-1, except that a Raw material 6 was used instead of IM C-1, where the main raw material used, IM D-X synthesized and their yields are shown in Table 4.

TABLE 4

| Raw material 6 | IM D-X | Yield/% |
|---|---|---|
| 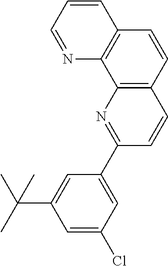 [SMILES: CC(C)(C)c1cc(Cl)cc(-c2ccc3ccc4cccnc4c3n2)c1] IM B-2 | 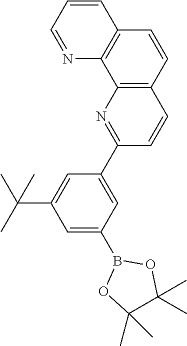 | 80 |
| | IM D-2 | |
| 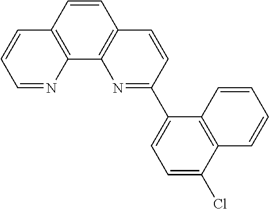 [SMILES: Clc1ccc(-c2ccc3ccc4cccnc4c3n2)c2ccccc12] IM B-3 | 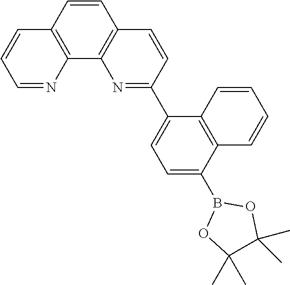 | 79 |
| | IM D-3 | |
| 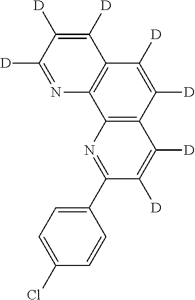 [SMILES: [2H]c1nc2c(c([2H])c1[2H])c([2H])c([2H])c1c([2H])c([2H])c(-c3ccc(Cl)cc3)nc12] IM B-6 | 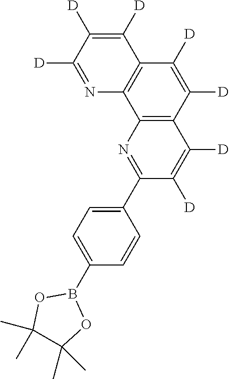 | 81 |
| | IM D-4 | |
| 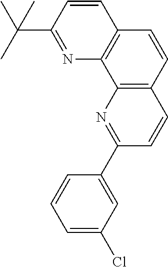 [SMILES: CC(C)(C)c1ccc2ccc3ccc(-c4cccc(Cl)c4)nc3c2n1] IM B-7 | 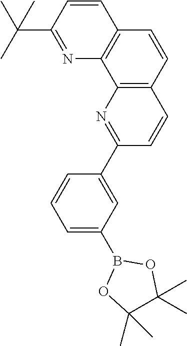 | 83 |
| | IM D-5 | |
| 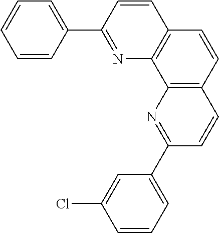 [SMILES: Clc1cccc(-c2ccc3ccc4ccc(-c5ccccc5)nc4c3n2)c1] | 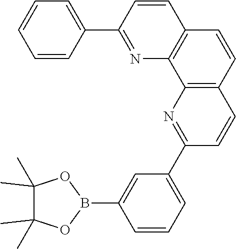 | 78 |
| IM C-2 | | |
| | IM D-6 | |
| 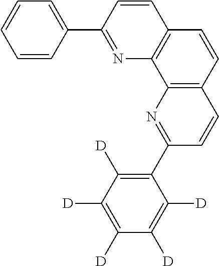 [SMILES: [2H]c1c([2H])c([2H])c(-c2ccc3ccc4ccc(-c5ccccc5)nc4c3n2)c([2H])c1[2H]] IM C-3 | 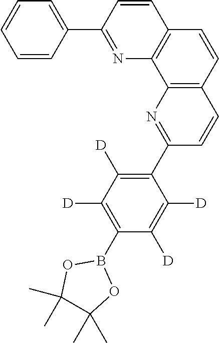 | 74 |
| | IM D-7 | |
| 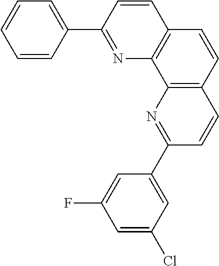 [SMILES: Fc1cc(Cl)cc(-c2ccc3ccc4ccc(-c5ccccc5)nc4c3n2)c1] IM C-4 | 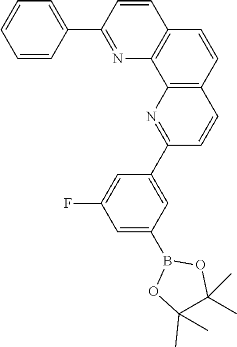 | 71 |
| | IM D-8 | |
| 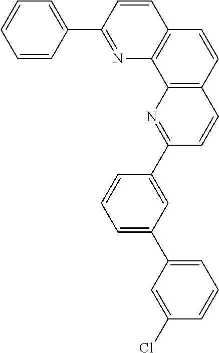 [SMILES: Clc1cccc(-c2cccc(-c3ccc4ccc5ccc(-c6ccccc6)nc5c4n3)c2)c1] IM C-5 | 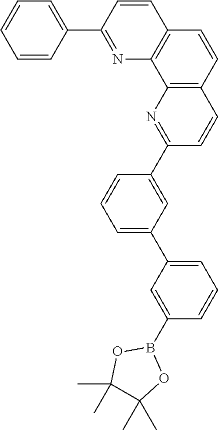 | 83 |
| | IM D-9 | |
| 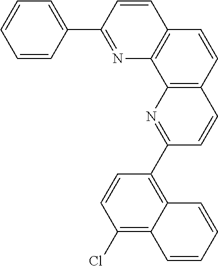 [SMILES: Clc1ccc(-c2ccc3ccc4ccc(-c5ccccc5)nc4c3n2)c2ccccc12] IM C-6 | 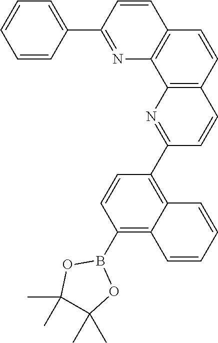 | 79 |
| | IM D-10 | |
| 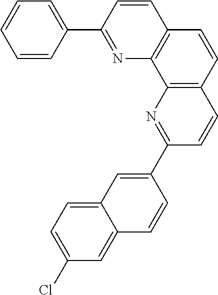 [SMILES: Clc1ccc2cc(-c3ccc4ccc5ccc(-c6ccccc6)nc5c4n3)ccc2c1] IM C-7 | 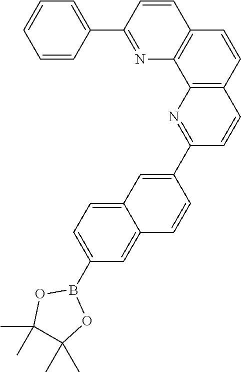 | 80 |
| | IM D-11 | |
| 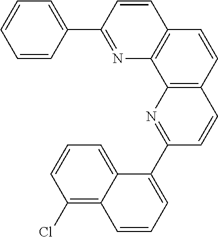 [SMILES: Clc1cccc2c(-c3ccc4ccc5ccc(-c6ccccc6)nc5c4n3)cccc12] IM C-8 | 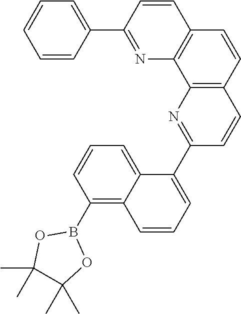 | 81 |
| | IM D-12 | |
| 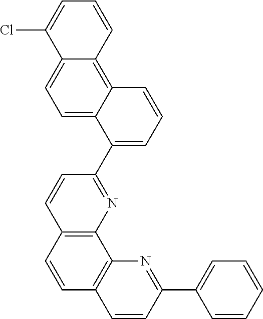 [SMILES: Clc1cccc2c1ccc1c(-c3ccc4ccc5ccc(-c6ccccc6)nc5c4n3)cccc12] IM C-9 | 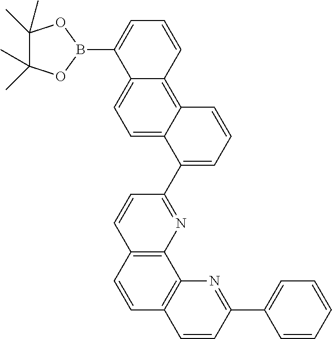 | 73 |
| | IM D-13 | |
| 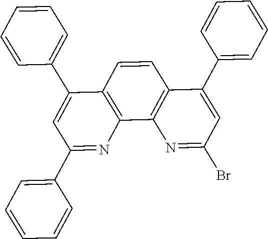 [SMILES: Brc1cc(-c2ccccc2)c2ccc3c(-c4ccccc4)cc(-c4ccccc4)nc3c2n1] IM B-8 | 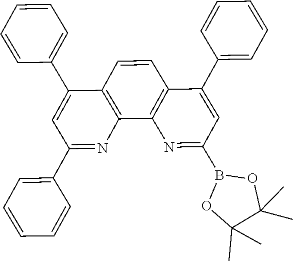 | 46 |
| | IM D-14 | |
| 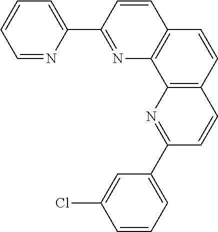 [SMILES: Clc1cccc(-c2ccc3ccc4ccc(-c5ccccn5)nc4c3n2)c1] IM C-10 | 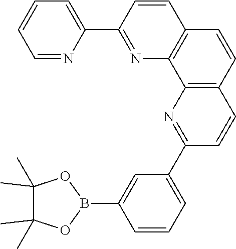 | 75 |
| | IM D-15 | |
| 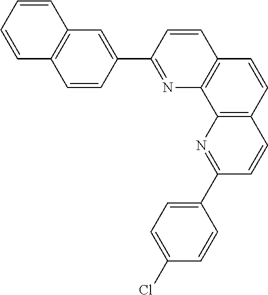 [SMILES: Clc1ccc(-c2ccc3ccc4ccc(-c5ccc6ccccc6c5)nc4c3n2)cc1] IM C-11 | 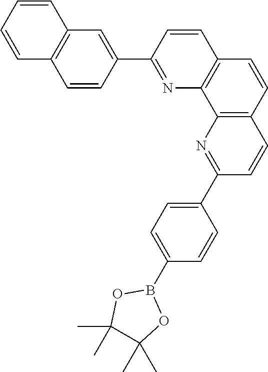 | 82 |
| | IM D-16 | |
| 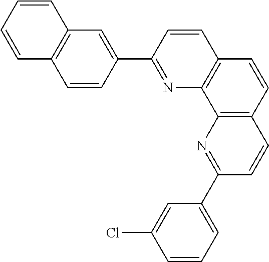 [SMILES: Clc1cccc(-c2ccc3ccc4ccc(-c5ccc6ccccc6c5)nc4c3n2)c1] | 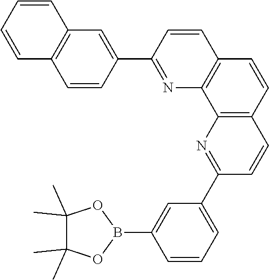 | 81 |
| IM C-12 | | |
| | IM D-17 | |
| 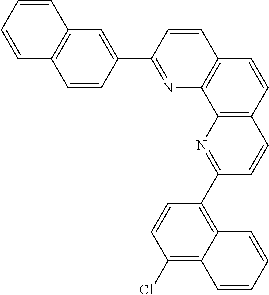 [SMILES: Clc1ccc(-c2ccc3ccc4ccc(-c5ccc6ccccc6c5)nc4c3n2)c2ccccc12] IM C-13 | 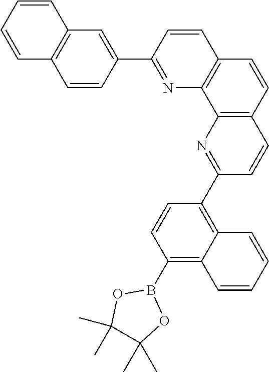 | 78 |
| | IM D-18 | |
| 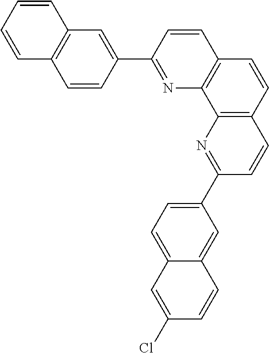 [SMILES: Clc1ccc2cc(-c3ccc4ccc5ccc(-c6ccc7ccccc7c6)nc5c4n3)ccc2c1] IM C-14 | 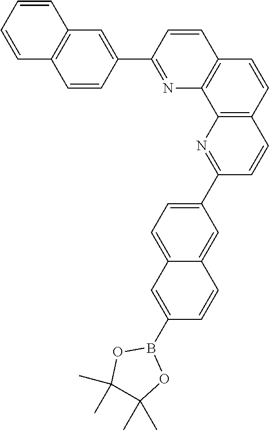 | 76 |
| | IM D-19 | |
| 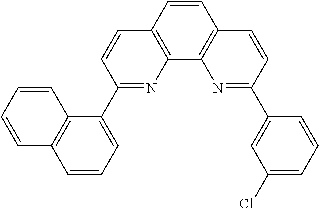 [SMILES: Clc1cccc(-c2ccc3ccc4ccc(-c5cccc6ccccc56)nc4c3n2)c1] IM C-15 | 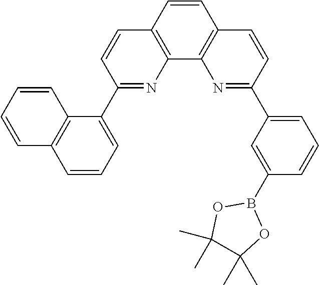 | 83 |
| | IM D-20 | |
| 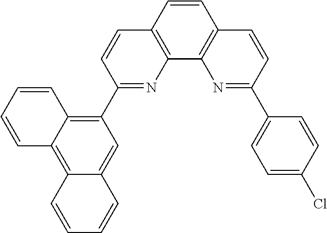 [SMILES: Clc1ccc(-c2ccc3ccc4ccc(-c5cc6ccccc6c6ccccc56)nc4c3n2)cc1] IM C-16 | 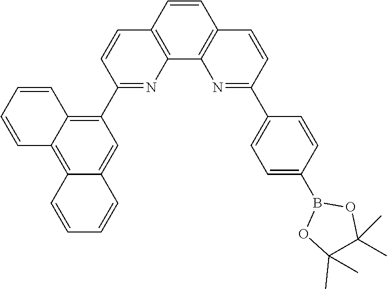 | 76 |
| | IM D-21 | |
| 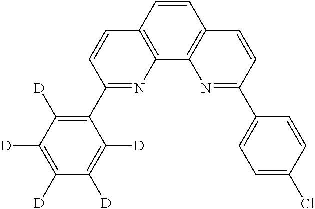 [SMILES: [2H]c1c([2H])c([2H])c(-c2ccc3ccc4ccc(-c5ccc(Cl)cc5)nc4c3n2)c([2H])c1[2H]] IM C-17 | 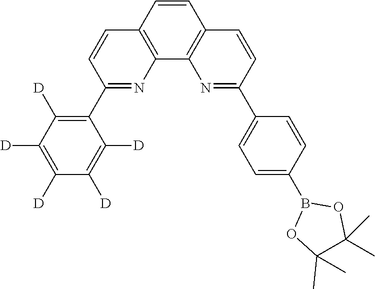 | 75 |
| | IM D-22 | |
| 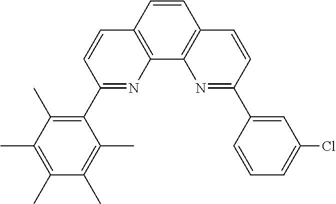 [SMILES: Cc1c(C)c(C)c(-c2ccc3ccc4ccc(-c5cccc(Cl)c5)nc4c3n2)c(C)c1C] | 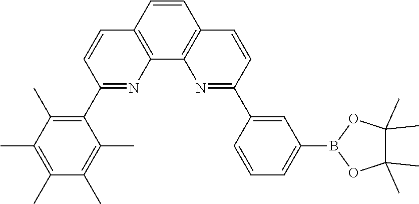 | 71 |
| IM C-18 | IM D-23 | |
| 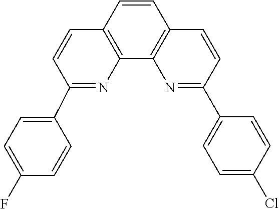 [SMILES: Fc1ccc(-c2ccc3ccc4ccc(-c5ccc(Cl)cc5)nc4c3n2)cc1] IM C-19 | 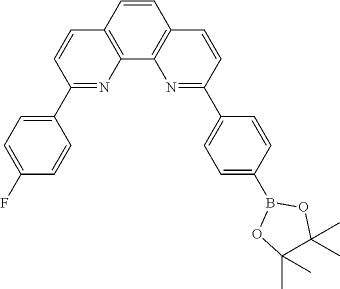 | 79 |
| | IM D-24 | |
| 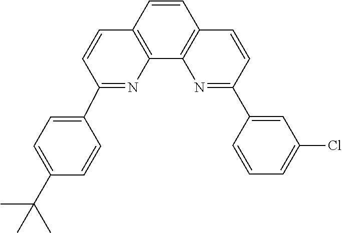 [SMILES: CC(C)(C)c1ccc(-c2ccc3ccc4ccc(-c5cccc(Cl)c5)nc4c3n2)cc1] | 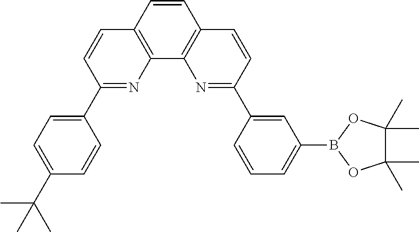 | 73 |
| IM C-20 | IM D-25 | |
| 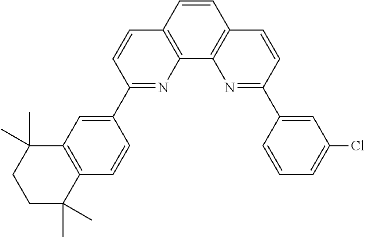 [SMILES: CC1(C)CCC(C)(C)c2cc(-c3ccc4ccc5ccc(-c6cccc(Cl)c6)nc5c4n3)ccc21] | 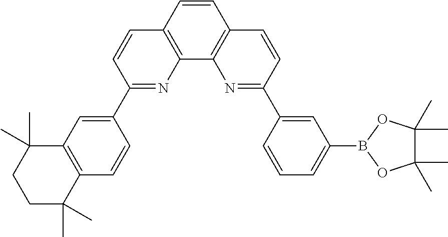 | 81 |
| IM C-21 | IM D-26 | |
| 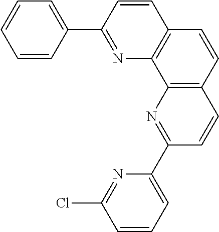 [SMILES: Clc1cccc(-c2ccc3ccc4ccc(-c5ccccc5)nc4c3n2)n1] | 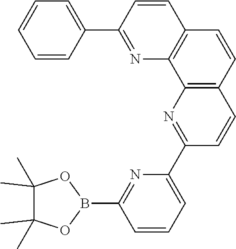 | 61 |
| IM C-22 | | |
| | IM D-27 | |
| 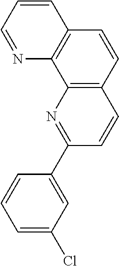 [SMILES: Clc1cccc(-c2ccc3ccc4cccnc4c3n2)c1] IM B-4 | 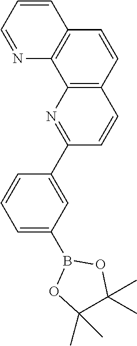 | 83 |
| | IM D-28 | |
| 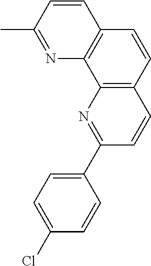 [SMILES: Cc1ccc2ccc3ccc(-c4ccc(Cl)cc4)nc3c2n1] IM B-19 | 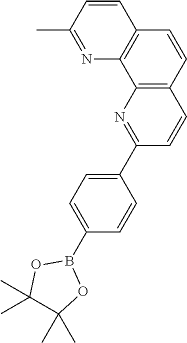 | 82 |
| | IM D-30 | |
| 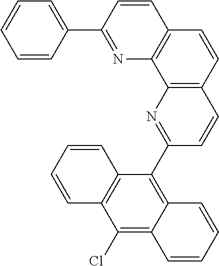 [SMILES: Clc1c2ccccc2c(-c2ccc3ccc4ccc(-c5ccccc5)nc4c3n2)c2ccccc12] IM C-23 | 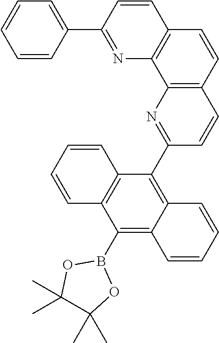 | 69 |
| | IM D-31 | |
| 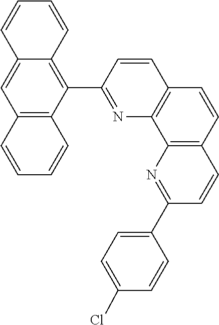 [SMILES: Clc1ccc(-c2ccc3ccc4ccc(-c5c6ccccc6cc6ccccc56)nc4c3n2)cc1] IM C-24 | 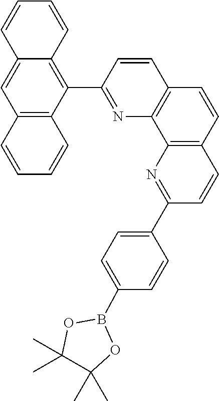 | 74 |
| | IM D-32 | |
| 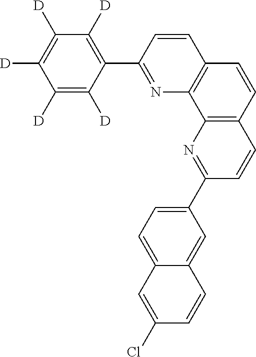 [SMILES: [2H]c1c([2H])c([2H])c(-c2ccc3ccc4ccc(-c5ccc6cc(Cl)ccc6c5)nc4c3n2)c([2H])c1[2H]] IM C-25 | 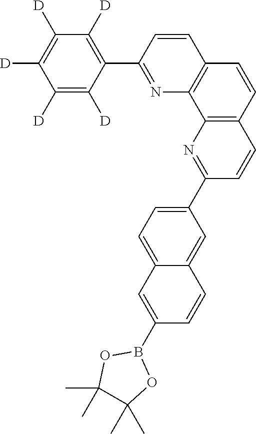 | 68 |
| | IM D-33 | |
| 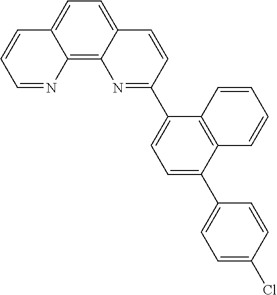 [SMILES: Clc1ccc(-c2ccc(-c3ccc4ccc5cccnc5c4n3)c3ccccc23)cc1] IM B-20 | 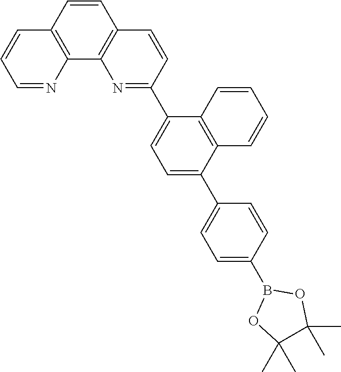 | 56 |
| | IM D-34 | |

### Synthesis Example 1 Synthesis of Compound 3

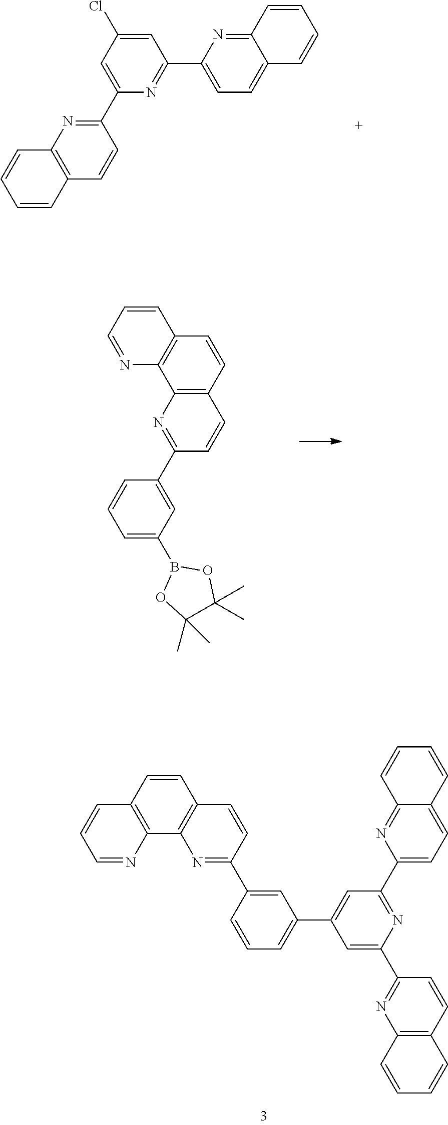 [SMILES: Clc1cc(-c2ccc3ccccc3n2)nc(-c2ccc3ccccc3n2)c1 ; CC1(C)OB(c2cccc(-c3ccc4ccc5cccnc5c4n3)c2)OC1(C)C ; c1cc(-c2cc(-c3ccc4ccccc4n3)nc(-c3ccc4ccccc4n3)c2)cc(-c2ccc3ccc4cccnc4c3n2)c1]

IM A-1 (5 g, 13.6 mmol) and IM D-28 (5.2 g, 13.6 mmol) were dissolved in 40 mL of toluene, and tetrakis(triphenylphosphine) palladium (0.16 g, 0.136 mmol), K₂CO₃ (3.8 g, 27.2 mmol), 5 mL of ethanol and 5 mL of water were then added. Heating reflux was performed under a nitrogen atmosphere for 12 hours. After cooling to room temperature, 100 ml of water was added, stirring was performed for 30 min, suction filtration was performed, a solid was collected, and the filtered solid was rinsed with 100 mL of ethanol for 3 times. The obtained solid was recrystallized by using dichloromethane to obtain a compound 3 (3.7 g, yield: 46%), mass spectrum (m/z)=588.21 [M+H]⁺.

Compounds X listed in Table 5 were synthesized by the same method as that of the compound 3, except that a Raw material 7 was used instead of IM A-1 and a Raw material 8 was used instead of IM D-28, where the main raw materials used, the synthesized compounds and their yields and mass spectra are shown in Table 5.

TABLE 5

| Synthesis | | |
|---|---|---|
| example | | |
| No. | Raw material 7 | Raw material 8 |
| 2 | 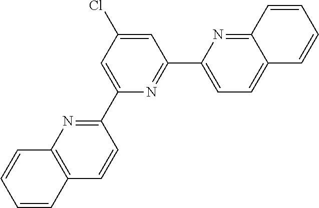 [SMILES: Clc1cc(-c2ccc3ccccc3n2)nc(-c2ccc3ccccc3n2)c1] | 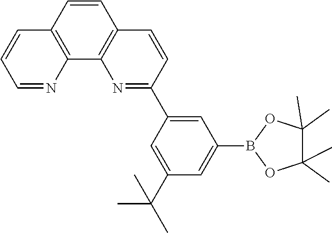 [SMILES: CC(C)(C)c1cc(B2OC(C)(C)C(C)(C)O2)cc(-c2ccc3ccc4cccnc4c3n2)c1] |
| | | IM D-2 |
| 3 | 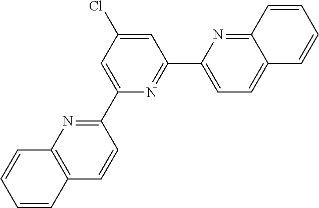 [SMILES: Clc1cc(-c2ccc3ccccc3n2)nc(-c2ccc3ccccc3n2)c1] | 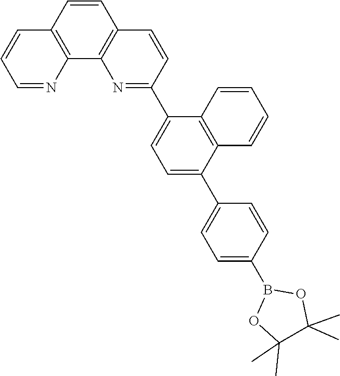 [SMILES: CC1(C)OB(c2ccc(-c3ccc(-c4ccc5ccc6cccnc6c5n4)c4ccccc34)cc2)OC1(C)C] |
| | | IM D-34 |
| 4 | 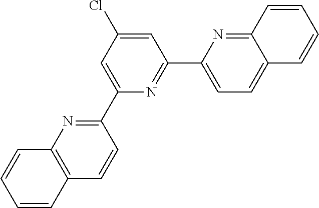 [SMILES: Clc1cc(-c2ccc3ccccc3n2)nc(-c2ccc3ccccc3n2)c1] | 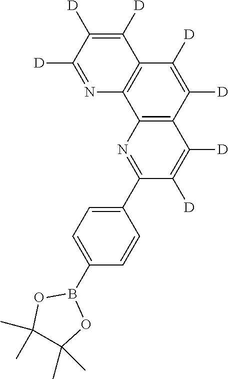 [SMILES: [2H]c1nc2c(c([2H])c1[2H])c([2H])c([2H])c1c([2H])c([2H])c(-c3ccc(B4OC(C)(C)C(C)(C)O4)cc3)nc12] |
| | | IM D-4 |
| 5 | 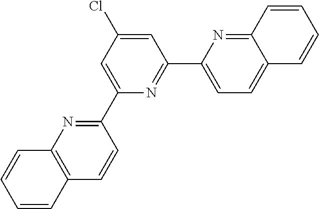 [SMILES: Clc1cc(-c2ccc3ccccc3n2)nc(-c2ccc3ccccc3n2)c1] | 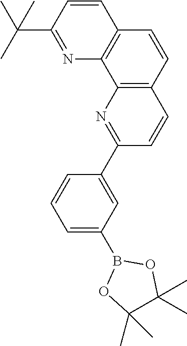 [SMILES: CC(C)(C)c1ccc2ccc3ccc(-c4cccc(B5OC(C)(C)C(C)(C)O5)c4)nc3c2n1] |
| | | IM D-5 |
| 6 | 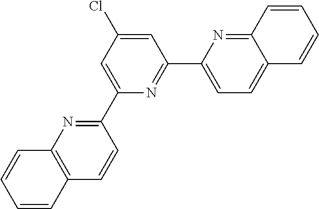 [SMILES: Clc1cc(-c2ccc3ccccc3n2)nc(-c2ccc3ccccc3n2)c1] | 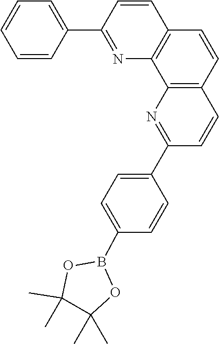 [SMILES: CC1(C)OB(c2ccc(-c3ccc4ccc5ccc(-c6ccccc6)nc5c4n3)cc2)OC1(C)C] |
| | | IM D-1 |
| 7 | 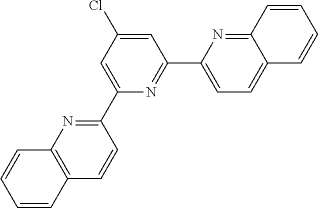 [SMILES: Clc1cc(-c2ccc3ccccc3n2)nc(-c2ccc3ccccc3n2)c1] | 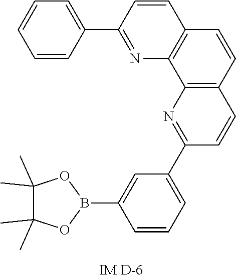 [SMILES: CC1(C)OB(c2cccc(-c3ccc4ccc5ccc(-c6ccccc6)nc5c4n3)c2)OC1(C)C] |
| 8 | 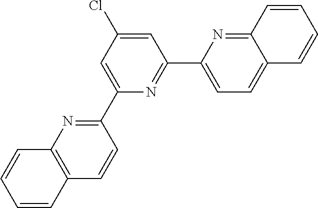 [SMILES: Clc1cc(-c2ccc3ccccc3n2)nc(-c2ccc3ccccc3n2)c1] | 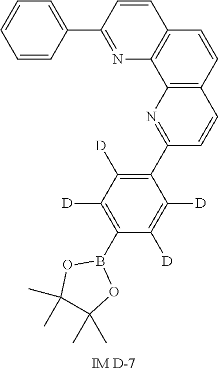 [SMILES: [2H]c1c([2H])c(-c2ccc3ccc4ccc(-c5ccccc5)nc4c3n2)c([2H])c([2H])c1B1OC(C)(C)C(C)(C)O1] |
| 9 | 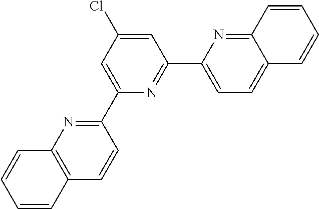 [SMILES: Clc1cc(-c2ccc3ccccc3n2)nc(-c2ccc3ccccc3n2)c1] | 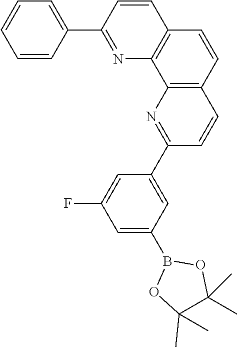 [SMILES: CC1(C)OB(c2cc(F)cc(-c3ccc4ccc5ccc(-c6ccccc6)nc5c4n3)c2)OC1(C)C] |
| | | IM D-8 |
| 10 | 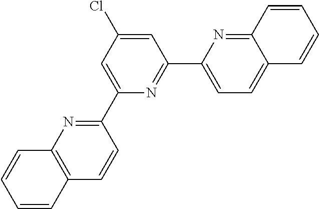 [SMILES: Clc1cc(-c2ccc3ccccc3n2)nc(-c2ccc3ccccc3n2)c1] | 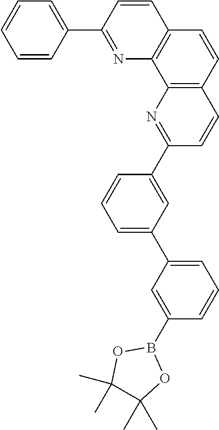 [SMILES: CC1(C)OB(c2cccc(-c3cccc(-c4ccc5ccc6ccc(-c7ccccc7)nc6c5n4)c3)c2)OC1(C)C] |
| | | IM D-9 |
| 11 | 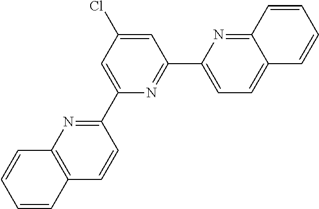 [SMILES: Clc1cc(-c2ccc3ccccc3n2)nc(-c2ccc3ccccc3n2)c1] | 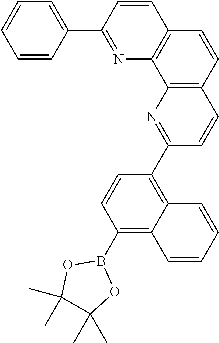 [SMILES: CC1(C)OB(c2ccc(-c3ccc4ccc5ccc(-c6ccccc6)nc5c4n3)c3ccccc23)OC1(C)C] |
| | | IM D-10 |
| 12 | 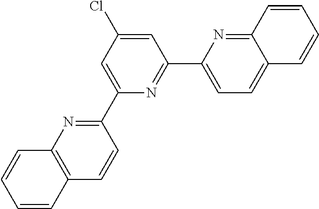 [SMILES: Clc1cc(-c2ccc3ccccc3n2)nc(-c2ccc3ccccc3n2)c1] | 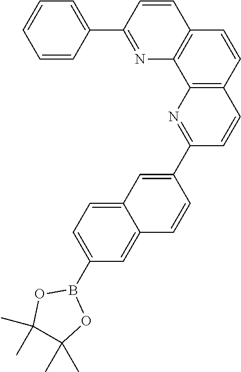 [SMILES: CC1(C)OB(c2ccc3cc(-c4ccc5ccc6ccc(-c7ccccc7)nc6c5n4)ccc3c2)OC1(C)C] |
| | | IM D-11 |
| 13 | 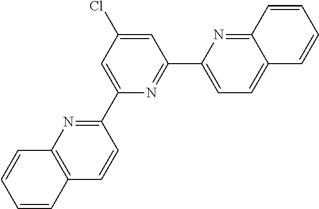 [SMILES: Clc1cc(-c2ccc3ccccc3n2)nc(-c2ccc3ccccc3n2)c1] | 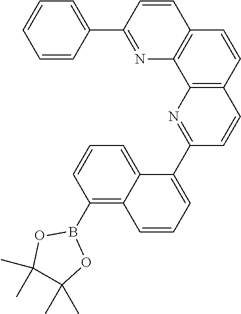 [SMILES: CC1(C)OB(c2cccc3c(-c4ccc5ccc6ccc(-c7ccccc7)nc6c5n4)cccc23)OC1(C)C] |
| | | IM D-12 |
| 14 | 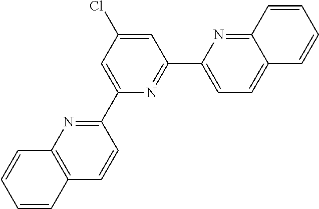 [SMILES: Clc1cc(-c2ccc3ccccc3n2)nc(-c2ccc3ccccc3n2)c1] | 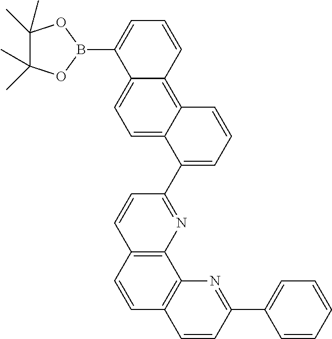 [SMILES: CC1(C)OB(c2cccc3c2ccc2c(-c4ccc5ccc6ccc(-c7ccccc7)nc6c5n4)cccc23)OC1(C)C] |
| | | IM D-13 |
| 15 | 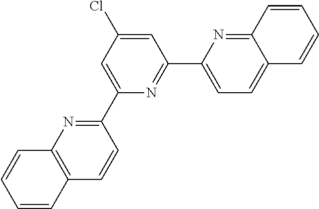 [SMILES: Clc1cc(-c2ccc3ccccc3n2)nc(-c2ccc3ccccc3n2)c1] | 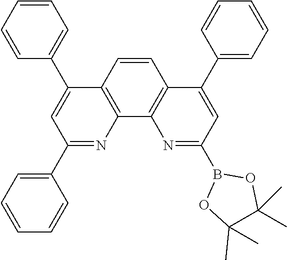 [SMILES: CC1(C)OB(c2cc(-c3ccccc3)c3ccc4c(-c5ccccc5)cc(-c5ccccc5)nc4c3n2)OC1(C)C] |
| | | IM D-14 |
| 16 | 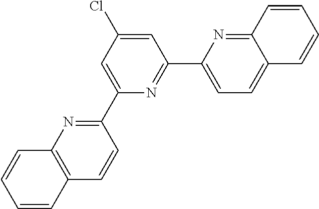 [SMILES: Clc1cc(-c2ccc3ccccc3n2)nc(-c2ccc3ccccc3n2)c1] | 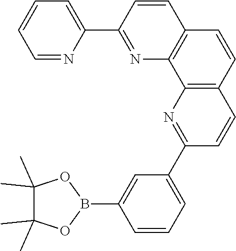 [SMILES: CC1(C)OB(c2cccc(-c3ccc4ccc5ccc(-c6ccccn6)nc5c4n3)c2)OC1(C)C] |
| | | IM D-15 |
| 17 | 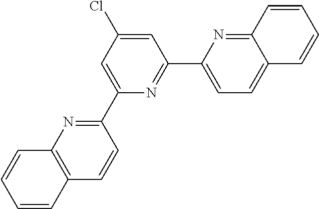 [SMILES: Clc1cc(-c2ccc3ccccc3n2)nc(-c2ccc3ccccc3n2)c1] | 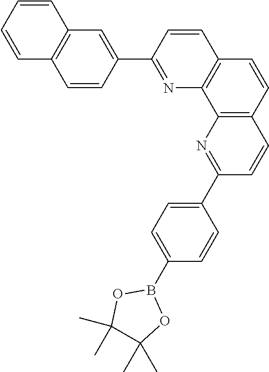 [SMILES: CC1(C)OB(c2ccc(-c3ccc4ccc5ccc(-c6ccc7ccccc7c6)nc5c4n3)cc2)OC1(C)C] |
| | | IM D-16 |
| 18 | 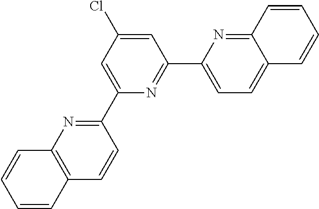 [SMILES: Clc1cc(-c2ccc3ccccc3n2)nc(-c2ccc3ccccc3n2)c1] | 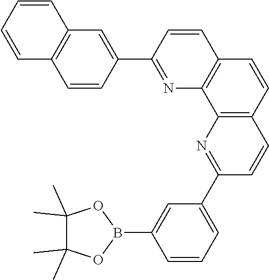 [SMILES: CC1(C)OB(c2cccc(-c3ccc4ccc5ccc(-c6ccc7ccccc7c6)nc5c4n3)c2)OC1(C)C] |
| | | IM D-17 |
| 19 | 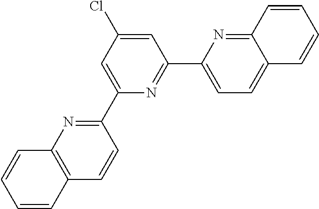 [SMILES: Clc1cc(-c2ccc3ccccc3n2)nc(-c2ccc3ccccc3n2)c1] | 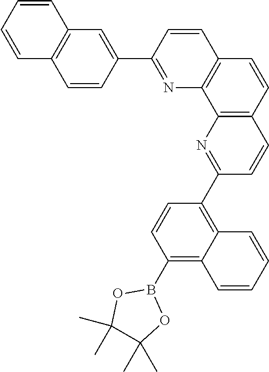 [SMILES: CC1(C)OB(c2ccc(-c3ccc4ccc5ccc(-c6ccc7ccccc7c6)nc5c4n3)c3ccccc23)OC1(C)C] |
| | | IM D-18 |
| 20 | 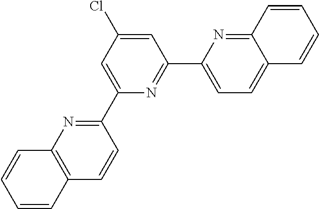 [SMILES: Clc1cc(-c2ccc3ccccc3n2)nc(-c2ccc3ccccc3n2)c1] | 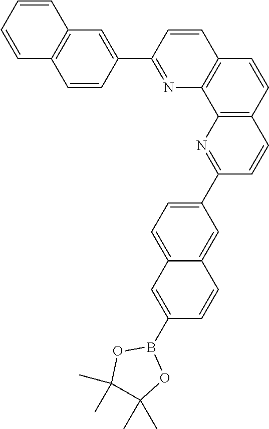 [SMILES: CC1(C)OB(c2ccc3cc(-c4ccc5ccc6ccc(-c7ccc8ccccc8c7)nc6c5n4)ccc3c2)OC1(C)C] |
| | | IM D-19 |
| 21 | 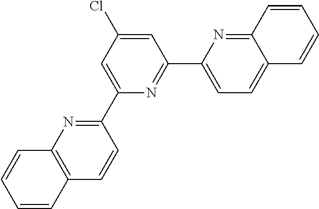 [SMILES: Clc1cc(-c2ccc3ccccc3n2)nc(-c2ccc3ccccc3n2)c1] | 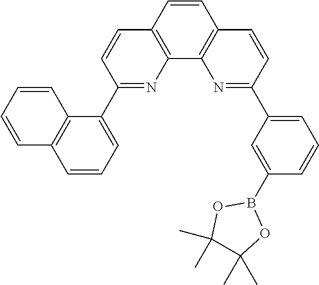 [SMILES: CC1(C)OB(c2cccc(-c3ccc4ccc5ccc(-c6cccc7ccccc67)nc5c4n3)c2)OC1(C)C] |
| | | IM D-20 |
| 22 | 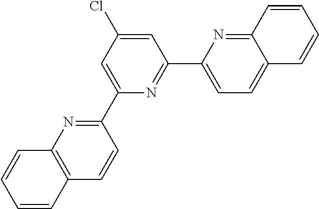 [SMILES: Clc1cc(-c2ccc3ccccc3n2)nc(-c2ccc3ccccc3n2)c1] | 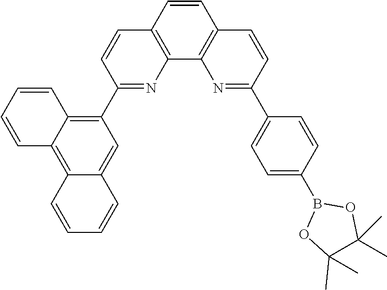 [SMILES: CC1(C)OB(c2ccc(-c3ccc4ccc5ccc(-c6cc7ccccc7c7ccccc67)nc5c4n3)cc2)OC1(C)C] |
| | | IM D-21 |
| 23 | 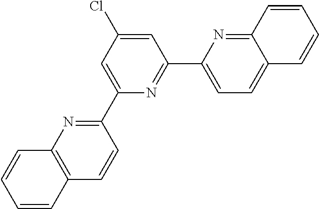 [SMILES: Clc1cc(-c2ccc3ccccc3n2)nc(-c2ccc3ccccc3n2)c1] | 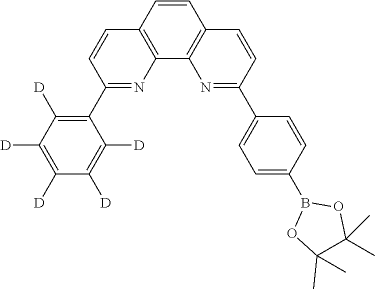 [SMILES: [2H]c1c([2H])c([2H])c(-c2ccc3ccc4ccc(-c5ccc(B6OC(C)(C)C(C)(C)O6)cc5)nc4c3n2)c([2H])c1[2H]] |
| | | IM D-22 |
| 24 | 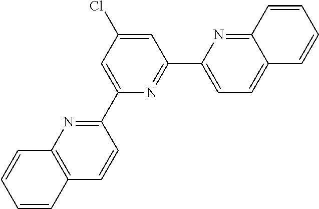 [SMILES: Clc1cc(-c2ccc3ccccc3n2)nc(-c2ccc3ccccc3n2)c1] | 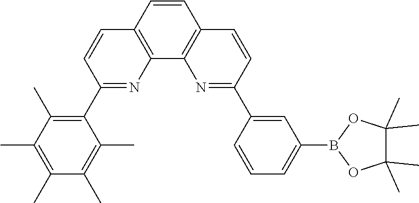 |
| | | IM D-23 |
| 25 | 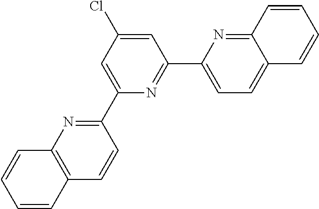 [SMILES: Clc1cc(-c2ccc3ccccc3n2)nc(-c2ccc3ccccc3n2)c1] | 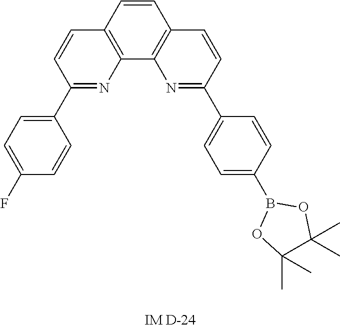 |
| 26 | 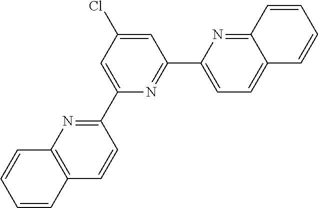 [SMILES: Clc1cc(-c2ccc3ccccc3n2)nc(-c2ccc3ccccc3n2)c1] | 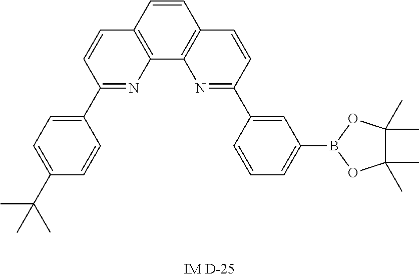 |
| 27 | 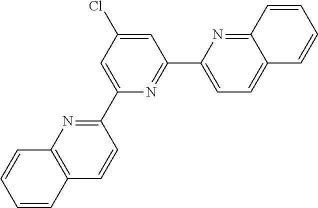 [SMILES: Clc1cc(-c2ccc3ccccc3n2)nc(-c2ccc3ccccc3n2)c1] | 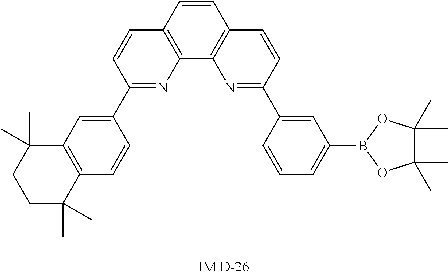 |
| 28 | 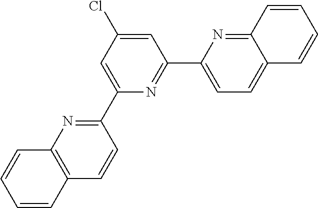 [SMILES: Clc1cc(-c2ccc3ccccc3n2)nc(-c2ccc3ccccc3n2)c1] | 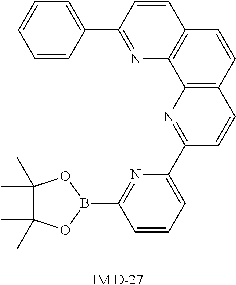 |
| 29 | 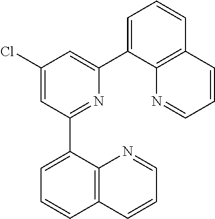 [SMILES: Clc1cc(-c2cccc3cccnc23)nc(-c2cccc3cccnc23)c1] | 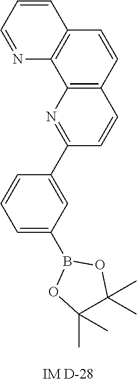 |
| 30 | 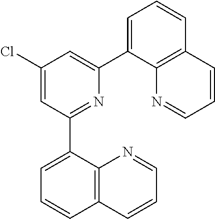 [SMILES: Clc1cc(-c2cccc3cccnc23)nc(-c2cccc3cccnc23)c1] | 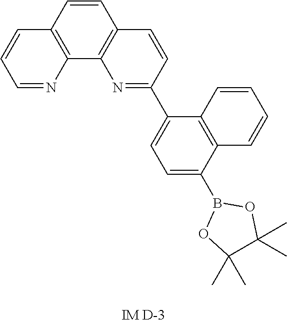 |
| 31 | 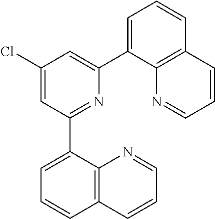 [SMILES: Clc1cc(-c2cccc3cccnc23)nc(-c2cccc3cccnc23)c1] | 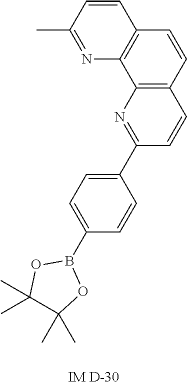 [SMILES: Cc1ccc2ccc3ccc(-c4ccc(B5OC(C)(C)C(C)(C)O5)cc4)nc3c2n1] |
| 32 | 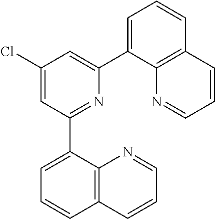 [SMILES: Clc1cc(-c2cccc3cccnc23)nc(-c2cccc3cccnc23)c1] | 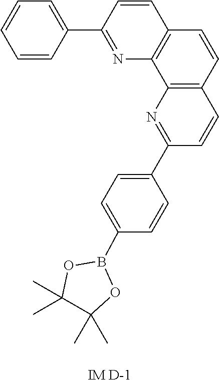 |
| 33 | 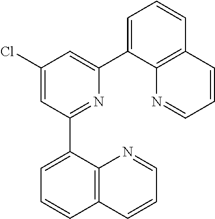 [SMILES: Clc1cc(-c2cccc3cccnc23)nc(-c2cccc3cccnc23)c1] | 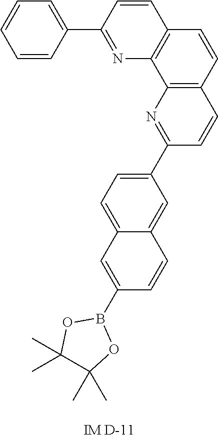 |
| 34 | 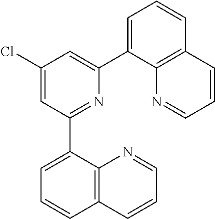 [SMILES: Clc1cc(-c2cccc3cccnc23)nc(-c2cccc3cccnc23)c1] | 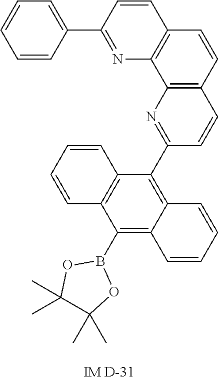 [SMILES: CC1(C)OB(c2c3ccccc3c(-c3ccc4ccc5ccc(-c6ccccc6)nc5c4n3)c3ccccc23)OC1(C)C] |
| 35 | 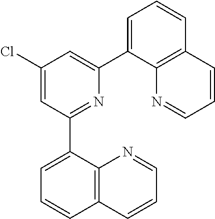 [SMILES: Clc1cc(-c2cccc3cccnc23)nc(-c2cccc3cccnc23)c1] | 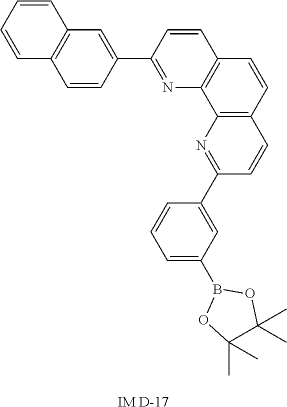 |
| 36 | 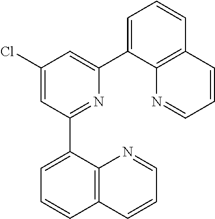 [SMILES: Clc1cc(-c2cccc3cccnc23)nc(-c2cccc3cccnc23)c1] | 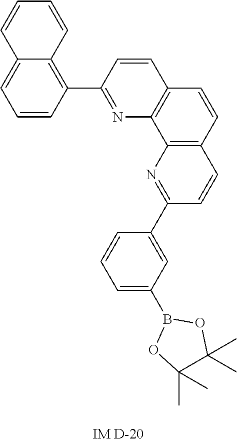 |
| 37 | 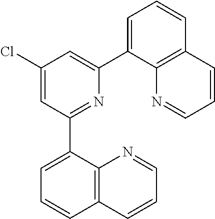 [SMILES: Clc1cc(-c2cccc3cccnc23)nc(-c2cccc3cccnc23)c1] | 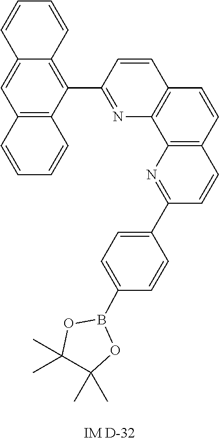 |
| 38 | 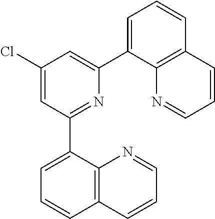 [SMILES: Clc1cc(-c2cccc3cccnc23)nc(-c2cccc3cccnc23)c1] | 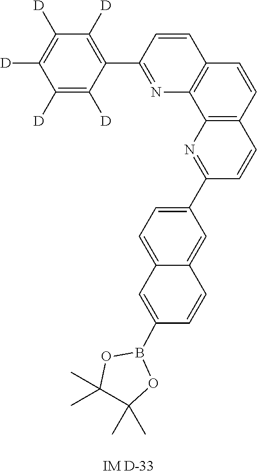 |
| 39 | 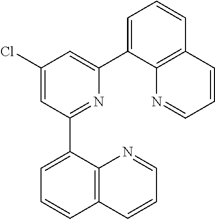 [SMILES: Clc1cc(-c2cccc3cccnc23)nc(-c2cccc3cccnc23)c1] | 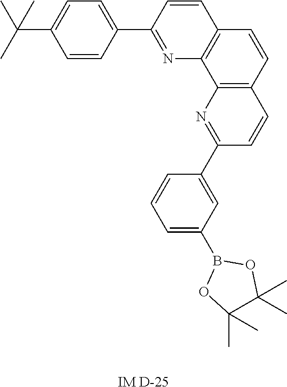 |
| 40 | 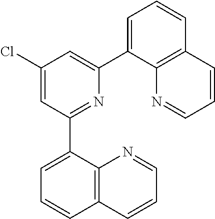 [SMILES: Clc1cc(-c2cccc3cccnc23)nc(-c2cccc3cccnc23)c1] | 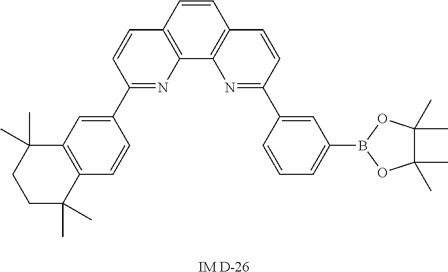 |
| 41 | 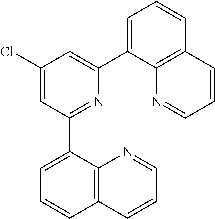 [SMILES: Clc1cc(-c2cccc3cccnc23)nc(-c2cccc3cccnc23)c1] | 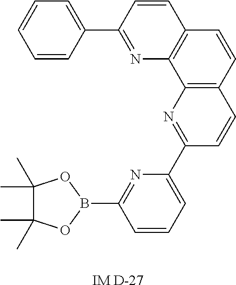 |

| | | | Mass |
|---|---|---|---|
| Synthesis | | | spectrum |
| example | | | (m/z)/ |
| No. | Compound X | Yield/% | [M + H]⁺ |
| 2 | 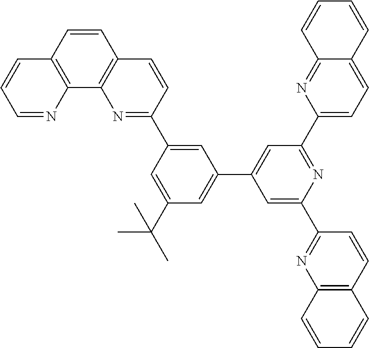 [SMILES: CC(C)(C)c1cc(-c2cc(-c3ccc4ccccc4n3)nc(-c3ccc4ccccc4n3)c2)cc(-c2ccc3ccc4cccnc4c3n2)c1] | 43 | 644.28 |
| | 7 | | |
| 3 | 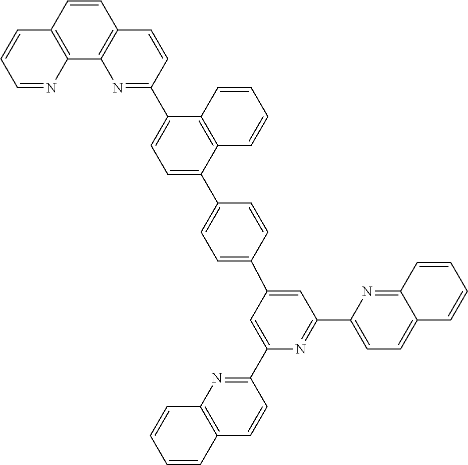 [SMILES: c1ccc2nc(-c3cc(-c4ccc(-c5ccc(-c6ccc7ccc8cccnc8c7n6)c6ccccc56)cc4)cc(-c4ccc5ccccc5n4)n3)ccc2c1] | 45 | 714.26 |
| | 43 | | |
| 4 | 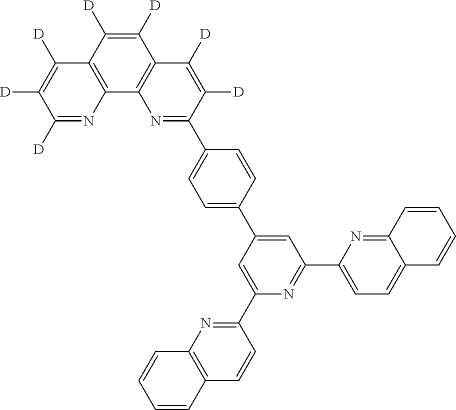 [SMILES: [2H]c1nc2c(c([2H])c1[2H])c([2H])c([2H])c1c([2H])c([2H])c(-c3ccc(-c4cc(-c5ccc6ccccc6n5)nc(-c5ccc6ccccc6n5)c4)cc3)nc12] | 42 | 589.22 |
| | 106 | | |
| 5 | 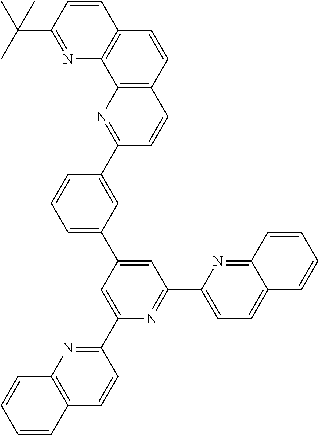 [SMILES: CC(C)(C)c1ccc2ccc3ccc(-c4cccc(-c5cc(-c6ccc7ccccc7n6)nc(-c6ccc7ccccc7n6)c5)c4)nc3c2n1] | 51 | 644.28 |
| | 107 | | |
| 6 | 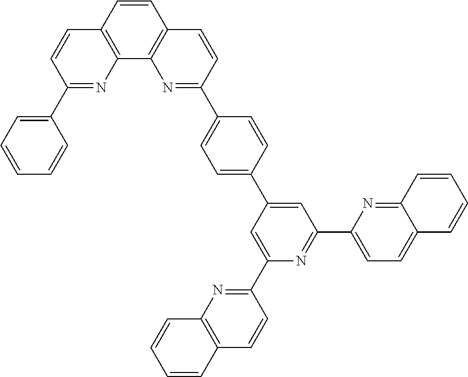 [SMILES: c1ccc(-c2ccc3ccc4ccc(-c5ccc(-c6cc(-c7ccc8ccccc8n7)nc(-c7ccc8ccccc8n7)c6)cc5)nc4c3n2)cc1] | 57 | 664.25 |
| | 116 | | |
| 7 | 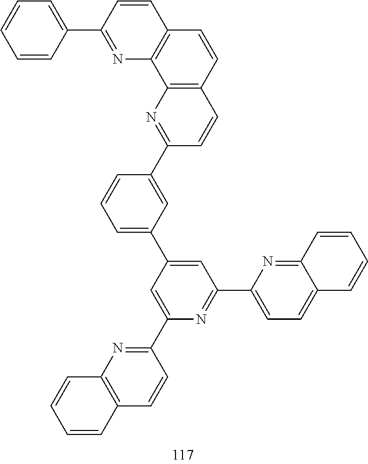 [SMILES: c1ccc(-c2ccc3ccc4ccc(-c5cccc(-c6cc(-c7ccc8ccccc8n7)nc(-c7ccc8ccccc8n7)c6)c5)nc4c3n2)cc1] | 54 | 664.25 |
| 8 | 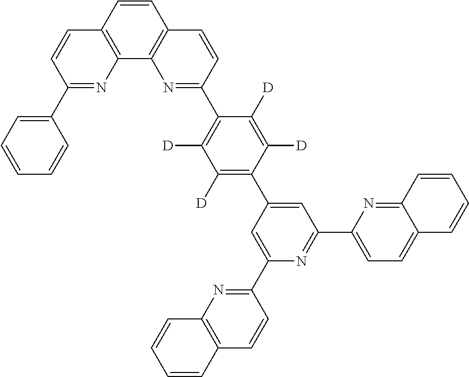 [SMILES: [2H]c1c([2H])c(-c2ccc3ccc4ccc(-c5ccccc5)nc4c3n2)c([2H])c([2H])c1-c1cc(-c2ccc3ccccc3n2)nc(-c2ccc3ccccc3n2)c1] | 48 | 668.27 |
| | 120 | | |
| 9 | 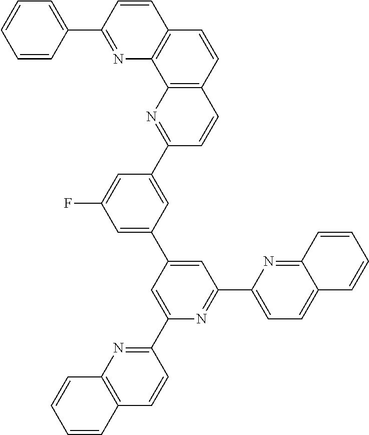 [SMILES: Fc1cc(-c2cc(-c3ccc4ccccc4n3)nc(-c3ccc4ccccc4n3)c2)cc(-c2ccc3ccc4ccc(-c5ccccc5)nc4c3n2)c1] | 54 | 682.24 |
| | 122 | | |
| 10 | 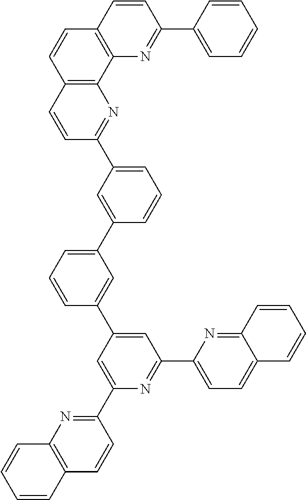 [SMILES: c1ccc(-c2ccc3ccc4ccc(-c5cccc(-c6cccc(-c7cc(-c8ccc9ccccc9n8)nc(-c8ccc9ccccc9n8)c7)c6)c5)nc4c3n2)cc1] | 51 | 740.28 |
| | 128 | | |
| 11 | 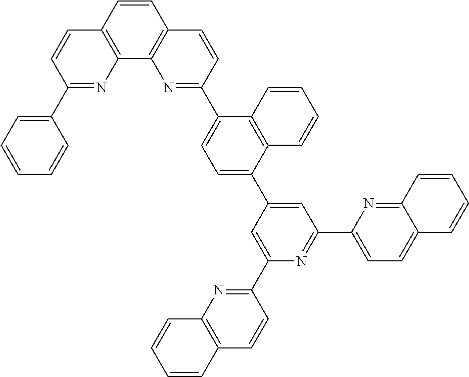 [SMILES: c1ccc(-c2ccc3ccc4ccc(-c5ccc(-c6cc(-c7ccc8ccccc8n7)nc(-c7ccc8ccccc8n7)c6)c6ccccc56)nc4c3n2)cc1] | 54 | 714.26 |
| | 132 | | |
| 12 | 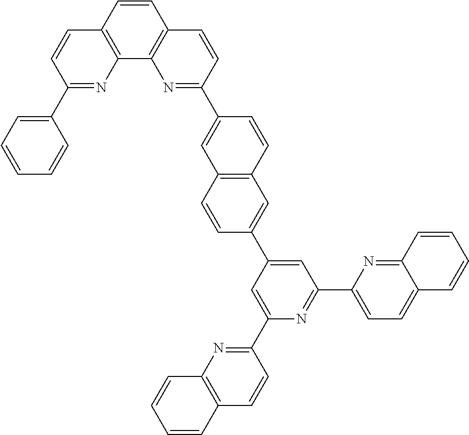 [SMILES: c1ccc(-c2ccc3ccc4ccc(-c5ccc6cc(-c7cc(-c8ccc9ccccc9n8)nc(-c8ccc9ccccc9n8)c7)ccc6c5)nc4c3n2)cc1] | 56 | 714.26 |
| | 134 | | |
| 13 | 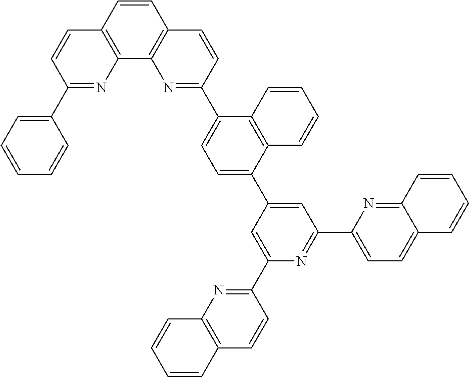 [SMILES: c1ccc(-c2ccc3ccc4ccc(-c5ccc(-c6cc(-c7ccc8ccccc8n7)nc(-c7ccc8ccccc8n7)c6)c6ccccc56)nc4c3n2)cc1] | 52 | 714.26 |
| | 135 | | |
| 14 | 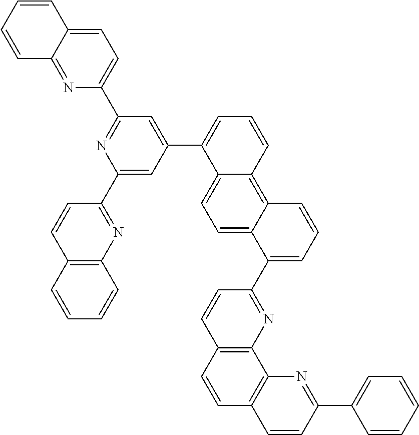 [SMILES: c1ccc(-c2ccc3ccc4ccc(-c5cccc6c5ccc5c(-c7cc(-c8ccc9ccccc9n8)nc(-c8ccc9ccccc9n8)c7)cccc56)nc4c3n2)cc1] | 51 | 764.28 |
| | 141 | | |
| 15 | 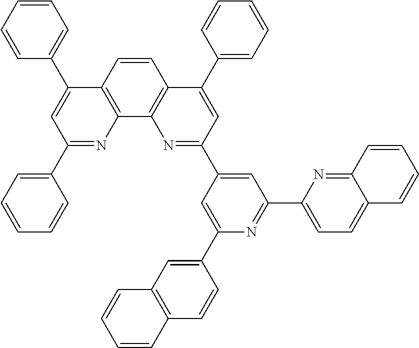 [SMILES: c1ccc(-c2cc(-c3ccccc3)c3ccc4c(-c5ccccc5)cc(-c5cc(-c6ccc7ccccc7c6)nc(-c6ccc7ccccc7n6)c5)nc4c3n2)cc1] | 52 | 740.28 |
| | 185 | | |
| 16 | 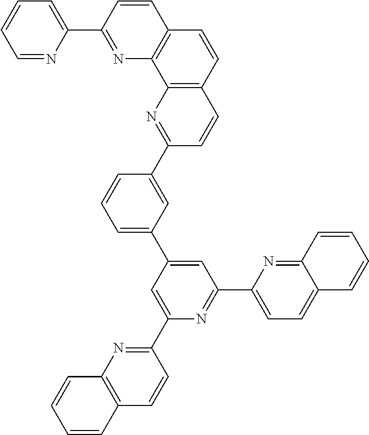 [SMILES: c1ccc(-c2ccc3ccc4ccc(-c5cccc(-c6cc(-c7ccc8ccccc8n7)nc(-c7ccc8ccccc8n7)c6)c5)nc4c3n2)nc1] | 51 | 665.24 |
| | 198 | | |
| 17 | 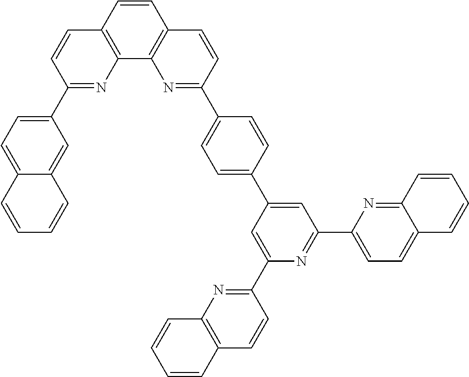 [SMILES: c1ccc2cc(-c3ccc4ccc5ccc(-c6ccc(-c7cc(-c8ccc9ccccc9n8)nc(-c8ccc9ccccc9n8)c7)cc6)nc5c4n3)ccc2c1] | 45 | 714.26 |
| | 234 | | |
| 18 | 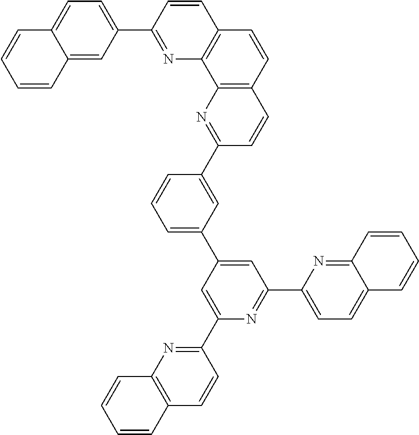 [SMILES: c1cc(-c2cc(-c3ccc4ccccc4n3)nc(-c3ccc4ccccc4n3)c2)cc(-c2ccc3ccc4ccc(-c5ccc6ccccc6c5)nc4c3n2)c1] | 53 | 714.26 |
| | 235 | | |
| 19 | 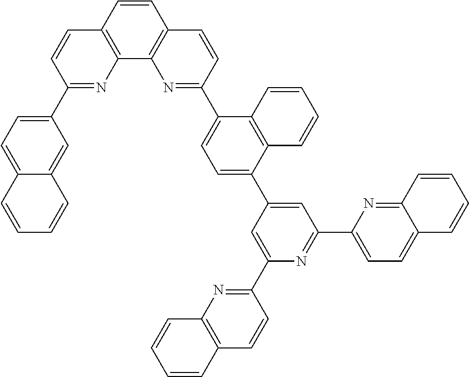 [SMILES: c1ccc2cc(-c3ccc4ccc5ccc(-c6ccc(-c7cc(-c8ccc9ccccc9n8)nc(-c8ccc9ccccc9n8)c7)c7ccccc67)nc5c4n3)ccc2c1] | 49 | 764.28 |
| | 236 | | |
| 20 | 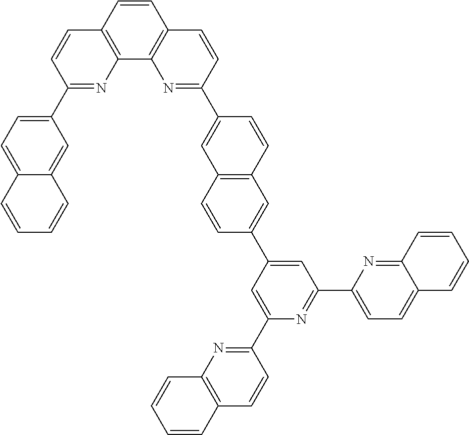 [SMILES: c1ccc2cc(-c3ccc4ccc5ccc(-c6ccc7cc(-c8cc(-c9ccc%10ccccc%10n9)nc(-c9ccc%10ccccc%10n9)c8)ccc7c6)nc5c4n3)ccc2c1] | 51 | 764.28 |
| | 237 | | |
| 21 | 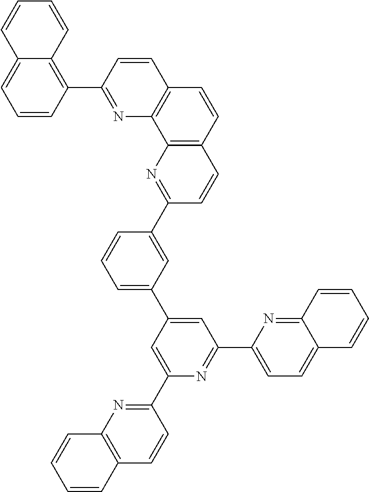 [SMILES: c1cc(-c2cc(-c3ccc4ccccc4n3)nc(-c3ccc4ccccc4n3)c2)cc(-c2ccc3ccc4ccc(-c5cccc6ccccc56)nc4c3n2)c1] | 48 | 714.26 |
| | 243 | | |
| 22 | 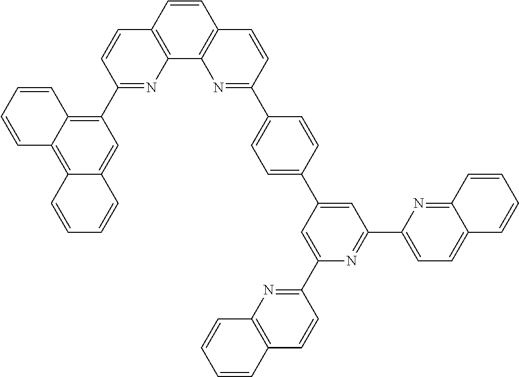 [SMILES: c1ccc2nc(-c3cc(-c4ccc(-c5ccc6ccc7ccc(-c8cc9ccccc9c9ccccc89)nc7c6n5)cc4)cc(-c4ccc5ccccc5n4)n3)ccc2c1] | 53 | 764.28 |
| | 244 | | |
| 23 | 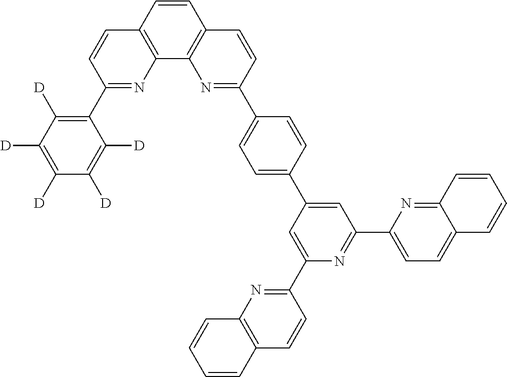 [SMILES: [2H]c1c([2H])c([2H])c(-c2ccc3ccc4ccc(-c5ccc(-c6cc(-c7ccc8ccccc8n7)nc(-c7ccc8ccccc8n7)c6)cc5)nc4c3n2)c([2H])c1[2H]] | 45 | 669.28 |
| | 246 | | |
| 24 | 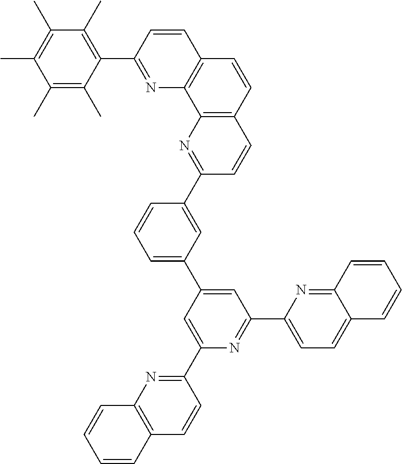 [SMILES: Cc1c(C)c(C)c(-c2ccc3ccc4ccc(-c5cccc(-c6cc(-c7ccc8ccccc8n7)nc(-c7ccc8ccccc8n7)c6)c5)nc4c3n2)c(C)c1C] | 46 | 734.32 |
| | 249 | | |
| 25 | 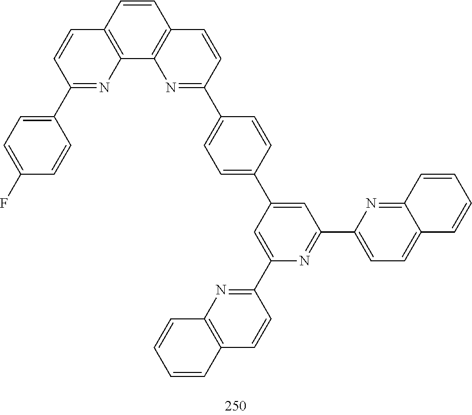 [SMILES: Fc1ccc(-c2ccc3ccc4ccc(-c5ccc(-c6cc(-c7ccc8ccccc8n7)nc(-c7ccc8ccccc8n7)c6)cc5)nc4c3n2)cc1] | 48 | 682.24 |
| 26 | 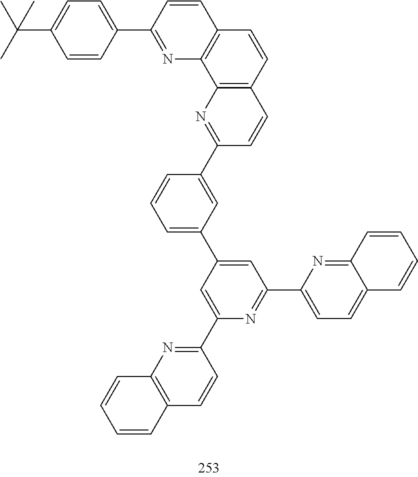 [SMILES: CC(C)(C)c1ccc(-c2ccc3ccc4ccc(-c5cccc(-c6cc(-c7ccc8ccccc8n7)nc(-c7ccc8ccccc8n7)c6)c5)nc4c3n2)cc1] | 52 | 720.31 |
| 27 | 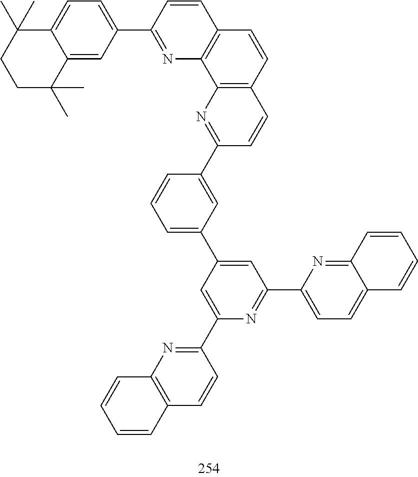 [SMILES: CC1(C)CCC(C)(C)c2cc(-c3ccc4ccc5ccc(-c6cccc(-c7cc(-c8ccc9ccccc9n8)nc(-c8ccc9ccccc9n8)c7)c6)nc5c4n3)ccc21] | 51 | 774.36 |
| 28 | 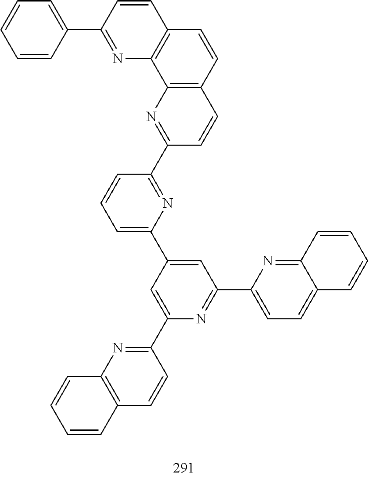 [SMILES: c1ccc(-c2ccc3ccc4ccc(-c5cccc(-c6cc(-c7ccc8ccccc8n7)nc(-c7ccc8ccccc8n7)c6)n5)nc4c3n2)cc1] | 53 | 665.24 |
| 29 | 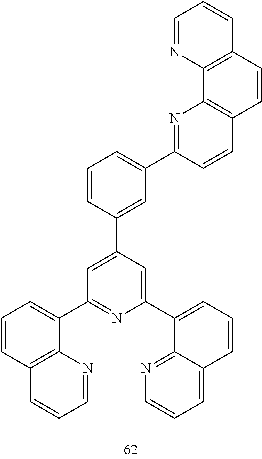 [SMILES: c1cc(-c2cc(-c3cccc4cccnc34)nc(-c3cccc4cccnc34)c2)cc(-c2ccc3ccc4cccnc4c3n2)c1] | 58 | 588.21 |
| 30 | 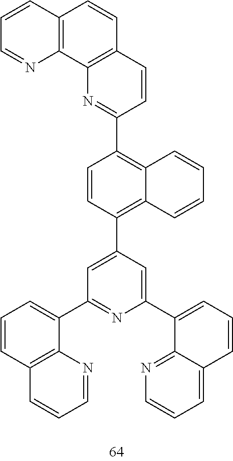 [SMILES: c1cnc2c(-c3cc(-c4ccc(-c5ccc6ccc7cccnc7c6n5)c5ccccc45)cc(-c4cccc5cccnc45)n3)cccc2c1] | 49 | 638.23 |
| 31 | 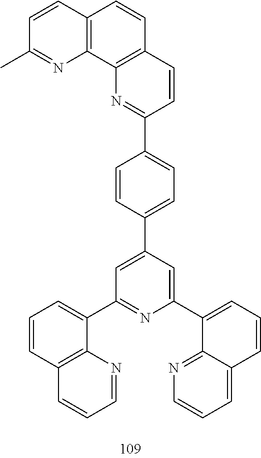 [SMILES: Cc1ccc2ccc3ccc(-c4ccc(-c5cc(-c6cccc7cccnc67)nc(-c6cccc7cccnc67)c5)cc4)nc3c2n1] | 47 | 602.23 |
| 32 | 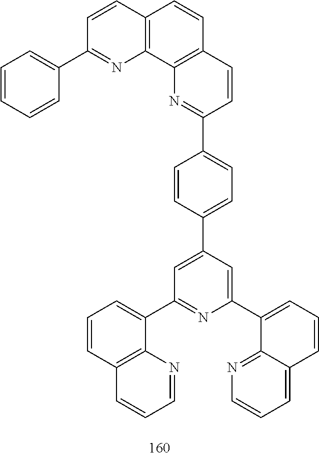 [SMILES: c1ccc(-c2ccc3ccc4ccc(-c5ccc(-c6cc(-c7cccc8cccnc78)nc(-c7cccc8cccnc78)c6)cc5)nc4c3n2)cc1] | 53 | 664.25 |
| 33 | 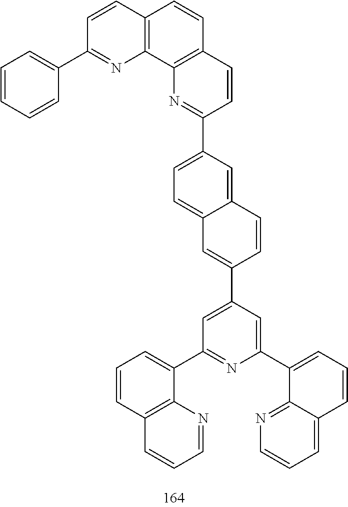 [SMILES: c1ccc(-c2ccc3ccc4ccc(-c5ccc6cc(-c7cc(-c8cccc9cccnc89)nc(-c8cccc9cccnc89)c7)ccc6c5)nc4c3n2)cc1] | 41 | 714.26 |
| 34 | 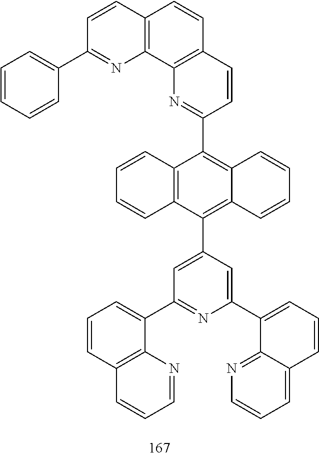 [SMILES: c1ccc(-c2ccc3ccc4ccc(-c5c6ccccc6c(-c6cc(-c7cccc8cccnc78)nc(-c7cccc8cccnc78)c6)c6ccccc56)nc4c3n2)cc1] | 45 | 764.28 |
| 35 | 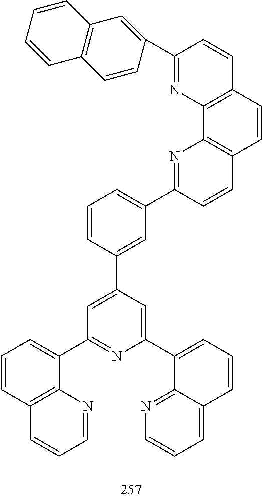 [SMILES: c1cc(-c2cc(-c3cccc4cccnc34)nc(-c3cccc4cccnc34)c2)cc(-c2ccc3ccc4ccc(-c5ccc6ccccc6c5)nc4c3n2)c1] | 42 | 714.26 |
| 36 | 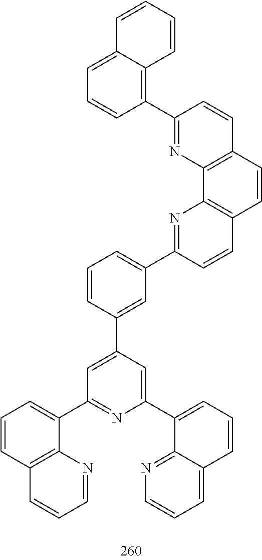 [SMILES: c1cc(-c2cc(-c3cccc4cccnc34)nc(-c3cccc4cccnc34)c2)cc(-c2ccc3ccc4ccc(-c5cccc6ccccc56)nc4c3n2)c1] | 41 | 714.26 |
| 37 | 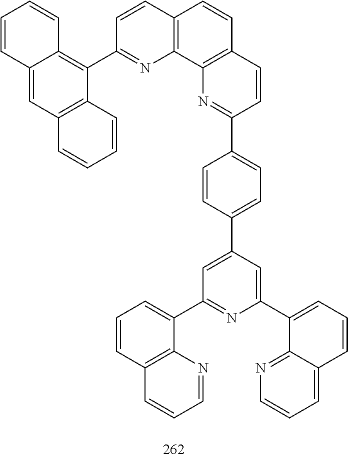 [SMILES: c1ccc2c(-c3ccc4ccc5ccc(-c6ccc(-c7cc(-c8cccc9cccnc89)nc(-c8cccc9cccnc89)c7)cc6)nc5c4n3)c3ccccc3cc2c1] | 39 | 764.28 |
| 38 | 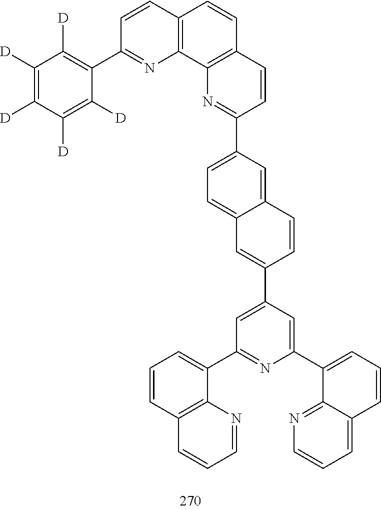 [SMILES: [2H]c1c([2H])c([2H])c(-c2ccc3ccc4ccc(-c5ccc6cc(-c7cc(-c8cccc9cccnc89)nc(-c8cccc9cccnc89)c7)ccc6c5)nc4c3n2)c([2H])c1[2H]] | 43 | 719.29 |
| 39 | 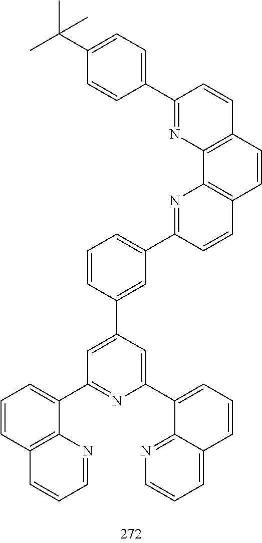 [SMILES: CC(C)(C)c1ccc(-c2ccc3ccc4ccc(-c5cccc(-c6cc(-c7cccc8cccnc78)nc(-c7cccc8cccnc78)c6)c5)nc4c3n2)cc1] | 43 | 720.31 |
| 40 | 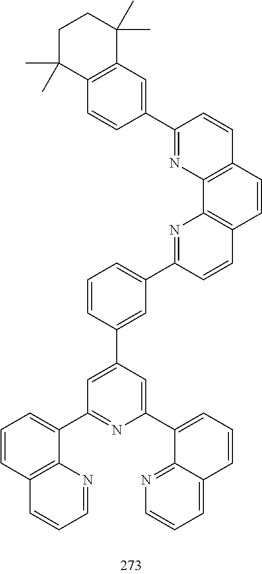 [SMILES: CC1(C)CCC(C)(C)c2cc(-c3ccc4ccc5ccc(-c6cccc(-c7cc(-c8cccc9cccnc89)nc(-c8cccc9cccnc89)c7)c6)nc5c4n3)ccc21] | 41 | 714.26 |
| 41 | 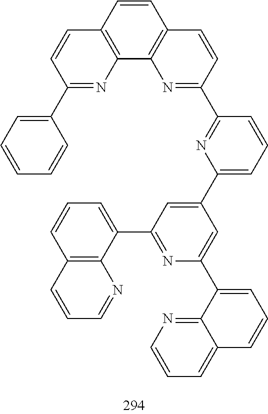 [SMILES: c1ccc(-c2ccc3ccc4ccc(-c5cccc(-c6cc(-c7cccc8cccnc78)nc(-c7cccc8cccnc78)c6)n5)nc4c3n2)cc1] | 39 | 665.24 |

### NMR Data for Some Compounds

NMR data for the Compound 3:

¹H-NMR (CD₃Cl, 400 MHz): 9.15 (d, 1H), 8.96 (s, 2H), 8.89 (s, 1H), 8.53 (d, 2H), 8.33-8.27 (m, 6H), 8.06 (d, 1H), 7.99-7.96 (m, 3H), 7.85-7.81 (m, 5H), 7.63-7.52 (m, 4H).

NMR data for the Compound 117:

¹H-NMR (CD₃Cl, 400 MHz): 8.96 (s, 2H), 8.89 (s, 1H), 8.53 (d, 2H), 8.35-8.27 (m, 8H), 8.19-8.11 (m, 2H), 7.99-7.96 (m, 3H), 7.87-7.79 (m, 5H), 7.62-7.42 (m, 6H).

### Example 1: Blue Organic Electroluminescent Device

An anode was prepared by the following process: an ITO/Ag/ITO substrate with a thickness of 100 Å/1000 Å/100 Å was cut into a size of 40 mm (length)×40 mm (width)×0.7 mm (thickness) to be prepared into an experimental substrate with an anode and an insulating layer pattern by adopting a photoetching process, and surface treatment was performed by using ultraviolet ozone and O₂:N₂ plasma to increase the work function of the anode, and the surface of the experimental substrate is cleaned with an organic solvent to remove impurities and oil on the surface of the experimental substrate.

A compound HT-1 and PD-1 were co-evaporated on the experimental substrate (the anode) at an evaporation rate ratio of 97:3 to form a hole injection layer having a thickness of 100 Å.

The compound HT-1 was vacuum evaporated on the hole injection layer to form a hole transport layer having a thickness of 980 Å.

A compound EB-1 was vacuum evaporated on the hole transport layer to form an electron blocking layer having a thickness of 70 Å.

A compound BH-1 and a compound BD-1 were co-evaporated on the electron blocking layer at an evaporation rate ratio of 98:2 to form an organic light-emitting layer with a thickness of 200 Å.

A Compound 3 and LiQ were co-evaporated on the organic light-emitting layer at an evaporation rate ratio of 1:1 to form an electron transport layer with a thickness of 330 Å.

Ytterbium (Yb) was vacuum evaporated on the electron transport layer to form an electron injection layer having a thickness of 15 Å; and magnesium (Mg) and silver (Ag) were then co-evaporated on the electron injection layer at an evaporation rate ratio of 1:9 to form a cathode with a thickness of 120 Å.

Finally, a compound CP-1 was vacuum evaporated on the cathode to form a cathode capping layer having a thickness of 660 Å, thus completing the manufacture of the blue organic electroluminescent device.

### Examples 2 to 10

An organic electroluminescent device was manufactured by the same method as that in Example 1, except that compounds of Table 6 were used instead of the Compound 3 in Example 1 when the electron transport layer was manufactured.

### Comparative Examples 1 to 3

An organic electroluminescent device was manufactured by the same method as that in Example 1, except that a Compound A, a Compound B, and a Compound C were used instead of the Compound 3 in Example 1 when the electron transport layer was manufactured.

When the devices in the Examples and Comparative examples were manufactured, structures of the compounds used are shown below:

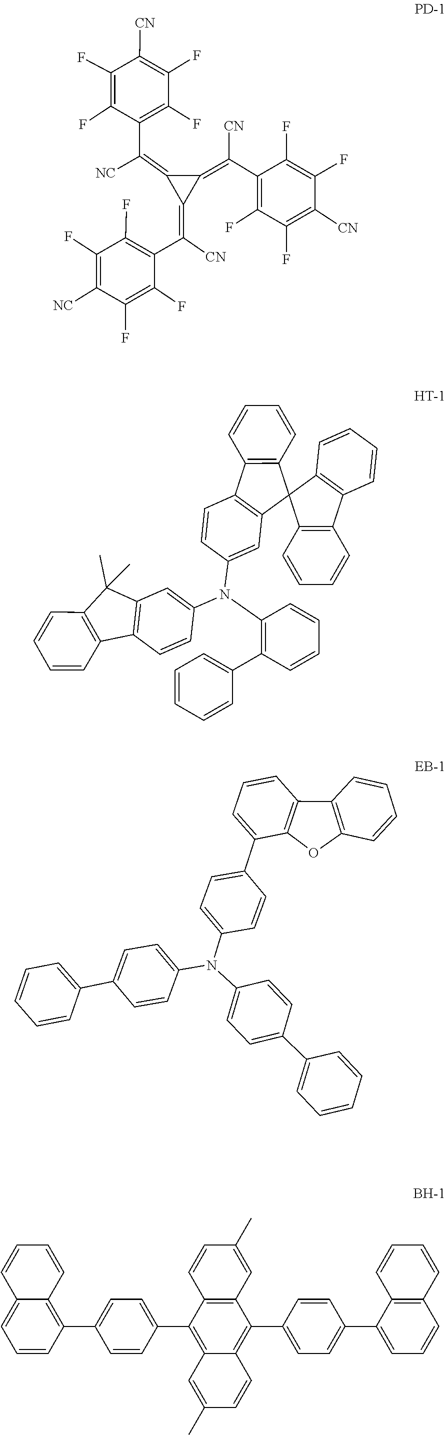

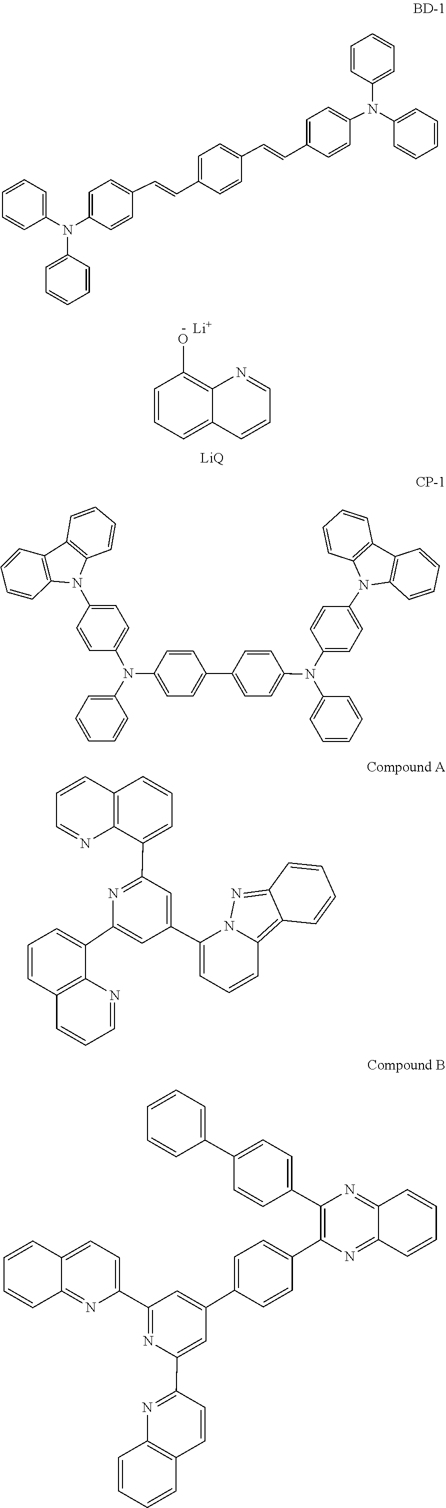

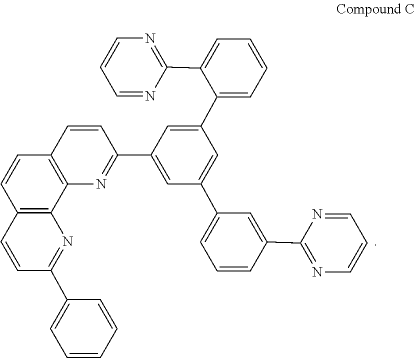 [SMILES: c1ccc(-c2ccc3ccc4ccc(-c5cc(-c6cccc(-c7ncccn7)c6)cc(-c6ccccc6-c6ncccn6)c5)nc4c3n2)cc1]

The blue organic electroluminescent devices manufactured in Examples 1 to 10 and Comparative examples 1 to 3 were subjected to performance tests, and specifically, the current-voltage-brightness (IVL) performance of the devices and the T95 device service life were tested under the condition of 15 mA/cm², and the test results are shown in Table 6 below.

TABLE 6

| | | | Current | T95 | Chroma- |
|---|---|---|---|---|---|
| | | Operating | effi- | service | ticity |
| Example | | voltage | ciency | life | coordinate |
| No. | Compound | (V) | (Cd/A) | (h) | CIEx, y |
| Example 1 | Compound 3 | 4.10 | 6.34 | 198 | 0.14, 0.05 |
| Example 2 | Compound 107 | 4.05 | 5.95 | 194 | 0.14, 0.05 |
| Example 3 | Compound 122 | 4.02 | 6.17 | 196 | 0.14, 0.05 |
| Example 4 | Compound 141 | 4.04 | 6.19 | 203 | 0.14, 0.05 |
| Example 5 | Compound 235 | 4.03 | 6.22 | 200 | 0.14, 0.05 |
| Example 6 | Compound 246 | 4.06 | 6.08 | 204 | 0.14, 0.05 |
| Example 7 | Compound 254 | 4.08 | 6.26 | 206 | 0.14, 0.05 |
| Example 8 | Compound 65 | 4.07 | 5.90 | 202 | 0.14, 0.05 |
| Example 9 | Compound 167 | 4.01 | 6.31 | 205 | 0.14, 0.05 |
| Example 10 | Compound 273 | 4.09 | 6.04 | 195 | 0.14, 0.05 |
| Comparative | Compound A | 4.31 | 4.97 | 165 | 0.14, 0.05 |
| example 1 | | | | | |
| Comparative | Compound B | 4.17 | 5.25 | 174 | 0.14, 0.05 |
| example 2 | | | | | |
| Comparative | Compound C | 4.23 | 5.18 | 169 | 0.14, 0.05 |
| example 3 | | | | | |

As can be seen from the above Table 6, when the compound of the present application is applied to the electron transport layer of the blue organic electroluminescent device, the performance of the device can be greatly improved. In particular, the current efficiency of the devices in Examples 1 to 10 is improved by at least 12.4% and the service life of the devices in Examples 1 to 10 is improved by at least 11.5% compared with the devices in Comparative examples 1 to 3.

In order to further illustrate use of the compound of the present application as a charge generation layer in a stacked organic electroluminescent device, the properties of the materials of the present application are studied below by constructing a stacked device.

### Example 11: Red Stacked Organic Electroluminescent Device

A device was manufactured by the following process: surface treatment was performed with UV, ozone, and O₂:N₂ plasma on an ITO/Ag/ITO experimental substrate with a thickness of 100/1000/100 Å in sequence to increase the work function of the anode, and the surface of the experimental substrate was cleaned with an organic solvent to remove impurities and oil on the surface of the experimental substrate.

HT-1 and PD-1 were co-evaporated on the above experimental substrate at an evaporation rate ratio of 98:2 to form a hole injection layer having a thickness of 100 Å.

A compound RP-1 was vacuum evaporated on the hole injection layer to form a first hole transport layer having a thickness of 150 Å.

The compound RP-1 was vacuum evaporated on the first hole transport layer to form a first hole adjustment layer having a thickness of 250 Å.

A compound RH-1 and a compound RD-1 were co-evaporated on the first hole adjustment layer at an evaporation rate ratio of 98:2 to form a first organic light-emitting layer having a thickness of 450 Å.

A compound ET-1 and LiQ were co-evaporated on the first organic light-emitting layer at an evaporation rate ratio of 1:1 to form a first electron transport layer with a thickness of 220 Å.

The above is a first light-emitting unit.

Then, a Compound 7 and Yb were co-evaporated on the first electron transport layer at an evaporation rate ratio of 99:1 to form an n-type charge generation layer with a thickness of 180 Å; and compounds HT-1 and PD-1 were then co-evaporated on the n-type charge generation layer at an evaporation rate ratio of 95:5 to form a p-type charge generation layer having a thickness of 100 Å.

The above are charge generation layers.

The compound HT-1 was vacuum evaporated on the p-type charge generation layer to form a second hole transport layer having a thickness of 280 Å.

The compound RP-1 was vacuum evaporated on the second hole transport layer to form a second hole adjustment layer having a thickness of 320 Å.

The compound RH-1 and the compound RD-1 were co-evaporated on the second hole adjustment layer at an evaporation rate ratio of 98:2 to form a second organic light-emitting layer having a thickness of 450 Å.

The compound ET-1 and LiQ were co-evaporated on the second organic light-emitting layer at an evaporation rate ratio of 1:1 to form a second electron transport layer having a thickness of 320 Å.

The above is a second light-emitting unit.

Finally, Yb was evaporated on the second electron transport layer to form an electron injection layer having a thickness of 15 Å, and then, magnesium (Mg) and silver (Ag) were co-evaporated on the electron injection layer at an evaporation rate ratio of 1:9 to form a cathode having a thickness of 120 Å.

Further, a compound CP-1 was vacuum evaporated on the cathode to form a cathode capping layer having a thickness of 660 Å, thus completing the manufacture of the red stacked organic electroluminescent device.

### Examples 12 to 41

An organic electroluminescent device was manufactured by the same method as that in Example 11, except that compounds of Table 7 were used instead of the Compound 7 in Example 11 when the n-type charge generation layer was manufactured.

### Comparative Examples 4 to 7

An organic electroluminescent device was manufactured by the same method as that in Example 11, except that Compounds D, E, F, and G were used instead of the Compound 7 in Example 11 when the n-type charge generation layer was manufactured.

When the devices in the Examples and Comparative examples were manufactured, structures of the compounds used are shown below:

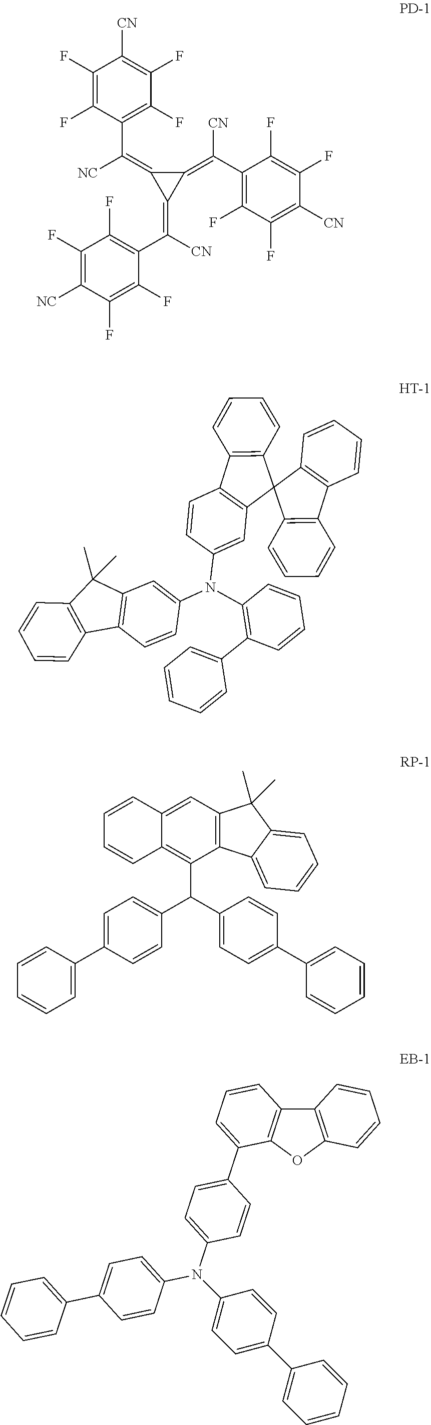

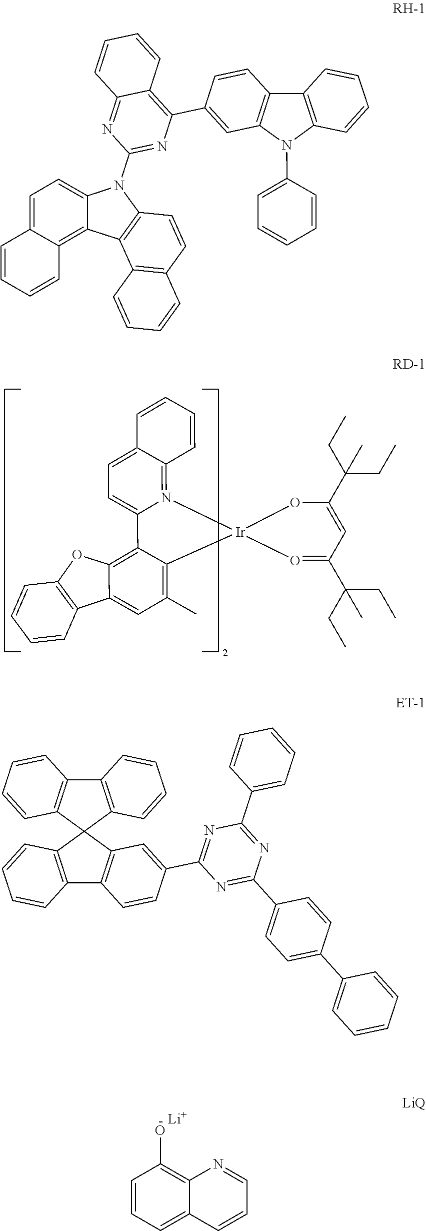

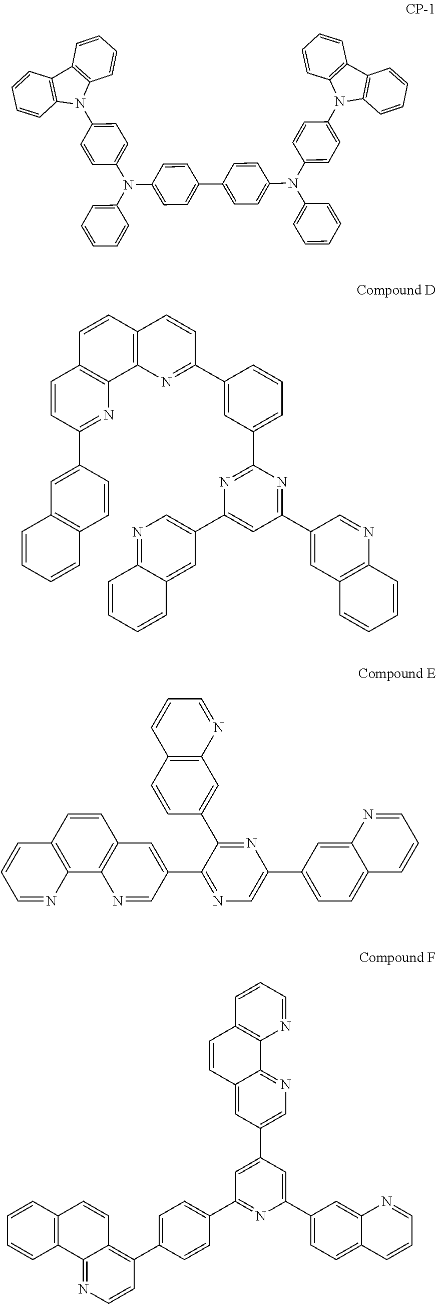 [SMILES: c1ccc(N(c2ccc(-c3ccc(N(c4ccccc4)c4ccc(-n5c6ccccc6c6ccccc65)cc4)cc3)cc2)c2ccc(-n3c4ccccc4c4ccccc43)cc2)cc1 ; c1cc(-c2ccc3ccc4ccc(-c5ccc6ccccc6c5)nc4c3n2)cc(-c2nc(-c3cnc4ccccc4c3)cc(-c3cnc4ccccc4c3)n2)c1 ; c1cnc2cc(-c3cnc(-c4cnc5c(ccc6cccnc65)c4)c(-c4ccc5cccnc5c4)n3)ccc2c1 ; c1cnc2cc(-c3cc(-c4cnc5c(ccc6cccnc65)c4)cc(-c4ccc(-c5ccnc6c5ccc5ccccc56)cc4)n3)ccc2c1]

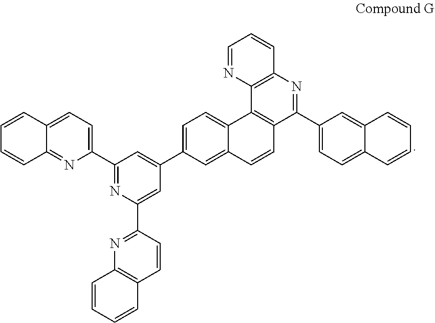 [SMILES: c1ccc2cc(-c3nc4cccnc4c4c3ccc3cc(-c5cc(-c6ccc7ccccc7n6)nc(-c6ccc7ccccc7n6)c5)ccc34)ccc2c1]

The red stacked organic electroluminescent devices manufactured in Examples 11 to 41 and Comparative examples 4 to 7 were subjected to performance tests, and specifically, the current-voltage-brightness (IVL) performance of the devices was tested under the condition of 15 mA/cm² and the T95 device service life was tested under the condition of 20 mA/cm², and the test results are shown in Table 7 below.

TABLE 7

| | | | Current | T95 | Chroma- |
|---|---|---|---|---|---|
| | | Operating | effi- | service | ticity |
| Example | | voltage | ciency | life | coordinate |
| No. | Compound | (V) | (Cd/A) | (h) | CIEx, y |
| Example 11 | Compound 7 | 6.84 | 120.66 | 556 | 0.68, 0.32 |
| Example 12 | Compound 43 | 7.08 | 118.47 | 567 | 0.68, 0.32 |
| Example 13 | Compound 106 | 7.14 | 118.12 | 568 | 0.68, 0.32 |
| Example 14 | Compound 116 | 7.01 | 119.28 | 570 | 0.68, 0.32 |
| Example 15 | Compound 117 | 7.07 | 119.43 | 554 | 0.68, 0.32 |
| Example 16 | Compound 120 | 7.15 | 118.25 | 563 | 0.68, 0.32 |
| Example 17 | Compound 128 | 7.10 | 119.94 | 553 | 0.68, 0.32 |
| Example 18 | Compound 132 | 7.06 | 118.91 | 566 | 0.68, 0.32 |
| Example 19 | Compound 134 | 6.91 | 120.42 | 559 | 0.68, 0.32 |
| Example 20 | Compound 135 | 6.86 | 120.31 | 561 | 0.68, 0.32 |
| Example 21 | Compound 185 | 6.98 | 119.92 | 555 | 0.68, 0.32 |
| Example 22 | Compound 198 | 6.96 | 120.33 | 564 | 0.68, 0.32 |
| Example 23 | Compound 234 | 7.05 | 119.36 | 558 | 0.68, 0.32 |
| Example 24 | Compound 236 | 6.99 | 119.17 | 569 | 0.68, 0.32 |
| Example 25 | Compound 237 | 6.94 | 118.87 | 572 | 0.68, 0.32 |
| Example 26 | Compound 243 | 7.00 | 118.86 | 562 | 0.68, 0.32 |
| Example 27 | Compound 244 | 6.83 | 120.49 | 571 | 0.68, 0.32 |
| Example 28 | Compound 249 | 7.11 | 119.89 | 560 | 0.68, 0.32 |
| Example 29 | Compound 250 | 6.89 | 120.64 | 557 | 0.68, 0.32 |
| Example 30 | Compound 253 | 6.97 | 119.97 | 552 | 0.68, 0.32 |
| Example 31 | Compound 291 | 6.90 | 118.45 | 565 | 0.68, 0.32 |
| Example 32 | Compound 62 | 7.09 | 120.14 | 639 | 0.68, 0.32 |
| Example 33 | Compound 64 | 6.88 | 120.53 | 640 | 0.68, 0.32 |
| Example 34 | Compound 160 | 6.85 | 118.22 | 642 | 0.68, 0.32 |
| Example 35 | Compound 164 | 6.93 | 118.11 | 636 | 0.68, 0.32 |
| Example 36 | Compound 257 | 7.12 | 118.75 | 635 | 0.68, 0.32 |
| Example 37 | Compound 260 | 6.87 | 119.96 | 637 | 0.68, 0.32 |
| Example 38 | Compound 262 | 7.04 | 119.47 | 643 | 0.68, 0.32 |
| Example 39 | Compound 270 | 6.95 | 120.71 | 641 | 0.68, 0.32 |
| Example 40 | Compound 272 | 7.02 | 118.96 | 634 | 0.68, 0.32 |
| Example 41 | Compound 294 | 6.92 | 118.18 | 638 | 0.68, 0.32 |
| Comparative | Compound D | 7.21 | 104.21 | 490 | 0.68, 0.32 |
| example 4 | | | | | |
| Comparative | Compound E | 7.59 | 100.47 | 475 | 0.68, 0.32 |
| example 5 | | | | | |
| Comparative | Compound F | 7.38 | 103.78 | 486 | 0.68, 0.32 |
| example 6 | | | | | |
| Comparative | Compound G | 7.43 | 102.36 | 481 | 0.68, 0.32 |
| Example 7 | | | | | |

As can be seen from the above Table 7, when the compound of the present application is applied to the n-type charge generation layer of the red stacked organic electroluminescent device, the performance of the device can be greatly improved. In particular, the current efficiency of the devices in Examples 11 to 41 is improved by at least 13.3% and the service life of the devices in Examples 11 to 41 is improved by at least 12.7% compared with the devices in Comparative examples 4 to 7.

1. An organic compound, having a structure represented by a Formula 1: 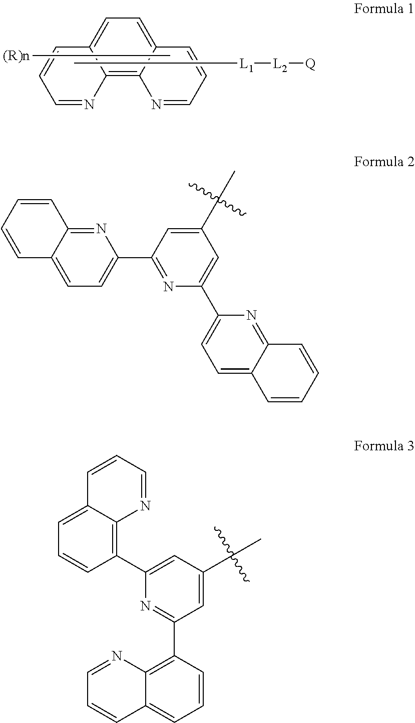
wherein Q is a Formula 2 or a Formula 3;
L₁ and L₂ are the same or different, and are each independently selected from a single bond, a substituted or unsubstituted arylene with 6 to 30 carbon atoms, or a substituted or unsubstituted heteroarylene with 3 to 30 carbon atoms;
substituent(s) of L₁ and L₂ are the same or different, and are each independently selected from a deuterium, a halogen group, a cyano, a haloalkyl with 1 to 10 carbon atoms, a trialkylsilyl with 3 to 12 carbon atoms, an alkyl with 1 to 10 carbon atoms, an aryl with 6 to 20 carbon atoms, or a heteroaryl with 3 to 20 carbon atoms;
each R is the same or different, and is independently selected from a deuterium, a halogen group, a cyano, an alkyl with 1 to 10 carbon atoms, a substituted or unsubstituted aryl with 6 to 20 carbon atoms, or a substituted or unsubstituted heteroaryl with 3 to 20 carbon atoms;
substituent(s) of R are the same or different, and are each independently selected from a deuterium, a halogen group, a cyano, an alkyl with 1 to 10 carbon atoms, an aryl with 6 to 12 carbon atoms, or a heteroaryl with 3 to 12 carbon atoms; and
n is the number of R, n is selected from 0, 1, 2, 3, 4, 5, 6 or 7, and in the case where n is greater than 1, any two Rs are the same or different; and alternatively, any two adjacent Rs form an aromatic ring with 6 to 14 carbon atoms.

2. The organic compound according to claim 1, wherein L₁ and L₂ are the same or different, and are each independently selected from a single bond, a substituted or unsubstituted arylene with 6 to 22 carbon atoms, or a substituted or unsubstituted heteroarylene with 3 to 18 carbon atoms; and
alternatively, the substituent(s) of L₁ and L₂ are the same or different, and are each independently selected from a deuterium, a fluorine, a cyano, a haloalkyl with 1 to 5 carbon atoms, a trialkylsilyl with 3 to 6 carbon atoms, an alkyl with 1 to 5 carbon atoms, an aryl with 6 to 16 carbon atoms or a heteroaryl with 3 to 12 carbon atoms.

3. The organic compound according to claim 1, wherein L₁ and L₂ are the same or different, and are each independently selected from a single bond, a substituted or unsubstituted phenylene, a substituted or unsubstituted naphthylene, a substituted or unsubstituted biphenylene, a substituted or unsubstituted anthrylene, a substituted or unsubstituted phenanthrylene, a substituted or unsubstituted pyrenylene, a substituted or unsubstituted dibenzofurylidene, a substituted or unsubstituted dibenzothienylidene, a substituted or unsubstituted carbazolylidene, a substituted or unsubstituted triazinylene, a substituted or unsubstituted pyrimidinylidene, a substituted or unsubstituted pyridylidene, and a substituted or unsubstituted pyrazinylene; and
alternatively, the substituent(s) of L₁ and L₂ are the same or different, and are each independently selected from a deuterium, a fluorine, a cyano, a trifluoromethyl, a trimethylsilyl, a methyl, an ethyl, an isopropyl, a tert-butyl, a phenyl, a naphthyl, a biphenyl, a phenanthryl, an anthryl, a pyrenyl, a dibenzofuranyl, a dibenzothienyl, a carbazolyl, a pyridyl, a pyrimidinyl, a pyrazinyl or a quinolyl.

4. The organic compound according to claim 1, wherein L₁ and L₂ are the same or different, and are each independently selected from a single bond or the group consisting of:

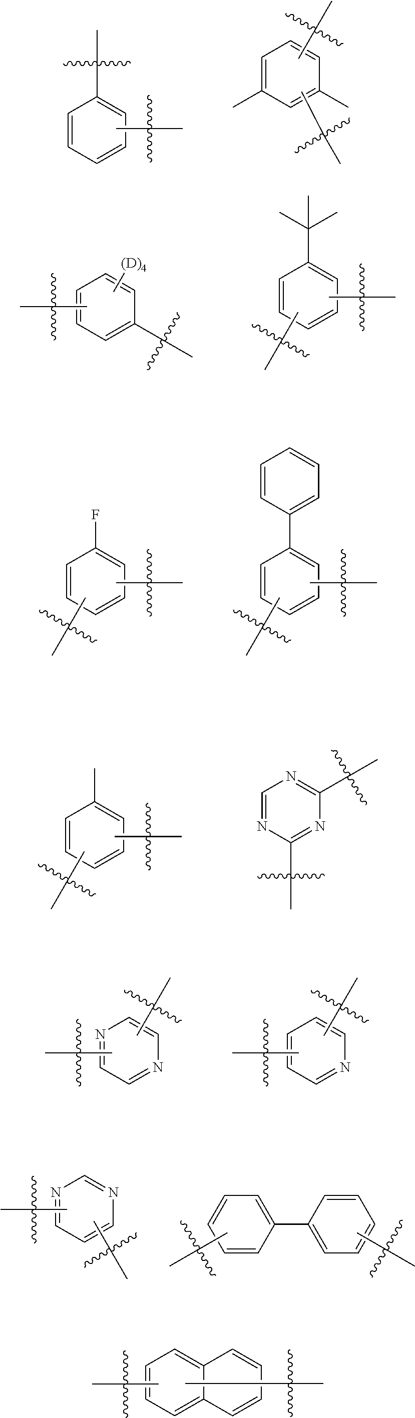

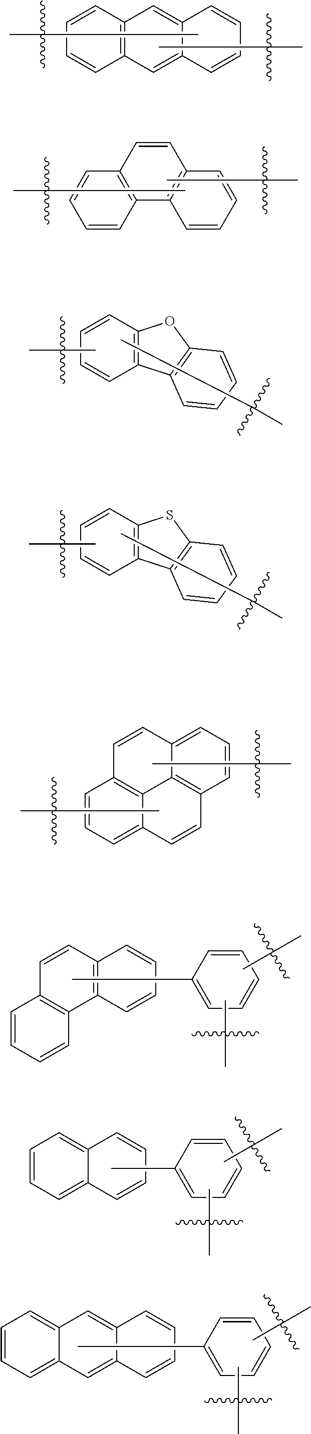

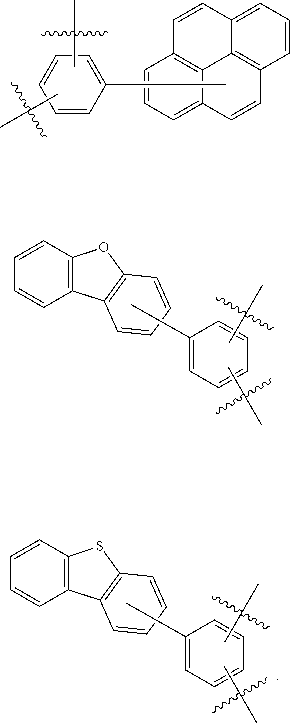

5. The organic compound according to claim 1, wherein 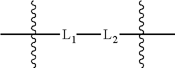
is selected from a single bond or the group consisting of:

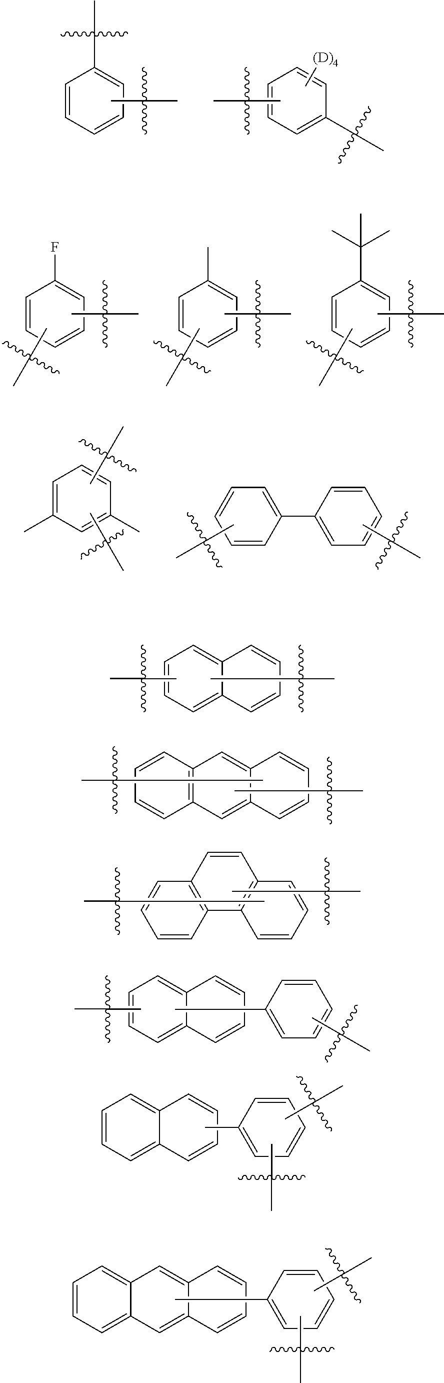

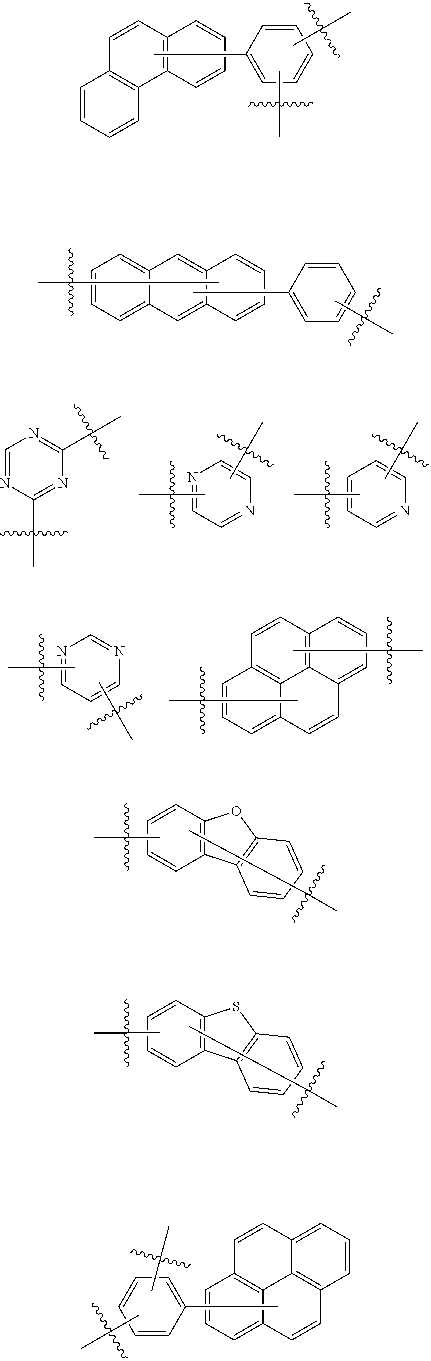

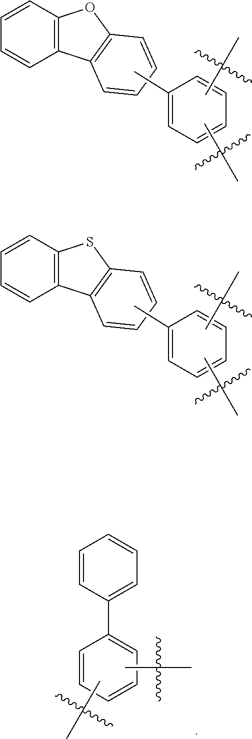

6. The organic compound according to claim 1, wherein each R is independently selected from a deuterium, a fluorine, a cyano, a methyl, an ethyl, an isopropyl, a tert-butyl, a substituted or unsubstituted phenyl, a substituted or unsubstituted naphthyl, a substituted or unsubstituted phenanthryl, a substituted or unsubstituted anthryl, a substituted or unsubstituted tetralinyl, a substituted or unsubstituted pyridyl, a substituted or unsubstituted pyrimidinyl, a substituted or unsubstituted triazinyl, a substituted or unsubstituted pyrazinyl, a substituted or unsubstituted quinolyl, or a substituted or unsubstituted isoquinolyl; and
alternatively, substituent(s) of R are the same or different, and are each independently selected from a deuterium, a fluorine, a cyano, a methyl, an ethyl, an isopropyl, a tert-butyl or a phenyl, and alternatively, any two adjacent Rs form a benzene ring or a naphthalene ring.

7. The organic compound according to claim 1, wherein each R is the same or different, and is independently selected from a deuterium, a fluorine, a cyano, a methyl, an ethyl, an isopropyl, a tert-butyl, a phenyl, a pyridyl, a pyrimidinyl, a triazinyl, a naphthyl, a phenanthryl, an anthryl, a deuterophenyl, a pentadeuterophenyl, a pentamethyl-substituted phenyl, a methyl-substituted phenyl, a pentafluoro-substituted phenyl, a fluoro-substituted phenyl, a cyano-substituted phenyl, a tert-butyl-substituted phenyl, or a tetramethyl-substituted tetralinyl.

8. The organic compound according to claim 1, wherein the organic compound is selected from the group consisting of the following compounds:

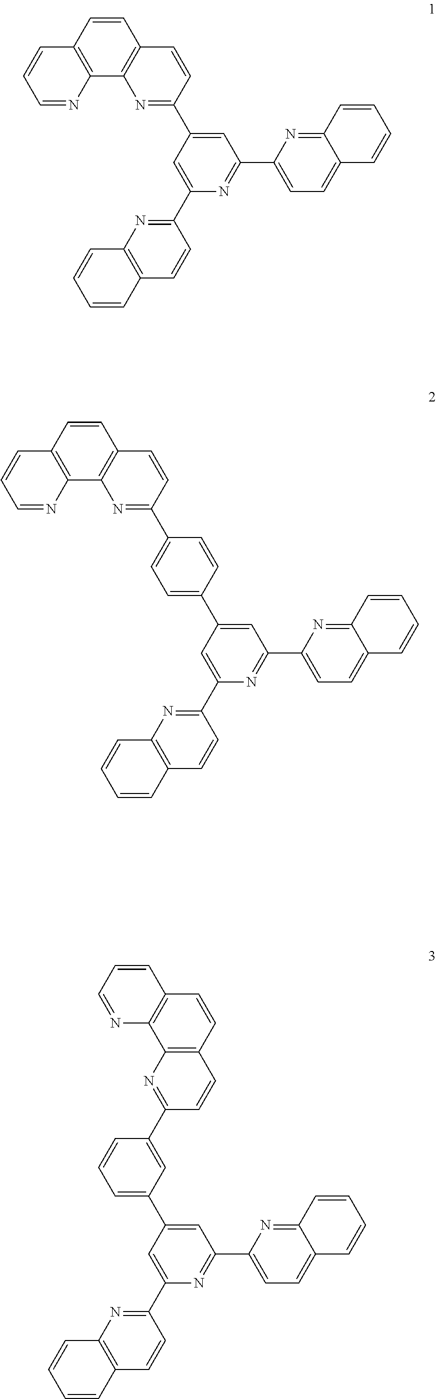 [SMILES: c1ccc2nc(-c3cc(-c4ccc5ccc6cccnc6c5n4)cc(-c4ccc5ccccc5n4)n3)ccc2c1 ; c1ccc2nc(-c3cc(-c4ccc(-c5ccc6ccc7cccnc7c6n5)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc3ccc4cccnc4c3n2)c1]

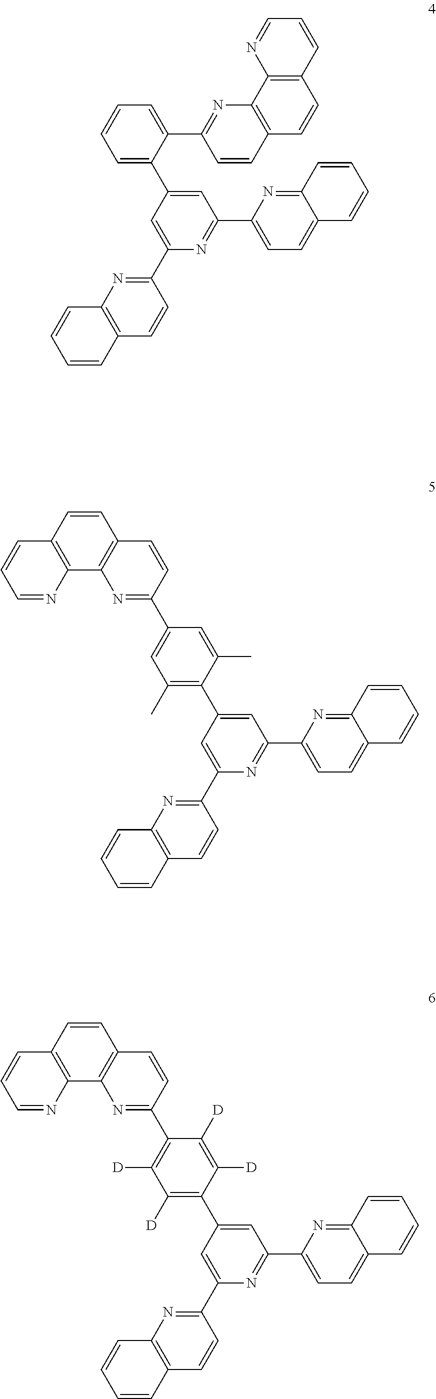 [SMILES: c1ccc(-c2ccc3ccc4cccnc4c3n2)c(-c2cc(-c3ccc4ccccc4n3)nc(-c3ccc4ccccc4n3)c2)c1 ; Cc1cc(-c2ccc3ccc4cccnc4c3n2)cc(C)c1-c1cc(-c2ccc3ccccc3n2)nc(-c2ccc3ccccc3n2)c1 ; [2H]c1c([2H])c(-c2ccc3ccc4cccnc4c3n2)c([2H])c([2H])c1-c1cc(-c2ccc3ccccc3n2)nc(-c2ccc3ccccc3n2)c1]

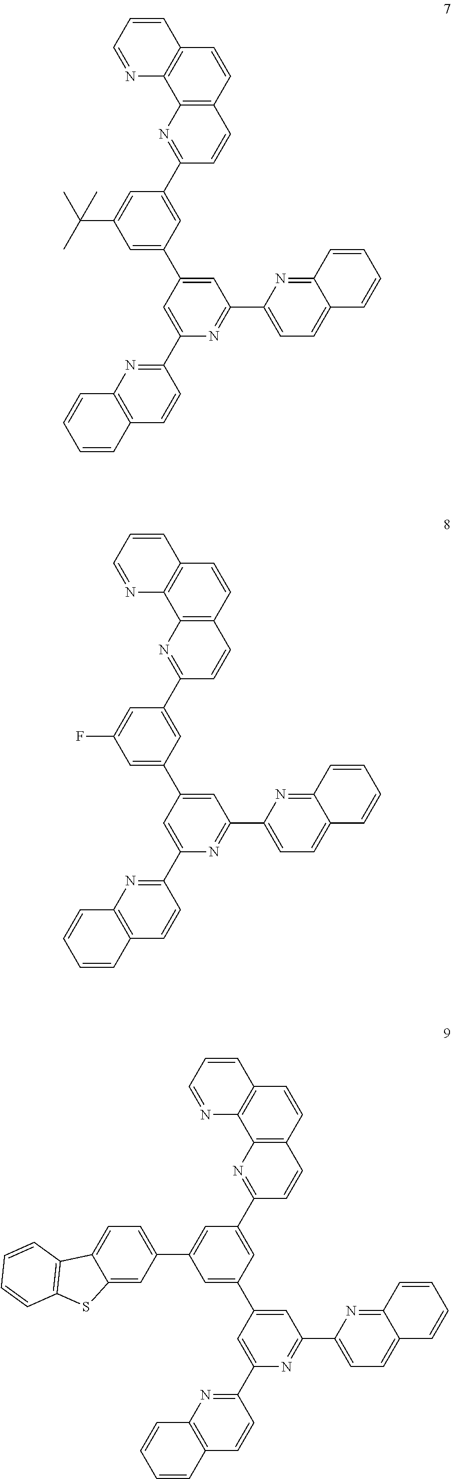 [SMILES: CC(C)(C)c1cc(-c2cc(-c3ccc4ccccc4n3)nc(-c3ccc4ccccc4n3)c2)cc(-c2ccc3ccc4cccnc4c3n2)c1 ; Fc1cc(-c2cc(-c3ccc4ccccc4n3)nc(-c3ccc4ccccc4n3)c2)cc(-c2ccc3ccc4cccnc4c3n2)c1 ; c1ccc2nc(-c3cc(-c4cc(-c5ccc6c(c5)sc5ccccc56)cc(-c5ccc6ccc7cccnc7c6n5)c4)cc(-c4ccc5ccccc5n4)n3)ccc2c1]

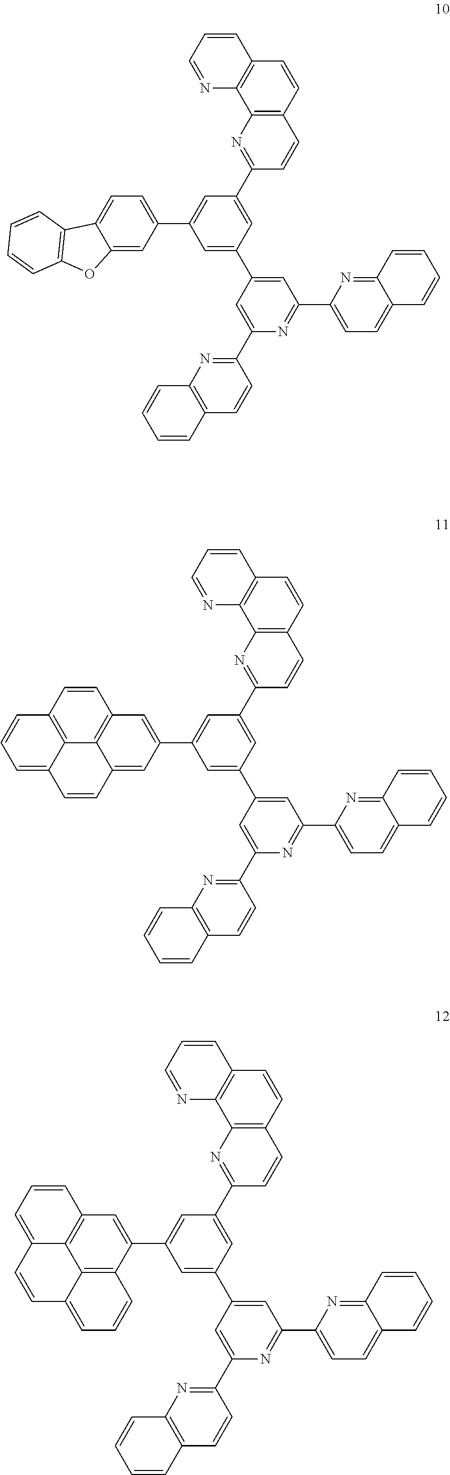 [SMILES: c1ccc2nc(-c3cc(-c4cc(-c5ccc6c(c5)oc5ccccc56)cc(-c5ccc6ccc7cccnc7c6n5)c4)cc(-c4ccc5ccccc5n4)n3)ccc2c1 ; c1ccc2nc(-c3cc(-c4cc(-c5cc6ccc7cccc8ccc(c5)c6c78)cc(-c5ccc6ccc7cccnc7c6n5)c4)cc(-c4ccc5ccccc5n4)n3)ccc2c1 ; c1ccc2nc(-c3cc(-c4cc(-c5ccc6ccc7cccnc7c6n5)cc(-c5cc6cccc7ccc8cccc5c8c76)c4)cc(-c4ccc5ccccc5n4)n3)ccc2c1]

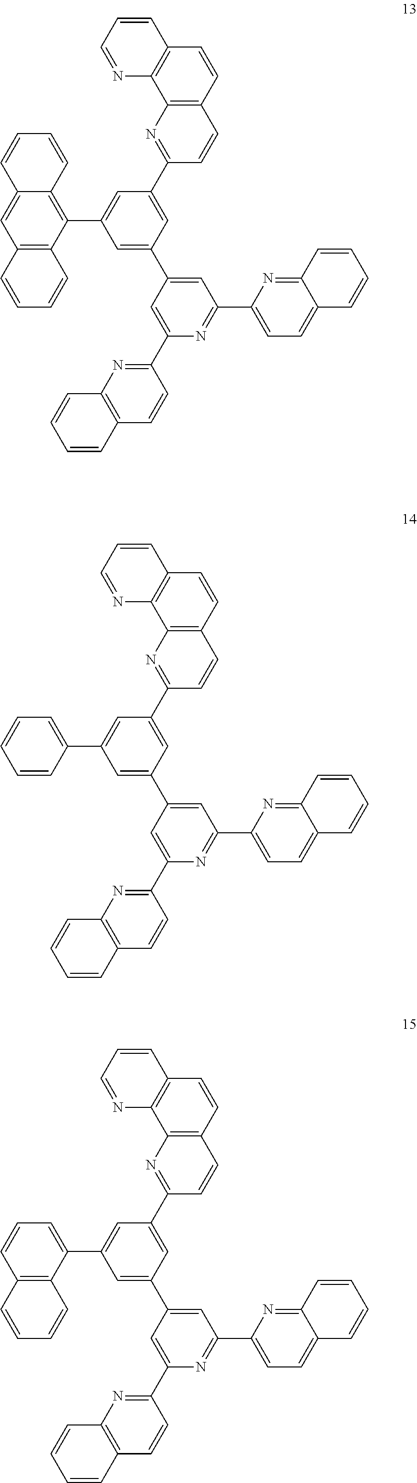 [SMILES: c1ccc2nc(-c3cc(-c4cc(-c5ccc6ccc7cccnc7c6n5)cc(-c5c6ccccc6cc6ccccc56)c4)cc(-c4ccc5ccccc5n4)n3)ccc2c1 ; c1ccc(-c2cc(-c3cc(-c4ccc5ccccc5n4)nc(-c4ccc5ccccc5n4)c3)cc(-c3ccc4ccc5cccnc5c4n3)c2)cc1 ; c1ccc2nc(-c3cc(-c4cc(-c5ccc6ccc7cccnc7c6n5)cc(-c5cccc6ccccc56)c4)cc(-c4ccc5ccccc5n4)n3)ccc2c1]

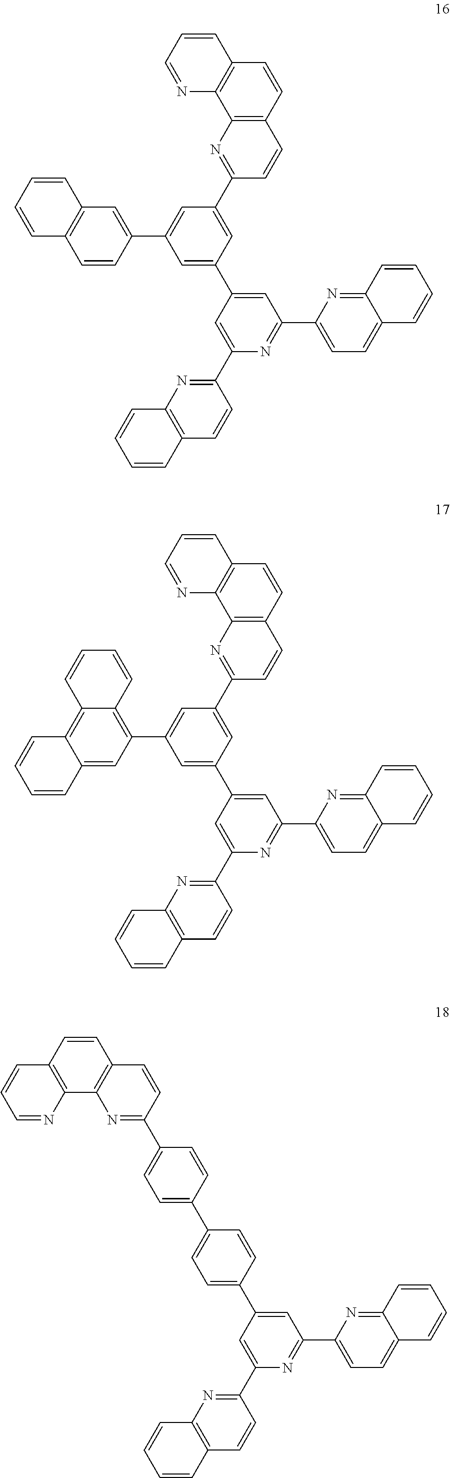 [SMILES: c1ccc2cc(-c3cc(-c4cc(-c5ccc6ccccc6n5)nc(-c5ccc6ccccc6n5)c4)cc(-c4ccc5ccc6cccnc6c5n4)c3)ccc2c1 ; c1ccc2nc(-c3cc(-c4cc(-c5ccc6ccc7cccnc7c6n5)cc(-c5cc6ccccc6c6ccccc56)c4)cc(-c4ccc5ccccc5n4)n3)ccc2c1 ; c1ccc2nc(-c3cc(-c4ccc(-c5ccc(-c6ccc7ccc8cccnc8c7n6)cc5)cc4)cc(-c4ccc5ccccc5n4)n3)ccc2c1]

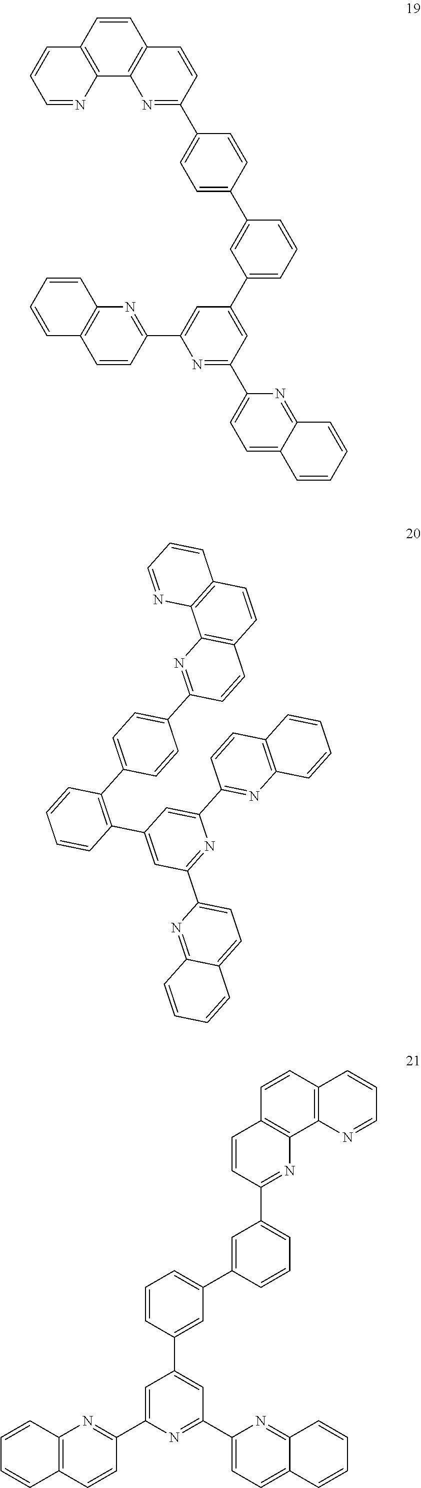 [SMILES: c1cc(-c2ccc(-c3ccc4ccc5cccnc5c4n3)cc2)cc(-c2cc(-c3ccc4ccccc4n3)nc(-c3ccc4ccccc4n3)c2)c1 ; c1ccc(-c2cc(-c3ccc4ccccc4n3)nc(-c3ccc4ccccc4n3)c2)c(-c2ccc(-c3ccc4ccc5cccnc5c4n3)cc2)c1 ; c1cc(-c2cccc(-c3ccc4ccc5cccnc5c4n3)c2)cc(-c2cc(-c3ccc4ccccc4n3)nc(-c3ccc4ccccc4n3)c2)c1]

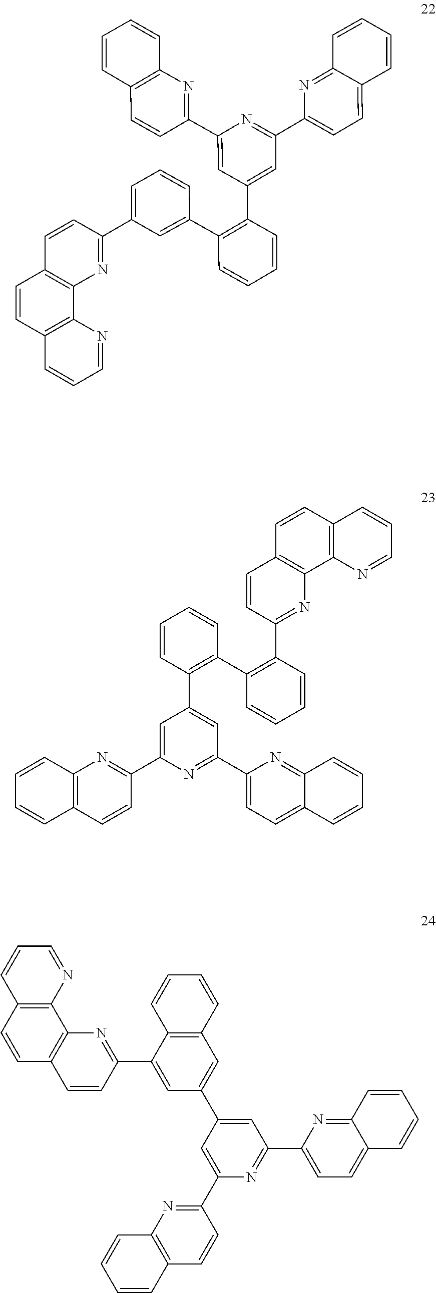 [SMILES: c1cc(-c2ccc3ccc4cccnc4c3n2)cc(-c2ccccc2-c2cc(-c3ccc4ccccc4n3)nc(-c3ccc4ccccc4n3)c2)c1 ; c1ccc(-c2ccccc2-c2ccc3ccc4cccnc4c3n2)c(-c2cc(-c3ccc4ccccc4n3)nc(-c3ccc4ccccc4n3)c2)c1 ; c1ccc2nc(-c3cc(-c4cc(-c5ccc6ccc7cccnc7c6n5)c5ccccc5c4)cc(-c4ccc5ccccc5n4)n3)ccc2c1]

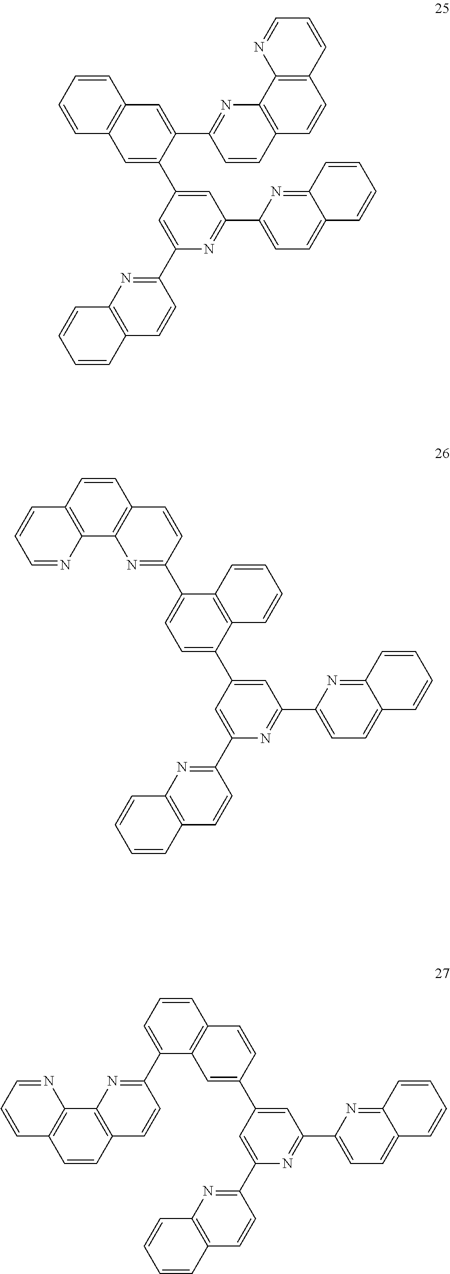 [SMILES: c1ccc2cc(-c3ccc4ccc5cccnc5c4n3)c(-c3cc(-c4ccc5ccccc5n4)nc(-c4ccc5ccccc5n4)c3)cc2c1 ; c1ccc2nc(-c3cc(-c4ccc(-c5ccc6ccc7cccnc7c6n5)c5ccccc45)cc(-c4ccc5ccccc5n4)n3)ccc2c1 ; c1ccc2nc(-c3cc(-c4ccc5cccc(-c6ccc7ccc8cccnc8c7n6)c5c4)cc(-c4ccc5ccccc5n4)n3)ccc2c1]

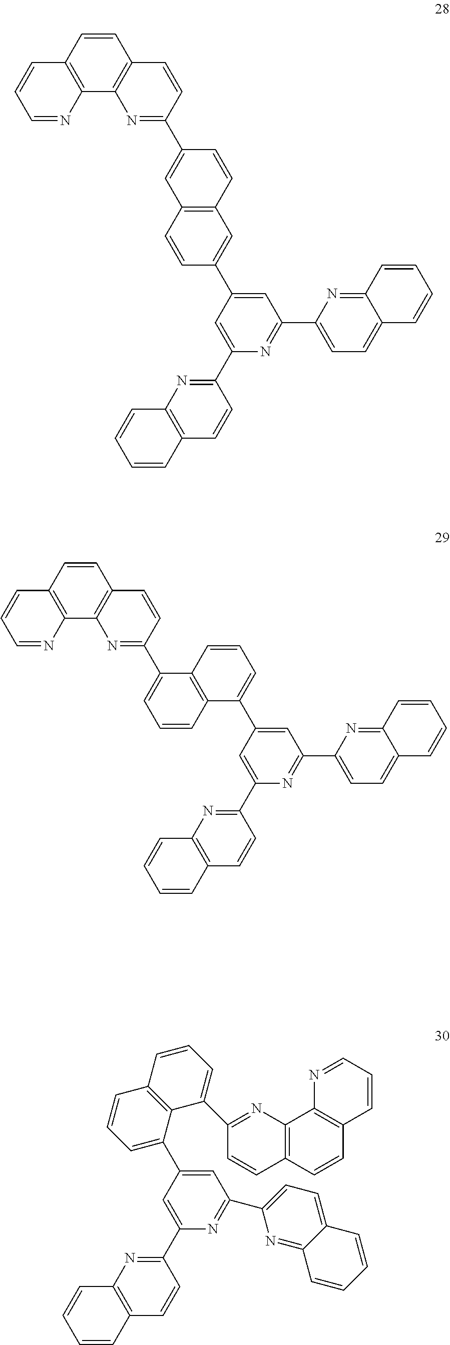 [SMILES: c1ccc2nc(-c3cc(-c4ccc5cc(-c6ccc7ccc8cccnc8c7n6)ccc5c4)cc(-c4ccc5ccccc5n4)n3)ccc2c1 ; c1ccc2nc(-c3cc(-c4cccc5c(-c6ccc7ccc8cccnc8c7n6)cccc45)cc(-c4ccc5ccccc5n4)n3)ccc2c1 ; c1ccc2nc(-c3cc(-c4cccc5cccc(-c6ccc7ccc8cccnc8c7n6)c45)cc(-c4ccc5ccccc5n4)n3)ccc2c1]

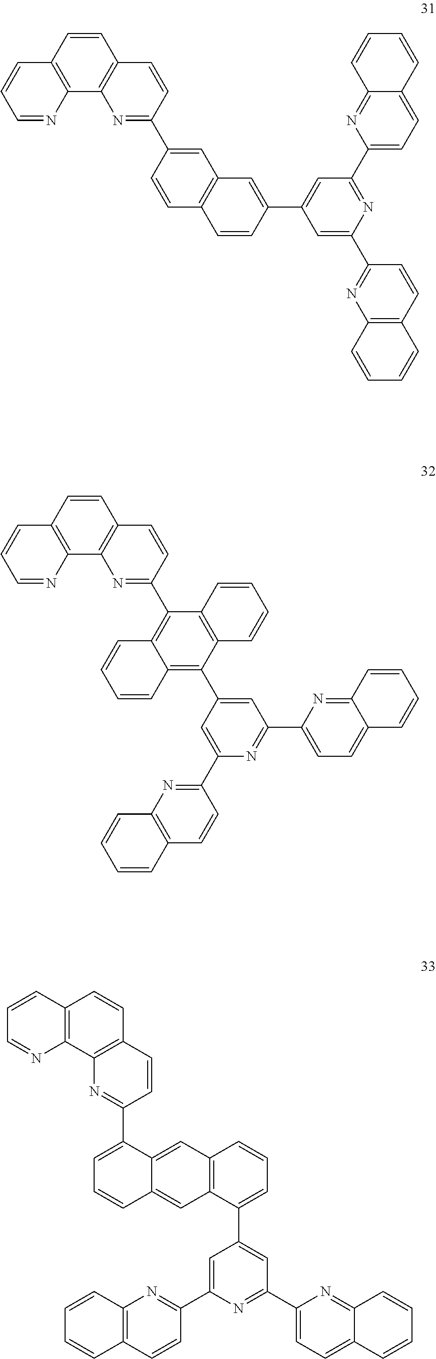 [SMILES: c1ccc2nc(-c3cc(-c4ccc5ccc(-c6ccc7ccc8cccnc8c7n6)cc5c4)cc(-c4ccc5ccccc5n4)n3)ccc2c1 ; c1ccc2nc(-c3cc(-c4c5ccccc5c(-c5ccc6ccc7cccnc7c6n5)c5ccccc45)cc(-c4ccc5ccccc5n4)n3)ccc2c1 ; c1ccc2nc(-c3cc(-c4cccc5cc6c(-c7ccc8ccc9cccnc9c8n7)cccc6cc45)cc(-c4ccc5ccccc5n4)n3)ccc2c1]

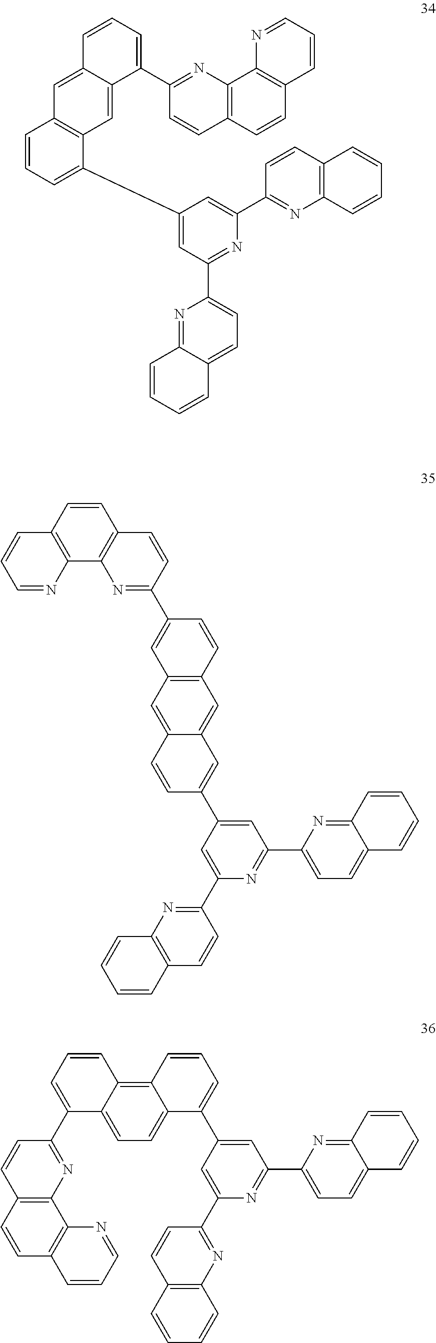 [SMILES: c1ccc2nc(-c3cc(-c4cccc5cc6cccc(-c7ccc8ccc9cccnc9c8n7)c6cc45)cc(-c4ccc5ccccc5n4)n3)ccc2c1 ; c1ccc2nc(-c3cc(-c4ccc5cc6cc(-c7ccc8ccc9cccnc9c8n7)ccc6cc5c4)cc(-c4ccc5ccccc5n4)n3)ccc2c1 ; c1ccc2nc(-c3cc(-c4cccc5c4ccc4c(-c6ccc7ccc8cccnc8c7n6)cccc45)cc(-c4ccc5ccccc5n4)n3)ccc2c1]

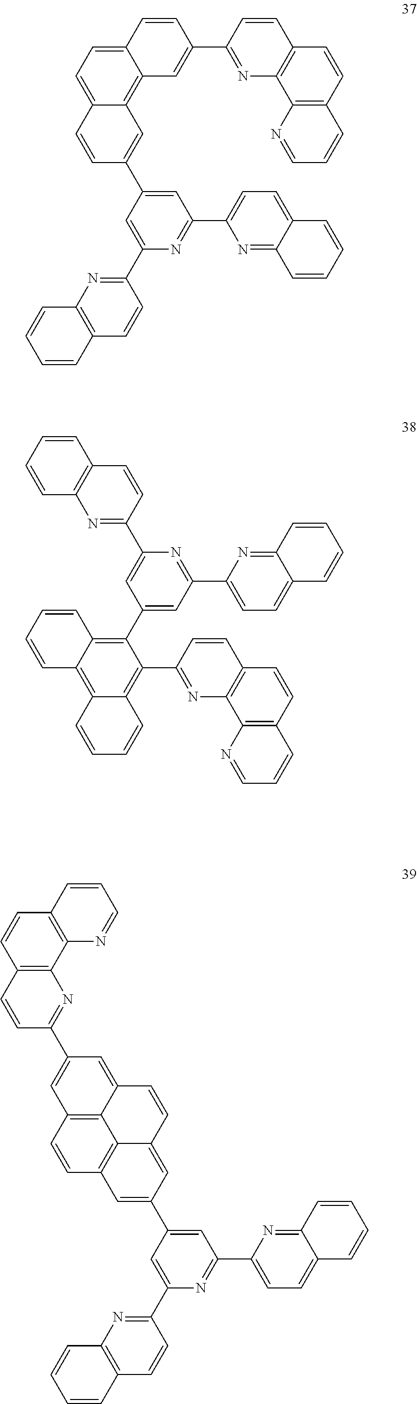 [SMILES: c1ccc2nc(-c3cc(-c4ccc5ccc6ccc(-c7ccc8ccc9cccnc9c8n7)cc6c5c4)cc(-c4ccc5ccccc5n4)n3)ccc2c1 ; c1ccc2nc(-c3cc(-c4c(-c5ccc6ccc7cccnc7c6n5)c5ccccc5c5ccccc45)cc(-c4ccc5ccccc5n4)n3)ccc2c1 ; c1ccc2nc(-c3cc(-c4cc5ccc6cc(-c7ccc8ccc9cccnc9c8n7)cc7ccc(c4)c5c67)cc(-c4ccc5ccccc5n4)n3)ccc2c1]

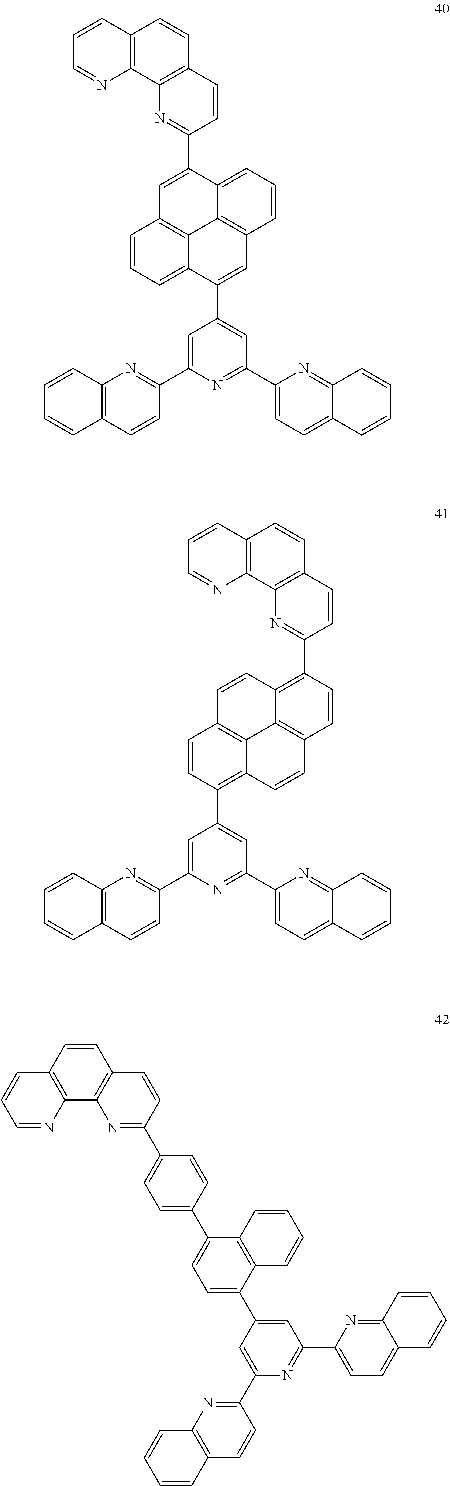 [SMILES: c1ccc2nc(-c3cc(-c4cc5cccc6c(-c7ccc8ccc9cccnc9c8n7)cc7cccc4c7c56)cc(-c4ccc5ccccc5n4)n3)ccc2c1 ; c1ccc2nc(-c3cc(-c4ccc5ccc6c(-c7ccc8ccc9cccnc9c8n7)ccc7ccc4c5c76)cc(-c4ccc5ccccc5n4)n3)ccc2c1 ; c1ccc2nc(-c3cc(-c4ccc(-c5ccc(-c6ccc7ccc8cccnc8c7n6)cc5)c5ccccc45)cc(-c4ccc5ccccc5n4)n3)ccc2c1]

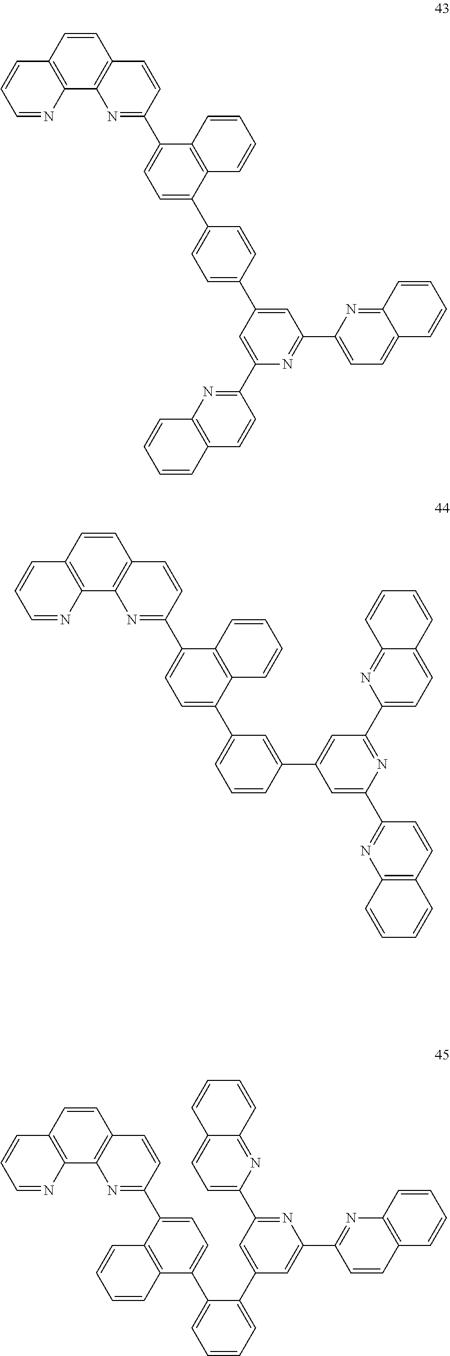 [SMILES: c1ccc2nc(-c3cc(-c4ccc(-c5ccc(-c6ccc7ccc8cccnc8c7n6)c6ccccc56)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc(-c3ccc4ccc5cccnc5c4n3)c3ccccc23)c1 ; c1ccc(-c2ccc(-c3ccc4ccc5cccnc5c4n3)c3ccccc23)c(-c2cc(-c3ccc4ccccc4n3)nc(-c3ccc4ccccc4n3)c2)c1]

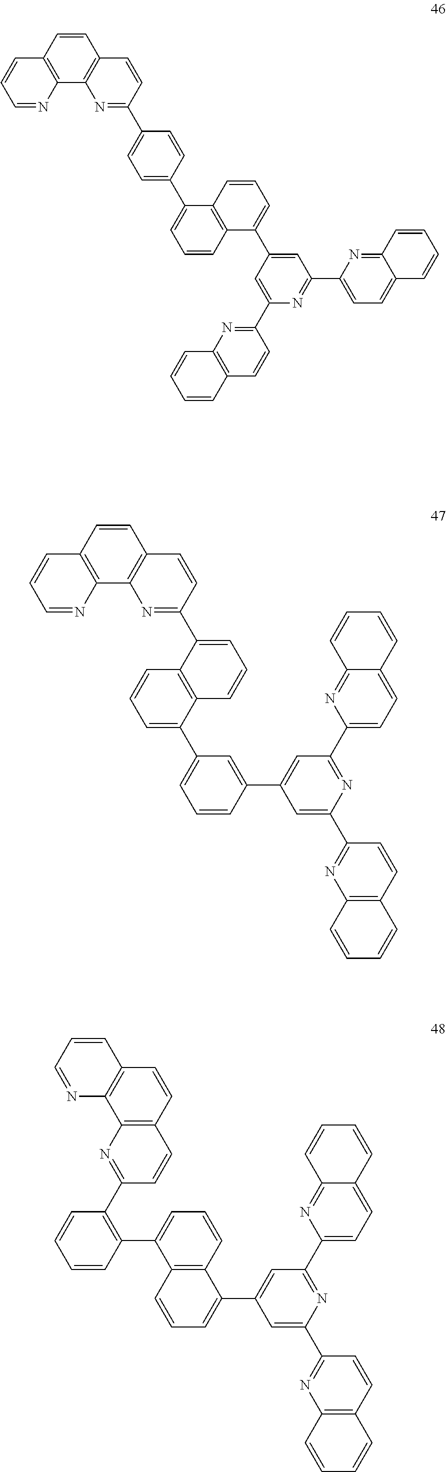 [SMILES: c1ccc2nc(-c3cc(-c4cccc5c(-c6ccc(-c7ccc8ccc9cccnc9c8n7)cc6)cccc45)cc(-c4ccc5ccccc5n4)n3)ccc2c1 ; c1cc(-c2cc(-c3ccc4ccccc4n3)nc(-c3ccc4ccccc4n3)c2)cc(-c2cccc3c(-c4ccc5ccc6cccnc6c5n4)cccc23)c1 ; c1ccc(-c2cccc3c(-c4cc(-c5ccc6ccccc6n5)nc(-c5ccc6ccccc6n5)c4)cccc23)c(-c2ccc3ccc4cccnc4c3n2)c1]

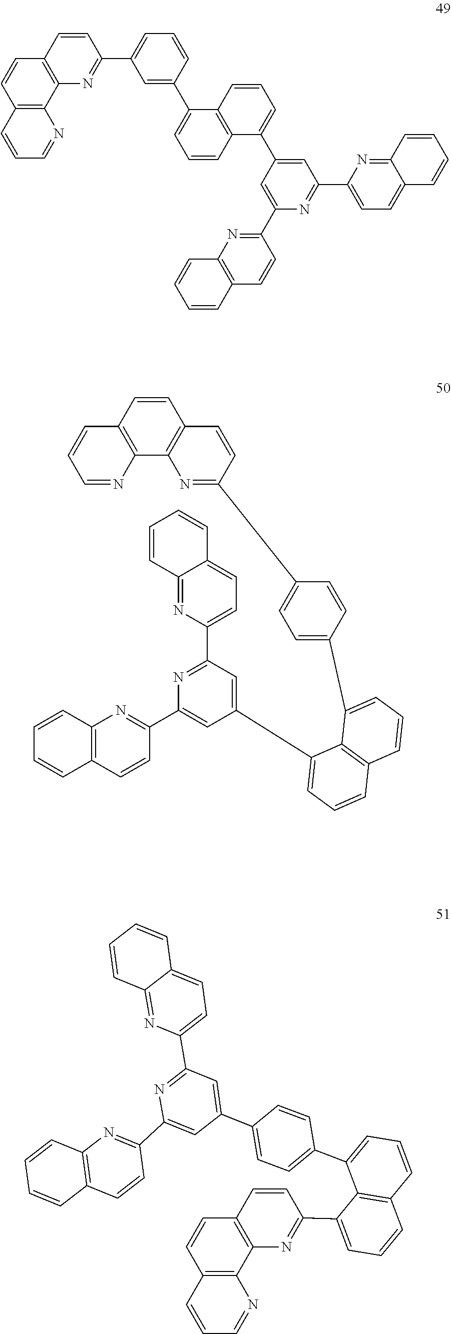 [SMILES: c1cc(-c2ccc3ccc4cccnc4c3n2)cc(-c2cccc3c(-c4cc(-c5ccc6ccccc6n5)nc(-c5ccc6ccccc6n5)c4)cccc23)c1 ; c1ccc2nc(-c3cc(-c4cccc5cccc(-c6ccc(-c7ccc8ccc9cccnc9c8n7)cc6)c45)cc(-c4ccc5ccccc5n4)n3)ccc2c1 ; c1ccc2nc(-c3cc(-c4ccc(-c5cccc6cccc(-c7ccc8ccc9cccnc9c8n7)c56)cc4)cc(-c4ccc5ccccc5n4)n3)ccc2c1]

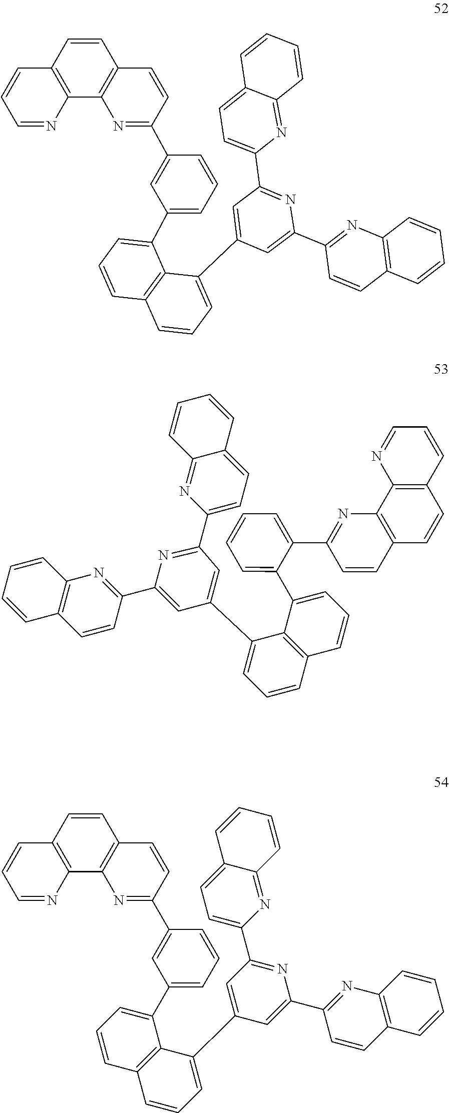 [SMILES: c1cc(-c2ccc3ccc4cccnc4c3n2)cc(-c2cccc3cccc(-c4cc(-c5ccc6ccccc6n5)nc(-c5ccc6ccccc6n5)c4)c23)c1 ; c1ccc(-c2cccc3cccc(-c4cc(-c5ccc6ccccc6n5)nc(-c5ccc6ccccc6n5)c4)c23)c(-c2ccc3ccc4cccnc4c3n2)c1 ; c1cc(-c2ccc3ccc4cccnc4c3n2)cc(-c2cccc3cccc(-c4cc(-c5ccc6ccccc6n5)nc(-c5ccc6ccccc6n5)c4)c23)c1]

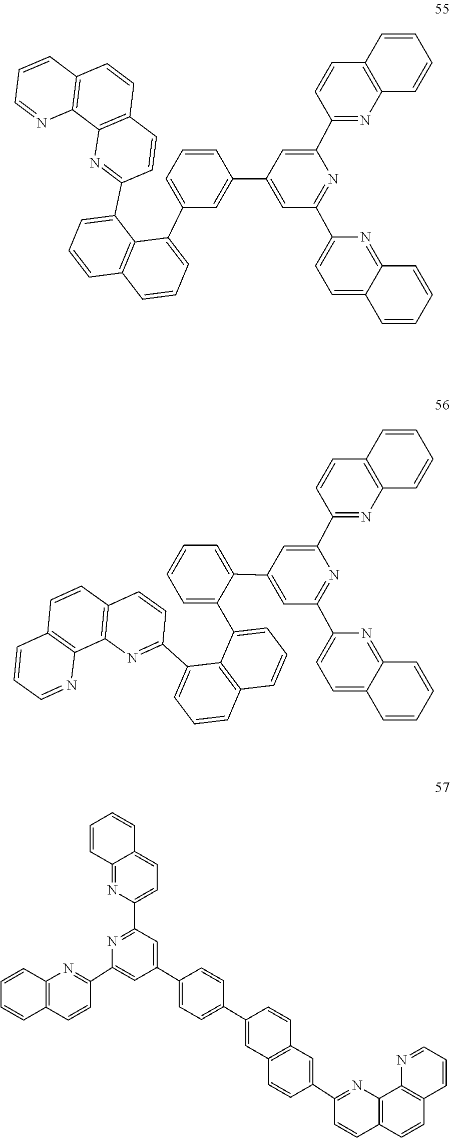 [SMILES: c1cc(-c2cc(-c3ccc4ccccc4n3)nc(-c3ccc4ccccc4n3)c2)cc(-c2cccc3cccc(-c4ccc5ccc6cccnc6c5n4)c23)c1 ; c1ccc(-c2cccc3cccc(-c4ccc5ccc6cccnc6c5n4)c23)c(-c2cc(-c3ccc4ccccc4n3)nc(-c3ccc4ccccc4n3)c2)c1 ; c1ccc2nc(-c3cc(-c4ccc(-c5ccc6cc(-c7ccc8ccc9cccnc9c8n7)ccc6c5)cc4)cc(-c4ccc5ccccc5n4)n3)ccc2c1]

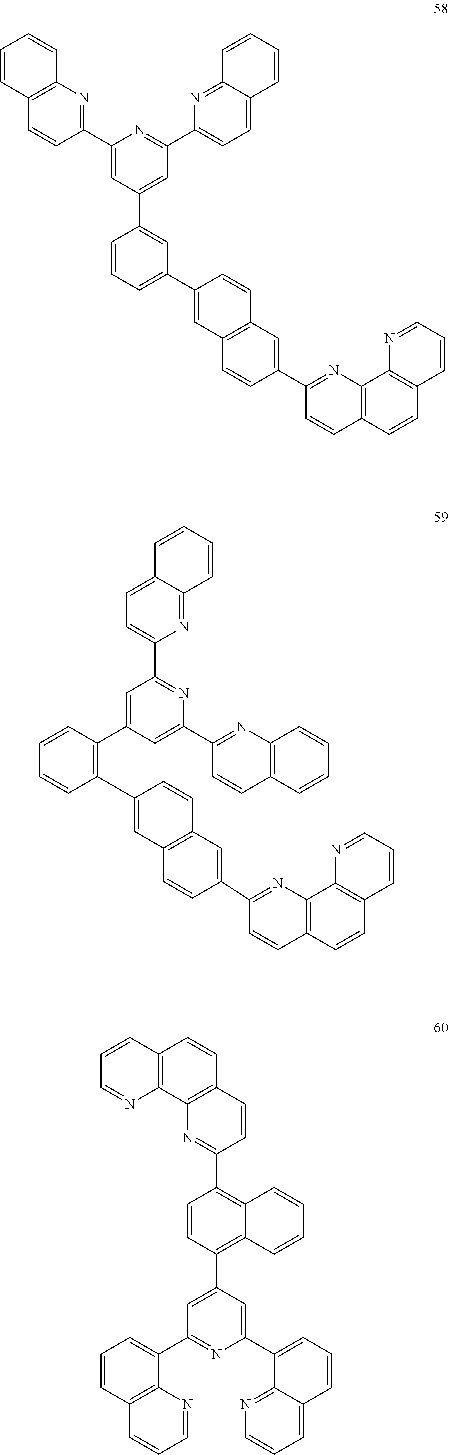 [SMILES: c1cc(-c2cc(-c3ccc4ccccc4n3)nc(-c3ccc4ccccc4n3)c2)cc(-c2ccc3cc(-c4ccc5ccc6cccnc6c5n4)ccc3c2)c1 ; c1ccc(-c2ccc3cc(-c4ccc5ccc6cccnc6c5n4)ccc3c2)c(-c2cc(-c3ccc4ccccc4n3)nc(-c3ccc4ccccc4n3)c2)c1 ; c1cnc2c(-c3cc(-c4ccc(-c5ccc6ccc7cccnc7c6n5)c5ccccc45)cc(-c4cccc5cccnc45)n3)cccc2c1]

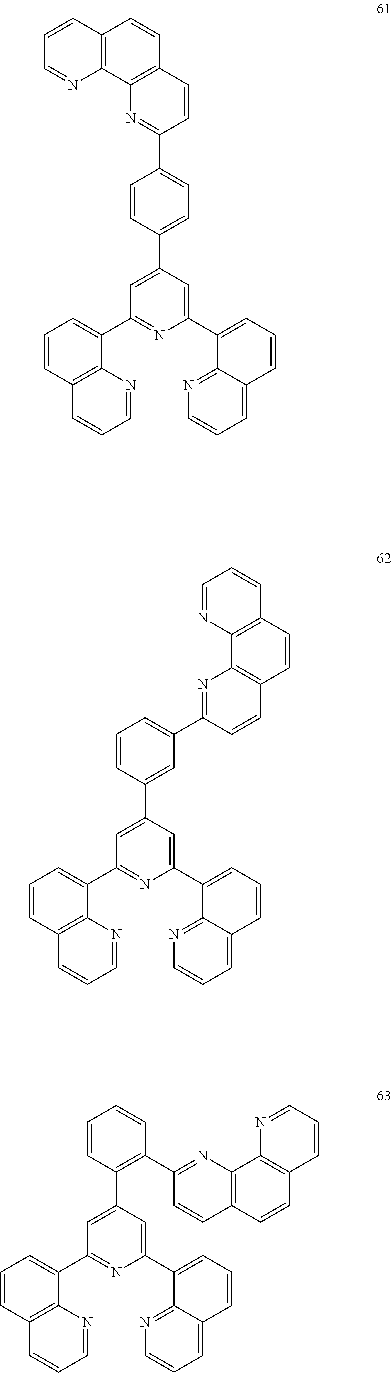 [SMILES: c1cnc2c(-c3cc(-c4ccc(-c5ccc6ccc7cccnc7c6n5)cc4)cc(-c4cccc5cccnc45)n3)cccc2c1 ; c1cc(-c2cc(-c3cccc4cccnc34)nc(-c3cccc4cccnc34)c2)cc(-c2ccc3ccc4cccnc4c3n2)c1 ; c1ccc(-c2ccc3ccc4cccnc4c3n2)c(-c2cc(-c3cccc4cccnc34)nc(-c3cccc4cccnc34)c2)c1]

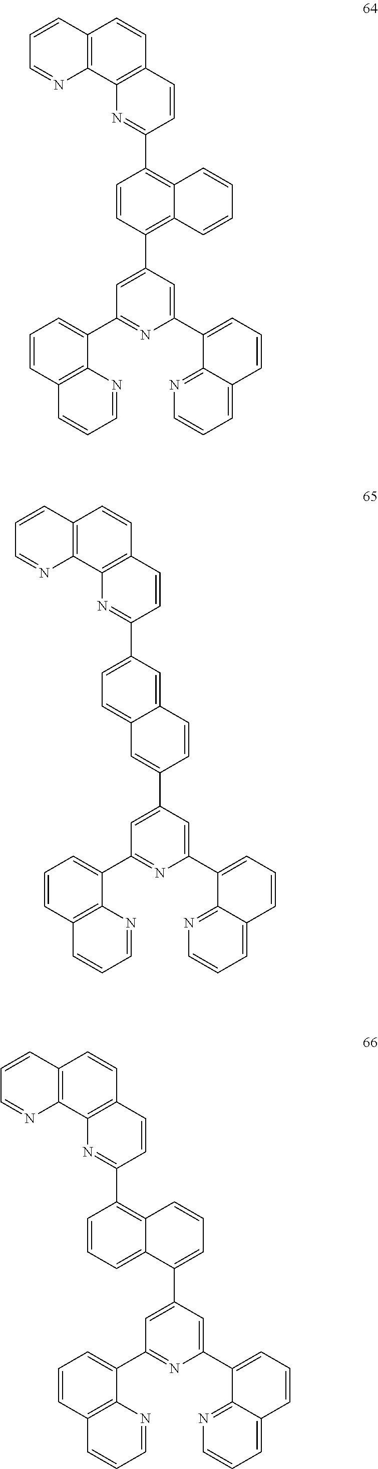 [SMILES: c1cnc2c(-c3cc(-c4ccc(-c5ccc6ccc7cccnc7c6n5)c5ccccc45)cc(-c4cccc5cccnc45)n3)cccc2c1 ; c1cnc2c(-c3cc(-c4ccc5cc(-c6ccc7ccc8cccnc8c7n6)ccc5c4)cc(-c4cccc5cccnc45)n3)cccc2c1 ; c1cnc2c(-c3cc(-c4cccc5c(-c6ccc7ccc8cccnc8c7n6)cccc45)cc(-c4cccc5cccnc45)n3)cccc2c1]

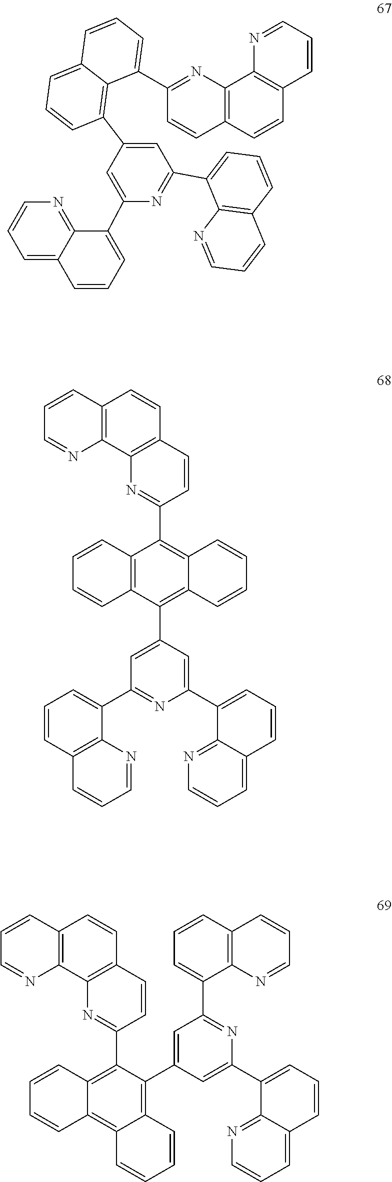 [SMILES: c1cnc2c(-c3cc(-c4cccc5cccc(-c6ccc7ccc8cccnc8c7n6)c45)cc(-c4cccc5cccnc45)n3)cccc2c1 ; c1cnc2c(-c3cc(-c4c5ccccc5c(-c5ccc6ccc7cccnc7c6n5)c5ccccc45)cc(-c4cccc5cccnc45)n3)cccc2c1 ; c1cnc2c(-c3cc(-c4c(-c5ccc6ccc7cccnc7c6n5)c5ccccc5c5ccccc45)cc(-c4cccc5cccnc45)n3)cccc2c1]

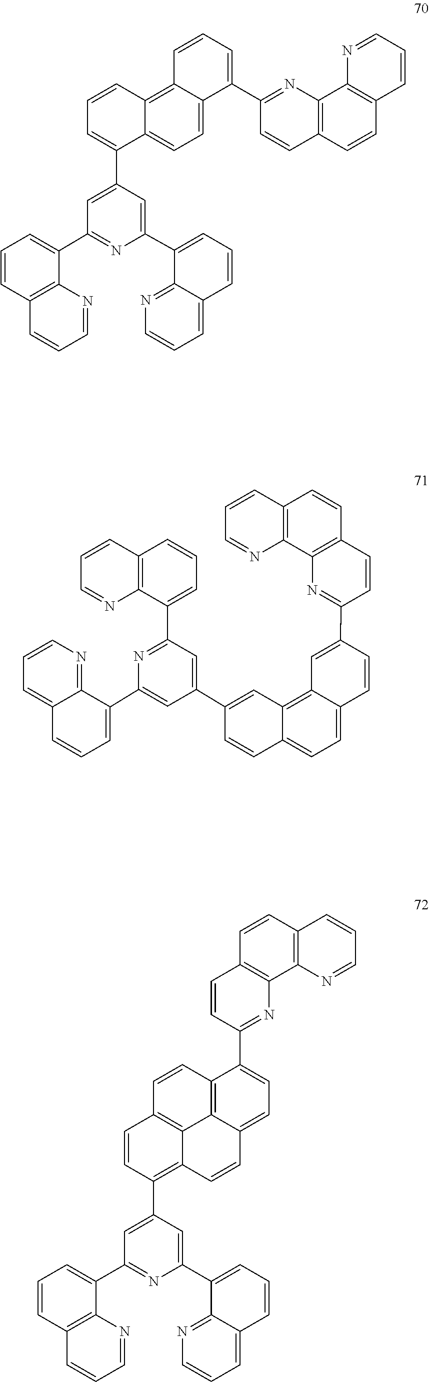 [SMILES: c1cnc2c(-c3cc(-c4cccc5c4ccc4c(-c6ccc7ccc8cccnc8c7n6)cccc45)cc(-c4cccc5cccnc45)n3)cccc2c1 ; c1cnc2c(-c3cc(-c4ccc5ccc6ccc(-c7ccc8ccc9cccnc9c8n7)cc6c5c4)cc(-c4cccc5cccnc45)n3)cccc2c1 ; c1cnc2c(-c3cc(-c4ccc5ccc6c(-c7ccc8ccc9cccnc9c8n7)ccc7ccc4c5c76)cc(-c4cccc5cccnc45)n3)cccc2c1]

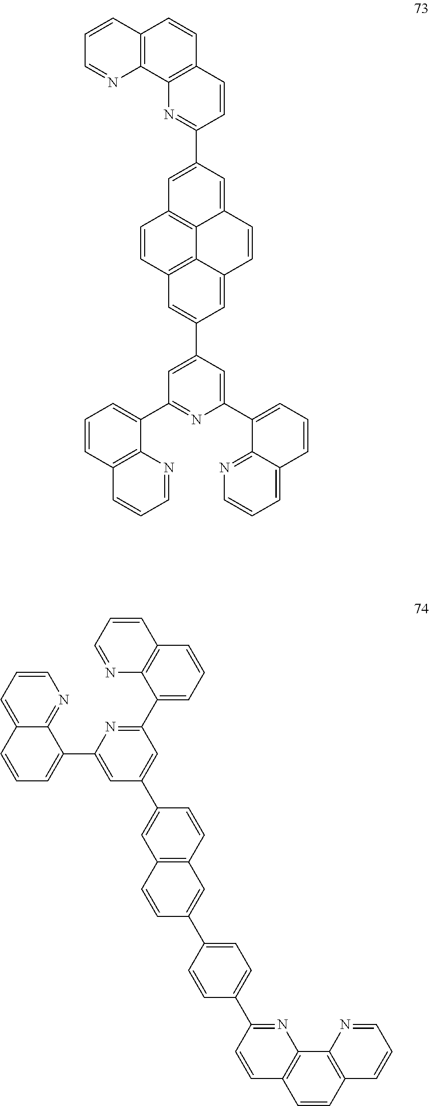

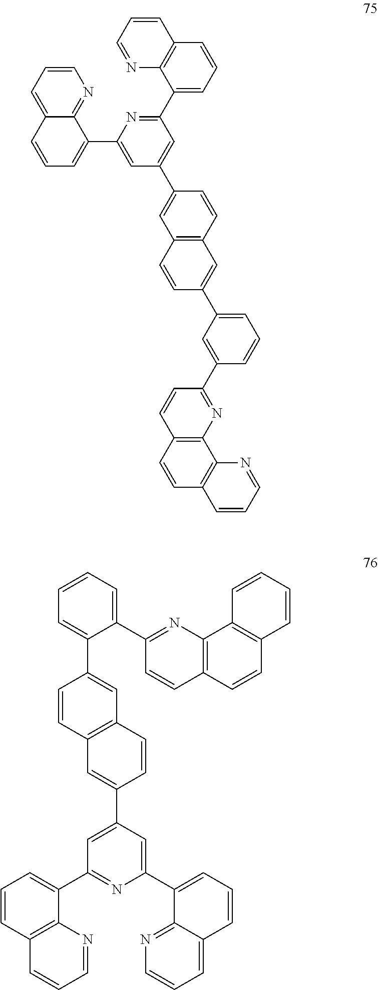 [SMILES: c1cc(-c2ccc3cc(-c4cc(-c5cccc6cccnc56)nc(-c5cccc6cccnc56)c4)ccc3c2)cc(-c2ccc3ccc4cccnc4c3n2)c1 ; c1ccc(-c2ccc3ccc4ccccc4c3n2)c(-c2ccc3cc(-c4cc(-c5cccc6cccnc56)nc(-c5cccc6cccnc56)c4)ccc3c2)c1]

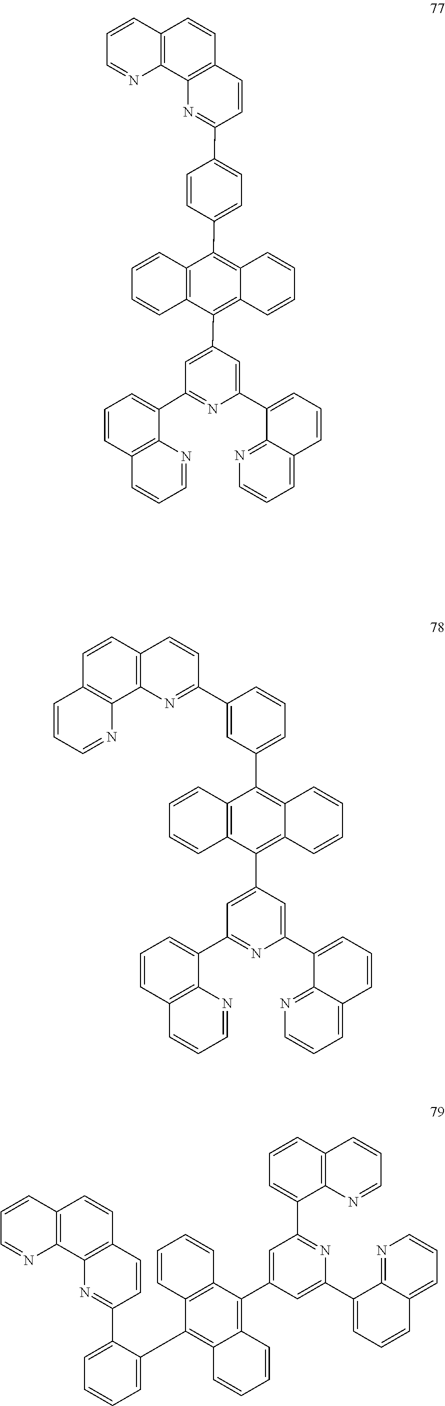 [SMILES: c1cnc2c(-c3cc(-c4c5ccccc5c(-c5ccc(-c6ccc7ccc8cccnc8c7n6)cc5)c5ccccc45)cc(-c4cccc5cccnc45)n3)cccc2c1 ; c1cc(-c2ccc3ccc4cccnc4c3n2)cc(-c2c3ccccc3c(-c3cc(-c4cccc5cccnc45)nc(-c4cccc5cccnc45)c3)c3ccccc23)c1 ; c1ccc(-c2c3ccccc3c(-c3cc(-c4cccc5cccnc45)nc(-c4cccc5cccnc45)c3)c3ccccc23)c(-c2ccc3ccc4cccnc4c3n2)c1]

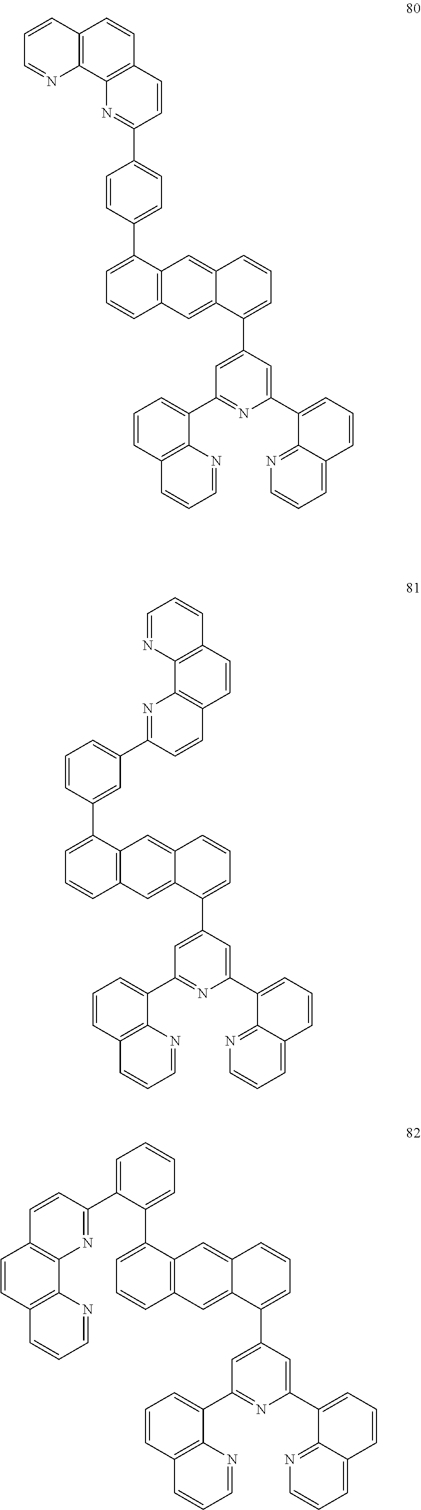 [SMILES: c1cc(-c2ccc(-c3ccc4ccc5cccnc5c4n3)cc2)c2cc3cccc(-c4cc(-c5cccc6cccnc56)nc(-c5cccc6cccnc56)c4)c3cc2c1 ; c1cc(-c2ccc3ccc4cccnc4c3n2)cc(-c2cccc3cc4c(-c5cc(-c6cccc7cccnc67)nc(-c6cccc7cccnc67)c5)cccc4cc23)c1 ; c1ccc(-c2cccc3cc4c(-c5cc(-c6cccc7cccnc67)nc(-c6cccc7cccnc67)c5)cccc4cc23)c(-c2ccc3ccc4cccnc4c3n2)c1]

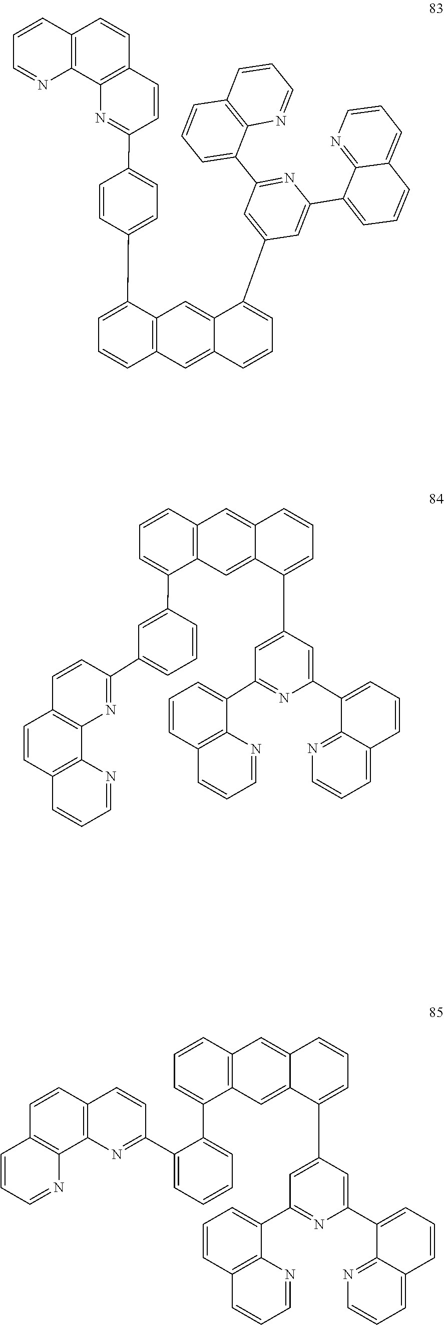 [SMILES: c1cc(-c2ccc(-c3ccc4ccc5cccnc5c4n3)cc2)c2cc3c(-c4cc(-c5cccc6cccnc56)nc(-c5cccc6cccnc56)c4)cccc3cc2c1 ; c1cc(-c2ccc3ccc4cccnc4c3n2)cc(-c2cccc3cc4cccc(-c5cc(-c6cccc7cccnc67)nc(-c6cccc7cccnc67)c5)c4cc23)c1 ; c1ccc(-c2cccc3cc4cccc(-c5cc(-c6cccc7cccnc67)nc(-c6cccc7cccnc67)c5)c4cc23)c(-c2ccc3ccc4cccnc4c3n2)c1]

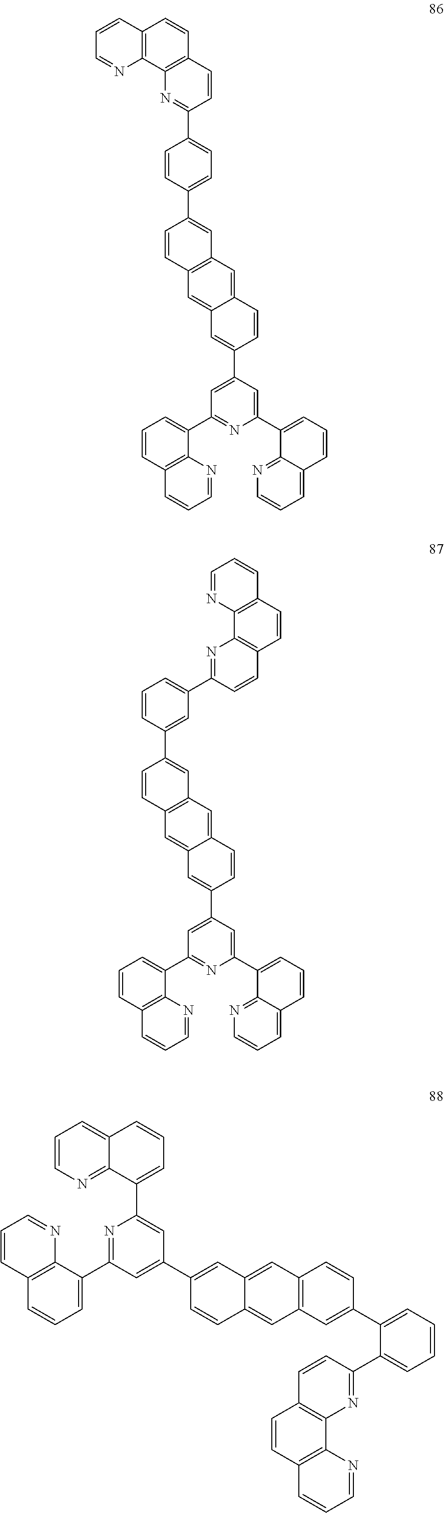 [SMILES: c1cnc2c(-c3cc(-c4ccc5cc6cc(-c7ccc(-c8ccc9ccc%10cccnc%10c9n8)cc7)ccc6cc5c4)cc(-c4cccc5cccnc45)n3)cccc2c1 ; c1cc(-c2ccc3cc4cc(-c5cc(-c6cccc7cccnc67)nc(-c6cccc7cccnc67)c5)ccc4cc3c2)cc(-c2ccc3ccc4cccnc4c3n2)c1 ; c1ccc(-c2ccc3ccc4cccnc4c3n2)c(-c2ccc3cc4cc(-c5cc(-c6cccc7cccnc67)nc(-c6cccc7cccnc67)c5)ccc4cc3c2)c1]

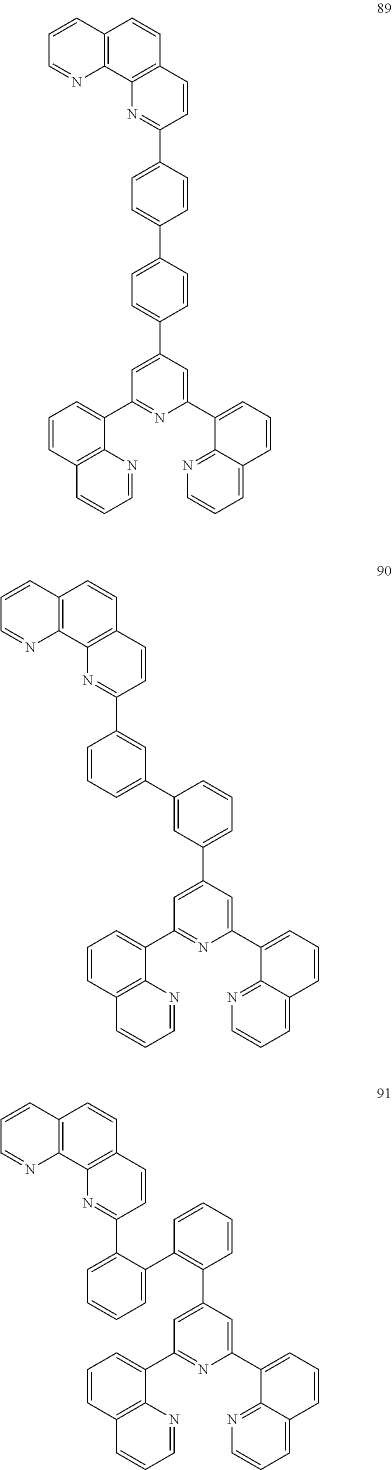 [SMILES: c1cnc2c(-c3cc(-c4ccc(-c5ccc(-c6ccc7ccc8cccnc8c7n6)cc5)cc4)cc(-c4cccc5cccnc45)n3)cccc2c1 ; c1cc(-c2cccc(-c3ccc4ccc5cccnc5c4n3)c2)cc(-c2cc(-c3cccc4cccnc34)nc(-c3cccc4cccnc34)c2)c1 ; c1ccc(-c2ccccc2-c2ccc3ccc4cccnc4c3n2)c(-c2cc(-c3cccc4cccnc34)nc(-c3cccc4cccnc34)c2)c1]

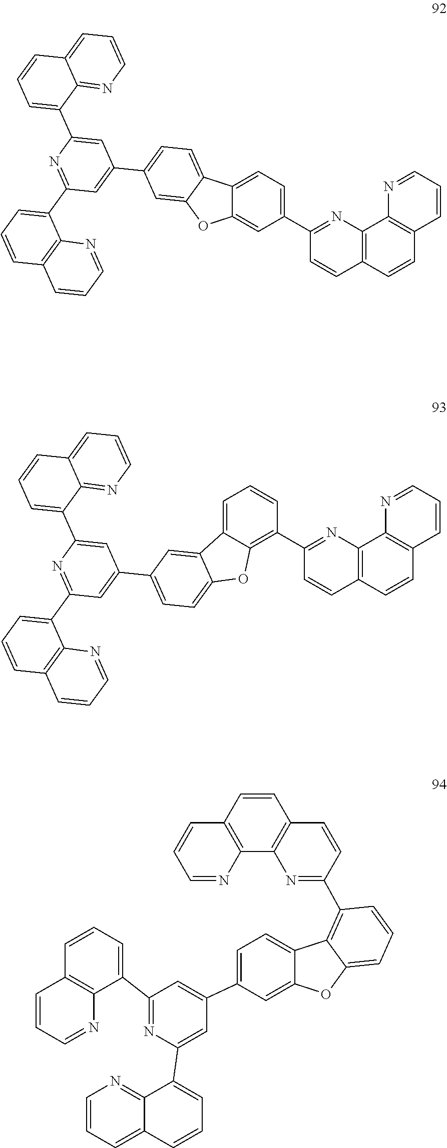 [SMILES: c1cnc2c(-c3cc(-c4ccc5c(c4)oc4cc(-c6ccc7ccc8cccnc8c7n6)ccc45)cc(-c4cccc5cccnc45)n3)cccc2c1 ; c1cnc2c(-c3cc(-c4ccc5oc6c(-c7ccc8ccc9cccnc9c8n7)cccc6c5c4)cc(-c4cccc5cccnc45)n3)cccc2c1 ; c1cnc2c(-c3cc(-c4ccc5c(c4)oc4cccc(-c6ccc7ccc8cccnc8c7n6)c45)cc(-c4cccc5cccnc45)n3)cccc2c1]

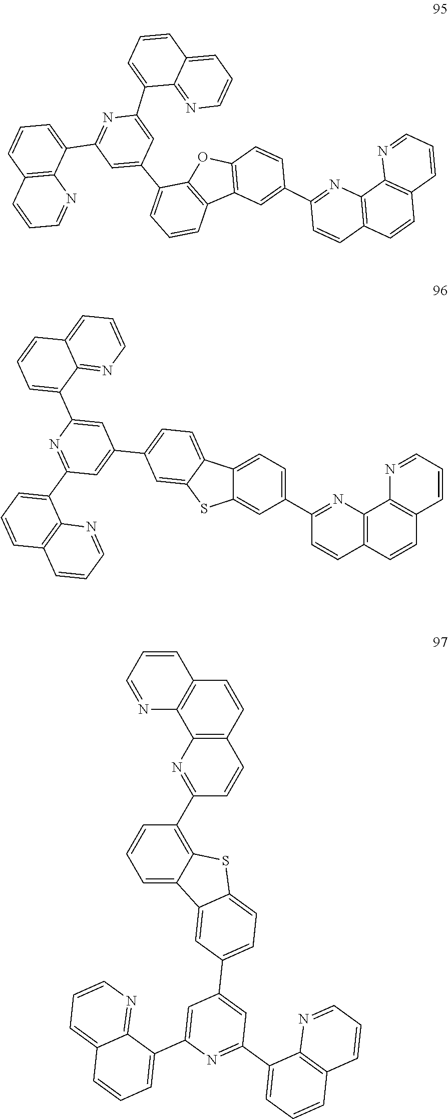 [SMILES: c1cnc2c(-c3cc(-c4cccc5c4oc4ccc(-c6ccc7ccc8cccnc8c7n6)cc45)cc(-c4cccc5cccnc45)n3)cccc2c1 ; c1cnc2c(-c3cc(-c4ccc5c(c4)sc4cc(-c6ccc7ccc8cccnc8c7n6)ccc45)cc(-c4cccc5cccnc45)n3)cccc2c1 ; c1cnc2c(-c3cc(-c4ccc5sc6c(-c7ccc8ccc9cccnc9c8n7)cccc6c5c4)cc(-c4cccc5cccnc45)n3)cccc2c1]

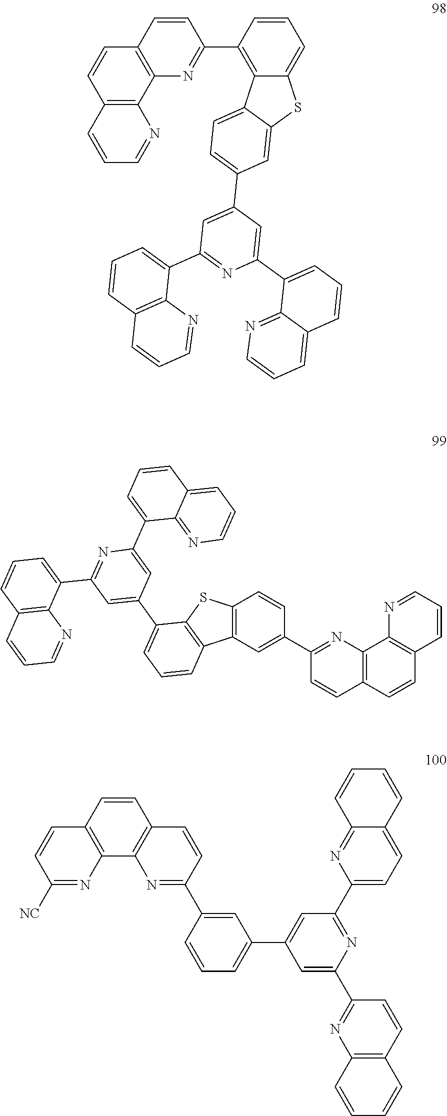 [SMILES: c1cnc2c(-c3cc(-c4ccc5c(c4)sc4cccc(-c6ccc7ccc8cccnc8c7n6)c45)cc(-c4cccc5cccnc45)n3)cccc2c1 ; c1cnc2c(-c3cc(-c4cccc5c4sc4ccc(-c6ccc7ccc8cccnc8c7n6)cc45)cc(-c4cccc5cccnc45)n3)cccc2c1 ; N#Cc1ccc2ccc3ccc(-c4cccc(-c5cc(-c6ccc7ccccc7n6)nc(-c6ccc7ccccc7n6)c5)c4)nc3c2n1]

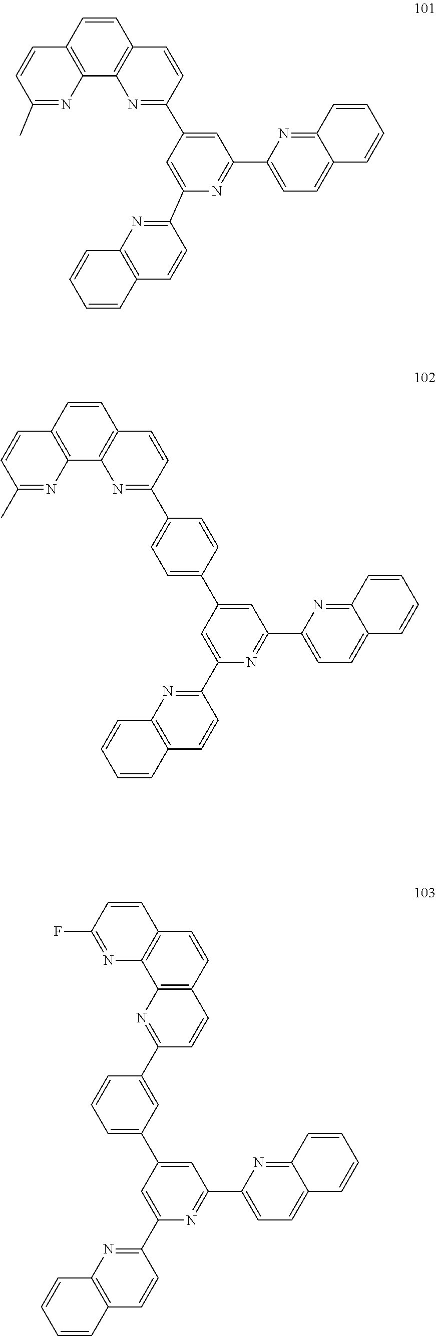 [SMILES: Cc1ccc2ccc3ccc(-c4cc(-c5ccc6ccccc6n5)nc(-c5ccc6ccccc6n5)c4)nc3c2n1 ; Cc1ccc2ccc3ccc(-c4ccc(-c5cc(-c6ccc7ccccc7n6)nc(-c6ccc7ccccc7n6)c5)cc4)nc3c2n1 ; Fc1ccc2ccc3ccc(-c4cccc(-c5cc(-c6ccc7ccccc7n6)nc(-c6ccc7ccccc7n6)c5)c4)nc3c2n1]

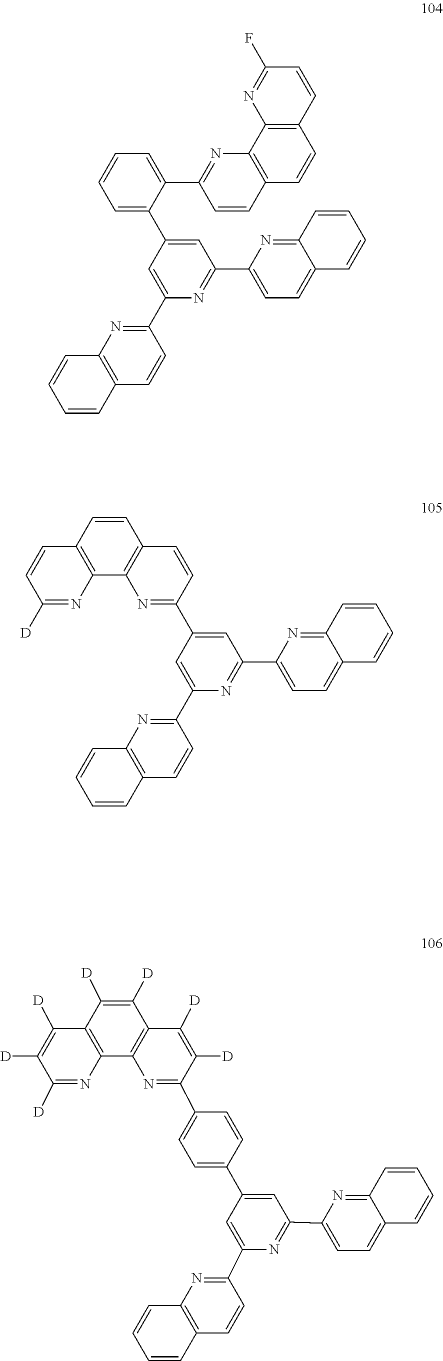 [SMILES: Fc1ccc2ccc3ccc(-c4ccccc4-c4cc(-c5ccc6ccccc6n5)nc(-c5ccc6ccccc6n5)c4)nc3c2n1 ; [2H]c1ccc2ccc3ccc(-c4cc(-c5ccc6ccccc6n5)nc(-c5ccc6ccccc6n5)c4)nc3c2n1 ; [2H]c1nc2c(c([2H])c1[2H])c([2H])c([2H])c1c([2H])c([2H])c(-c3ccc(-c4cc(-c5ccc6ccccc6n5)nc(-c5ccc6ccccc6n5)c4)cc3)nc12]

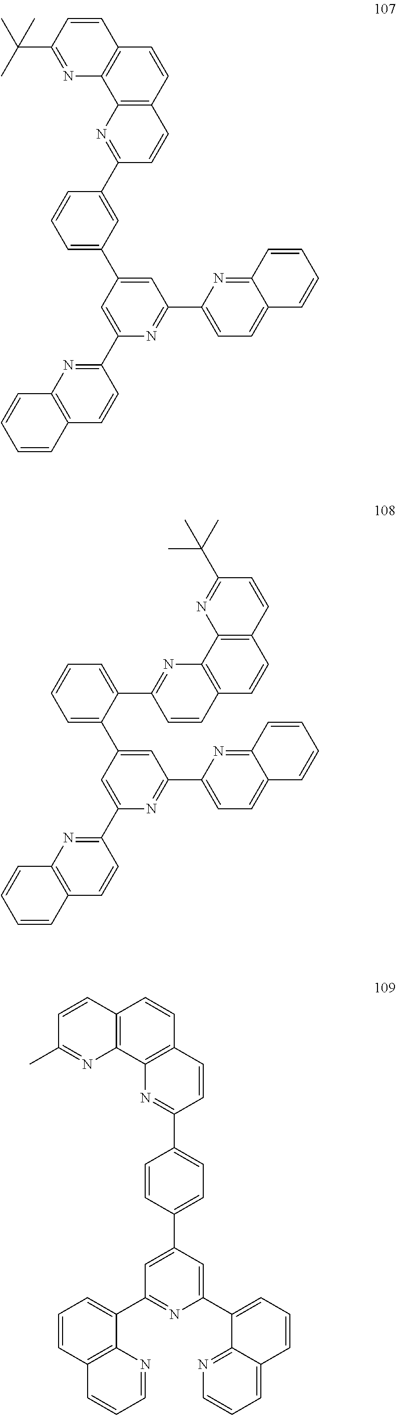 [SMILES: CC(C)(C)c1ccc2ccc3ccc(-c4cccc(-c5cc(-c6ccc7ccccc7n6)nc(-c6ccc7ccccc7n6)c5)c4)nc3c2n1 ; CC(C)(C)c1ccc2ccc3ccc(-c4ccccc4-c4cc(-c5ccc6ccccc6n5)nc(-c5ccc6ccccc6n5)c4)nc3c2n1 ; Cc1ccc2ccc3ccc(-c4ccc(-c5cc(-c6cccc7cccnc67)nc(-c6cccc7cccnc67)c5)cc4)nc3c2n1]

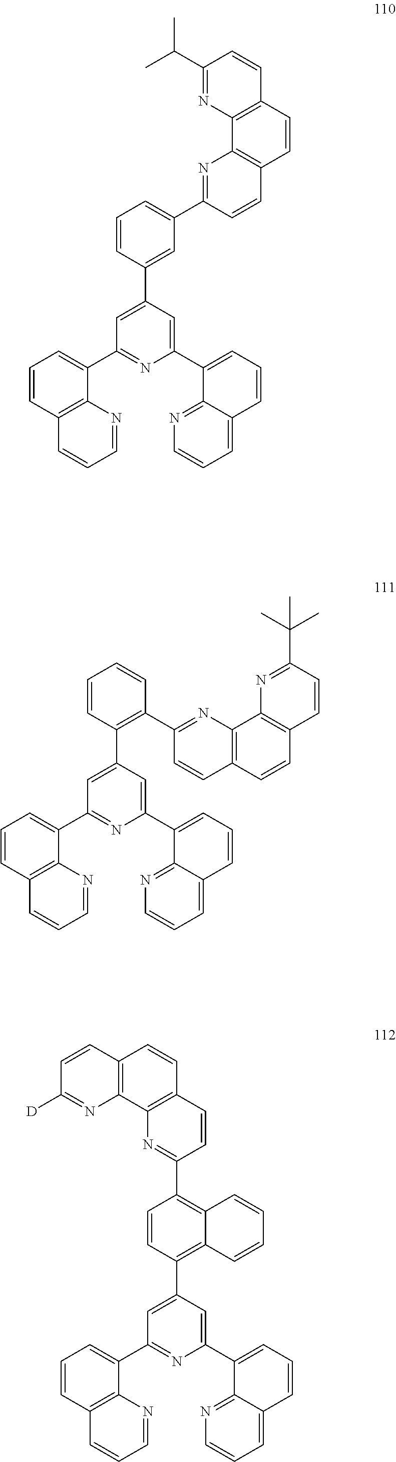 [SMILES: CC(C)c1ccc2ccc3ccc(-c4cccc(-c5cc(-c6cccc7cccnc67)nc(-c6cccc7cccnc67)c5)c4)nc3c2n1 ; CC(C)(C)c1ccc2ccc3ccc(-c4ccccc4-c4cc(-c5cccc6cccnc56)nc(-c5cccc6cccnc56)c4)nc3c2n1 ; [2H]c1ccc2ccc3ccc(-c4ccc(-c5cc(-c6cccc7cccnc67)nc(-c6cccc7cccnc67)c5)c5ccccc45)nc3c2n1]

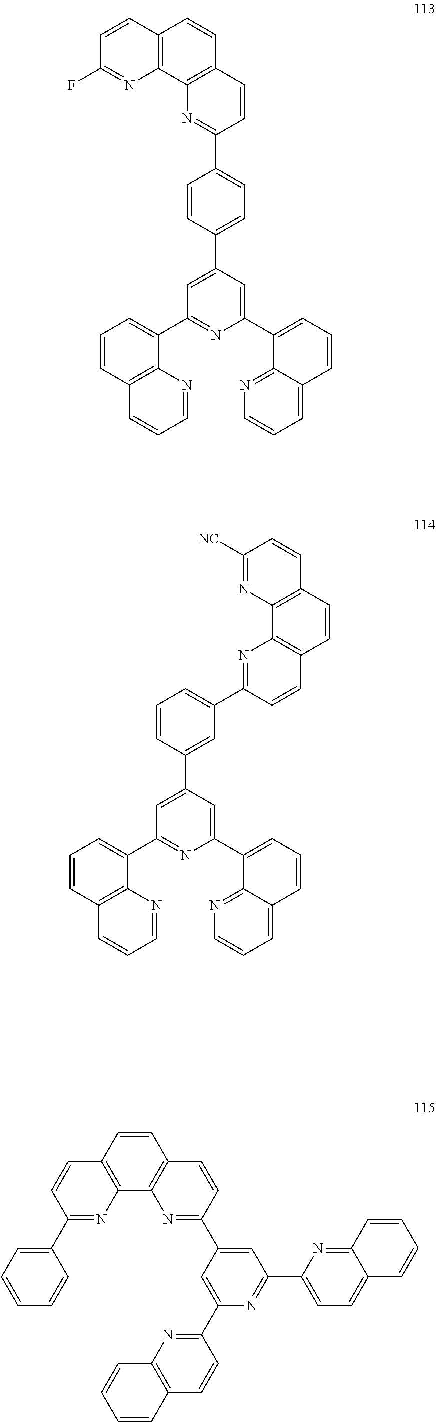 [SMILES: Fc1ccc2ccc3ccc(-c4ccc(-c5cc(-c6cccc7cccnc67)nc(-c6cccc7cccnc67)c5)cc4)nc3c2n1 ; N#Cc1ccc2ccc3ccc(-c4cccc(-c5cc(-c6cccc7cccnc67)nc(-c6cccc7cccnc67)c5)c4)nc3c2n1 ; c1ccc(-c2ccc3ccc4ccc(-c5cc(-c6ccc7ccccc7n6)nc(-c6ccc7ccccc7n6)c5)nc4c3n2)cc1]

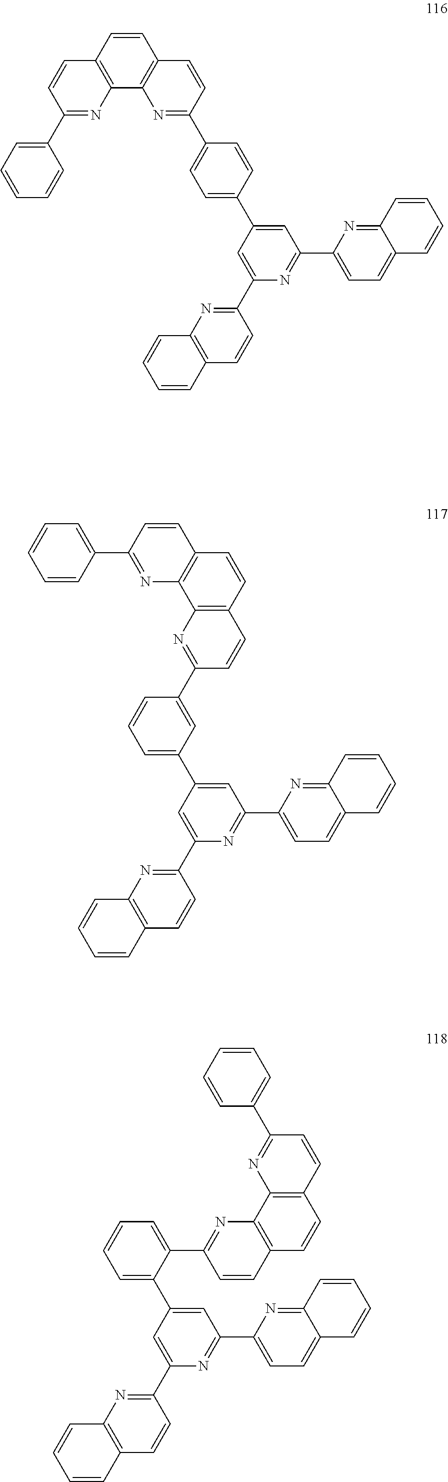 [SMILES: c1ccc(-c2ccc3ccc4ccc(-c5ccc(-c6cc(-c7ccc8ccccc8n7)nc(-c7ccc8ccccc8n7)c6)cc5)nc4c3n2)cc1 ; c1ccc(-c2ccc3ccc4ccc(-c5cccc(-c6cc(-c7ccc8ccccc8n7)nc(-c7ccc8ccccc8n7)c6)c5)nc4c3n2)cc1 ; c1ccc(-c2ccc3ccc4ccc(-c5ccccc5-c5cc(-c6ccc7ccccc7n6)nc(-c6ccc7ccccc7n6)c5)nc4c3n2)cc1]

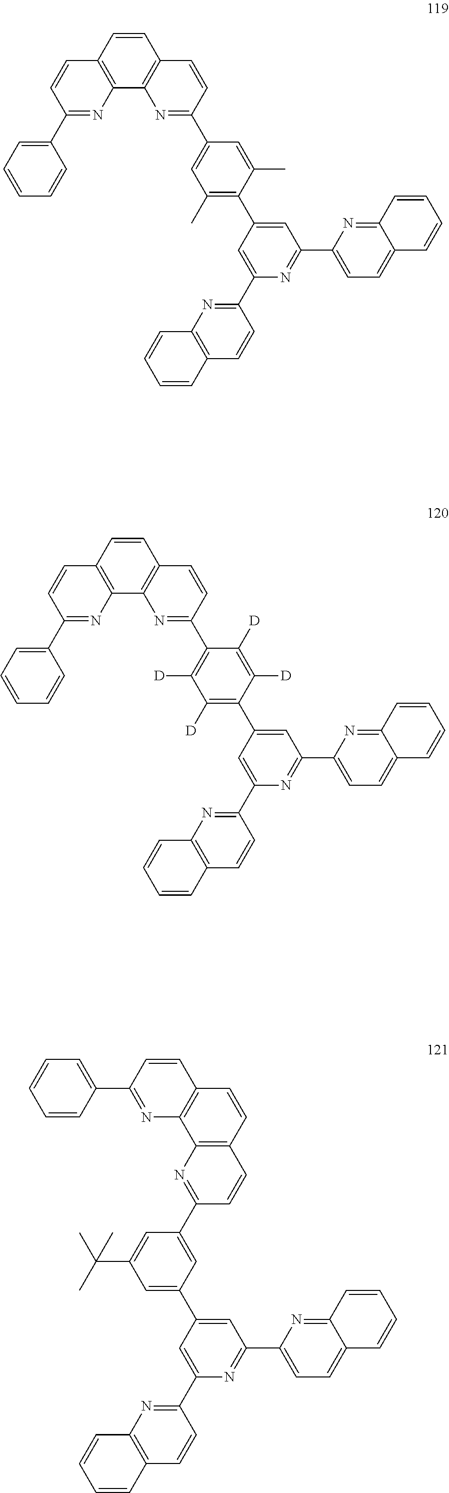 [SMILES: Cc1cc(-c2ccc3ccc4ccc(-c5ccccc5)nc4c3n2)cc(C)c1-c1cc(-c2ccc3ccccc3n2)nc(-c2ccc3ccccc3n2)c1 ; [2H]c1c([2H])c(-c2ccc3ccc4ccc(-c5ccccc5)nc4c3n2)c([2H])c([2H])c1-c1cc(-c2ccc3ccccc3n2)nc(-c2ccc3ccccc3n2)c1 ; CC(C)(C)c1cc(-c2cc(-c3ccc4ccccc4n3)nc(-c3ccc4ccccc4n3)c2)cc(-c2ccc3ccc4ccc(-c5ccccc5)nc4c3n2)c1]

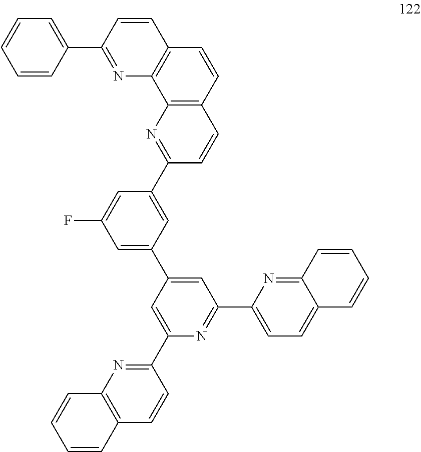 [SMILES: Fc1cc(-c2cc(-c3ccc4ccccc4n3)nc(-c3ccc4ccccc4n3)c2)cc(-c2ccc3ccc4ccc(-c5ccccc5)nc4c3n2)c1]

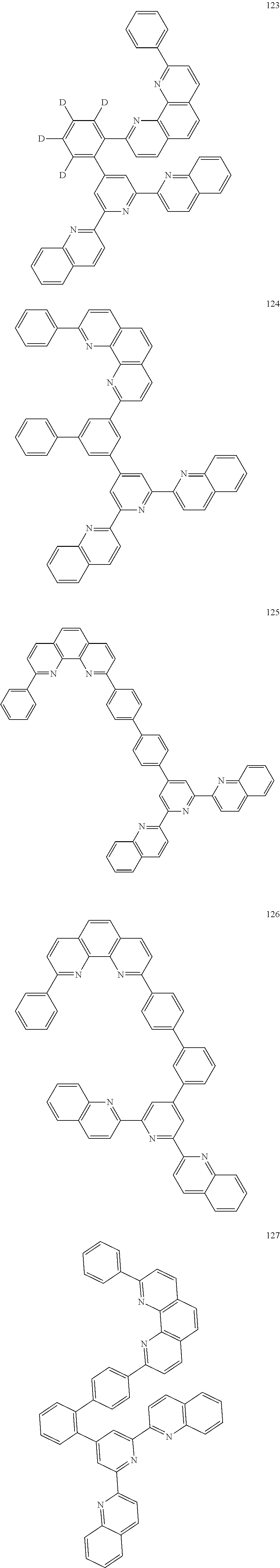

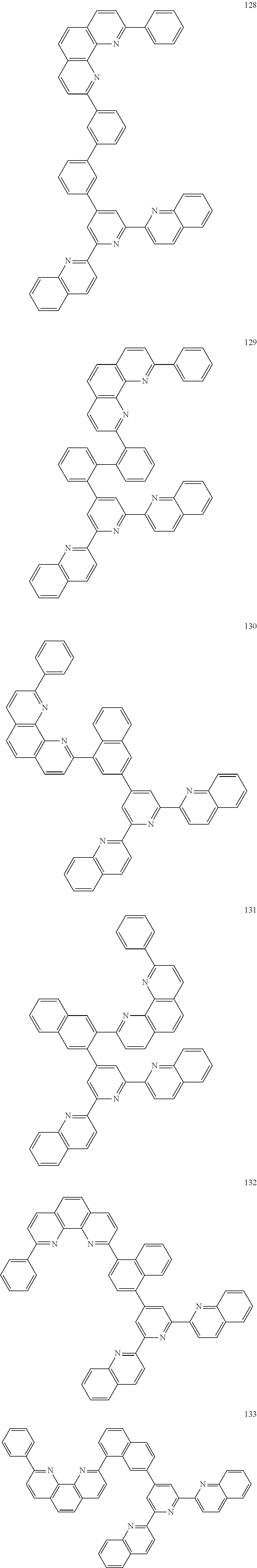

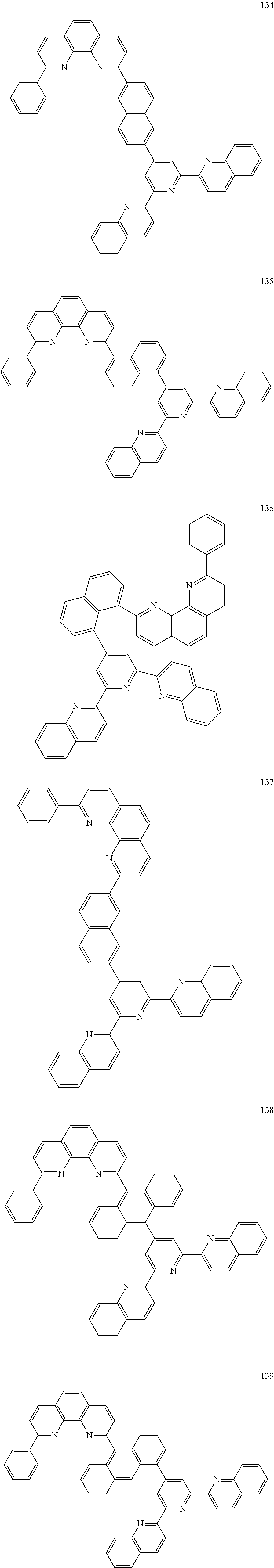

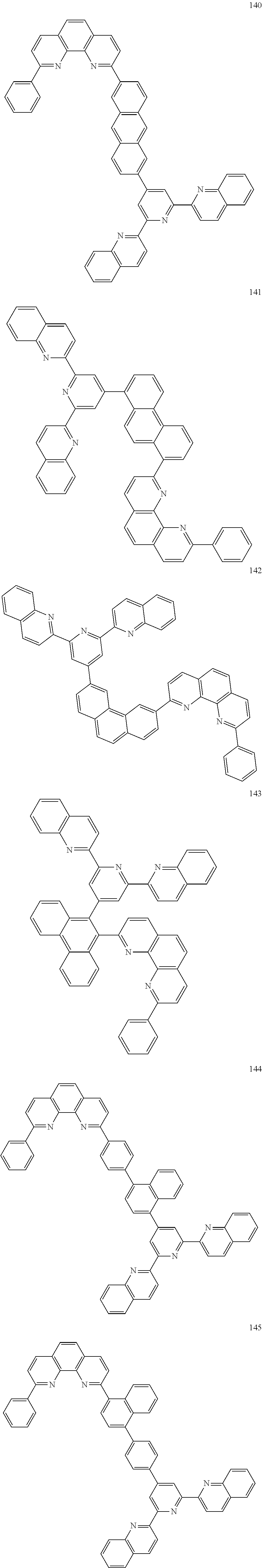

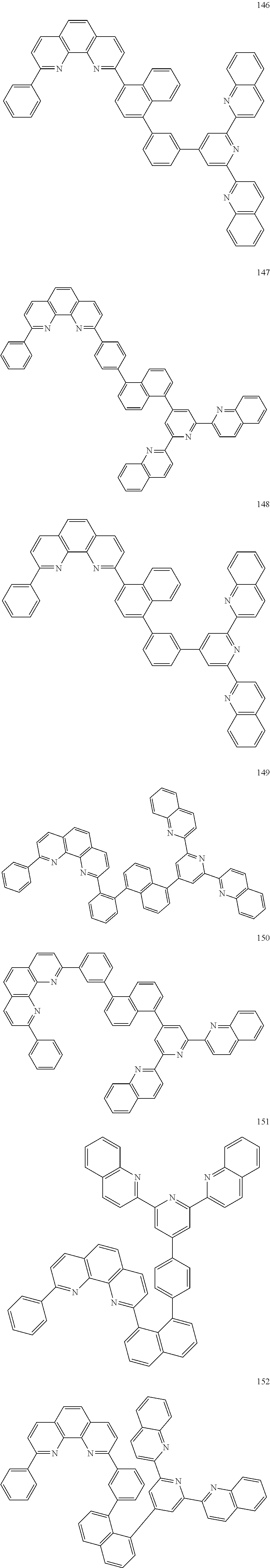

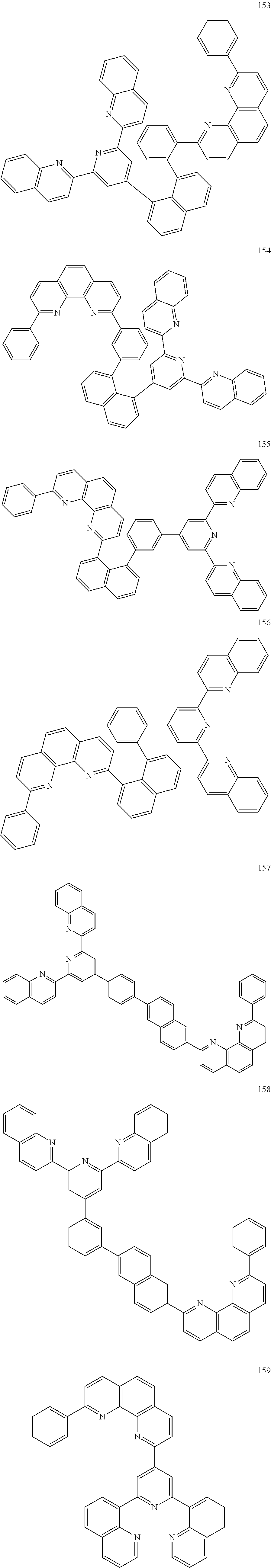

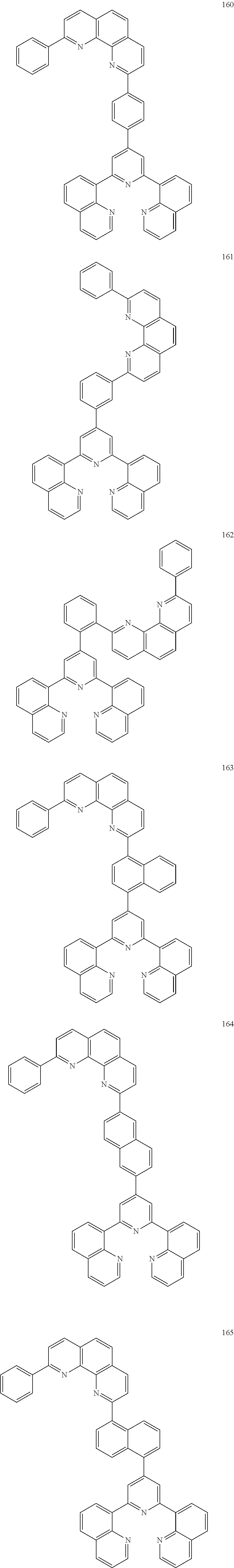

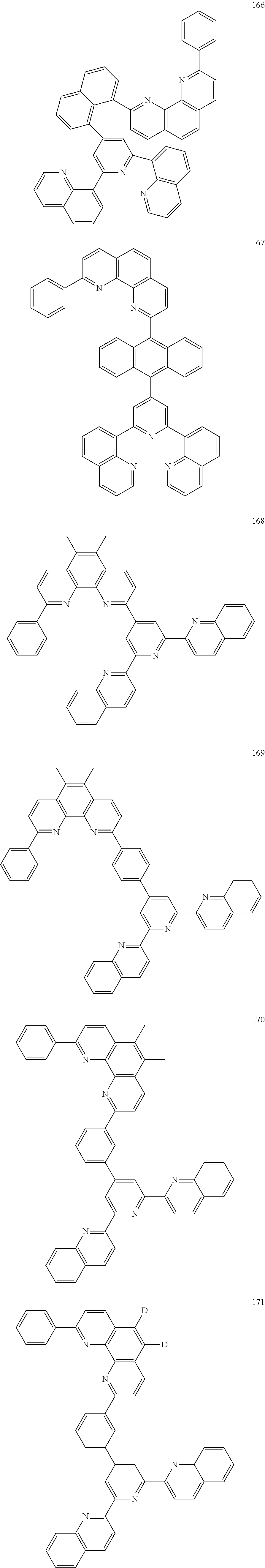

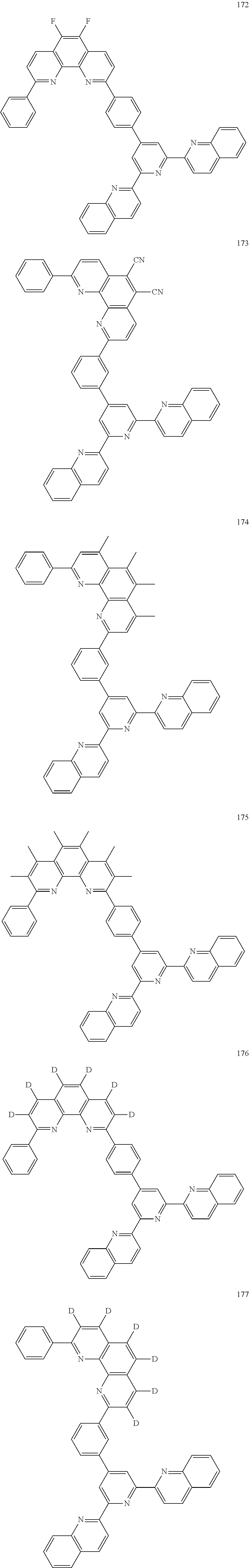

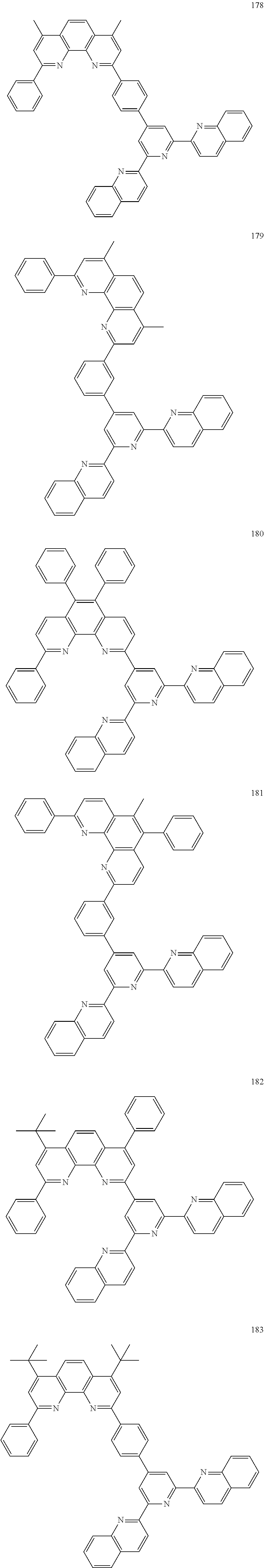

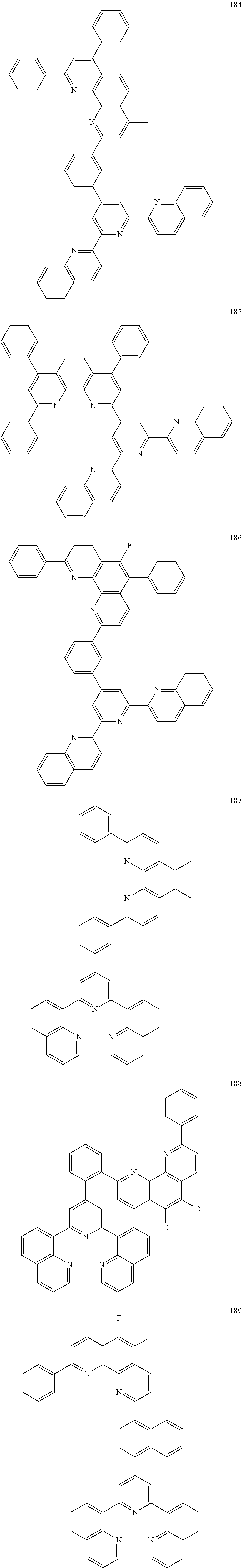

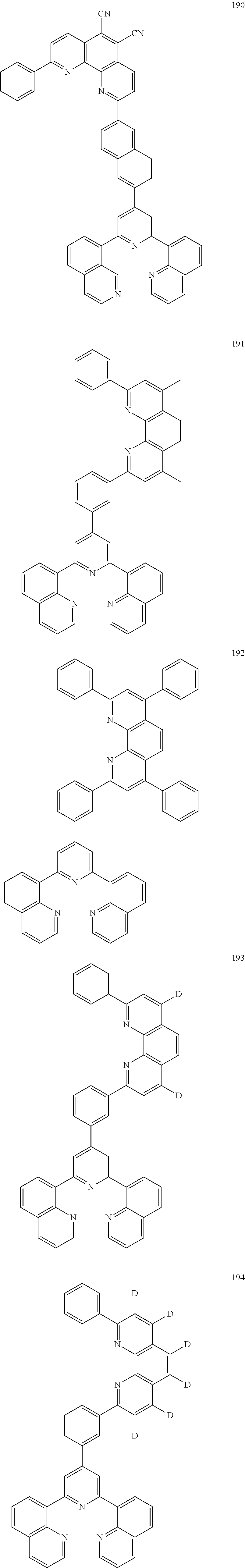

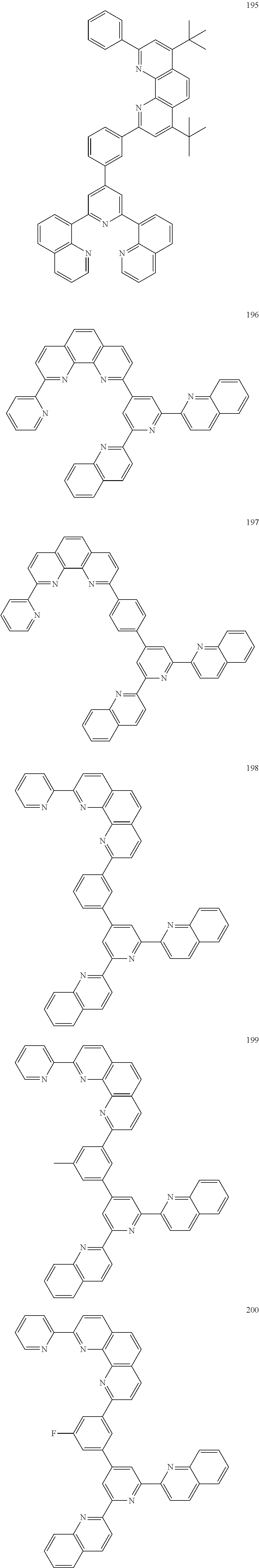

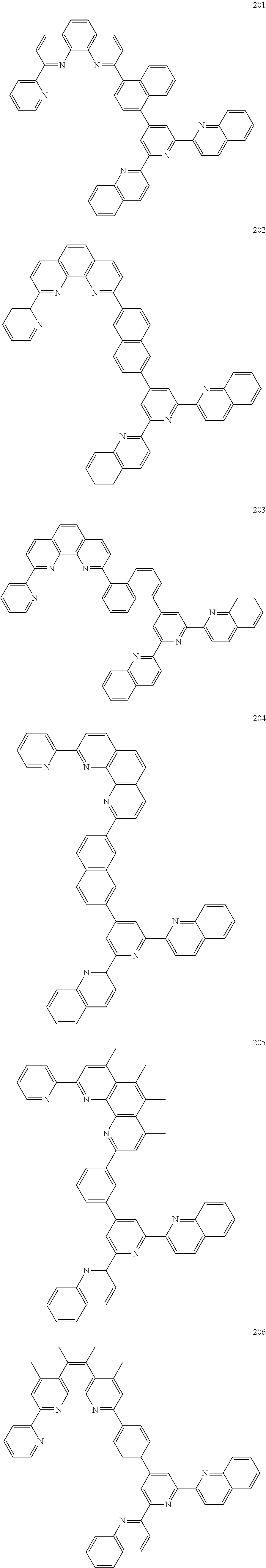

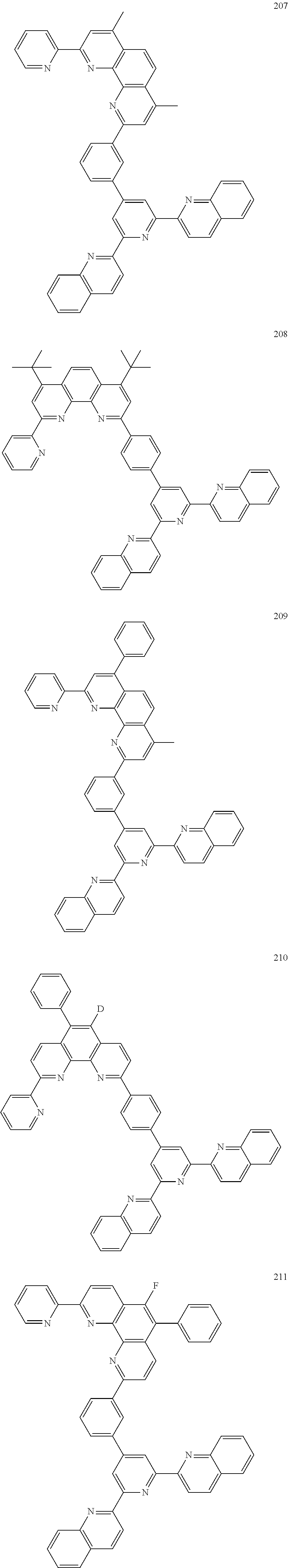

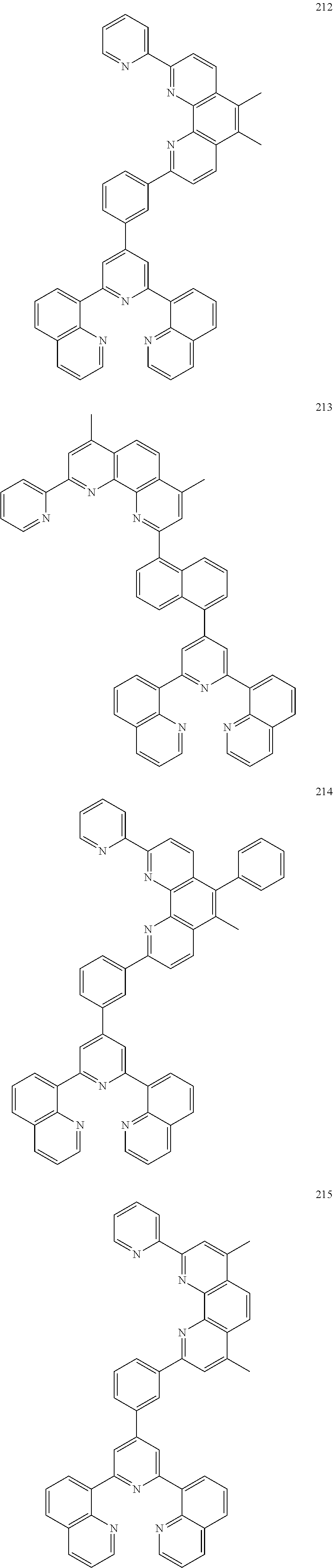

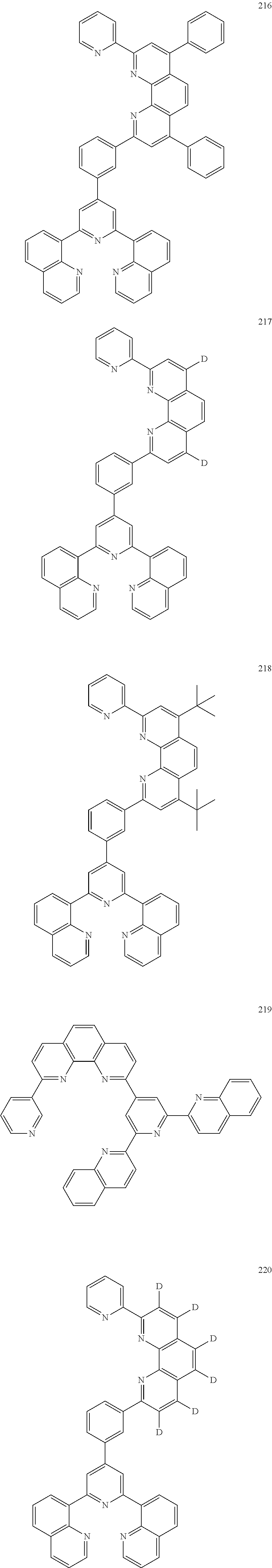

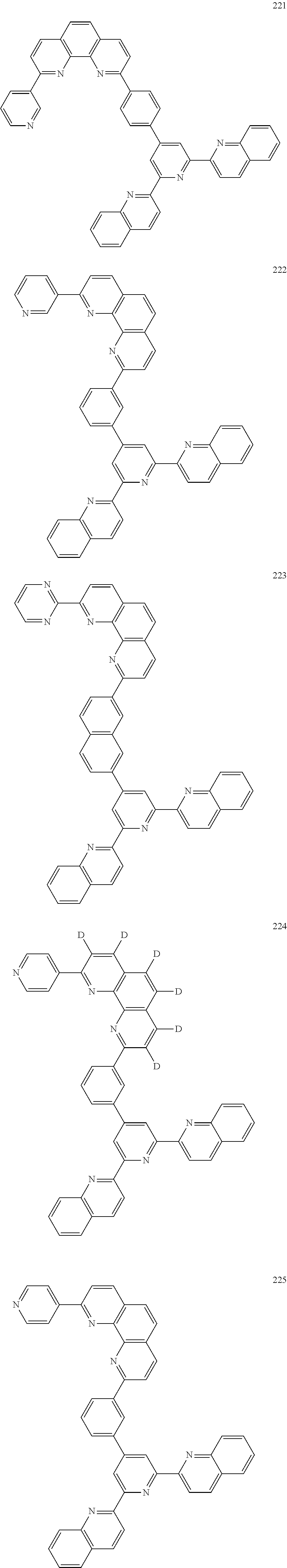

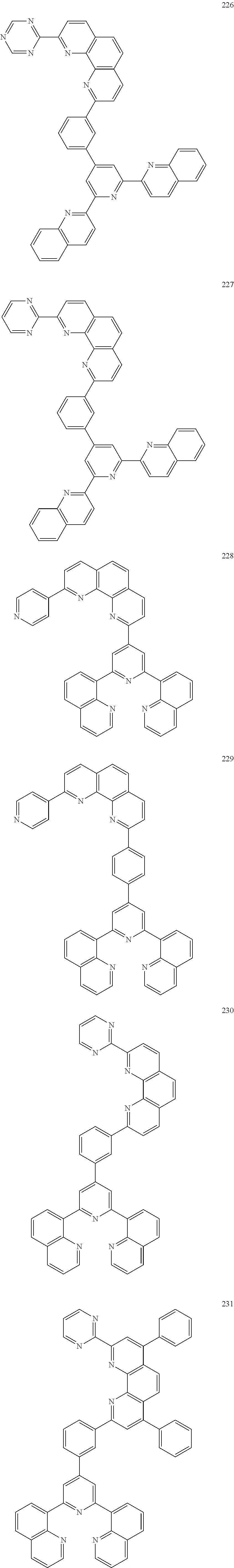

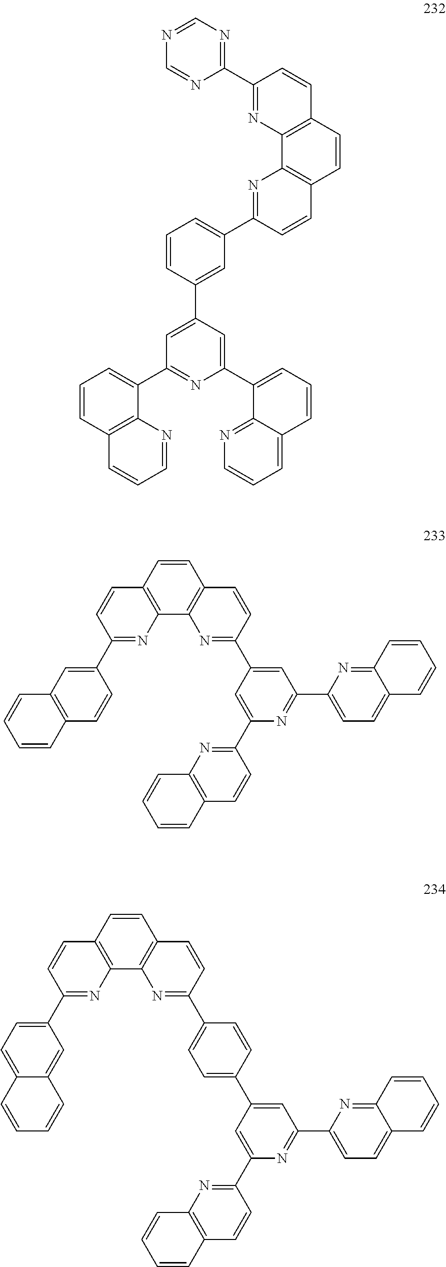 [SMILES: c1cc(-c2cc(-c3cccc4cccnc34)nc(-c3cccc4cccnc34)c2)cc(-c2ccc3ccc4ccc(-c5ncncn5)nc4c3n2)c1 ; c1ccc2cc(-c3ccc4ccc5ccc(-c6cc(-c7ccc8ccccc8n7)nc(-c7ccc8ccccc8n7)c6)nc5c4n3)ccc2c1 ; c1ccc2cc(-c3ccc4ccc5ccc(-c6ccc(-c7cc(-c8ccc9ccccc9n8)nc(-c8ccc9ccccc9n8)c7)cc6)nc5c4n3)ccc2c1]

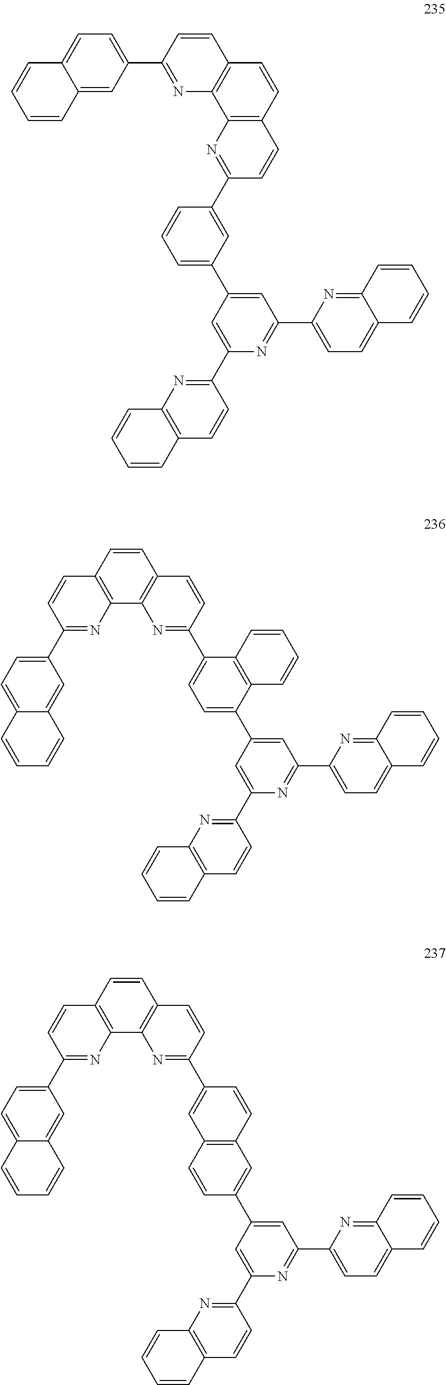 [SMILES: c1cc(-c2cc(-c3ccc4ccccc4n3)nc(-c3ccc4ccccc4n3)c2)cc(-c2ccc3ccc4ccc(-c5ccc6ccccc6c5)nc4c3n2)c1 ; c1ccc2cc(-c3ccc4ccc5ccc(-c6ccc(-c7cc(-c8ccc9ccccc9n8)nc(-c8ccc9ccccc9n8)c7)c7ccccc67)nc5c4n3)ccc2c1 ; c1ccc2cc(-c3ccc4ccc5ccc(-c6ccc7cc(-c8cc(-c9ccc%10ccccc%10n9)nc(-c9ccc%10ccccc%10n9)c8)ccc7c6)nc5c4n3)ccc2c1]

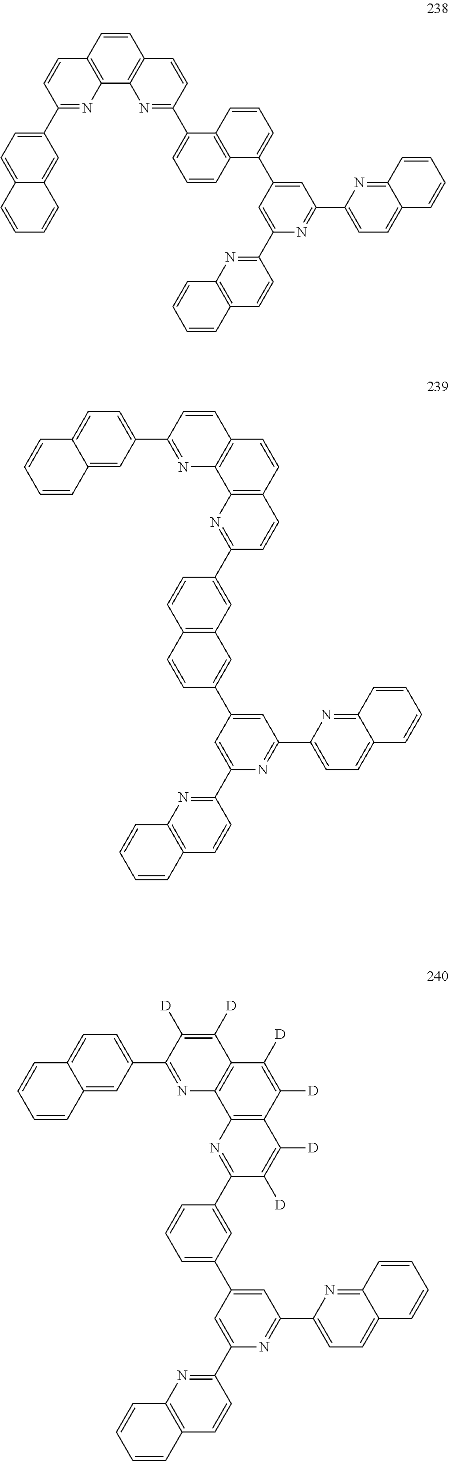 [SMILES: c1ccc2cc(-c3ccc4ccc5ccc(-c6cccc7c(-c8cc(-c9ccc%10ccccc%10n9)nc(-c9ccc%10ccccc%10n9)c8)cccc67)nc5c4n3)ccc2c1 ; c1ccc2cc(-c3ccc4ccc5ccc(-c6ccc7ccc(-c8cc(-c9ccc%10ccccc%10n9)nc(-c9ccc%10ccccc%10n9)c8)cc7c6)nc5c4n3)ccc2c1 ; [2H]c1c(-c2cccc(-c3cc(-c4ccc5ccccc5n4)nc(-c4ccc5ccccc5n4)c3)c2)nc2c(c1[2H])c([2H])c([2H])c1c([2H])c([2H])c(-c3ccc4ccccc4c3)nc12]

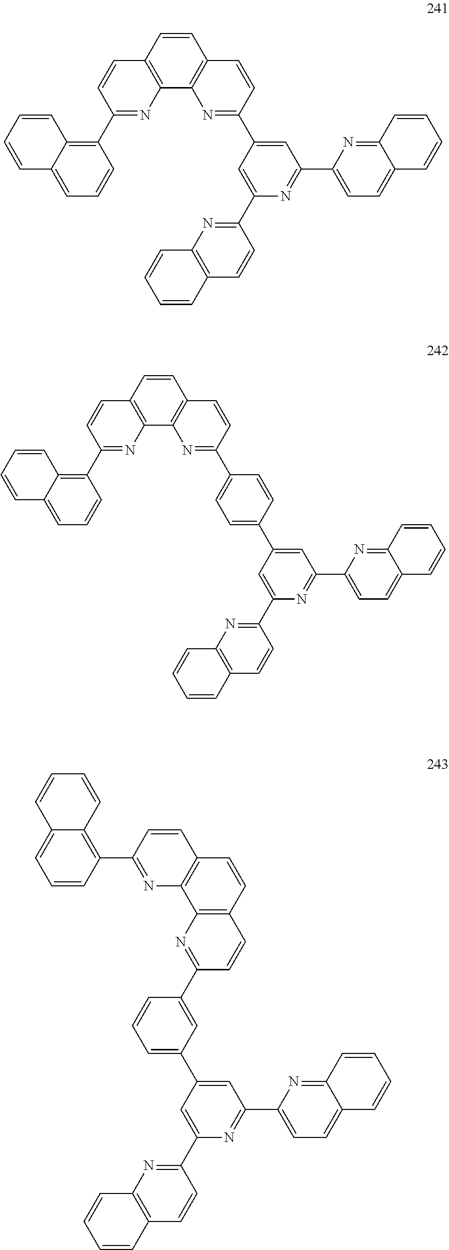 [SMILES: c1ccc2nc(-c3cc(-c4ccc5ccc6ccc(-c7cccc8ccccc78)nc6c5n4)cc(-c4ccc5ccccc5n4)n3)ccc2c1 ; c1ccc2nc(-c3cc(-c4ccc(-c5ccc6ccc7ccc(-c8cccc9ccccc89)nc7c6n5)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc3ccc4ccc(-c5cccc6ccccc56)nc4c3n2)c1]

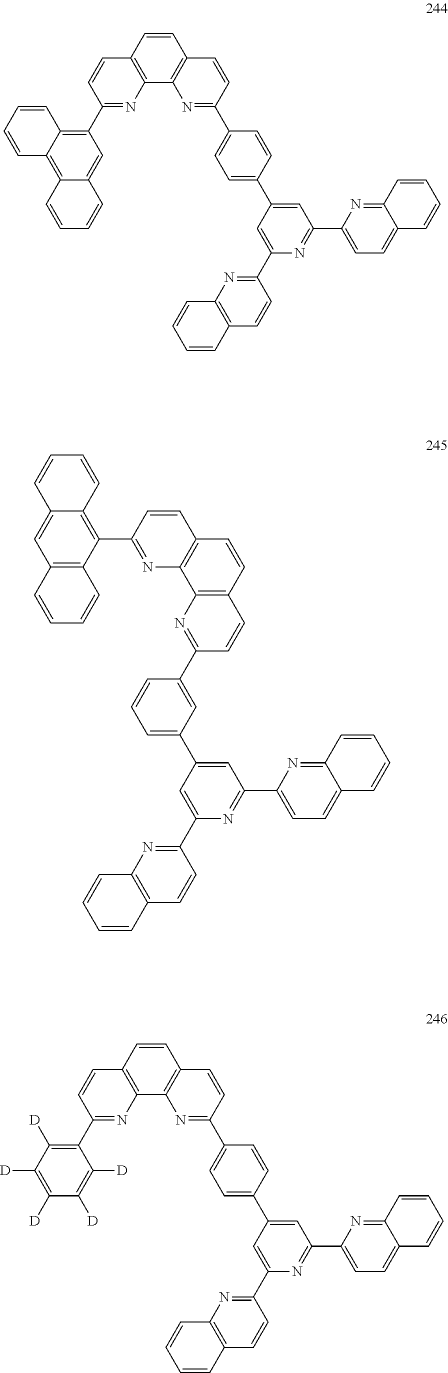 [SMILES: c1ccc2nc(-c3cc(-c4ccc(-c5ccc6ccc7ccc(-c8cc9ccccc9c9ccccc89)nc7c6n5)cc4)cc(-c4ccc5ccccc5n4)n3)ccc2c1 ; c1cc(-c2cc(-c3ccc4ccccc4n3)nc(-c3ccc4ccccc4n3)c2)cc(-c2ccc3ccc4ccc(-c5c6ccccc6cc6ccccc56)nc4c3n2)c1 ; [2H]c1c([2H])c([2H])c(-c2ccc3ccc4ccc(-c5ccc(-c6cc(-c7ccc8ccccc8n7)nc(-c7ccc8ccccc8n7)c6)cc5)nc4c3n2)c([2H])c1[2H]]

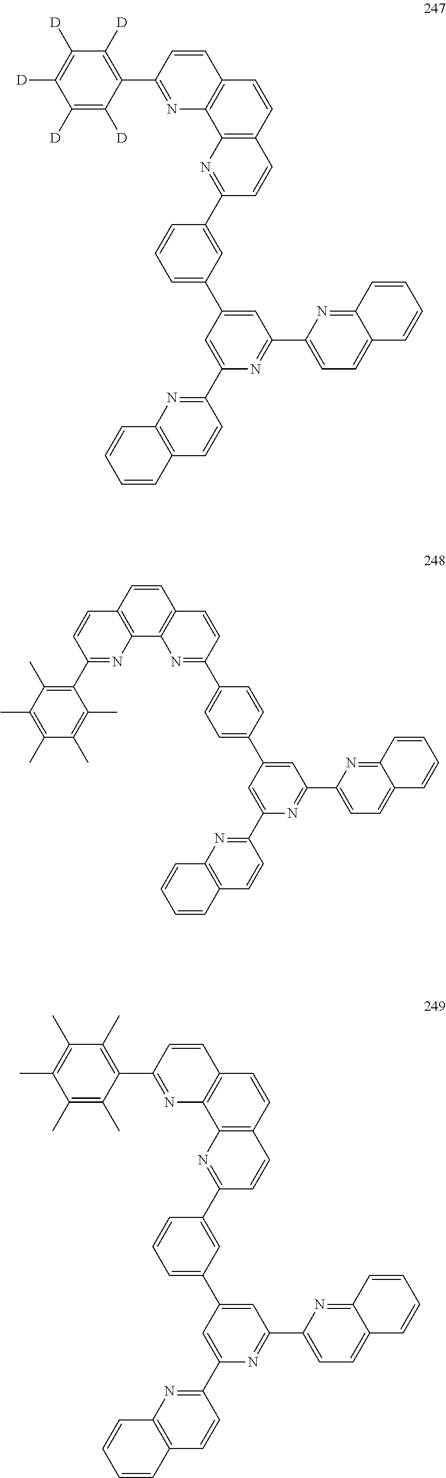 [SMILES: [2H]c1c([2H])c([2H])c(-c2ccc3ccc4ccc(-c5cccc(-c6cc(-c7ccc8ccccc8n7)nc(-c7ccc8ccccc8n7)c6)c5)nc4c3n2)c([2H])c1[2H] ; Cc1c(C)c(C)c(-c2ccc3ccc4ccc(-c5ccc(-c6cc(-c7ccc8ccccc8n7)nc(-c7ccc8ccccc8n7)c6)cc5)nc4c3n2)c(C)c1C ; Cc1c(C)c(C)c(-c2ccc3ccc4ccc(-c5cccc(-c6cc(-c7ccc8ccccc8n7)nc(-c7ccc8ccccc8n7)c6)c5)nc4c3n2)c(C)c1C]

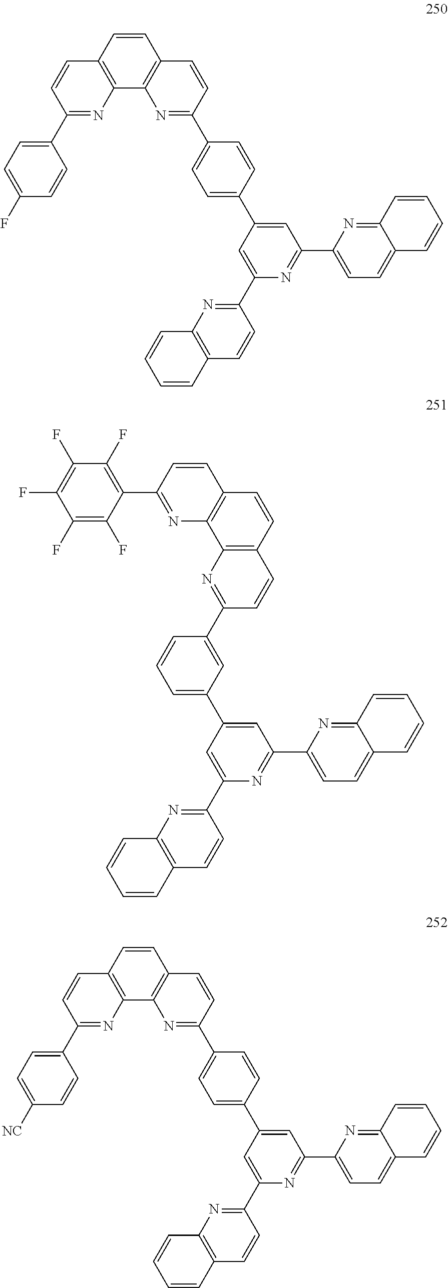 [SMILES: Fc1ccc(-c2ccc3ccc4ccc(-c5ccc(-c6cc(-c7ccc8ccccc8n7)nc(-c7ccc8ccccc8n7)c6)cc5)nc4c3n2)cc1 ; Fc1c(F)c(F)c(-c2ccc3ccc4ccc(-c5cccc(-c6cc(-c7ccc8ccccc8n7)nc(-c7ccc8ccccc8n7)c6)c5)nc4c3n2)c(F)c1F ; N#Cc1ccc(-c2ccc3ccc4ccc(-c5ccc(-c6cc(-c7ccc8ccccc8n7)nc(-c7ccc8ccccc8n7)c6)cc5)nc4c3n2)cc1]

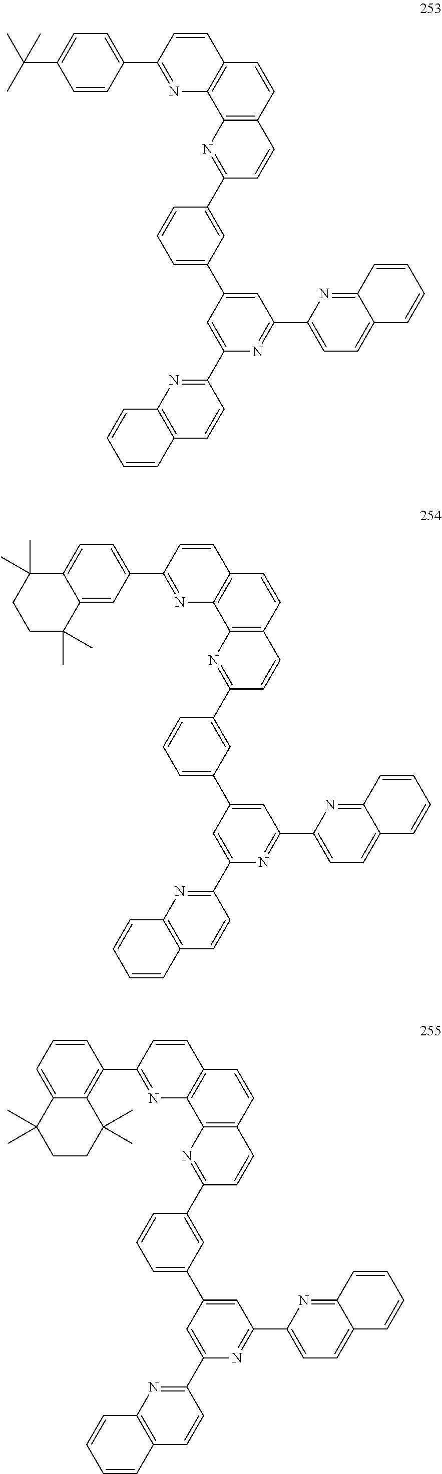 [SMILES: CC(C)(C)c1ccc(-c2ccc3ccc4ccc(-c5cccc(-c6cc(-c7ccc8ccccc8n7)nc(-c7ccc8ccccc8n7)c6)c5)nc4c3n2)cc1 ; CC1(C)CCC(C)(C)c2cc(-c3ccc4ccc5ccc(-c6cccc(-c7cc(-c8ccc9ccccc9n8)nc(-c8ccc9ccccc9n8)c7)c6)nc5c4n3)ccc21 ; CC1(C)CCC(C)(C)c2c(-c3ccc4ccc5ccc(-c6cccc(-c7cc(-c8ccc9ccccc9n8)nc(-c8ccc9ccccc9n8)c7)c6)nc5c4n3)cccc21]

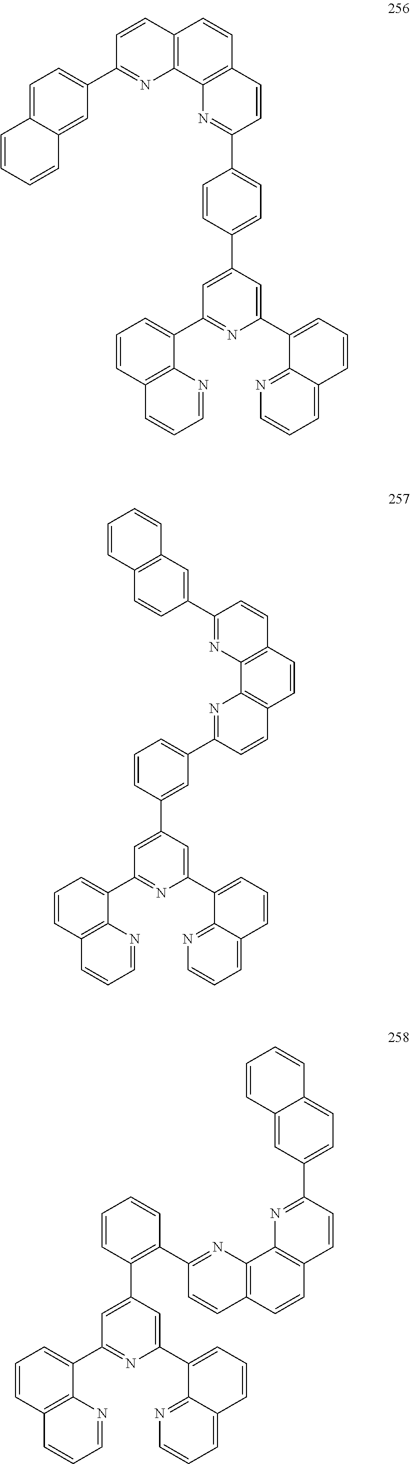 [SMILES: c1ccc2cc(-c3ccc4ccc5ccc(-c6ccc(-c7cc(-c8cccc9cccnc89)nc(-c8cccc9cccnc89)c7)cc6)nc5c4n3)ccc2c1 ; c1cc(-c2cc(-c3cccc4cccnc34)nc(-c3cccc4cccnc34)c2)cc(-c2ccc3ccc4ccc(-c5ccc6ccccc6c5)nc4c3n2)c1 ; c1ccc(-c2ccc3ccc4ccc(-c5ccc6ccccc6c5)nc4c3n2)c(-c2cc(-c3cccc4cccnc34)nc(-c3cccc4cccnc34)c2)c1]

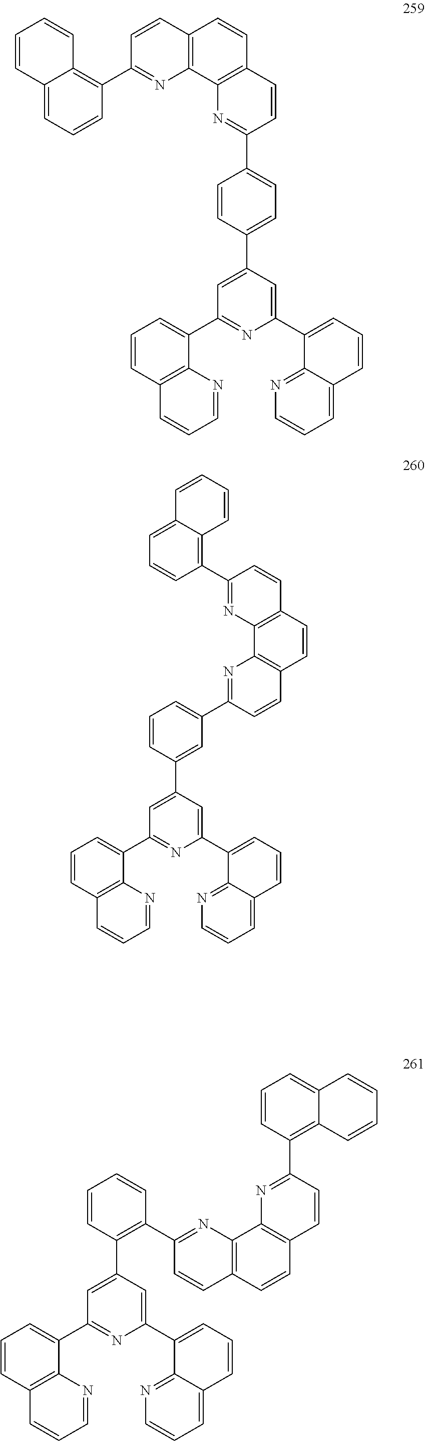

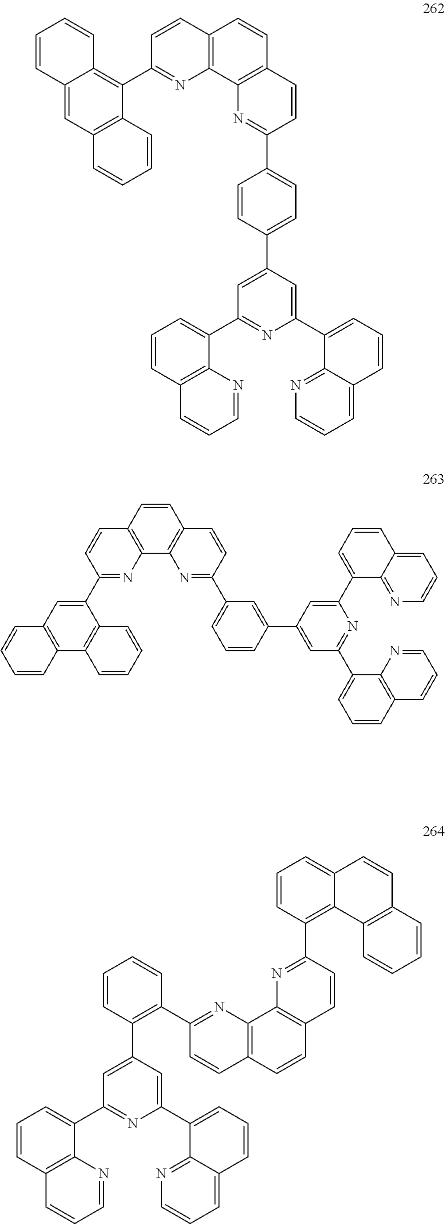 [SMILES: c1ccc2c(-c3ccc4ccc5ccc(-c6ccc(-c7cc(-c8cccc9cccnc89)nc(-c8cccc9cccnc89)c7)cc6)nc5c4n3)c3ccccc3cc2c1 ; c1cc(-c2cc(-c3cccc4cccnc34)nc(-c3cccc4cccnc34)c2)cc(-c2ccc3ccc4ccc(-c5cc6ccccc6c6ccccc56)nc4c3n2)c1 ; c1ccc(-c2ccc3ccc4ccc(-c5cccc6ccc7ccccc7c56)nc4c3n2)c(-c2cc(-c3cccc4cccnc34)nc(-c3cccc4cccnc34)c2)c1]

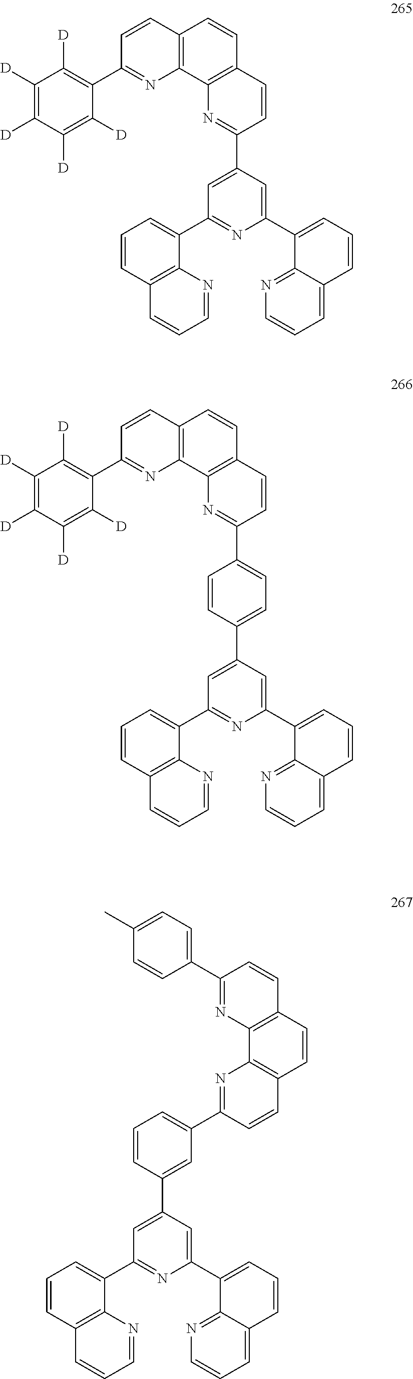 [SMILES: [2H]c1c([2H])c([2H])c(-c2ccc3ccc4ccc(-c5cc(-c6cccc7cccnc67)nc(-c6cccc7cccnc67)c5)nc4c3n2)c([2H])c1[2H] ; [2H]c1c([2H])c([2H])c(-c2ccc3ccc4ccc(-c5ccc(-c6cc(-c7cccc8cccnc78)nc(-c7cccc8cccnc78)c6)cc5)nc4c3n2)c([2H])c1[2H] ; Cc1ccc(-c2ccc3ccc4ccc(-c5cccc(-c6cc(-c7cccc8cccnc78)nc(-c7cccc8cccnc78)c6)c5)nc4c3n2)cc1]

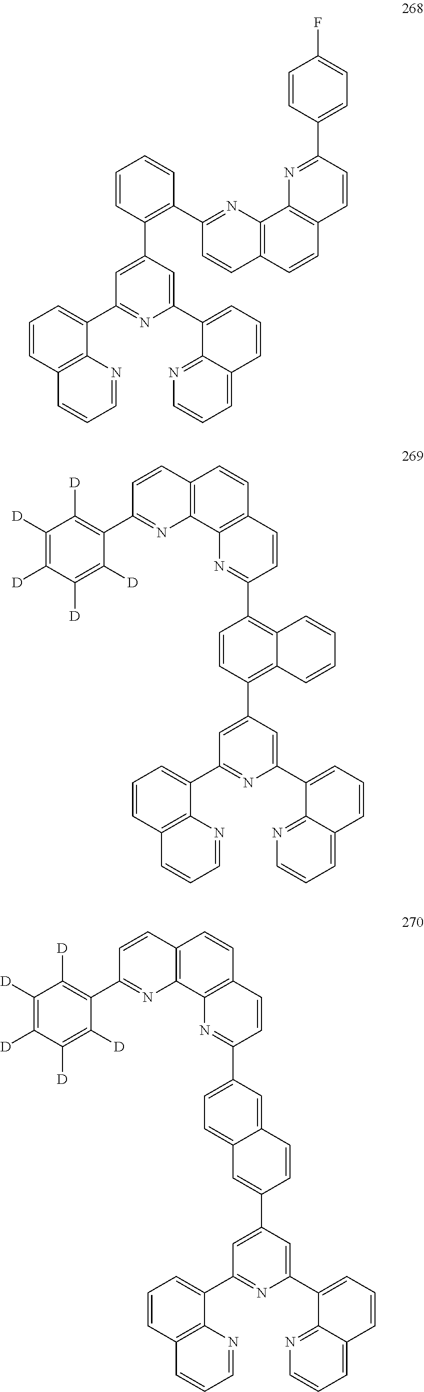

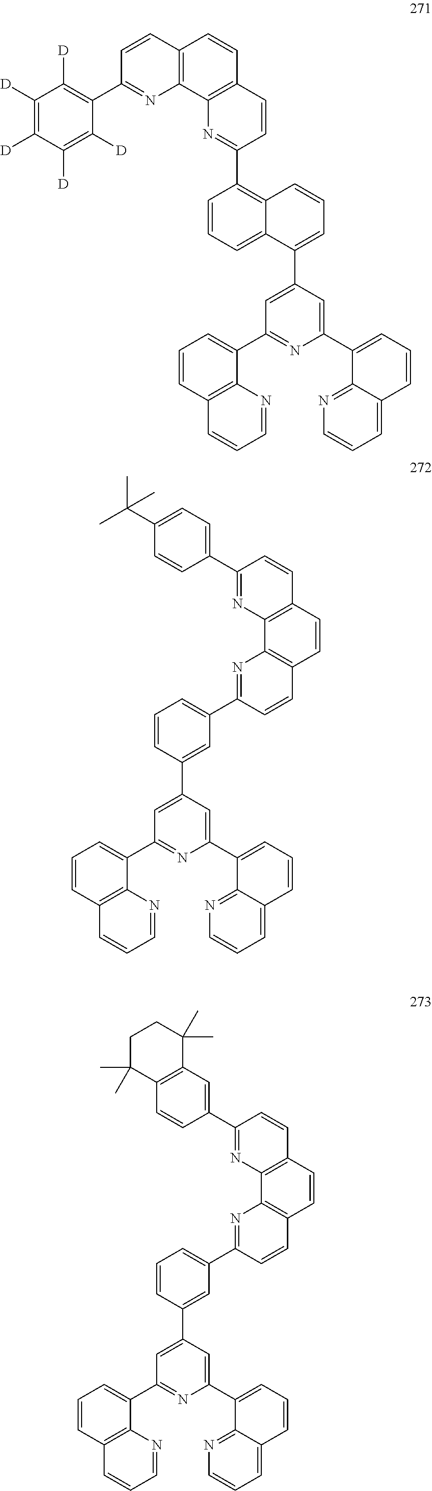 [SMILES: [2H]c1c([2H])c([2H])c(-c2ccc3ccc4ccc(-c5cccc6c(-c7cc(-c8cccc9cccnc89)nc(-c8cccc9cccnc89)c7)cccc56)nc4c3n2)c([2H])c1[2H] ; CC(C)(C)c1ccc(-c2ccc3ccc4ccc(-c5cccc(-c6cc(-c7cccc8cccnc78)nc(-c7cccc8cccnc78)c6)c5)nc4c3n2)cc1 ; CC1(C)CCC(C)(C)c2cc(-c3ccc4ccc5ccc(-c6cccc(-c7cc(-c8cccc9cccnc89)nc(-c8cccc9cccnc89)c7)c6)nc5c4n3)ccc21]

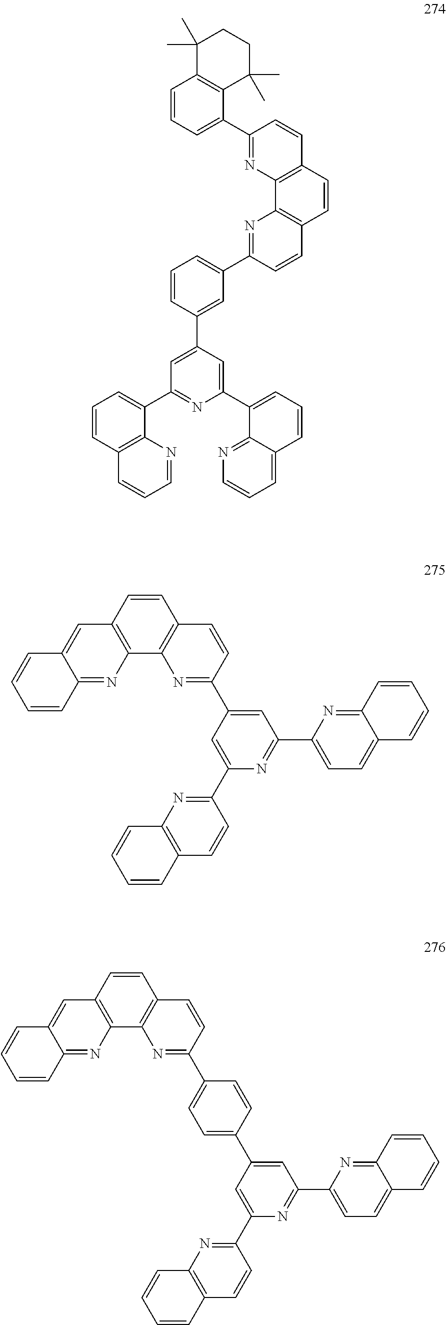 [SMILES: CC1(C)CCC(C)(C)c2c(-c3ccc4ccc5ccc(-c6cccc(-c7cc(-c8cccc9cccnc89)nc(-c8cccc9cccnc89)c7)c6)nc5c4n3)cccc21 ; c1ccc2nc(-c3cc(-c4ccc5ccc6cc7ccccc7nc6c5n4)cc(-c4ccc5ccccc5n4)n3)ccc2c1 ; c1ccc2nc(-c3cc(-c4ccc(-c5ccc6ccc7cc8ccccc8nc7c6n5)cc4)cc(-c4ccc5ccccc5n4)n3)ccc2c1]

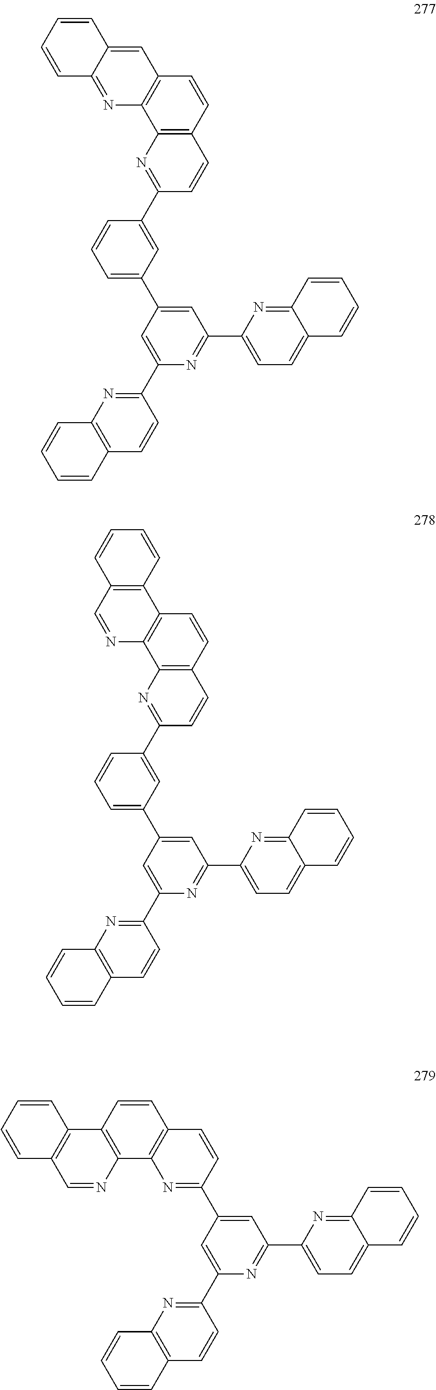 [SMILES: c1cc(-c2cc(-c3ccc4ccccc4n3)nc(-c3ccc4ccccc4n3)c2)cc(-c2ccc3ccc4cc5ccccc5nc4c3n2)c1 ; c1cc(-c2cc(-c3ccc4ccccc4n3)nc(-c3ccc4ccccc4n3)c2)cc(-c2ccc3ccc4c5ccccc5cnc4c3n2)c1 ; c1ccc2nc(-c3cc(-c4ccc5ccc6c7ccccc7cnc6c5n4)cc(-c4ccc5ccccc5n4)n3)ccc2c1]

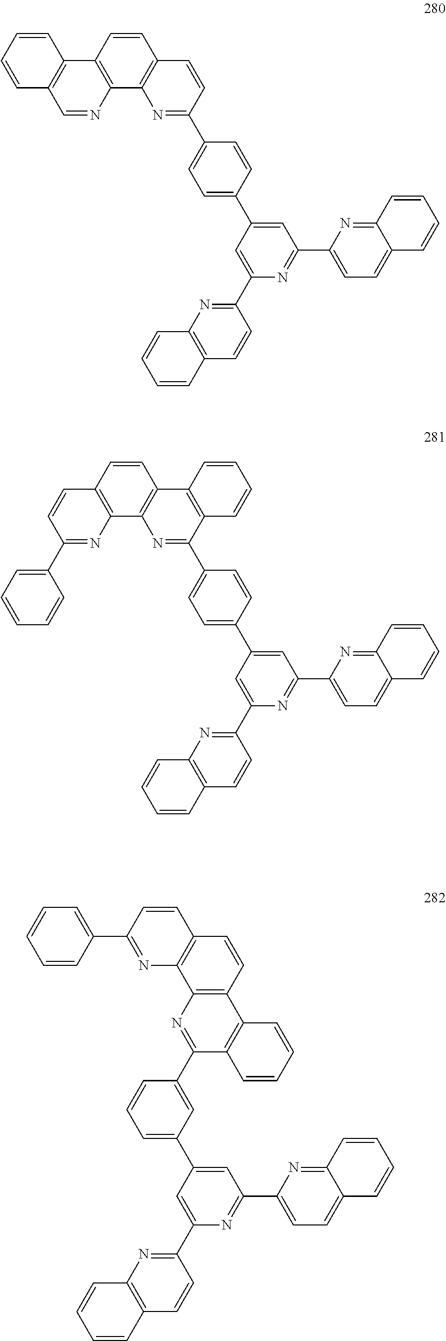 [SMILES: c1ccc2nc(-c3cc(-c4ccc(-c5ccc6ccc7c8ccccc8cnc7c6n5)cc4)cc(-c4ccc5ccccc5n4)n3)ccc2c1 ; c1ccc(-c2ccc3ccc4c5ccccc5c(-c5ccc(-c6cc(-c7ccc8ccccc8n7)nc(-c7ccc8ccccc8n7)c6)cc5)nc4c3n2)cc1 ; c1ccc(-c2ccc3ccc4c5ccccc5c(-c5cccc(-c6cc(-c7ccc8ccccc8n7)nc(-c7ccc8ccccc8n7)c6)c5)nc4c3n2)cc1]

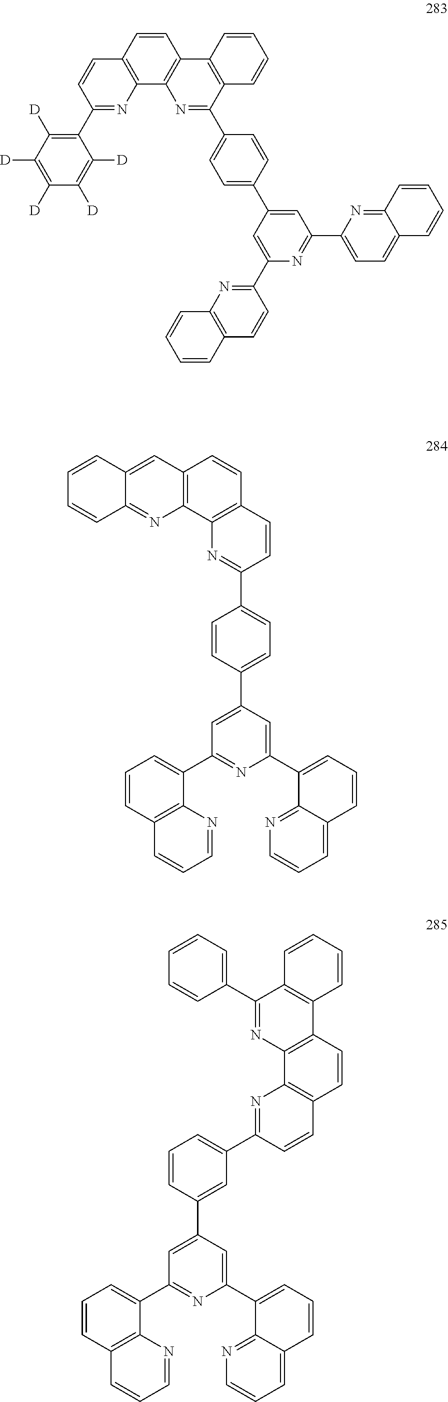 [SMILES: [2H]c1c([2H])c([2H])c(-c2ccc3ccc4c5ccccc5c(-c5ccc(-c6cc(-c7ccc8ccccc8n7)nc(-c7ccc8ccccc8n7)c6)cc5)nc4c3n2)c([2H])c1[2H] ; c1ccc2nc3c(ccc4ccc(-c5ccc(-c6cc(-c7cccc8cccnc78)nc(-c7cccc8cccnc78)c6)cc5)nc43)cc2c1 ; c1ccc(-c2nc3c(ccc4ccc(-c5cccc(-c6cc(-c7cccc8cccnc78)nc(-c7cccc8cccnc78)c6)c5)nc43)c3ccccc23)cc1]

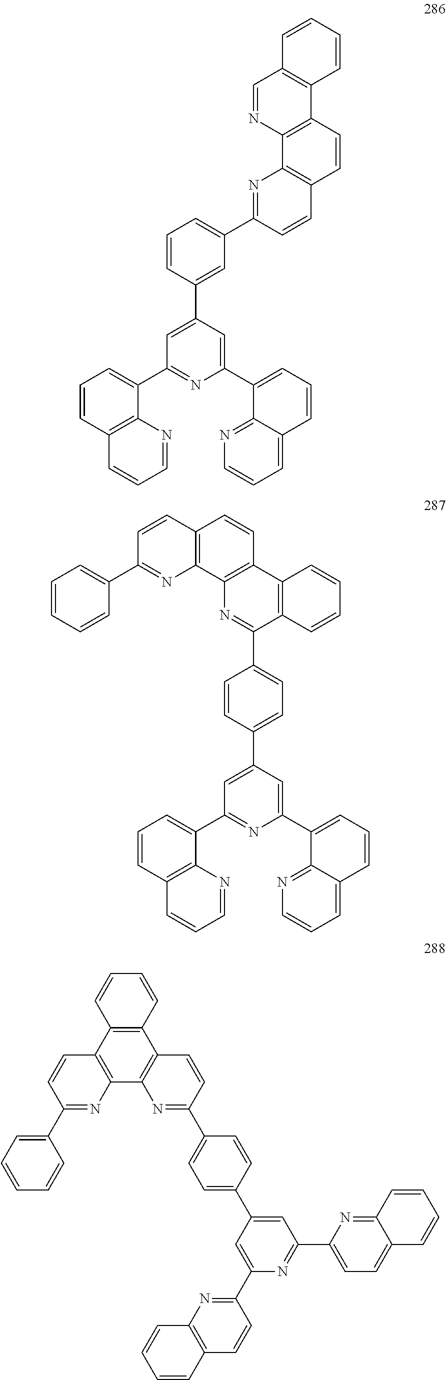

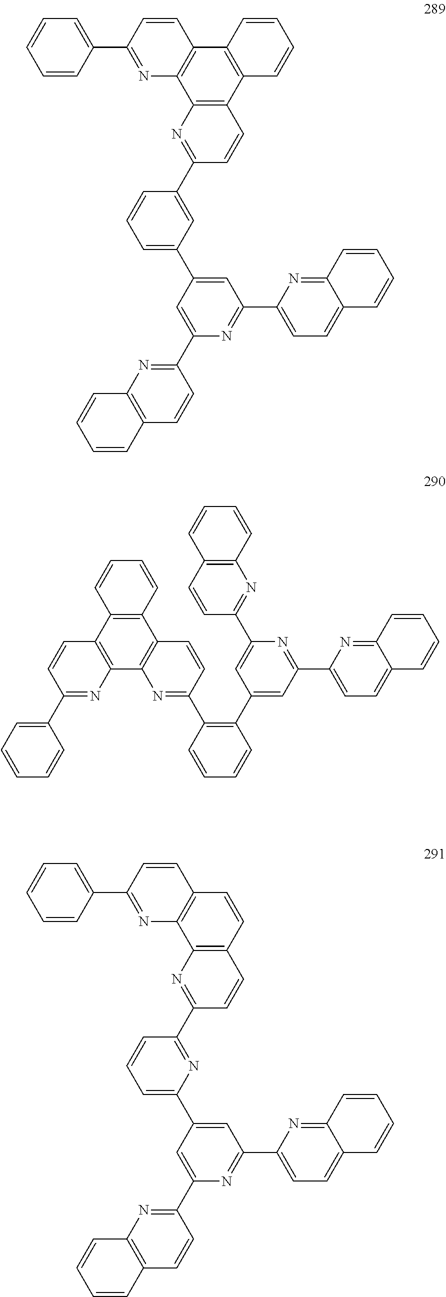 [SMILES: c1ccc(-c2ccc3c4ccccc4c4ccc(-c5cccc(-c6cc(-c7ccc8ccccc8n7)nc(-c7ccc8ccccc8n7)c6)c5)nc4c3n2)cc1 ; c1ccc(-c2ccc3c4ccccc4c4ccc(-c5ccccc5-c5cc(-c6ccc7ccccc7n6)nc(-c6ccc7ccccc7n6)c5)nc4c3n2)cc1 ; c1ccc(-c2ccc3ccc4ccc(-c5cccc(-c6cc(-c7ccc8ccccc8n7)nc(-c7ccc8ccccc8n7)c6)n5)nc4c3n2)cc1]

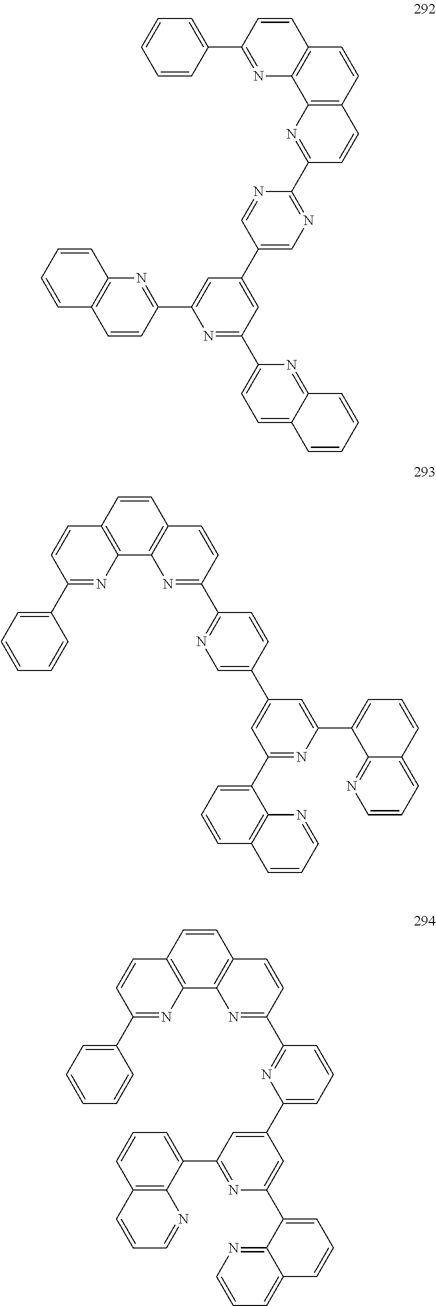 [SMILES: c1ccc(-c2ccc3ccc4ccc(-c5ncc(-c6cc(-c7ccc8ccccc8n7)nc(-c7ccc8ccccc8n7)c6)cn5)nc4c3n2)cc1 ; c1ccc(-c2ccc3ccc4ccc(-c5ccc(-c6cc(-c7cccc8cccnc78)nc(-c7cccc8cccnc78)c6)cn5)nc4c3n2)cc1 ; c1ccc(-c2ccc3ccc4ccc(-c5cccc(-c6cc(-c7cccc8cccnc78)nc(-c7cccc8cccnc78)c6)n5)nc4c3n2)cc1]

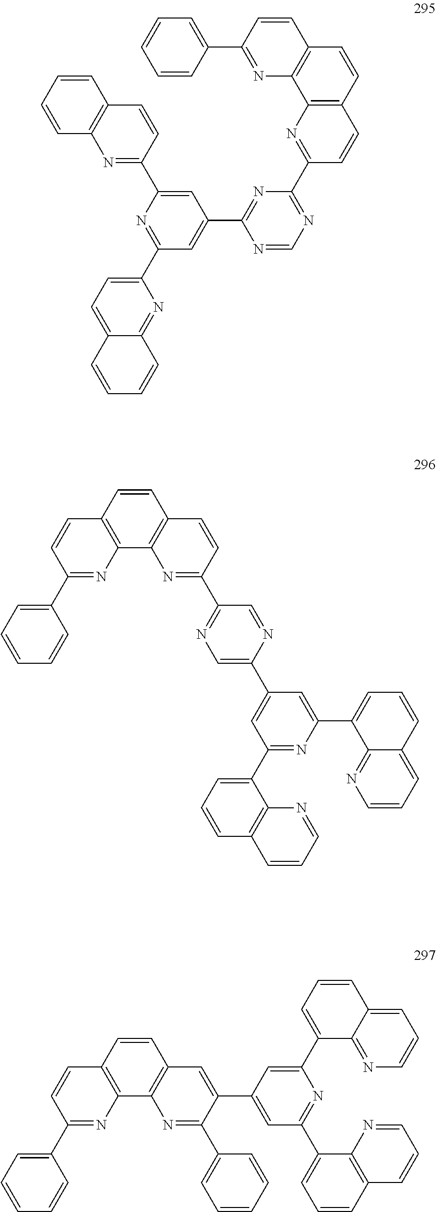 [SMILES: c1ccc(-c2ccc3ccc4ccc(-c5ncnc(-c6cc(-c7ccc8ccccc8n7)nc(-c7ccc8ccccc8n7)c6)n5)nc4c3n2)cc1 ; c1ccc(-c2ccc3ccc4ccc(-c5cnc(-c6cc(-c7cccc8cccnc78)nc(-c7cccc8cccnc78)c6)cn5)nc4c3n2)cc1 ; c1ccc(-c2ccc3ccc4cc(-c5cc(-c6cccc7cccnc67)nc(-c6cccc7cccnc67)c5)c(-c5ccccc5)nc4c3n2)cc1]

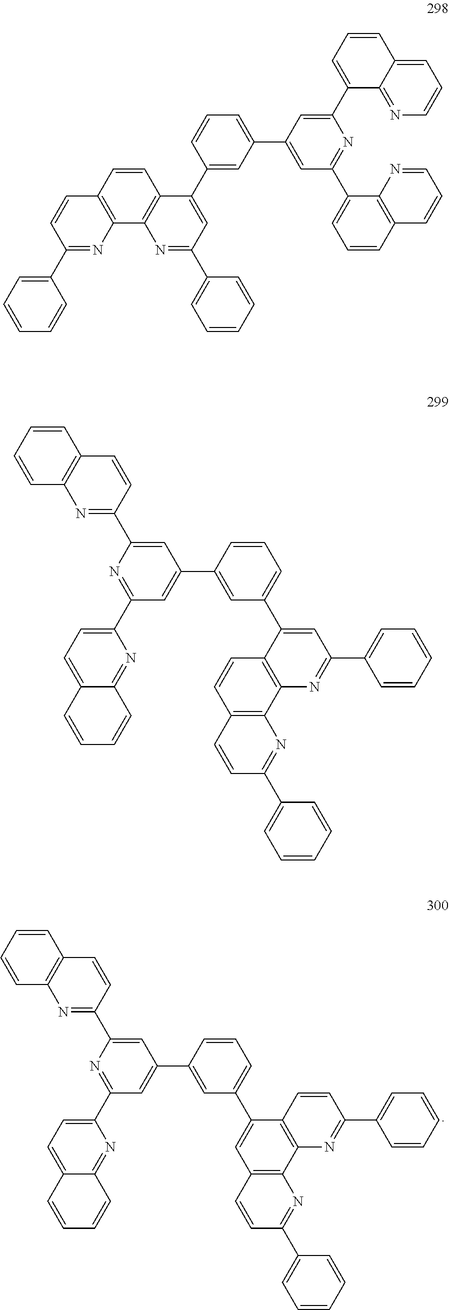 [SMILES: c1ccc(-c2ccc3ccc4c(-c5cccc(-c6cc(-c7cccc8cccnc78)nc(-c7cccc8cccnc78)c6)c5)cc(-c5ccccc5)nc4c3n2)cc1 ; c1ccc(-c2ccc3ccc4c(-c5cccc(-c6cc(-c7ccc8ccccc8n7)nc(-c7ccc8ccccc8n7)c6)c5)cc(-c5ccccc5)nc4c3n2)cc1 ; c1ccc(-c2ccc3cc(-c4cccc(-c5cc(-c6ccc7ccccc7n6)nc(-c6ccc7ccccc7n6)c5)c4)c4ccc(-c5ccccc5)nc4c3n2)cc1]

9. An organic electroluminescent device, comprising an anode and a cathode, and a functional layer disposed between the anode and the cathode, wherein the functional layer comprises the organic compound according to claim 1.

10. The organic electroluminescent device according to claim 9, wherein the functional layer comprises an electron transport layer comprising the organic compound.

11. The organic electroluminescent device according to claim 9, wherein the functional layer comprises a first light-emitting unit, a second light-emitting unit and a charge generation layer; and the charge generation layer comprises the organic compound.

12. An electronic apparatus, comprising the organic electroluminescent device according to claim 9.
